(19) United States
(12) Patent Application Publication
(10) Pub. No.: US 2009/0275606 A1
(43) Pub. Date: Nov. 5, 2009

(54) Heterocyclic Compounds as MEK Inhibitors

(75) Inventors: Dinesh Chikkanna, Bangalore (IN); Clive McCarthy, Froidefontaine (FR); Henrik Moebitz, Freiburg (DE); Chetan Pandit, New Delhi (IN); Ramesh Sistla, Bangalore (IN); Hosahalli Subramanya, Bangalore (IN)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/427,137

(22) Filed: Apr. 21, 2009

(30) Foreign Application Priority Data
Apr. 21, 2008 (IN) 968/CHE/08

(51) Int. Cl. A61K 31/437; C07D 487/04; C07D 455/02; A61K 31/4375; A61P 35/00

(52) U.S. Cl. 514/306; 546/183; 546/138; 514/299

(57) ABSTRACT

The present invention relates to compounds of formula I

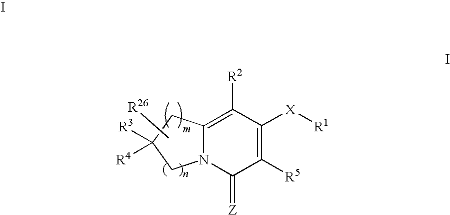

and pharmaceutically acceptable salts. These compounds can act as potential MEK inhibitors in the treatment of hyperproliferative diseases, like cancer and inflammation. The present invention also reveals methods of preparation thereof.

This application claims priority to IN Provisional Application Serial No. 968/CHE/08 filed 21 Apr. 2008, the contents of which are incorporated herein by reference in their entirety.

The invention relates to compounds which are specific inhibitors of kinase activity of MEK. The invention also relates to the use of the compounds, their pro-drugs or pharmaceutically acceptable composition comprising the compound or their prodrug in the management of hyperproliferative diseases like cancer and inflammation.

Hyperproliferative diseases like cancer and inflammation are attracting the scientific community to provide therapeutic benefits. In this regard efforts have been made to identify and target specific mechanisms which play a role in proliferating the diseases.

Over-activation of mitogen-activated protein (MAP) kinase cascade is known to play an important role in cell proliferation and differentiation. This pathway can be activated when a growth factor binds to its receptor tyrosine kinase. This interaction promotes RAS association with RAF and initiates a phosphorylation cascade through MEK (MAP kinase kinase) to ERK. Inhibition of this pathway is known to be beneficial in hyperproliferative diseases. MEK is an attractive therapeutic target because the only known substrates for MEK phosphorylation are the MAP kinases, ERK1 and ERK2. Constitutive activation of MEK/ERK was been found in pancreatic, colon, lung, kidney and ovarian primary tumor samples.

Phosphorylation of MEK appears to increase its affinity and its catalytic activity toward ERK as well as is affinity for ATP. This invention describes compounds that inhibit MEK activity by modulation of ATP binding, association of MEK with ERK by mechanisms that are competitive, and/or allosteric and/or uncompetitive.

Activation of MEK has been demonstrated in many models of disease models thus suggesting that inhibition of MEK could have potential therapeutic benefit in various diseases such as
Pain: Evidence of Efficacy in Pain Models (J. Neurosci. 22:478, 2002; Acta Pharmacol Sin. 26:789 2005; Expert Opin Ther Targets. 9:699, 2005; Mol. Pain. 2:2, 2006)
Stroke: Evidence of Efficacy in Stroke Models Significant Neuroprotection against Ischemic Brain Injury by Inhibition of the MEK (J. Pharmacol. Exp. Ther. 304:172, 2003; Brain Res. 996:55, 2004)
Diabetes: Evidence In Diabetic Complications. (Am. J. Physiol. Renal. 286, F120 2004)
Inflammation: Evidence of Efficacy in Inflammation Models. (Biochem Biophy. Res. Com. 268:647, 2000)
Arthritis Evidence of efficacy in experimental osteoarthritis. (Arthritis & (J. Clin. Invest. 116:163. 2006)

Inhibition of MEK has been shown to have potential therapeutic benefit in several studies.

Thus, as a first embodiment, the invention provides a compound of formula I

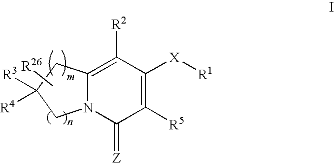

and pharmaceutically acceptable salts thereof, wherein
X represents C₁₋₃-alkylene, —N(R⁶)—, —O—, or —S(O)ₚ—;
R¹ represents aryl, heteroaryl, cycloalkyl or heterocycloalkyl, wherein said rings are optionally substituted by one or more groups independently selected from List 1;
R² represents H, cyano, or the group —Y—R⁷;
R³ and R⁴ independently represent H, C₁₋₆-alkyl, C₁₋₆-haloalkyl, C₁₋₆-hydroxyalkyl, hydroxyl, C₁₋₆-alkoxy, amino, C₁₋₆-alkylamino, diC₁₋₆-alkylamino, or R³ additionally represents monocyclic cycloalkyl or monocyclic heterocycloalkyl, where said rings are optionally substituted by one or more groups independently selected from List 1;
R⁵ represents H, halogen, C₁₋₃alkyl, C₁₋₃alkoxy, —SC₁₋₃alkyl, or C₁₋₃-haloalkyl;
Y represents a group selected from -D-, -E-, -D-E-, or E-D-;
D represents a group selected from N(R⁸)—, —CO—, —CO₂—, —SO—, —SO₂—, CON(R⁹)O—, —CON(R¹⁰), —N(R¹¹) SO₂—, —N(R²⁴) SO₂NR²⁵—, —SO₂N(R¹²)—, —N(R¹³)CO—, —N(R¹⁴)CON(R¹⁵)—-N(R¹⁶)CO—, or —C(═NH)N(R¹⁷)—;
E represents a monocyclic arylene, heteroarylene, cycloalkylene or heterocycloalkylene, wherein said rings are optionally substituted by one or more groups independently selected from List 1;
R⁷ represents H, C₁₋₆-alkyl, C₂₋₆-alkenyl C₂₋₆-alkynyl, cycloalkyl, aryl, heterocycloalkyl, or heteroaryl, wherein R⁷ when not H is optionally substituted by one to three groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆haloalkyl, amino, C₁₋₆alkylamino, di-C₁₋₆alkylamino, C₁₋₆acylamino, C₁₋₆acylC₁₋₆ alkylamino, monocyclic cycloalkyl or monocyclic heterocycloalkyl, where said rings may be optionally substituted by one or two groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆-haloalkyl, amino, C₁₋₆-alkylamino, di-C₁₋₆-alkylamino, C₁₋₆-acylamino and C₁₋₆-acylC₁₋₆-alkylamino;

### Z is O or N(R¹⁸);

List 1 is selected from hydroxyl, cyano, nitro, C₁₋₆-alkyl, C₂₋₆-alkenyl, C₂₋₆-alkynyl, C₁₋₆-alkoxy, C₂₋₆-alkenyloxy, C₁₋₆-alkynyloxy, halogen, C₁₋₆-alkylcarbonyl, carboxy, C₁₋₆-alkoxycarbonyl, amino, C₁₋₆-alkylamino, di-C₁₋₆-alkylamino, C₁₋₆-alkylaminocarbonyl, di-C₁₋₆-alkylaminocarbonyl, C₁₋₆-alkylcarbonylamino, C₁₋₆-alkylcarbonyl(C₁₋₆-alkyl)amino, C₁₋₆-alkylsulfonylamino, C₁₋₆ alkylsulfonyl(C₁₋₆-alkyl)amino, C₁₋₆-thioalkyl, C₁₋₆-alkylsulfinyl, C₁₋₆-alkylsulfanyl, C₁₋₆-alkylsulfonyl, aminosulfonyl, C₁₋₆-alkylaminosulfonyl and di-C₁₋₆-alkylaminosulfonyl, where each of the afore-mentioned hydrocarbon groups may be optionally substituted by one or more halogen, hydroxyl, C₁₋₆-alkoxy, amino, C₁₋₆-alkylamino, di-C₁₋₆-alkylamino or cyano;

R²⁶ represents H, C₁₋₆-alkyl, C₁₋₆-haloalkyl, C₁₋₆-hydroxyalkyl, hydroxyl, C₁₋₆-alkoxy, amino, C₁₋₆-alkylamino, or diC₁₋₆-alkylamino;
R⁶, R⁸, R⁹, R¹⁰, R¹¹, R¹², R¹³, R¹⁴, R¹⁵, R¹⁶R¹⁷, R¹⁸, R²⁴, and R²⁵ are independently H or C₁₋₆-alkyl;
m and n are independently 0, 1, 2, or 3; and m+n=2 or 3;
p is 0, 1, or 2; and wherein

Alkyl or alkylene means a straight chain, branched and/or cyclic hydrocarbon from 1 to 20 carbon atoms. Alkyl moieties having from 1 to 5 carbons are referred to as “lower alkyl” and examples include, but are not limited to, methyl, ethyl, propyl, isopropyl, n-butyl, t-butyl, and isobutyl.

Cycloalkyl or cycloalkylene represents a 3-14 membered monocyclic or bicyclic carbocyclic ring, wherein the monocyclic or one of the bicyclic rings is saturated or partially unsaturated and may optionally further comprise a —C(O)— ring member, and the other ring may be aromatic, saturated or partially unsaturated and may include one to three ring members selected from —C(═O), —N(R²⁰)q-, —O— and S(O)r where R²⁰ is H or C₁₋₆-alkyl, q is 0-1 and r is 0-2;

Aryl or arylene represents a 6-14 membered monocyclic or bicyclic carbocyclic ring, wherein the monocyclic or one of the bicyclic rings is aromatic and the other ring may be aromatic, saturated or partially unsaturated and may include one to three ring members selected from —C(O), —N(R¹⁹)q-, —O— and S(O)r where R¹⁹ is H or C₁₋₆-alkyl, q is 0-1 and r is 0-2;

Heteroaryl or heteroarylene represents a 5-14 membered monocyclic or bicyclic ring, wherein the monocyclic or one of the bicyclic rings is an aromatic group comprising either (a) 1 to 4 nitrogen atoms, (b) one oxygen or one sulphur atom or (c) 1 oxygen atom or 1 sulphur atom and 1 or 2 nitrogen atoms, and the other ring may be aromatic, saturated or partially unsaturated, and may include one to three ring members selected from —C(O), —N(R²¹)q-, —O— and S(O)r where R²¹ is H or C₁₋₆-alkyl, q is 0-1 and r is 0-2; and

Heterocycloalkyl or heterocycloalkylene represents a 3-14 membered monocyclic or bicyclic ring, wherein the monocyclic or one of the bicyclic rings is a saturated or partially unsaturated group comprising one or two ring members selected from N(R²²)—, —O— and —S(O)ᵣ— and may optionally further comprise a —C(O)— ring member, and the other ring may be aromatic, saturated or partially unsaturated, and may include one to three ring members selected from —C(═O), —N(R²³)q-, —O— and S(O)r where R²² or R²³ is H or C₁₋₆-alkyl, q is 0-1 and r is 0-2.

The following specific embodiments of the invention according to formula (I) may be incorporated into the definition of formula (I) and combined in any number of suitable ways.

In one embodiment, X represents —N(H)—.

In another embodiment, R¹ represents optionally substituted phenyl.

In another embodiment, optional substitution on R¹ is represented by one to three groups independently selected from halogen, e.g. fluoro, bromo or iodo, C₁₋₆-alkyl e.g. ethyl, C₂₋₆-alkynyl, e.g. ethynyl, C₁₋₆-haloalkyl, e.g trifluoromethyl and C₁₋₆-thioalkyl, e.g. thiomethyl.

In another embodiment, R¹ is represented by phenyl substituted in the 2-, 4- and optionally 6-positions, suitably the 2- and 4-positions. In a further embodiment, R¹ is represented by phenyl substituted by 2-fluoro and 4-bromo, or, 4-iodo-2-fluorophenyl, or any combination of 2- and 4-substitutions of iodo, trifluoromethyl, thiomethyl, ethynyl or ethyl.

In another embodiment, -D- represents a group selected from —C(O)—, —CO₂—, C(O)N(H)O—, —C(O)N(C₁₋₆-alkyl)O—, —C(O)N(H)— and —C(O)N(C₁₋₆-alkyl)-.

In another embodiment, -E- represents a 5-membered heteroarylene or 5-membered heterocycloalkylene. In a further embodiment, E represents a ring selected from;

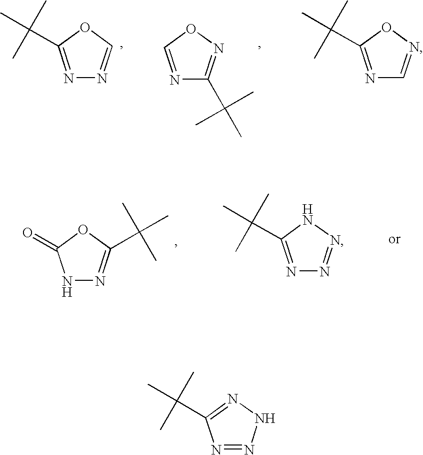

In another embodiment, where Y represents -D-E-, -D- may represent —C(O)N(H)— and -E- may represent optionally substituted cycloalkyl, e.g. cyclopentyl or optionally substituted heteroaryl, e.g. thiazole.

In another embodiment, where Y represents -E-D-, -E- may represent optionally substituted heteroaryl, e.g. oxadiazole and -D- may represent —C(O)N(H)—

In another embodiment, Y represents the groups D- or E-.

In another embodiment, R⁷ represents H, C₁₋₆-alkyl, e.g. methyl or ethyl, substituted C₁₋₆-alkyl, e.g. by one to three, in another embodiment one to two, groups selected from hydroxyl, including di-hydroxyl, C₁₋₆-alkoxy, e.g. methoxy, C₂-C₆-alkenyloxy, e.g. ethenyloxy, di-C₁₋₆-alkylamino, e.g. dimethylamino, C₁₋₆-acylamino, e.g. acetylamino, and optionally substituted monocyclic cycloalkyl, e.g. cyclopropyl.

In another embodiment, R⁷ represents H, methyl, ethyl, cyclopropylmethyl, 2-hydroxyethyl, 2-ethenyloxyethyl, 3-hydroxypropyl, 2-methoxyethyl, acetylaminomethyl, 2-dimethylaminoethyl or 2,3-dihydroxypropyl.

In another embodiment, R² represents —CO₂H, COH, —CO₂Et, C(O)N(H or CH₃)OR^{7a}, where R^{7a} represents methyl, ethyl, cyclopropylmethyl, 2-ethenyloxyethyl, 2-hydroxyethyl and 2,3-dihydroxypropyl, —C(O)N(H or CH₃)R^{7b}, where R^{7b} represents H, methyl, ethyl, cyclopropylmethyl, 2-methoxyethyl, 2-hydroxyethyl, 3-hydroxypropyl, acetylaminomethyl, 2-dimethylaminoethyl, cyclopentyl or 2-thiazolyl, or R² represents oxadiazolylamino.

In another embodiment, the present invention includes compounds of formula I where R² represents CONHOR^{7a} where R^{7a} represents cyclopropylmethyl, or 2-hydroxyethyl.

In another embodiment, m and n are both 1 or one of m and n is 1 and the other is 2.

In another embodiment, R³ and R⁴ represent H.

In another embodiment, R⁵ represents H, halogen, e.g. fluoro or chloro, C₁₋₃alkoxy, e.g., methoxy, or ethoxy, —SC₁₋₃alkyl, e.g., SCH₃, or C₁₋₃alkyl, e.g. methyl or ethyl. In a further embodiment, R⁵ is fluoro. In a further embodiment, R⁵ is methyl.

In another embodiment, Z represents O.

In another embodiment, aryl or arylene represent an optionally substituted phenyl or phenylene, respectively.

In another embodiment, cycloalkyl or cycloalkylene represent an optionally substituted 3-7 membered saturated monocyclic carbocyclic ring, e.g. cyclopropyl or cyclopentyl.

In another embodiment, heteroaryl or heteroarylene represent an optionally substituted 5-6 membered monocyclic aromatic group comprising either (a) 1 to 4 nitrogen atoms, (b) one oxygen or one sulphur atom or (c) 1 oxygen atom or 1 sulphur atom and 1 or 2 nitrogen atoms, e.g. tetrazolyl, thiazolyl or oxadiazolyl.

In another embodiment, heterocycloalkyl or heterocycloalkylene represent an optionally substituted 5-6 membered saturated monocyclic ring comprising one or two ring members selected from N(R²²)—, —O— and —S(O)ᵣ—.

In still another embodiment of the present invention, the compound forms a pharmaceutically acceptable salt, selected from a group comprising acid addition salts and base addition salts.

In another embodiment, the present invention includes a pharmaceutical composition comprising a compound of formula I or Id and a pharmaceutically acceptable carrier or excipient. In another embodiment, the present invention includes a pharmaceutical composition comprising a compound of formula I or Id in combination with a second active agent and a pharmaceutically acceptable carrier or excipient.

In another embodiment, the present invention includes compounds of formula Id:

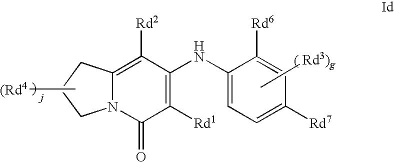

and salts thereof, where
Rd¹ represents H, halogen, C₁₋₃-alkyl, or C₁₋₃-haloalkyl;
Rd² represents H, cyano, or the group —Y-Rd⁵;
Rd³ and Rd⁴ independently represent hydroxyl, cyano, nitro, C₁₋₆-alkyl, C₂₋₆-alkenyl, C₂₋₆ alkynyl, C₁₋₆-alkoxy, C₂₋₆-alkenyloxy, C₂₋₆-alkynyloxy, halogen, C₁₋₆-alkylcarbonyl, carboxy, C₁₋₆-alkoxycarbonyl, amino, C₁₋₆-alkylamino, di-C₁₋₆-alkylamino, C₁₋₆-alkylaminocarbonyl, di-C₁₋₆ alkylaminocarbonyl, C₁₋₆-alkylcarbonylamino, C₁₋₆-alkylcarbonyl(C₁₋₆-alkyl)amino, C₁₋₆-alkylsulfonylamino, C₁₋₆-alkylsulfonyl(C₁₋₆-alkyl)amino, C₁₋₆-thioalkyl, C₁₋₆-alkylsulfanyl, C₁₋₆ alkylsulfanyl, C₁₋₆-alkylsulfonyl, aminosulfonyl, C₁₋₆-alkylaminosulfonyl and di-C₁₋₆ alkylaminosulfonyl, where each of the afore-mentioned hydrocarbon groups may be optionally substituted by one or more halogen, hydroxyl, C₁₋₆-alkoxy, amino, C₁₋₆-alkylamino, di-C₁₋₆-alkylamino or cyano;
Y represents a group selected from D-, -E-, -D-E-, or E-D-;
D represents a group selected from —N(Rd⁸)—, —CO—, —CO₂—, —SO—, —SO₂—, CON(Rd⁹)O—, —CON(Rd¹⁰)-, —N(Rd¹¹)SO₂—, —N(R¹²)SO₂NRd¹³-, —SO₂N(Rd¹⁴)-, —N(Rd¹⁵)CO—, —N(Rd¹⁶)CON(Rd¹⁷)—, —N(Rd¹⁸)CO—, or —C(═NH)N(Rd¹⁹)-;
E represents a monocyclic arylene, heteroarylene, cycloalkylene or heterocycloalkylene, wherein said rings are optionally substituted by one or more groups independently selected from List 1 as defined herein;
Rd⁵ represents H, C₁₋₆-alkyl, C₂₋₆-alkenyl C₂₋₆-alkynyl, cycloalkyl, aryl, heterocycloalkyl, or heteroaryl, wherein Rd⁵ when not H is optionally substituted by one to three groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆haloalkyl, amino, C₁₋₆alkylamino, di-C₁₋₆alkylamino, C₁₋₆acylamino, C₁₋₆acylC₁₋₆ alkylamino, monocyclic cycloalkyl or monocyclic heterocycloalkyl, where said rings may be optionally substituted by one or two groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆-haloalkyl, amino, C₁₋₆-alkylamino, di-C₁₋₆-alkylamino, C₁₋₆-acylamino and C₁₋₆-acylC₁₋₆-alkylamino;
Rd⁶ and Rd⁷ independently represent hydroxyl, cyano, nitro, C₁₋₆-alkyl, C₂₋₆-alkenyl, C₂₋₆ alkynyl, C₁₋₆-alkoxy, C₂₋₆-alkenyloxy, C₂₋₆-alkynyloxy, halogen, C₁₋₆-alkylcarbonyl, carboxy, C₁₋₆ alkoxycarbonyl, amino, C₁₋₆-alkylamino, di-C₁₋₆-alkylamino, C₁₋₆-alkylaminocarbonyl, di-C₁₋₆-alkylaminocarbonyl, C₁₋₆-alkylcarbonylamino, C₁₋₆-alkylcarbonyl(C₁₋₆-alkyl)amino, C₁₋₆ alkylsulfonylamino, C₁₋₆-alkylsulfonyl(C₁₋₆-alkyl)amino, C₁₋₆-thioalkyl, C₁₋₆-alkylsulfinyl, C₁₋₆ alkylsulfanyl, C₁₋₆-alkylsulfonyl, aminosulfonyl, C₁₋₆-alkylaminosulfonyl and di-C₁₋₆ alkylaminosulfonyl, where each of the afore-mentioned hydrocarbon groups may be optionally substituted by one or more halogen, hydroxyl, C₁₋₆-alkoxy, amino, C₁₋₆-alkylamino, di-C₁₋₆ alkylamino or cyano;
j and g independently represent 0, 1, 2, or 3; and
Rd⁸, Rd⁹, Rd¹⁰, Rd¹¹, Rd¹², Rd¹³, Rd¹⁴, Rd¹⁵, Rd¹⁶, Rd¹⁷, Rd¹⁸, and Rd¹⁹ are independently H or C₁₋₆-alkyl.

In an embodiment, j and g are independently 0, 1, 2, or 3, and j+g=2, 3, or 4. In another embodiment, j is 0, 1, or 2, and g is 1, 2, or 3. In a further embodiment, j is 0, and g is 0, 1, or 2.

With reference to formulas (I) and (Id), alkyl, alkenyl, alkynyl, and alkoxy groups, containing the requisite number of carbon atoms, can be unbranched or branched. Examples of alkyl include methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy.

“Halogen” or “halo” may be fluorine, chlorine, bromine or iodine.

C₁₋₆-haloalkyl refers to an alkyl group substituted by up to seven halogen groups, e.g. fluoro groups. For example, where the substituent is fluoro, common haloalkyl groups are trifluoroalkyl, 2,2,2-trifluoroethyl or 2,2,2,1,1-pentafluoroethyl groups.

The term “alkenyl” refers to a monovalent group derived from a hydrocarbon having at least one carbon-carbon double bond. The term “C₂-C₆-alkenyl” refers to a monovalent group derived from a hydrocarbon having two to six carbon atoms and comprising at least one carbon-carbon double bond.

The term “alkynyl” refers to a monovalent group derived from a hydrocarbon having at least one carbon-carbon triple bond. The term “C₂-C₆-alkynyl” refers to a monovalent group derived from a hydrocarbon having two to six carbon atoms and comprising at least one carbon-carbon triple bond.

The term “alkoxy” refers to a group in which an alkyl group is attached to oxygen, wherein alkyl is as previously defined.

It is to be understood that the terminology C(O) refers to a —C═O group, whether it be ketone, aldehyde or acid or acid derivative. Similarly, S(O) refers to a —S═O group.

Examples of cycloalkyl groups as defined in formula (I) include cyclopropyl, cyclobutyl, cyclopentyl, cyclohexyl, cycloheptyl or cyclooctyl.

Examples of aryl groups as defined in formula (I) include phenyl, naphthyl, anthracyl and phenanthryl.

Examples of heterocycloalkyl groups as defined in formula I include [1,3]dioxolane, [1,4]dioxane, oxiranyl, aziridinyl, oxetanyl, azetidinyl, tetrahydrofuranyl, pyrrolidinyl, tetrahydropyranyl, piperidinyl, morpholino, thiomorpholinyl, piperazinyl, azepinyl, oxapinyl, oxazepinyl and diazepinyl.

Examples of monocyclic heteroaryl groups as defined in formula (I) groups include pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. Examples of bicyclic heteroaryl groups include indolyl, benzofuranyl, quinolyl, isoquinolyl indazolyl, indolinyl, isoindolyl, indolizinyl, benzimidazolyl, and quinolinyl.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

In still another embodiment of the present invention, the compound is a stereoisomer or a tautomer.

A suitable individual compound of the invention is selected from:
7-(4-Bromo-2-fluoro-phenylamino)-5-oxo-1,2,3,5-tetrahydro-indolizine-8-carboxylic acid ethyl ester;
7-(4-Bromo-2-fluoro-phenylamino)-5-oxo-1,2,3,5-tetrahydro-indolizine-8-carboxylic acid
7-(4-Bromo-2-fluoro-phenylamino)-5-oxo-1,2,3,5-tetrahydro-indolizine-8-carboxylic acid cyclopropyl-methoxy-amide;
7-(4-Bromo-2-fluoro-phenylamino)-5-oxo-1,2,3,5-tetrahydro-indolizine-8-carboxylic acid dimethyl amide;
7-(4-bromo 2-fluoro-phenylamino)-5-oxo-1,2,3,5-tetrahydro indolizine 8-carboxylic acid methoxyl amide;
7-(4-bromo 2-fluoro-phenylamino)-5-oxo-1,2,3,5-tetrahydro indolizine 8-carboxylic acid amide;
7-(4-bromo 2-fluoro-phenylamino)-5-oxo-1,2,3,5-tetrahydro indolizine 8-carboxylic acid ethoxy amide;
7-(4-bromo 2-fluoro-phenylamino)-5-oxo-1,2,3,5-tetrahydro indolizine 8-carboxylic acid (2-hydroxy ethyl) amide;
7-(4-bromo-2-fluoro-phenylamino)-5-oxo-1,2,3,5-tetrahydro-indolizine 8-carboxylic acid methyl amide;
7-(4-Bromo-2-fluoro-phenylamino)-5-oxo-1,2,3,5-tetrahydro-indolizine-8-carboxylic acid methoxy-ethyl-amide;
7-(4-Bromo-2-fluoro-phenylamino)-5-oxo-1,2,3,5-tetrahydro-indolizine-8-carbaldehyde
6-Fluoro-7-(2-fluoro-4-iodo-phenylamino)-5-oxo-1,2,3,5-tetrahydro-indolizine-8-carboxylic acid cyclopropyl methoxy-amide;
6-Fluoro-7-(2-fluoro-4-iodo-phenylamino)-5-oxo-1,2,3,5-tetrahydro-indolizine-8-carboxylic acid amide;
7-(4-Bromo-2-fluoro-phenylamino)-6-fluoro-5-oxo-1,2,3,5-tetrahydro-indolizine-8-carboxylic acid;
7-(4-Bromo-2-fluoro-phenylamino)-6-fluoro-5-oxo-1,2,3,5-tetrahydro-indolizine-8-carboxylic acid cyclopropyl methoxy-amide;
7-(4-Bromo-2-fluoro-phenylamino)-6-fluoro-5-oxo-1,2,3,5-tetrahydro-indolizine-8-carboxylic acid amide;
7-(4-Bromo-2-fluoro-phenylamino)-6-methyl-5-oxo-1,2,3,5-tetrahydro-indolizine-8-carboxylic acid;
7-(4-Bromo-2-fluoro-phenylamino)-6-methyl-5-oxo-1,2,3,5-tetrahydro-indolizine-8-carboxylic acid cyclopropyl methoxy-amide;
7-(4-Bromo-2-fluoro-phenylamino)-6-methyl-5-oxo-1,2,3,5-tetrahydro-indolizine-8-carboxylic acid (2-vinyl oxy-ethoxy)-amide;
7-(4-Bromo-2-fluoro-phenylamino)-6-methyl-5-oxo-1,2,3,5-tetrahydro-indolizine-8-carboxylic acid amide;
7-(4-Bromo-2-fluoro-phenylamino)-6-methyl-5-oxo-1,2,3,5-tetrahydro-indolizine-8-carboxylic acid methoxy-amide;
7-(4-Bromo-2-fluoro-phenylamino)-6-methyl-5-oxo-1,2,3,5-tetrahydro-indolizine-8-carboxylic acid ethoxy-amide;
2-(4-bromo-2-fluoro-phenylamino)-4-oxo-6,7,8,9-tetrahydro-4H-quinolizine-1-carboxylic acid ethyl ester;
2-(4-bromo-2-fluoro-phenyl-amino)-4-oxo-6,7,8,9-tetrahydro-4H-quinolizine-1-carboxylic acid;
2-(4-bromo-2-fluoro-phenyl-amino)-4-oxo-6,7,8,9-tetrahydro-4H-quinolizine-1-carboxylic acid-cyclopropyl-methoxyamide;
2-(4-bromo-2-fluoro-phenylamino)-4-oxo-6,7,8,9-tetrahydro-4H-quinolizine-1-carboxylic acid amide;
2-(4-bromo-2-fluoro-phenylamino)-4-oxo-6,7,8,9-tetrahydro-4H-quinolizine-1-carboxylic acid-(2-vinyloxy-ethoxy)-amide;
2-(4-bromo-2-fluoro-phenylamino)-4-oxo-6,7,8,9-tetrahydro-4H-quinolizine-1-carboxylic acid methoxy amide;
2-(4-bromo-2-fluoro-phenylamino)-4-oxo-6,7,8,9-tetrahydro-4H-quinolizine-1-carboxylic acid ethoxy amide;
7-(4-bromo-2-fluorophenylamino)-6-methyl-5-oxo-1,2,3,5-tetrahydro-indolizine-8-carboxylic acid (2-hydroxyethoxy)amide;
7-(4-Bromo-2-fluoro-phenylamino)-5-oxo-1,2,3,5-tetrahydro-indolizine-8-carboxylic acid (3-hydroxy-propyl)-amide;
7-(4-Bromo-2-fluoro-phenylamino)-5-oxo-1,2,3,5-tetrahydro-indolizine-8-carboxylic acid (2-methoxy-ethyl)-amide;
7-(4-Bromo-2-fluoro-phenylamino)-5-oxo-1,2,3,5-tetrahydro-indolizine-8-carboxylic acid (2-acetylamino-ethyl)-amide;
7-(4-Bromo-2-fluoro-phenylamino)-5-oxo-1,2,3,5-tetrahydro-indolizine-8-carboxylic acid (2-dimethylamino-ethyl)-amide;
6-Fluoro-7-(2-fluoro-4-iodo-phenylamino)-5-oxo-1,2,3,5-tetrahydro-indolizine-8-carboxylic acid (2-hydroxy-ethoxy)-amide;
7-(4-Bromo-2-fluoro-phenylamino)-6-methyl-5-oxo-1,2,3,5-tetrahydro-indolizine-8-carboxylic acid cyclopentylamide;
7-(4-Bromo-2-fluoro-phenylamino)-6-methyl-5-oxo-1,2,3,5-tetrahydro-indolizine-8-carboxylic acid ethylamide;
7-(4-Bromo-2-fluoro-phenylamino)-6-methyl-5-oxo-1,2,3,5-tetrahydro-indolizine-8-carboxylic acid (3-methoxy-propyl)-amide;
7-(2-Fluoro-4-iodo-phenylamino)-5-oxo-1,2,3,5-tetrahydro-indolizine-8-carboxylic acid;
7-(2-Fluoro-4-iodo-phenylamino)-5-oxo-1,2,3,5-tetrahydro-indolizine-8-carboxylic acid amide;
7-(2-Fluoro-4-iodo-phenylamino)-5-oxo-1,2,3,5-tetrahydro-indolizine-8-carboxylic acid cyclopropylmethoxy-amide;
7-(2-Fluoro-4-iodo-phenylamino)-5-oxo-1,2,3,5-tetrahydro-indolizine-8-carboxylic acid ethyl ester;
7-(2-Fluoro-4-iodo-phenylamino)-5-oxo-1,2,3,5-tetrahydro-indolizine-8-carboxylic acid (2-hydroxy-ethoxy)-amide;
7-(2-Fluoro-4-iodo-phenylamino)-5-oxo-1,2,3,5-tetrahydro-indolizine-8-carboxylic acid methoxy-amide;
7-(2-Fluoro-4-iodo-phenylamino)-5-oxo-1,2,3,5-tetrahydro-indolizine-8-carboxylic acid ethoxy-amide;
7-(4-Bromo-2-fluoro-phenylamino)-5-oxo-1,2,3,5-tetrahydro-indolizine-8-carboxylic acid (2,3-dihydroxy-propoxy)-amide;
7-(4-Bromo-2-fluoro-phenylamino)-6-chloro-5-oxo-1,2,3,5-tetrahydro-indolizine-8-carboxylic acid;
7-(4-Bromo-2-fluoro-phenylamino)-6-chloro-5-oxo-1,2,3,5-tetrahydro-indolizine-8-carboxylic acid cyclopropylmethoxy-amide;
7-(4-Bromo-2-fluoro-phenylamino)-6-chloro-5-oxo-1,2,3,5-tetrahydro-indolizine-8-carboxylic acid methoxy-amide;
7-(4-Bromo-2-fluoro-phenylamino)-6-chloro-5-oxo-1,2,3,5-tetrahydro-indolizine-8-carboxylic acid amide;
7-(4-Bromo-2-fluoro-phenylamino)-6-chloro-5-oxo-1,2,3,5-tetrahydro-indolizine-8-carboxylic acid ethoxy-amide;
7-(4-Bromo-2-fluoro-phenylamino)-6-chloro-5-oxo-1,2,3,5-tetrahydro-indolizine-8-carboxylic acid (2,3-dihydroxy-propoxy)-amide;
6-Chloro-7-(2-fluoro-4-iodo-phenylamino)-5-oxo-1,2,3,5-tetrahydro-indolizine-8-carboxylic acid;
6-Chloro-7-(2-fluoro-4-iodo-phenylamino)-5-oxo-1,2,3,5-tetrahydro-indolizine-8-carboxylic acid cyclopropylmethoxy-amide;
6-Chloro-7-(2-fluoro-4-iodo-phenylamino)-5-oxo-1,2,3,5-tetrahydro-indolizine-8-carboxylic acid methoxy-amide;
6-Chloro-7-(2-fluoro-4-iodo-phenylamino)-5-oxo-1,2,3,5-tetrahydro-indolizine-8-carboxylic acid amide;
6-Chloro-7-(2-fluoro-4-iodo-phenylamino)-5-oxo-1,2,3,5-tetrahydro-indolizine-8-carboxylic acid ethoxy-amide;
6-Chloro-7-(2-fluoro-4-iodo-phenylamino)-5-oxo-1,2,3,5-tetrahydro-indolizine-8-carboxylic acid (2,3-dihydroxy-propoxy)-amide;
7-(4-Bromo-2-fluoro-phenylamino)-6-methyl-5-oxo-1,2,3,5-tetrahydro-indolizine-8-carboxylic acid thiazol-2-ylamide;
7-(4-Bromo-2-fluoro-phenylamino)-6-methyl-5-oxo-1,2,3,5-tetrahydro-indolizine-8-carboxylic acid (3-hydroxy-propyl)-amide;
7-(4-Bromo-2-fluoro-phenylamino)-6-methyl-5-oxo-1,2,3,5-tetrahydro-indolizine-8-carboxylic acid dimethylamide;
7-(4-Bromo-2-fluoro-phenylamino)-6-methyl-5-oxo-1,2,3,5-tetrahydro-indolizine-8-carboxylic acid (2-methoxy-ethyl)-amide;
7-(4-Bromo-2-fluoro-phenylamino)-6-methyl-5-oxo-1,2,3,5-tetrahydro-indolizine-8-carboxylic acid (2-acetylamino-ethyl)-amide;
7-(4-Bromo-2-fluoro-phenylamino)-6-methyl-5-oxo-1,2,3,5-tetrahydro-indolizine-8-carboxylic acid (pyridin-2-ylmethyl)-amide;
6-Fluoro-7-(2-fluoro-4-trifluoromethyl-phenylamino)-5-oxo-1,2,3,5-tetrahydro-indolizine-8-carboxylic acid;
6-Fluoro-7-(2-fluoro-4-trifluoromethyl-phenylamino)-5-oxo-1,2,3,5-tetrahydro-indolizine-8-carboxylic acid cyclopropylmethoxy-amide;
6-Fluoro-7-(2-fluoro-4-methylsulfanyl-phenylamino)-5-oxo-1,2,3,5-tetrahydro-indolizine-8-carboxylic acid cyclopropylmethoxy-amide;
7-(4-Bromo-2-fluoro-phenylamino)-6-fluoro-8-[5-(2-hydroxy-ethylamino)-[1,3,4]oxadiazol-2-yl]-2,3-dihydro-1H-indolizin-5-one;
7-(4-Bromo-2-fluoro-phenylamino)-6-fluoro-5-oxo-1,2,3,5-tetrahydro-indolizine-8-carboxylic acid methoxy-amide;
7-(4-Bromo-2-fluoro-phenylamino)-6-fluoro-5-oxo-1,2,3,5-tetrahydro-indolizine-8-carboxylic acid ethoxy-amide;
7-(4-Bromo-2-fluoro-phenylamino)-6-fluoro-5-oxo-1,2,3,5-tetrahydro-indolizine-8-carboxylic acid (2-hydroxy-ethoxy)-amide;
7-(4-Bromo-2-methyl-phenylamino)-6-fluoro-5-oxo-1,2,3,5-tetrahydro-indolizine-8-carboxylic acid;
7-(4-Bromo-2-methyl-phenylamino)-6-fluoro-5-oxo-1,2,3,5-tetrahydro-indolizine-8-carboxylic acid cyclopropylmethoxy-amide;
7-(4-Bromo-2-methyl-phenylamino)-5-oxo-1,2,3,5-tetrahydro-indolizine-8-carboxylic acid;
7-(4-Bromo-2-methyl-phenylamino)-5-oxo-1,2,3,5-tetrahydro-indolizine-8-carboxylic acid cyclopropylmethoxy-amide;
6-Fluoro-7-(2-fluoro-4-iodo-phenylamino)-8-(3-hydroxy-3-piperidin-2-yl-azetidine-1-carbonyl)-2,3-dihydro-1H-indolizin-5-one hydrochloride;
7-(4-Bromo-2-fluoro-phenylamino)-6-fluoro-8-(3-hydroxy-3-piperidin-2-yl-azetidine-1-carbonyl)-2,3-dihydro-1H-indolizin-5-one;
6-Fluoro-7-(2-fluoro-4-iodo-phenylamino)-8-(3-hydroxy-3-piperidin-2-yl-azetidine-1-carbonyl)-2,3-dihydro-1H-indolizin-5-one;
Cyclopropanesulfonic acid [7-(4-bromo-2-fluoro-phenylamino)-6-fluoro-5-oxo-1,2,3,5-tetrahydro-indolizin-8-yl]-amide;
N-[7-(4-Bromo-2-fluoro-phenylamino)-6-fluoro-5-oxo-1,2,3,5-tetrahydro-indolizin-8-yl]-N,N-dimethyl-amino-sulfonamide;
2,3-Dihydroxy-propane-amino-sulfonicacid-[7-(4-bromo-2-fluoro-phenylamino)-6-fluoro-5-oxo-1,2,3,5-tetrahydro-indolizin-8-yl]-amide;
1-[7-(4-Bromo-2-fluoro-phenylamino)-6-fluoro-5-oxo-1,2,3,5-tetrahydro-indolizin-8-ylsulfamoyl]-pyrrolidine-2-carboxylic acid;
2-Hydroxymethyl-pyrrolidine-1-sulfonic acid [7-(4-bromo-2-fluoro-phenylamino)-6-fluoro-5-oxo-1,2,3,5-tetrahydro-indolizin-8-yl]-amide;
7-(4-Bromo-2-fluoro-phenylamino)-6-fluoro-8-(1-hydroxy-but-3-enyl)-2,3-dihydro-1H-indolizin-5-one;
7-(4-Bromo-2-fluoro-phenylamino)-6-fluoro-8-(1-hydroxy-allyl)-2,3-dihydro-1H-indolizin-5-one;
7-(4-Bromo-2-fluoro-phenylamino)-8-(2,3-dihydroxy-propionyl)-6-fluoro-2,3-dihydro-1H-indolizin-5-one;
7-(4-Bromo-2-fluoro-phenylamino)-6-fluoro-8-(2-hydroxy-acetyl)-2,3-dihydro-1H-indolizin-5-one;
7-(2-Fluoro-4-trifluoromethyl-phenylamino)-5-oxo-1,2,3,5-tetrahydro-indolizine-8-carboxylic acid ethyl ester;
7-(2-Fluoro-4-trifluoromethyl-phenylamino)-5-oxo-1,2,3,5-tetrahydro-indolizine-8-carboxylic acid;
7-(2-Fluoro-4-trifluoromethyl-phenylamino)-5-oxo-1,2,3,5-tetrahydro-indolizine-8-carboxylic acid cyclopropylmethoxy-amide;
7-(2-Fluoro-4-methoxy-phenylamino)-5-oxo-1,2,3,5-tetrahydro-indolizine-8-carboxylic acid cyclopropylmethoxy-amide;
7-(4-Bromo-2-fluoro-phenylamino)-5-oxo-1,2,3,5-tetrahydro-indolizine-8-carbaldehyde oxime;
7-(4-Bromo-2-fluoro-phenylamino)-8-(3-hydroxy-3-pyridin-2-yl-azetidine-1-carbonyl)-2,3-dihydro-1H-indolizin-5-one;
7-(4-Bromo-2-fluoro-phenylamino)-8-(3-hydroxy-azetidine-1-carbonyl)-2,3-dihydro-1H-indolizin-5-one;
3-(4-Bromo-2-fluoro-phenyl)-1-methanesulfonyl-1,6,7,8-tetrahydro-3H-1,3,5a-triaza-as-indacene-2,5-dione;
Cyclopropanesulfonic acid [7-(4-bromo-2-fluoro-phenylamino)-5-oxo-1,2,3,5-tetrahydro-indolizin-8-yl]-amide;
N-[7-(4-Bromo-2-fluoro-phenylamino)-5-oxo-1,2,3,5-tetrahydro-indolizin-8-yl]-4-fluoro-benzenesulfonamide;
[7-(4-Bromo-2-fluoro-phenylamino)-5-oxo-1,2,3,5-tetrahydro-indolizin-8-yl]-carbamic acid tert-butyl ester;
Cyclohexanesulfonic acid [7-(4-bromo-2-fluoro-phenylamino)-5-oxo-1,2,3,5-tetrahydro-indolizin-8-yl]-amide;
N-[7-(4-Bromo-2-fluoro-phenylamino)-5-oxo-1,2,3,5-tetrahydro-indolizin-8-yl]-4-trifluoromethyl-benzenesulfonamide;
N-[7-(4-Bromo-2-fluoro-phenylamino)-5-oxo-1,2,3,5-tetrahydro-indolizin-8-yl]-N,N-dimethylaminosulfonamide;
5-(4-Bromo-2-fluoro-phenylamino)-2,2-dimethyl-7-oxo-3a,7,8,8a-tetrahydro-1,3-dioxa-7a-aza-cyclopenta[a]indene-4-carboxylic acid;
7-(4-Bromo-2-fluoro-phenylamino)-1,2-dihydroxy-5-oxo-1,2,3,5-tetrahydro-indolizine-8-carboxylic acid ethyl ester;
7-(4-Bromo-2-fluoro-phenylamino)-1,2-dihydroxy-5-oxo-1,2,3,5-tetrahydro-indolizine-8-carboxylic acid cyclopropylmethoxy-amide;
7-(4-Bromo-2-fluoro-phenylamino)-1,2-dihydroxy-5-oxo-1,2,3,5-tetrahydro-indolizine-8-carboxylic acid (2-hydroxy-ethoxy)-amide;
7-(4-Bromo-2-fluoro-phenylamino)-1,2-dihydroxy-5-oxo-1,2,3,5-tetrahydro-indolizine-8-carboxylic acid cyclobutylmethoxy-amide;
7-(4-Bromo-2-fluoro-phenylamino)-1,2-dihydroxy-5-oxo-1,2,3,5-tetrahydro-indolizine-8-carboxylic acid (3-hydroxy-2-methyl-propoxy)-amide;
1-(2,3-Dihydroxy-propyl)-cyclopropanesulfonic acid [6-fluoro-7-(2-fluoro-4-iodo-phenylamino)-5-oxo-1,2,3,5-tetrahydro-indolizin-8-yl]-amide;
1-(2,3-Dihydroxy-propyl)-cyclopropanesulfonic acid [7-(4-bromo-2-fluoro-phenylamino)-6-methyl-5-oxo-1,2,3,5-tetrahydro-indolizin-8-yl]-amide;
1-(2,3-Dihydroxy-propyl)-cyclopropanesulfonic acid [7-(4-bromo-2-fluoro-phenylamino)-6-fluoro-5-oxo-1,2,3,5-tetrahydro-indolizin-8-yl]-amide;
6-Fluoro-7-(2-fluoro-4-iodo-phenylamino)-5-oxo-1,2,3,5-tetrahydro-indolizine-8-carboxylic acid (2,3-dihydroxy-propoxy)-amide;
7-(2-Fluoro-4-iodo-phenylamino)-6-methyl-5-oxo-1,2,3,5-tetrahydro-indolizine-8-carboxylic acid cyclopropyl methoxy amide;
7-(2-Fluoro-4-iodo-phenylamino)-6-methyl-5-oxo-1,2,3,5-tetrahydro-indolizine-8-carboxylic acid (2-hydroxy-ethoxy)-amide;
7-(2-Fluoro-4-iodo-phenylamino)-6-methyl-5-oxo-1,2,3,5-tetrahydro-indolizine-8-carboxylic acid cyclobutylmethoxy-amide;
1-(2,3-Dihydroxy-propyl)-cyclopropanesulfonic acid [7-(2-fluoro-4-iodo-phenylamino)-6-methyl-5-oxo-1,2,3,5-tetrahydro-indolizin-8-yl]-amide; and
Cyclopropanesulfonic acid [7-(4-bromo-2-fluoro-phenylamino)-6-methyl-5-oxo-1,2,3,5-tetrahydro-indolizin-8-yl]-amide, or a pharmaceutically acceptable salt thereof.

Many of the compounds represented by formula I and Id are capable of forming acid addition salts, particularly pharmaceutically acceptable acid addition salts. Pharmaceutically acceptable acid addition salts of the compound of formula I include those of inorganic acids, for example, hydrohalic acids such as hydrochloric acid, hydrobromic acid or hydroiodic acid, nitric acid, sulfuric acid, phosphoric acid; and organic acids, for example aliphatic monocarboxylic acids such as formic acid, acetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid. These salts may be prepared from compounds of formula I and Id by known salt-forming procedures.

Compounds of formula I and Id which contain acidic, e.g. carboxyl, groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine. These salts may be prepared from compounds of formula I and Id by known salt-forming procedures.

In those compounds where there is an asymmetric carbon atom the compounds exist in individual optically active isomeric forms or as mixtures thereof, e.g. as racemic or diastereomeric mixtures. The present invention embraces both individual optically active R and S isomers as well as mixtures, e.g. racemic or diastereomeric mixtures, thereof.

The present invention includes all pharmaceutically acceptable isotopically-labeled compounds of formula (I) and (Id) wherein one or more atoms are replaced by atoms having the same atomic number, but an atomic mass or mass number different from the atomic mass or mass number usually found in nature.

Examples of isotopes suitable for inclusion in the compounds of the invention comprises

isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labeled compounds of formula (I) and (Id) can generally be prepared by conventional techniques known to those skilled in the art or by processes analogous to those described in the accompanying Examples and Preparations Sections using an appropriate isotopically-labeled reagent in place of the non-labeled reagent previously employed.

The invention provides, in another aspect, a process for preparing a compound of formula (I) and (Id). The schemes detailed below show general schemes for synthesizing compounds of formula (I) and (Id). It is recognized that the compounds corresponding to the Roman numerals in the schemes do not correspond to the Roman numerals of claimed compounds.

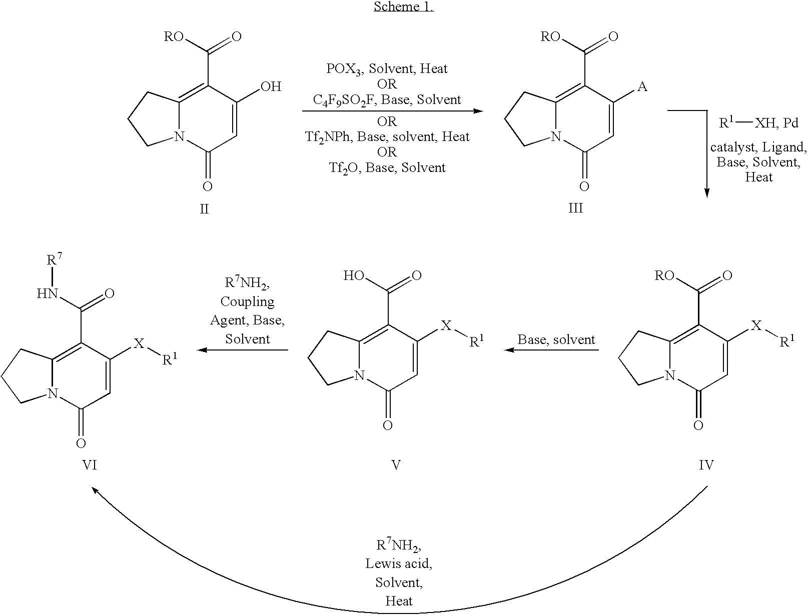

Compounds of formula II may be prepared using published methods described in J. Org. Chem., 1995, 60, 2912 and Tetrahedron, 2002, 58, 2821,

Compounds of formula II may be converted into compounds of formula III by reaction with a halogenating agent such as phosphorus oxybromide, neat or in a suitable solvent such as toluene, at temperatures ranging from room temperature to 140° C.

Alternatively, compounds of formula II may be reacted with nonafluorobutane sulphonyl fluoride in the presence of a base such as diisopropyl ethylamine and a catalyst, such as N,N-dimethyl-4-aminopyridine, in a solvent such as dichloromethane, at room temperature, or with N-phenyltrifluoromethanesulfonimide in the presence of a base, such as diisopropylethyl amine, in a suitable solvent, such as 1,2-dimethoxyethane, at temperatures ranging from room temperature to the refluxing temperature of the solvent. In addition, compounds of formula II may be treated with trifluoromethanesulphonic acid anhydride in the presence of base, such as pyridine, in a solvent, such as dichloromethane, at temperatures ranging from −20° C. to ambient temperature.

Compounds of formula IV may be obtained from compounds of formula III by reaction with appropriate anilines or phenols or thiophenols, using Buchwald-Hartwig C—N/S/O coupling conditions. The Buchwald-Hartwig reactions may be performed in presence of a catalyst such as tris(dibenzylidineacetone)dipalladium (0) or palladium acetate, a base such as potassium phosphate, sodium tert-butoxide, 1,8-diazobicyclo[5.4.1]undec-7-ene or cesium carbonate, a ligand such as 9,9′-dimethyl-4,5-bis(diphenylphosphino)-xanthene, 2,2′-bis(diphenylphosphino)-1-1′-binaphthyl, 2-dicyclohexylphosphino-2′-(N,N-dimethylamino)biphenyl, 2-dicyclohexylphosphino-2′,6′-(dimethoxy)biphenyl or tributylphosphine, in a suitable solvent such as toluene, 1,2-dimethoxyethane, tetrahydrofuran or dioxane, at temperatures ranging from room temperature to the refluxing temperature of the solvent, or under microwave irradiation at a temperature ranging from 70° C. to 150° C.

Compounds of formula V can be obtained from compounds of formula IV by reaction with a base such as sodium hydroxide in a protic solvent such as ethanol or methanol, at temperatures ranging from room temperature to the refluxing temperature of the solvent.

Compounds of formula V can be treated with a functionalized hydroxylamine or an amine and a suitable coupling agent, such as O-(7-azabenzo-triazol-1-yl)-N,N,N′,N′-tetra-methyluronium hexafluorophosphate, N-(3-dimethylaminopropyl)-N′-ethylcarbodimidime hydrochloride or N,N-dicyclohexylcarbodiimide in the presence of N-hydroxybenzotriazole, with a suitable base such as diisopropylethylamine or triethylamine, in an aprotic solvent such as tetrahydrofuran, N,N-dimethylformamide, or dichloromethane, at temperatures ranging from 0° C. to room temperature, to obtain the compounds of formula VI. Alternatively, compounds of formula VI can be obtained directly from compounds of formula IV by reaction with an amine or hydroxylamine in the presence of a Lewis acid such as trimethyl aluminum, in a solvent such as dichloromethane, at temperatures ranging from room temperature to the refluxing temperature of the solvent.

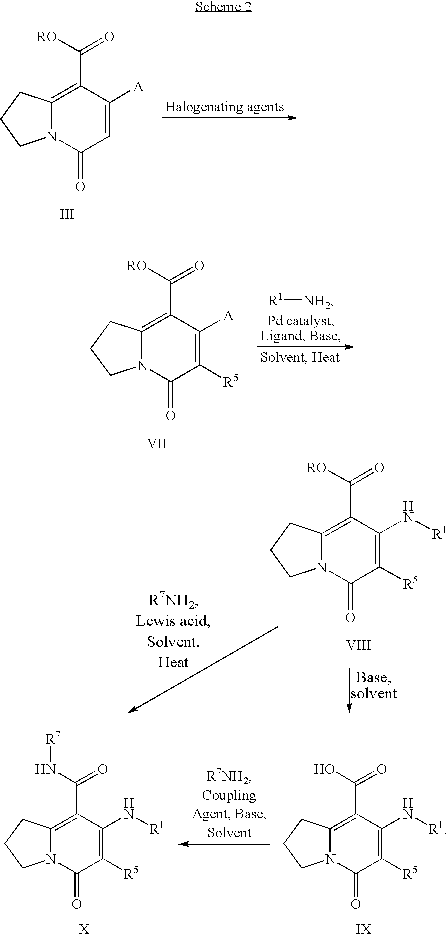

Compounds of formula III can be converted to compounds of formula VII by electrophilic halogenation using reagents such as [1-(chloromethyl)-4-fluoro-1,4-diazoniabicyclo[2.2.2]octane-bis(tetrafluoroborate)] in a suitable solvent, such as acetonitrile, at temperatures ranging from room temperature to 70° C.

Compounds of formula VII can be converted into compounds of formula VIII using the conditions as described for the preparation of compounds of formula IV (scheme 1). Compounds of formula VIII can be converted into compounds of formula IX using the conditions as described for the preparation of compounds of formula V (scheme 1). Compounds of formula IX can be converted into compounds of formula X using the conditions as described for the preparation of compounds of formula VI (scheme 1). Alternatively, compounds of formula X can be obtained directly from compounds of formula VIII by reaction with an amine or a hydroxylamine using the conditions as described for the preparation of compounds of formula VI (scheme 1).

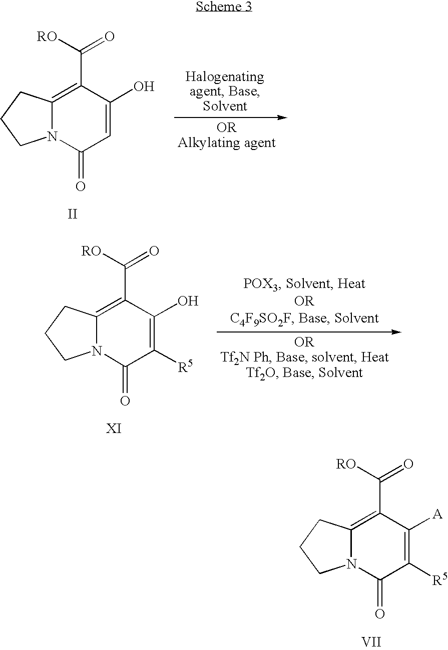

Compounds of formula XI may be prepared from compounds of formula II by reacting the latter with a base such as NaH and an alkylating agent such as methyl iodide or a halogenating agents such as deoxyfluor, NCS, NBS, NIS, in a suitable solvent such as THF or DMF, at temperatures ranging from room temperature to 100° C.

Compounds of formula XI can be transformed to compounds of formula VII using the same conditions as described for the preparation of compounds of formula II (scheme 1).

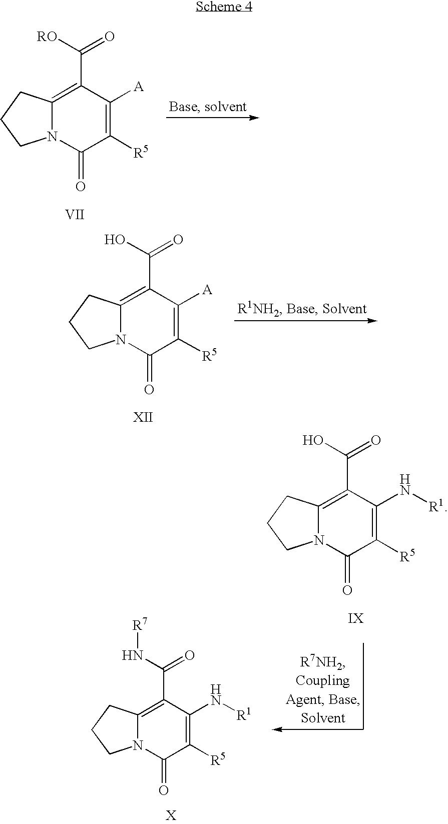

Compounds of formula XI can be obtained from compounds of formula VII by reaction with a base such as sodium or lithium hydroxide in a protic solvent such as ethanol or methanol, at temperatures ranging from room temperature to the refluxing temperature of the solvent.

Compounds of formula XII can then be converted into compounds of formula IX using an S_{N}AR reaction. The latter is carried out in a suitable solvent such as THF, using an amide base such as LDA, LiHMDS, NaHMDS, or KHMDS at appropriate temperatures, typically ranging from −78° C. to room temperature.

Compounds of formula IX can be converted into compounds of formula X using the same conditions as described for the preparation of compounds of formula VI (scheme 1).

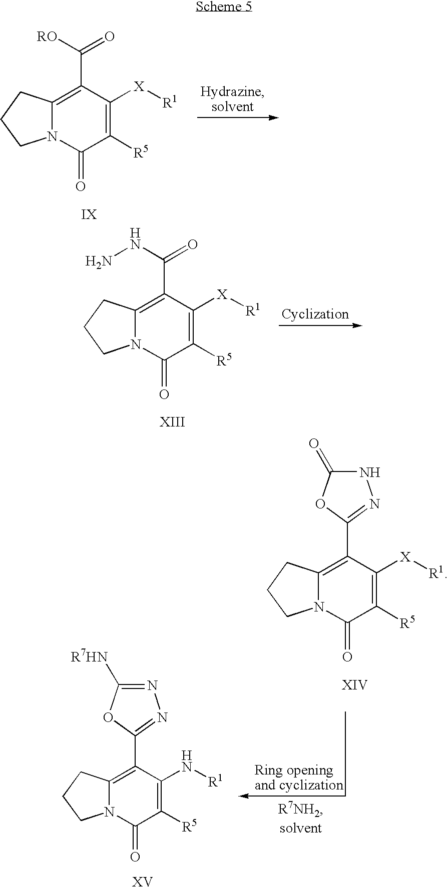

Compounds of formula IX can be converted into compounds of formula XIII with hydrazine by standard coupling procedures using reagents like EDCI or PyBOP in the presence of HOBt in a suitable organic solvent such as DMF, THF or dichloromethane.

Compounds of formula XIII can then be converted into compounds of formula XIV using either carbonyldiimidazole, phosgene, or a phosgene equivalent, in a suitable organic solvent such as DMF, toluene, or dichloromethane.

Compounds of formula XV are accessible from compounds of formula XIV by the addition of an appropriate amine followed by re-cyclization of the intermediate acyl hydrazide using triphenylphosphine, triethylamine, and CCl4 in dichloromethane.

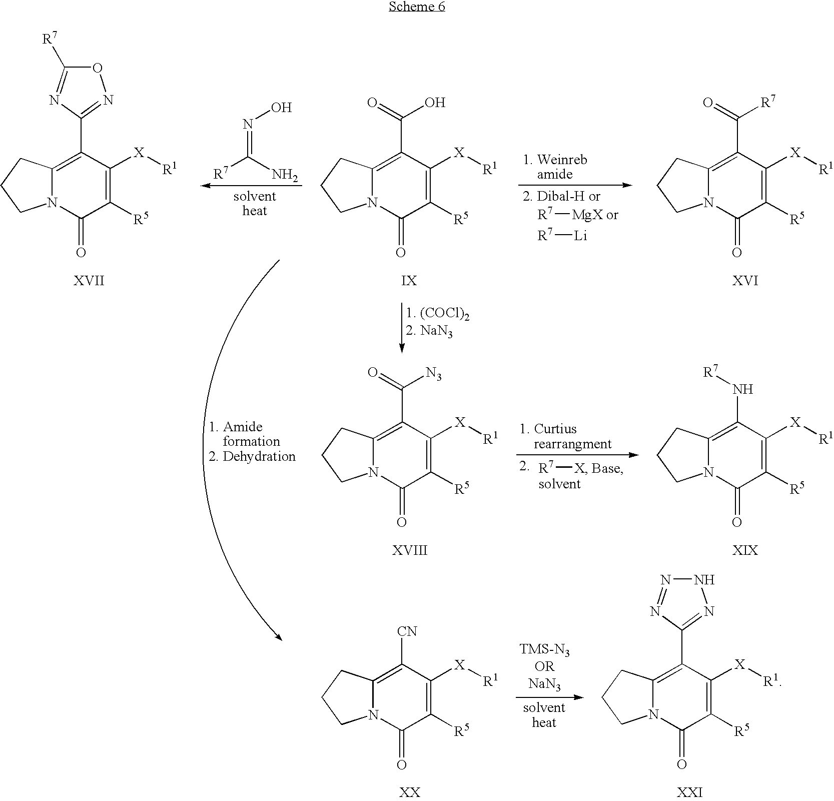

Aldehydes and ketones of formula XVI can be prepared from acids of formula IX using the standard methods, such as converting the acids into corresponding Weinreb amide, followed by treatment with appropriate organo-metallic reagents.

Oxadiazoles of formula XVII can be prepared by acylating the respective amidoxime, followed by dehydrative cyclization.

Acyl azides of formula XVIII can be prepared from compounds of the general formula IX via the acid halide, for example the acid chloride using standard conditions. The formula XVIII compounds can then be transformed via the Curtius rearrangement to give compounds of the general formula XIX.

Following standard methodology, acids of formula IX are converted to the corresponding amides, which are then dehydrated to give the corresponding nitriles of formula XX. Formula XX compounds can be treated with trimethylsilyl azide or NaN₃ in a suitable aprotic solvent such as N,N-dimethylformamide, at temperatures ranging from room temperature to 100° C. to yield compounds of formula XX.

The inhibitory properties of compounds of formula I and Id may be demonstrated using the following test procedures:

A BRAF-MEK-ERK cascade assay is used to evaluate the effects of these compounds as inhibitors of the MAP kinase pathway. An enzymatic cascade assay is set up using recombinant human activated BRAF (V599E) kinase (Cat No. 14-557), human full length unactive MEK1 kinase (Cat No. 14-706) and human full length unactive MAP Kinase 2 ERK2 (Cat No. 14-536) enzymes procured from Upstate. TR-FRET (Time resolved fluorescence resonance energy transfer) detection technology is used for the read out. The assay buffer solution contains 50 mM Tris pH 7.5, 10 mM MgCl2, 1 mM DTT, 0.01% Tween 20, 0.1 nM activated BRAF, 2 nM unactive MEK1, 10 nM unactive ERK2, 100 μM ATP and 500 nM long chain biotin-peptide substrate (LCB-FFKNIVTPRTPPP) in a 384 well format. The kinase reaction is stopped after 90 minutes with 10 mM EDTA and Lance detection mix (2 nM Eu-labeled phospho-serine/threonine antibody (Cat. No. AD0176-Perkin Elmer), 20 nM SA-APC (Cat No. CR130-100-Perkin Elmer) is added. The TR-FRET signal (Excitation at 340 nm, Emission at 615 nm and 665 nm) is read with 50 μs delay time on a Victor3 V fluorimeter. The data is calculated using the ratio of readings at 665 nm to 615 nm. The final concentration of DMSO is 2.5% in the assay. Compounds are screened at 10 μM concentration with pre-incubation of the enzymes in the presence of test compound for 45 minutes.

Each individual IC50 is determined using a 10 point dose response curve generated by GraphPad Prism software Version 4 (San Diego, Calif., USA) using non linear regression curve fit for sigmoidal dose response (variable slope).

An in-vitro MAP kinase assay is set up using activated MAP kinase 2/ERK2 (Cat. No. 14-550) obtained from Upstate. TR-FRET detection technology is used for the read out. The assay buffer solution contains 50 mM Tris pH 7.5, 10 mM MgCl₂, 1 mM DTT, 0.01% Tween 20, 1 nM activated ERK2, 100 μM ATP and 500 nM long chain biotin-peptide substrate (LCB-FFKNIVTPRTPPP) in a 384 well format. The kinase reaction is stopped after 90 minutes with 10 mM EDTA and Lance detection mix (2 nM Eu-labeled phospho-serine/threonine antibody (Cat. No. AD0176-Perkin Elmer), 20 nM SA-APC (Cat. No. CR130-100-Perkin Elmer) is added. The TR-FRET signal (excitation at 340 nm, emission at 615 nm and 665 nm) is read with 50 μs delay time on Victor3 V fluorimeter. The data is calculated using the ratio of readings at 665 nm to 615 nm. The final concentration of DMSO is 2.5% in the assay. Compounds are screened at 10 μM concentration with pre-incubation of the enzymes in the presence of test compound for 45 minutes.

The radioactive filter binding assay is standardized using recombinant human activated BRAF (V599E) kinase (Cat No. 14-557) and kinase dead MEK1 (K97R) (Cat No. 14-737) procured from Upstate. The incorporation of 32P into MEK1 (K97R) by BRAF (V599E) is measured with final assay buffer conditions of 50 mM Tris pH 7.5, 10 mM MgCl2, 1 mM DTT, 100 mM sucrose, 100 μM sodium orthovanadate, 5 μM ATP and 2 μCi[γ 32P] ATP and 500 mg MEK1 Kinase dead substrate. The enzymatic reaction is stopped after 120 minutes with 8N HCl (hydrochloric acid) and 1 mM ATP. The solution is spotted on PSI filter paper and washed 4 times with 0.75% orthophosphoric acid and lastly with acetone. The dried P81 filter papers are read in a Micro-beta Trilux scintillation counter. The final concentration of DMSO is 1% in the assay. Compounds are screened at 10 μM concentration with pre-incubation of the enzymes in the presence of test compound for 45 minutes.

These assays described above are fully detailed in Han, Shulin, et. al., Bioorganic & Medicinal Chemistry Letters (2005) 15, 5467-5473, and in Yeh, et. al., Clin Cancer Res (2007) 13 (5), 1576-1583.

The cell viability assay in A375 cells is set up in a 96-well plate format using XTT.

XTT is a yellow tetrazolium salt that is cleaved to an orange formazan dye by the mitochondria of metabolically active cells. The procedure allows for rapid determination in a microtitre plate, to give reproducible and sensitive results.

A375 cells are grown in DMEM media containing 10% FBS and 1 mM sodium pyruvate. Cells are trypsinized and seeded at 1000 cells/well. After allowing the cells to adhere overnight, compound is added to the wells at the following final concentrations: 10, 3, 1, 0.3, 0.1, 0.03, 0.01, 0.001, and 0.0001 μM. The assay is set up in triplicates for each concentration. DMSO concentrations are kept at 0.5%/well. Three days after compound addition, the XTT assay is performed. Wells are washed once with PBS. 100 μL of DMEM media without phenol red or FBS is added to each well. A working solution of XTT containing 1 mg/ml XTT and 100 μL of PMS (stock concentration 0.383 mg/ml) per 5 ml is prepared. 50 μL of the working solution of XTT is added to each well. Absorbance of the plate is read at 465 nm using a Spectramax 190 (Molecular Devices). The absorbance from wells with media and XTT alone, but without cells is considered the blank and subtracted from readings from all wells.

Percentage viability is calculated considering the blank subtracted value from wells treated with DMSO alone as 100% viable. GI50 values are calculated using Graphpad Prism, using non-linear regression curve fit for sigmoidal dose response (variable slope).

The cell viability assay is further described in Scudiero, et. al., Cancer Research (1988) 48, 4827-4833; Weislow, et. al., J. Natl. Cancer Institute, (1989) 81, 577-586; and Roehm, et. al., J. Immunol. Methods [1991]142:257-265.

The compounds of the present invention are useful as both prophylactic and therapeutic treatments for diseases or conditions related to the hyperactivity of MEK, as well as diseases or conditions modulated by the Raf/Ras/Mek pathway.

Thus, as a further aspect, the invention relates to a method for treating a disease or condition related to the hyperactivity of MEK, or a disease or condition modulated by the MEK cascade, comprising administration of an effective therapeutic amount of a compound of formula (I) or (Id) or a pharmaceutically acceptable salt thereof.

As a further aspect, the invention relates to a method for treating proliferative diseases, such as cancer, comprising administration of an effective amount of a compound of formula (I) or (Id) or a pharmaceutically acceptable salt thereof.

Examples of cancers include but are not limited to: angiosarcoma, fibrosarcoma, rhabdomyosarcoma, liposarcoma, myxoma, rhabdomyoma, fibroma, lipoma, teratoma; bronchogenic carcinoma, squamous cell carcinoma, undifferentiated small cell carcinoma, undifferentiated large cell carcinoma, alveolar (bronchiolar) carcinoma, bronchial adenoma, lymphoma, chondromatous hanlartoma, inesothelioma, esophageal squamous cell carcinoma, leiomyosarcoma, leiomyosarcoma, ductal adenocarcinoma, insulinoma, glucagonoma, gastrinoma, vipoma, stomach and small bowel carcinoid tumors, adenocarcinoma, Karposi's sarcoma, leiomyoma, hemangioma, lipoma, neurofibroma, fibroma, tubular adenoma, villous adenoma, hamartoma, Wilm's tumor [nephroblastoma, leukemia, bladder and urethra squamous cell carcinoma, transitional cell carcinoma, adenocarcinoma, seminoma, teratoma, embryonal carcinoma, teratocarcinoma, choriocarcinoma, interstitial cell carcinoma, fibroadenoma, adenomatoid tumors, hepatoma (hepatocellular carcinoma), cholangiocarcinoma, hepatoblastoma, hepatocellular adenoma, hemangioma, osteogenic sarcoma (osteosarcoma), malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors, osteoma, granuloma, xanthoma, osteitis defornians, meningioma, meningiosarcoma, gliomatosis, astrocytoma, medulloblastoma, glioma, ependymoma, germinoma [pinealoma], glioblastoma multiform, oligodendroglioma, schwannoma, retinoblastoma, congenital tumors, spinal cord neurofibroma, meningioma, glioma, endometrial carcinoma, cervical carcinoma, pre-tumor cervical dysplasia, ovarian carcinoma, serous cystadenocarcinoma, mucinous cystadenocarcinoma, granulosa-thecal cell tumors, Sertoli-Leydig cell tumors, dysgerminoma, malignant teratoma, intraepithelial carcinoma, adenocarcinoma, melanoma), vaginal clear cell carcinoma, botryoid sarcoma (embryonal rhabdomyosarcoma), fallopian tube carcinoma, acute and chronic myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma, malignant melanoma, basal cell carcinoma, moles, dysplastic nevi, angioma, dermatofibroma, keloids, psoriasis, and neuroblastoma.

The compounds of the present invention may also be useful in the treatment of other diseases or conditions related to the hyperactivity of MEK. Thus, as a further aspect, the invention relates to a method of treatment of a disorder selected from: xenograft (cellos), skin, limb, organ or bone marrow transplant) rejection; osteoarthritis; rheumatoid arthritis; cystic fibrosis; complications of diabetes (including diabetic retinopathy and diabetic nephropathy); hepatomegaly; cardiomegaly; stroke (such as acute focal ischemic stroke and global cerebral ischemia); heart failure; septic shock; asthma; chronic obstructive pulmonary disorder; Alzheimer's disease; and chronic or neuropathic pain.

The term “chronic pain” for purposes of the present invention includes, but is not limited to, idiopathic pain, and pain associated with chronic alcoholism, vitamin deficiency, uremia, or hypothyroidism. Chronic pain is associated with numerous conditions including, but not limited to, inflammation, and post-operative pain.

As used herein, the term “neuropathic pain” is associated with numerous conditions which include, but are not limited to, inflammation, postoperative pain, phantom limb pain, burn pain, gout, trigeminal neuralgia, acute herpetic and postherpetic pain, causalgia, diabetic neuropathy, plexus avulsion, neuroma, vasculitis, viral infection, crush injury, constriction injury, tissue injury, limb amputation, and nerve injury between the peripheral nervous system and the central nervous system.

Compounds of the invention may also be useful as antiviral agents for treating viral infections such as HIV, hepatitis (B) virus (HBV) human papilloma virus (HPV), cytomegalovirus (CMV], and Epstein-Barr virus (EBV).

Compounds of the invention may also be useful in the treatment of restenosis, psoriasis, allergic contact dermatitis, autoimmune disease, atherosclerosis and inflammatory bowel diseases, e.g. Crohn's disease and ulcerative colitis.

An MEK inhibitor of the present invention may be usefully combined with another pharmacologically active compound, or with two or more other pharmacologically active compounds, particularly in the treatment of cancer. For example, a compound of the formula (I), or a pharmaceutically acceptable salt thereof, as defined above, may be administered simultaneously, sequentially or separately in combination with one or more agents selected from chemotherapy agents, e.g. mitotic inhibitors such as a taxane, a vinca alkaloid, paclitaxel, docetaxel, vincristine, vinblastine, vinorelbine or vinflunine, and other anticancer agents, e.g. cisplatin, 5-fluorouracil or 5-fluoro-2-4(1H,3H)-pyrimidinedione (5FU), flutamide or gemcitabine.

Such combinations may offer significant advantages, including synergistic activity, in therapy.

A compound of the formula (I) or (Id) may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors, such as LBH589; compounds which induce cell differentiation processes; cyclooxygenase inhibitors; MMP inhibitors; mTOR inhibitors, such as RAD001; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3, such as PKC412; Hsp90 inhibitors such as 17-AAG (17-allylamino-gelda-namycin, NSC330507), 17-DMAG (17-dimethylaminoethylamino-17-demethoxy-geldana-mycin, NSC707545), IPI-504, CNF1010, CNF2024, CNF1010 from Conforma Therapeutics and AUY922; temozolomide (TEMODAL); kinesin spindle protein inhibitors, such as SB715992 or SB743921 from GlaxoSmithKline, or pentamidine/chlorpromazine from CombinatoRx; PI3K inhibitors, such as BEZ235; RAF inhibitors, such as RAF265; EDG binders, antileukemia compounds, ribonucleotide reductase inhibitors, S-adenosylmethionine decarboxylase inhibitors, antiproliferative anti-bodies or other chemotherapeutic compounds. Further, alternatively or in addition they may be used in combination with other tumor treatment approaches, including surgery, ionizing radiation, photodynamic therapy, implants, e.g. with corticosteroids, hormones, or they may be used as radiosensitizers. Also, in anti-inflammatory and/or antiproliferative treatment, combination with anti-inflammatory drugs is included. Combination is also possible with antihistamine drug substances, bronchodilatatory drugs, NSAID or antagonists of chemokine receptors.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atame-stane, exemestane and formestane and, in particular, non-steroids, especially aminoglutethimide, roglethimide, pyridoglutethimide, trilostane, testolactone, ketoconazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Amino glutethimide can be administered, e.g., in the form as it is marketed, e.g. under the trademark, ORIMETEN. A combination of the invention comprising a chemotherapeutic agent which is an aromatase inhibitor is particularly useful for the treatment of hormone receptor positive tumors, e.g., breast tumors.

The term “anti-estrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an anti-estrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecin and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark M 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating compound” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g., under the trademark HOLOXAN.

The term “histone deacetylase inhibitors” or “HDAC inhibitors” relates to compounds which inhibit the histone deacetylase and which possess antiproliferative activity. This includes compounds such as sodium butyrate, LDH589 disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof, especially the lactate salt. It further especially includes suberoylanilide hydroxamic acid (SAHA), MS275, FK228 (formerly FR901228), trichostatin A and compounds disclosed in U.S. Pat. No. 6,552,065, in particular, N-hydroxy-3-[4-[[[2-(2-methyl-1H-indol-3-yl)-ethyl]-amino]-methyl]phenyl]-2E-2-propenamide, or a pharmaceutically acceptable salt thereof.

The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity”; or a “protein or lipid phosphatase activity”; or “further anti-angiogenic compounds” as used herein includes, but is not limited to, protein tyrosine kinase and/or serine and/or threonine kinase inhibitors or lipid kinase inhibitors, e.g.,

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin B4 inhibitors;
e) compounds targeting, decreasing or inhibiting the activity of the Ax1 receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, i.e C-kit receptor tyrosine kinases —(part of the PDGFR family), such as compounds which target, decrease or inhibit the activity of the c-Kit receptor tyrosine kinase family, especially compounds which inhibit the c-Kit receptor, e.g. imatinib;
h) compounds targeting, decreasing or inhibiting the activity of members of the c-Abl family, their gene-fusion products (e.g. BCR-Ab1 kinase) and mutants, such as compounds which target decrease or inhibit the activity of c-AbI family members and their gene fusion products, e.g. a N-phenyl-2-pyrimidine-amine derivative, e.g. imatinib or nilotinib (AMN107); PD180970; AG957; NSC 680410; PD173955 from ParkeDavis; or dasatinib (BMS-354825)
i) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; BEZ235 (a PI3K inhibitor) or AT7519 (CDK inhibitor);
j) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (mw<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
k) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyro sine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin), cetuximab (Erbitux), Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
1) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g., inhibitors of phosphatase 1, phosphatase 2A, or CDC25, e.g. okadaic acid or a derivative thereof.

Compounds which induce cell differentiation processes are e.g. retinoic acid, or tocopherol or tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid.

“Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA.

“Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “mTOR inhibitors” relates to compounds which inhibit the mammalian target of rapamycin (mTOR) and which possess antiproliferative activity such as sirolimus (Rapamune), everolimus (CerticanÔ), CCI-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon.

The term “inhibitor of Ras oncogenic isoforms”, e.g. H-Ras, K-Ras, or N-Ras, as used herein refers to compounds which target decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g. PKC412, TKI258, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17AAG), a geldanamycin derivative; other geldanamycin related compounds, and radicicol.

The term “antiproliferative antibodies” as used herein includes, but is not limited to, trastuzumab (Herceptin), Trastuzumab-DM1, erbitux, bevacizumab (Avastin), rituximab (Rituxan), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2-alpha-hydroxy ribose (arabinoside) derivative of deoxycytidine. Also included is the purine analog of hypoxanthine, 6-mercaptopurine (6-MP) and fludarabine phosphate.

Somatostatin receptor antagonists as used herein refers to compounds which target, treat or inhibit the somatostatin receptor such as octreotide, and SOM230 (pasireotide).

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4th Edition, Vol. 1, pp. 248-275 (1993).

The term “EDG binders” as used herein refers a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin).

Photodynamic therapy as used herein refers to therapy which uses certain chemicals known as photosensitizing compounds to treat or prevent cancers. Examples of photodynamic therapy includes treatment with compounds, such as e.g. VISUDYNE and porfimer sodium.

Angiostatic steroids as used herein refers to compounds which block or inhibit angiogenesis, such as, e.g., anecortave, triamcinolone, hydrocortisone, 11-epihydrocotisol, cortexolone, 17-hydroxyprogesterone, corticosterone, desoxycorticosterone, testosterone, estrone and dexamethasone.

Implants containing corticosteroids refers to compounds, such as e.g. fluocinolone, dexamethasone.

“Other chemotherapeutic compounds” include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code nos., generic or trade names may be taken from the actual edition of the standard compendium “The Merck Index” or from databases, e.g. Patents International (e.g. IMS World Publications).

None of the quotations of references made within the present disclosure is to be understood as an admission that the references cited are prior art that would negatively affect the patentability of the present invention.

The compounds of the invention may also be administered simultaneously, separately or sequentially in combination with one or more other suitable active agents selected from the following classes of agents: Anti IL-1 agents, e.g: Anakinra; anti cytokine and anti-cytokine receptor agents, e.g. anti IL-6 R Ab, anti IL-15 Ab, anti IL-17 Ab, anti IL-12 Ab; B-cell and T-cell modulating drugs, e.g. anti CD20 Ab; CTL4-Ig, disease-modifying anti-rheumatic agents (DMARDs), e.g. methotrexate, leflunamide, sulfasalazine; gold salts, penicillamine, hydroxychloroquine and chloroquine, azathioprine, glucocorticoids and non-steroidal anti-inflammatories (NSAIDs), e.g. cyclooxygenase inhibitors, selective COX-2 inhibitors, agents which modulate migration of immune cells, e.g. chemokine receptor antagonists, modulators of adhesion molecules, e.g. inhibitors of LFA-1, VLA-4.

The present invention is also in relation to a pharmaceutical composition comprising a compound of formula I or Id or its prodrug and pharmaceutically acceptable excipients.

In still another embodiment of the present invention, the prodrug is selected from a group comprising, esters and hydrates.

The term pro-drug is also meant to include any covalently bonded carries which release the active compound of the invention in vivo when such prodrug is administered to a mammalian subject. Pro-drugs of a compound of the invention may be prepared by modifying functional groups present in the compound of the invention in such a way that the modifications are cleaved, either in routine manipulation or in vivo, to the parent compound of the invention.

In still another embodiment of the present invention, the excipients are selected from a group comprising, binders, anti-adherents, disintegrants, fillers, diluents, flavors, colors, glidants, lubricants, preservatives, sorbents and sweeteners or combinations thereof.

In still another embodiment of the present invention, the composition is formulated into various dosage forms selected from a group comprising tablet, troches, lozenges, aqueous or oily suspensions, ointment, patch, gel, lotion, dentifrice, capsule, emulsion, creams, spray, drops, dispersible powders or granules, emulsion in hard or soft gel capsules, syrups and elixirs.

Dosages of agents of the invention employed in practicing the present invention will of course vary depending, for example, on the particular condition to be treated, the effect desired and the mode of administration. In general, suitable daily dosages for oral administration are of the order of 0.1 to 10 mg/kg.

The invention is further illustrated by the following non-limiting examples, where the following abbreviations are employed:

### TEA: Triethylamine

### DPPA: Diphenylphosphorylazide

LDA: Lithium diisopropylamide
EDCI: 1-Ethyl-3-(3′-dimethylaminopropyl)carbodiimide

### DMAP: 4-Dimethylaminopyridine

### HOBt: 1-Hydroxybenzotriazole

Selectfluor: 1-Chloromethyl-4-fluoro-1,4-diazoniabicyclo[2.2.2]octane bis(tetrafluoroborate)
Dess-martin periodinane: 1,1,1-Triacetoxy-1′-dihydro-1,2-benziodoxol-3(1H)-one

### DCM: Dichloromethane

### THF: Tetrahydrofuran

### DMF: Dimethylformamide

DIBAL-H: Diisobutylaluminum hydride

### EtOH: Ethanol

### EtOAc: Ethylacetate

triflic anhydride: trifluoromethanesulfonic anhydride;
DCM: dichloromethane;
Pd(OAc)2: palladium acetate;
Cs₂CO₃: cesium carbonate;
BINAP: 2,2′-bis(diphenylphosphino)-1,1-binaphthyl;
LiOH: lithium hydroxide;
EDCI: 1-ethyl-3-(3′-dimethylaminopropyl)carbodiimide;
RT: room temperature;
TLC: thin layer chromatography,

### NCS: N-chlorosuccinimide,

### NBS: N-bromosuccinamide,

### NIS: N-iodosuccinimide,

LiHMDS: lithium bis(trimethylsilyl)amide,
LDA: lithium diisopropylamide,
NaHMDS: sodium bis(trimethylsilyl)amide,
KHMDS: potassium bis(trimethylsilyl)amide,
ByBOP: benzotriazol-1-yl-oxytripyrrolidinophosphonium hexafluorophosphate,
TMS: trimethylsilyl,
MgCl₂; magnesium chloride,
TBTU: O-(benzotriazol-1-yl)-N,N,N′,N′-tetramethyluronium tetrafluoroborate,
NMR: nuclear magnetic resonance,
DMSO-d6: deuterated dimethyl sulfoxide,
CDCl₃: deuterated chloroform,
LC-MS: liquid chromatography-mass spectrometry,
HPLC: high pressure liquid chromatography or high performance liquid chromatography.

The examples provided in the description are only to describe the invention, hence they should not be construed to limit the scope of the invention.

### EXAMPLE 1

### Step 1

Compounds of steps I & II may be prepared using published methods described in J. Org. Chem., 1995, 60, 2912 and Tetrahedron, 2002, 58, 2821,

### Synthesis of 5-Methoxy-3,4-dihydro-2H-pyrrole

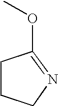

Pyrrolidin-2-one (85 g, 1 mol) is added dropwise over a period of 2 hours to a stirred solution of dimethyl sulphate (126 g, 1 mol) under a nitrogen atmosphere. The reaction mixture is stirred for 16 hours at 60° C. The reaction mixture is poured onto ice and saturated potassium carbonate solution, extracted with diethyl ether (2×500 ml), washed with brine, and dried (anhydrous sodium sulphate). The organic extracts are removed under reduced pressure at 20° C. to give 73 g of crude 5-methoxy-3,4-dihydro-2H-pyrrole as a light yellow color liquid. This compound is used in the next step without further purification. The NMR spectrum of the title compound is according to theory.

¹H NMR (CDCl₃, 300 MHZ): δ 3.80 (s, 3H), 3.66 (t, 2H), 2.48 (t, 2H), 2.08-1.95 (m, 2H).

### Step 2

### Synthesis of 7-Hydroxy-5-oxo-1,2,3,5-tetrahydro-indolizine-8-carboxylic acid ethyl ester

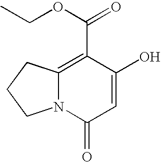

Triethylamine is added to a mixture of 5-methoxy-3,4-dihydro-2H-pyrrole (73 g, 0.73 mmol) and 3-oxopentanedioic acid diethyl ester (200 g, 0.99 mmol) at room temperature. The resulting solution is stirred for 5 days after which the reaction mixture is filtered to give 39 g (24% yield) of 7-hydroxy-5-oxo-1,2,3,5-tetrahydro-indolizine-8-carboxylic acid ethyl ester as a white solid. The NMR spectrum of the title compound is according to theory.

¹H NMR (CDCl₃, 300 MHZ): δ 11.4 (s, 1H), 5.80 (s, 1H), 4.40 (q, 2H), 4.15 (t, 2H), 3.50 (t, 2H), 2.3-2.15 (m, 2H), 1.40 (t, 3H).

### Step 3

### Synthesis of 5-Oxo-7-trifluoromethanesulfonyloxy-1,2,3,5-tetrahydro-indolizine-8-carboxylic acid ethyl ester

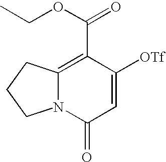

A stirred solution of 7-hydroxy-5-oxo-1,2,3,5-tetrahydro-indolizine-8-carboxylic acid ethyl ester (60 mg, 0.2 mmol) and triethylamine (30 mg, 0.4 mmol) in 5 ml of dichloromethane is cooled to −78° C. Triflic anhydride (91 mg, 0.32 mmol) is then added dropwise over 20 minutes and the resulting reaction mixture is stirred for 12 hours at ambient temperature with TLC monitoring (100% EtOAc). The reaction mixture is washed with aqueous sodium bicarbonate solution (4 ml) and water (4 ml). The organic layer is dried over anhydrous Na₂SO₄, concentrated and the resulting product is purified via column chromatography on silica gel (60-120 mesh) using 15% ethyl acetate in hexane as eluant to afford 40 mg (48% yield) of the title compound.

LC-MS purity: 95%, m/z 356 (M+1).

¹H NMR (CDCl₃, 300 MHZ): δ 6.15 (s, 1H), 4.40 (q, 2H), 4.20 (t, 2H), 3.58 (t, 2H), 2.32-2.2 (m, 2H), 1.40 (t, 3H).

### Step 4

### Synthesis of 7-(4-Bromo-2-fluoro-phenylamino)-5-oxo-1,2,3,5-tetrahydro-indolizine-8-carboxylic acid ethyl ester

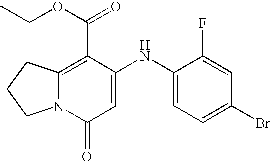

A stirred suspension of 5-oxo-7-trifluoromethanesulfonyloxy-1,2,3,5-tetrahydro-indolizine-8-carboxylic acid ethyl ester (2.3 g, 6.4 mmol), 2-fluoro-4-bromo-aniline (1.25 g, 6.5 mmol), cesium carbonate (3.17 g, 9.7 mmol), BINAP (0.6 g, 0.97 mmol), and Pd(OAc)₂ (0.15 g, 0.64 mmol) in toluene (100 ml) is heated at 80° C. for 16 hours. The reaction is monitored by the TLC (9:1 CHCl₃-MeOH v/v). The reaction mixture is diluted with ethyl acetate (60 ml) and filtered. The filtrate is washed with water (100 ml) and the aqueous layer is re-extracted with ethyl acetate (30 ml). The combined organic extracts are dried (anhydrous Na₂SO₄), concentrated, and the crude product is purified by column chromatography on silica gel (60-120 mesh) using 0.1-0.5% MeOH in chloroform to afford 336 mg (13% yield) of the title compound.

LC-MS purity: 98%, m/z 395, 397 (M+, Br pattern).

¹H NMR (DMSO-D₆, 300 MHZ): δ 9.48 (s, 1H), 7.70 (d, 1H), 7.49-7.39 (m, 2H), 5.32 (s, 1H), 4.30 (q, 2H), 3.95 (t, 2H), 3.48 (t, 2H), 2.14-2.0 (m, 2H), 1.30 (t, 3H).

### EXAMPLE 2

### Synthesis of 7-(4-Bromo-2-fluoro-phenylamino)-5-oxo-1,2,3,5-tetrahydro-indolizine-8-carboxylic acid

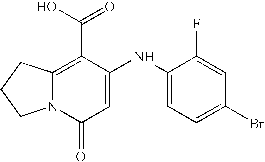

To the solution of 7-(4-bromo-2-fluoro-phenylamino)-5-oxo-1,2,3,5-tetrahydro-indolizine-8-carboxylic acid ethyl ester (280 mg, 0.71 mmol) in, THF:MeOH (4:1, v/v, 6 ml), is added, 1N aqueous LiOH solution (2 ml). The resulting mixture is stirred for 3 hours at room temperature with TLC monitoring (CHCl₃-MeOH, 8:2). The pH of the reaction mixture is adjusted to 1 with 10% aqueous HCl solution and the resulting precipitate is filtered, washed with water (20 ml) and ethyl acetate (10 ml) to afford 240 mg (92% yield) of the title compound.

LC-MS purity: 96%, m/z 367, 369 (M+, Br pattern).

¹H NMR (DMSO-D₆, 300 MHZ): δ 13.40 (s, 1H), 9.90 (s, 1H) 7.70 (d, 1H), 7.45 (s, 2H), 5.35 (s, 1H), 3.95 (t, 2H), 3.49 (t, 2H), 2.15-2.0 (m, 2H).

### EXAMPLE 3

### Synthesis of 7-(4-Bromo-2-fluoro-phenylamino)-5-oxo-1,2,3,5-tetrahydro-indolizine-8-carboxylic acid cyclopropyl-methoxy-amide

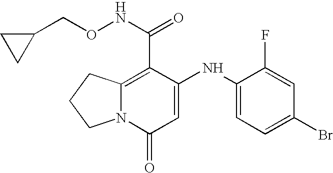

To a stirred solution of 7-(4-bromo-2-fluoro-phenylamino)-5-oxo-1,2,3,5-tetrahydro-indolizine-8-carboxylic acid (140 mg, 0.38 mmol) in 10 ml of dry DMF are added EDCI (220 mg, 1.14 mmol) and HOBt (160 mg, 1.14 mmol). The reaction mixture is stirred for 30 minutes and then treated with O-cyclopropylmethylhydroxylamine (141 mg, 1.14 mmol) and TEA (232 mg, 1.14 mmol). The resulting reaction mixture is stirred for 16 hours with TLC monitoring (MeOH—CHCl₃ 2:8, v/v). The reaction mixture is diluted with ethyl acetate (20 ml) and washed with saturated aqueous NH₄Cl solution (25 ml), saturated aqueous NaHCO₃ solution (25 ml), and brine (25 ml). The combined organic extracts are dried (anhydrous Na₂SO₄) and concentrated. The residual material is purified by column chromatography on silica gel (1% MeOH in CHCl₃) to afford the title compound in 36% yield.

LC-MS purity: 97%, m/z 436, 438 (M+, Br Pattern).

¹H NMR (DMSO-D₆, 300 MHZ): δ 11.20 (s, 1H), 8.25 (s, 1 μl), 7.65 (d, 1H), 7.45-7.3 (m, 2H), 5.38 (s, 1H), 3.91 (t, 2H), 3.72 (d, 2H), 3.25 (t 2H), 2.15-2.0 (m, 2H), 1.18-1.02 (m, 1H), 0.6-0.5 (m, 2H), 0.31-0.25 (m, 2H).

### EXAMPLE 4

### Synthesis of 7-(4-bromo 2-fluoro-phenylamino)-5-oxo-1,2,3,5-tetrahydro indolizine 8-carboxylic acid methoxyl amide

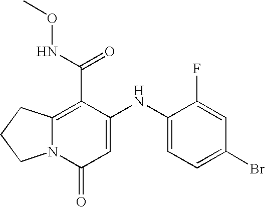

Using the same reaction conditions as in Example 3,7-(4-bromo 2-fluoro-phenylamino)-5-oxo-1,2,3,5-tetrahydro indolizine 8-carboxylic acid (0.2 g, 0.544 mmol) is reacted with methoxylamine hydrochloride (136 mg, 1.63 mmol) to yield the crude product. Purification of the product by column chromatography on silica gel (4% methanol in CHCl₃) and then preparative HPLC gives the title compound in 16% yield.

LC-MS purity: 99.27%, m/z=396, 398.9, (M⁺ Br pattern).

H¹ NMR: (DMSO-D₆ 300 MHZ): 11.4 (s, 1H), 8.4 (s, 1H), 7.7-7.6 (m, 1H), 7.5-7.3 (m, 2H), 7.223 (t, 1H), 5.3 (s, 1H), 3.9 (t, 2H), 3.3-3.1 (m, 4H), 3.0 (s, 3H), 2.1 (t, 2H).

### EXAMPLE 5

### Synthesis of 7-(4-bromo 2-fluoro-phenylamino)-5-oxo-1,2,3,5-tetrahydro indolizine 8-carboxylic acid amide

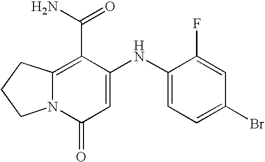

Using the same reaction conditions as in Example 3,7-(4-bromo 2-fluoro-phenylamino)-5-oxo-1,2,3,5-tetrahydro indolizine 8-carboxylic acid (0.2 g, 0.544 mmol) is converted to the title compound with ammonium chloride and triethyl amine. The test compound is obtained as a white solid in 30% yield, following purification by column chromatography on silica gel (5% methanol in CHCl₃).

LC-MS purity: 98.74%, m/z=366, 368, (M⁺ Br pattern)

H¹ NMR: (DMSO-D₆ 300 MHZ): 9.0 (s, 1H), 7.7-7.6 (m, 2H), 7.4 (t, 2H), 5.3 (s, 1H), 3.9 (t, 2H), 3.2-3.1 (m, 2H), 3.0 (s, 3H), 2.1 (t, 2H)

### EXAMPLE 6

### Synthesis of 7-(4-bromo 2-fluoro-phenylamino)-5-oxo-1,2,3,5-tetrahydro indolizine 8-carboxylic acid ethoxy amide

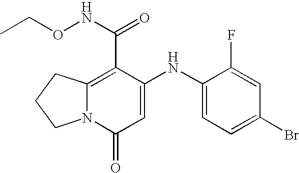

Using the same reaction conditions as in Example 3,7-(4-bromo 2-fluoro-phenylamino)-5-oxo-1,2,3,5-tetrahydro indolizine 8-carboxylic acid (0.2 g, 0.54 mmol) is converted with ethoxylamine hydrochloride to the title compound. Purification of the product by column chromatography on silica gel (5% methanol in CHCl₃), followed by preparative HPLC affords the test compound as a white solid in 28% yield.

LC-MS purity: 99.27%, m/z=410, 412, (M⁺ Br pattern).

H¹ NMR: (DMSO-D₆ 300 MHZ): 11.4 (s, 1H), 8.3 (s, 1H), 7.7-7.5 (m, 1H), 7.5-7.3 (m, 2H), 5.3 (s, 1H), 4.0-3.8 (m, 4H), 3.4-3.2 (m, 2H), 2.1 (t, 2H), 1.2 (t, 3H).

### EXAMPLE 7

### Synthesis of 7-(4-Bromo-2-fluoro-phenylamino)-5-oxo-1,2,3,5-tetrahydro-indolizine-8-carbaldehyde

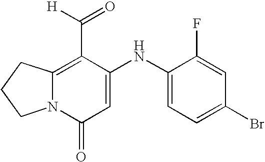

To a solution of 7-(4-bromo-2-fluoro-phenylamino)-5-oxo-1,2,3,5-tetrahydro-indolizine-8-carboxylic acid methoxymethylamide (0.1 g, 0.25 mmol) in 5 ml dry THF under nitrogen is added dropwise 0.76 ml (0.76 mmol) of DIBAH-H (1.0 M solution in diethyl ether) over a period of 5 minutes at −78 C.°. The reaction mixture is stirred at −78 C.° for 4 hr and quenched with a saturated aqueous ammonium chloride solution (10 ml). The reaction mixture is extracted with ethyl acetate and the combined organic extracts are washed with water and brine, then dried (anhydrous sodium sulfate) and concentrated. The title compound is obtained in 41% yield, following purification by column chromatography on silica gel (2% methanol in CH₂Cl₂).

LC-MS purity: 90%, m/z=350, (M+2 Br pattern)

HPLC: 92%

H¹ NMR: (DMSO-D₆, 300 MHZ): δ 10.0 (s, 1H), 9.56 (s, 1H), 7.54 (d, 1H), 7.50 (s, 2H), 5.35 (s, 1H), 4.0 (t, 2H), 3.55 (t, 2H), 2.4-2.2 (m, 2H).

### EXAMPLE 8

### Step 1

### Synthesis of 7-Chloro-5-oxo-1,2,3,5-tetrahydro-indolizine-8-carboxylic acid ethyl ester

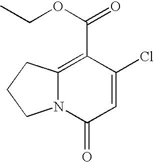

To a suspension of 7-hydroxy-5-oxo-1,2,3,5-tetrahydro-indolizine-8-carboxylic acid ethyl ester (500 mg, 2.2 mmol) in POCl₃ (2 g, 13.4 mmol) is added TEA (0.226 g, 2.2 mmol) and the reaction mixture is stirred for 14 hours. The reaction mass is poured into ice water and the pH of the mixture is adjusted to 7 with an aqueous K₂CO₃ solution. The reaction mixture is extracted with EtOAc and the combined organic extracts are dried (anhydrous Na₂SO₄) and concentrated. The title compound is obtained in 74% yield, following purification by column chromatography on silica gel (1:1 EtOAc-hexane, v/v).

LC-MS purity: 100%, m/z=242, (M+)

¹H NMR (CDCl₃, 300 MHZ): 6.60 (s, 1H), 4.40 (q, 2H), 4.12 (t, 2H), 3.50 (t, 2H), 2.32-2.19 (m, 2H), 1.40 (t, 3H).

### Step 2

### Synthesis of 7-Chloro-6-fluoro-5-oxo-1,2,3,5-tetrahydro-indolizine-8-carboxylic acid ethyl ester

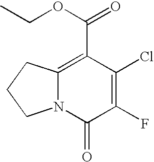

A mixture of 7-chloro-5-oxo-1,2,3,5-tetrahydro-indolizine-8-carboxylic acid ethyl ester (14.5 g, 0.06 mol) and 1-(chloromethyl)-4-fluoro-1,4-diazoniabicyclo[2.2.2]octane-bis(tetrafluoroborate) (44.7 g, 0.13 mol) in 700 ml of CH₃CN is heated at 80° C. with stirring for 5-10 minutes; the heat source is then removed. The volatile components are removed under reduced pressure and the residual material is dissolved in water and extracted with EtOAc. The combined organic extracts are dried (anhydrous Na₂SO₄) and concentrated to give 14 g of the crude product. The title compound is obtained in 33% yield after column chromatography on silica gel (40% EtOAc in hexane) and is used in the next step without further purification.

LC-MS purity: 87%, m/z=260, (M+).

### Step 3

### Synthesis of 7-Chloro-6-fluoro-5-oxo-1,2,3,5-tetrahydro-indolizine-8-carboxylic acid

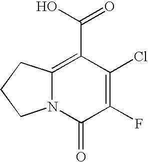

To a solution of 120 ml of THF:MeOH (4:1, v/v) containing 5.2 g of 7-chloro-6-fluoro-5-oxo-1,2,3,5-tetrahydro-indolizine-8-carboxylic acid ethyl ester (0.02 mol) is added 30 ml of 1N aqueous LiOH solution and the whole is stirred at ambient temperature for 3 hours. The pH of the reaction mixture is adjusted to 1 and it is extracted with EtOAc. The combined organic extracts are dried (anhydrous Na₂SO₄) and concentrated to yield the crude product. Trituration with EtOAc gives the title compound in 78% yield as a gray solid.

LC-MS purity: 95%, m/z=232, (M+)

¹H NMR (DMSO-D₆, 300 MHZ): 13.4 (s, 1H), s 4.08 (t, 2H), 3.30 (t, 2H), 2.18-2.06 (m, 2H).

### Step 4

### Synthesis of 6-Fluoro-7-(2-fluoro-4-iodo-phenylamino)-5-oxo-1,2,3,1-tetrahydro-indolizine-8-carboxylic acid

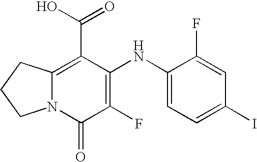

A stirred solution of 2-fluoro-4-iodo-phenylamine (6.4 g, 0.027 mol) in 60 ml THF is treated with LDA (4 g, 0.037 mol) at −78° C. under nitrogen. After 20 minutes, a solution of THF (330 ml) containing 7-chloro-6-fluoro-5-oxo-1,2,3,5-tetrahydro-indolizine-8-carboxylic acid (2.5 g, 0.011 mol) is added. The reaction mixture is stirred for 30 minutes more at −78° C. and then allowed to warm to ambient temperature. The reaction mixture is stirred for 3 days and the volatile components are removed under reduced pressure. The residual material is partitioned between 50 ml of 3N HCl solution and 50 ml of diethyl ether. After stirring for 15 minutes, the resulting solids are collected to give a 72% yield of the title compound as a light brown solid

LC-MS purity: 96%, m/z=433, (M+)

¹H NMR (DMSO-D₆, 300 MHZ): δ 13.8-13.6 (br s, 1H), 9.42 (s, 1H), 7.62 (d, 1H), 7.48 (d, 1H), 6.9-6.8 (m, 1H), 4.04 (t 2H), 3.46 (t 2H), 2.18-2.04 (m, 2H).

### EXAMPLE 9

### Synthesis of 6-Fluoro-7-(2-fluoro-4-iodo-phenylamino)-5-oxo-1,2,3,5-tetrahydro-indolizine-8-carboxylic acid cyclopropylmethoxy-amide

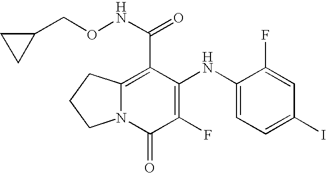

Using the same reaction conditions as in Example 3, a mixture of 6-fluoro-7-(2-fluoro-4-iodo-phenylamino)-5-oxo-1,2,3,5-tetrahydro-indolizine-8-carboxylic acid (100 mg, 0.23 mmol), EDCI (133 mg, 0.69 mmol), and HOBt (93 mg, 0.69 mmol) in 6 ml of dry DMF is stirred for 30 minutes and combined with O-cyclopropylmethyl-hydroxylamine (86 mg, 0.69 mmol) and TEA (70 mg, 0.69 mmol) to yield the title compound as a gray solid in 34% yield after column chromatography on silica gel (0-2% MeOH in CHCl₃).

LC-MS purity: 91%, m/z 500, (M−)

¹H NMR (DMSO-D₆, 300 MHZ): δ 11.40 (s, 1H), 8.08 (s, 1H), 7.58 (d, 1H), 7.42 (d, 1H), 6.86-6.76 (m, 1H), 4.00 (t, 2H), 3.52 (d, 2H), 3.18 (t, 2H), 2.2-2.0 (m, 2H), 1.08-0.98 (m, 1H), 0.57-0.47 (m, 2H), 0.27-0.19 (m, 2H).

### EXAMPLE 10

### Synthesis of 6-Fluoro-7-(2-fluoro-4-iodo-phenylamino)-5-oxo-1,2,3,5-tetrahydro-indolizine-8-carboxylic acid amide

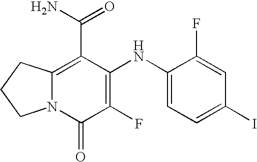

Using the same reaction conditions and reagents as described in Example 5, 6-fluoro-7-(2-fluoro-4-iodo-phenylamino)-5-oxo-1,2,3,5-tetrahydro-indolizine-8-carboxylic acid (300 mg, 0.69 mmol) is converted to 200 mg of the crude title compound. Trituration with EtOAc and diethyl ether gives the test compound in 40% yield as a pale yellow solid.

LC-MS purity: 96%, m/z 432, (M+)

¹H NMR (DMSO-D₆, 300 MHZ): δ 8.58 (s, 1H), 7.75 (d, 2H), 7.60 (d, 1H), 7.45 (d, 1H) 6.82-6.7 (m, 1H), 4.02 (t, 2H), 3.3 (t, 2H), 2.2-2.08 (m, 2H).

### EXAMPLE 11

### Synthesis of 7-(4-Bromo-2-fluoro-phenylamino)-6-fluoro-5-oxo-1,2,3,5-tetrahydro-indolizine-8-carboxylic acid

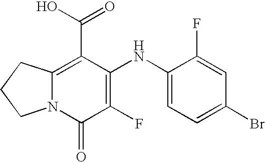

Using the identical reaction conditions and reagents as those in Example 8, Step 4,4-bromo-2-fluoro-phenylamine (925 mg, 4.8 mmol) is combined with 7-chloro-6-fluoro-5-oxo-1,2,3,5-tetrahydro-indolizine-8-carboxylic acid (450 mg, 1.94 mol) to give the title compound in 60% yield as a light brown solid.

LC-MS purity: 94%, m/z=385, 387 (M+, Br Pattern)

¹H NMR (DMSO-D₆, 300 MHZ): δ 13.9-13.80 (br s, 1H), 9.44 (s, 1H), 7.56 (d, 1H), 7.34 (d, 1H), 7.18-6.9 (m, 1H), 4.04 (t, 2H), 3.48 (t, 2H), 2.18-2.05 (m, 2H).

### EXAMPLE 12

### Synthesis of 7-(4-Bromo-2-fluoro-phenylamino)-6-fluoro-5-oxo-1,2,3,5-tetrahydro-indolizine-8-carboxylic acid cyclopropylmethoxy-amide

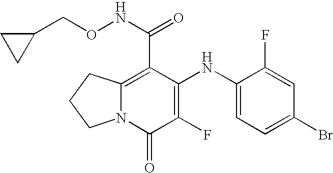

Using identical reaction conditions and reagents as to those in Example 9,7-(4-bromo-2-fluoro-phenylamino)-6-fluoro-5-oxo-1,2,3,5-tetrahydro-indolizine-8-carboxylic acid (100 mg, 0.25 mmol) is converted to the title compound as a white solid in 32% yield following silica gel chromatography (0-2% MeOH in CHCl₃).

LC-MS purity: 98%, m/z=454, 456 (M+, Br pattern).

¹H NMR (DMSO-D₆, 300 MHZ): δ 11.40 (s, 1H), 8.06 (s, 1H), 7.54 (d, 1H), 7.28 (d, 1H), 7.04-6.94 (m, 1H), 4.00 (t, 2H), 3.52 (d, 2H), 3.20 (t, 2H), 2.18-2.04 (m, 2H), 1.08-0.98 (m, 1H), 0.55-0.45 (m, 2H), 0.28-0.19 (, 2H).

### EXAMPLE 13

### Synthesis of 7-(4-Bromo-2-fluoro-phenylamino)-6-fluoro-5-oxo-1,2,3,5-tetrahydro-indolizine-8-carboxylic acid amide

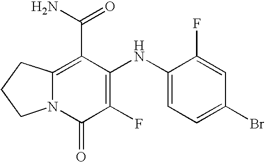

Using the same reaction conditions and reagents as described in Example 5,7-(4-bromo-2-fluoro-phenylamino)-6-fluoro-5-oxo-1,2,3,5-tetrahydro-indolizine-8-carboxylic acid (250 mg, 0.64 mmol)) is converted to 140 mg of the crude title compound. Trituration with EtOAc and diethyl ether gives the test compound in 25% yield as a light yellow solid.

LC-MS purity: 99%, m/z 384, 386 (M+, Br Pattern).

¹H NMR (DMSO-D₆, 300 MHZ): δ 8.58 (s, 1H), 7.74 (d, 2H), 7.54 (d, 1H), 7.29 (d, 1H) 7.0-6.9 (m, 1H), 4.00 (t, 2H), 3.3 (t, 2H), 2.19-2.08 (m, 2H).

### EXAMPLE 14

### Step 1

### Synthesis of 7-Hydroxy-6-methyl-5-oxo-1,2,3,5-tetrahydro-indolizine-8-carboxylic acid ethyl ester

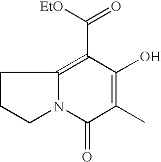

A solution of 7-hydroxy-5-oxo-1,2,3,5-tetrahydro-indolizine-8-carboxylic acid ethyl ester (7.5 g, 33.6 mmol) in THF is added dropwise at 0° C. to a stirred suspension of sodium hydride in THF under an inert atmosphere. The reaction mixture is allowed to warm to ambient temperature and is then treated with iodomethane. The resulting reaction mixture is stirred for 2 days (TLC monitoring, 100% EtOAc) and then is quenched with ice. The volatiles are removed under reduced pressure and the remaining aqueous phase is extracted with ethyl acetate. The combined organic extracts are washed with brine and concentrated. Column chromatography of the crude product on silica gel (70% ethyl acetate in hexane) yields 38% of the title compound.

¹H NMR (DMSO-D₆): δ 11.5 (s, 1H), 4.3 (q, 2H), 4.0 (t, 2H), 3.48 (t, 2H), 2.1 (q, 2H), 1.8, (s, 3H), 1.3 (t, 3H).

### Step-2

### Synthesis of 6-Methyl-5-oxo-7-trifluoro methanesulfonyloxy-1,2,3,5-tetrahydro-indolizine-8-carboxylic acid ethyl ester

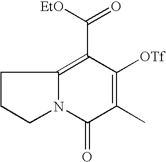

Using reaction conditions identical to those in Example 1, Step 3,7-hydroxy-6-methyl-5-oxo-1,2,3,5-tetrahydro-indolizine-8-carboxylic acid ethyl ester (0.75 g, 3.16 mmol) is converted to the title compound with triflic anhydride (1.06 g, 3.79 mmol) after stirring for 12 hours at ambient temperature. The test compound is obtained in 40% yield after silica gel column chromatography (25% ethyl acetate in hexane).

¹H NMR (DMSO-D₆): 4.40 (q, 2H), 4.20 (t, 2H), 3.5 (t 2H), 2.3-2.2 (m, 2H), 2.05 (s, 3H), 1.35 (t, 3H).

### Step 3

### Synthesis of 7-(4-Bromo-2-fluoro-phenylamino)-6-methyl-5-oxo-1,2,3,5-tetrahydro-indolizine-8-carboxylic acid ethyl ester

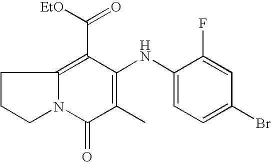

A stirred solution of toluene (100 ml) containing 6-methyl 5-oxo-7-trifluoromethanesulfonyloxy-1,2,3,5-tetrahydro-indolizine-8-carboxylic acid ethyl ester (0.47 g, 1.26 mmol), 2-fluoro-4-bromo-aniline (0.287 g, 1.26 mmol), cesium carbonate (0.617 g, 1.89 mmol), BINAP (0.6 g, 0.19 mmol), and Pd(OAc)₂ (0.028 g, 0.126 mmol) is heated for 4 hours at 90° C. The reaction mixture is filtered and the filtrate is concentrated. The residual material is taken up in ethyl acetate and washed twice with brine, then dried (anhydrous Na₂SO₄) and concentrated. Column chromatography of the crude product on silica gel (75% ethyl acetate in hexane) affords the title compound in 19% yield.

LC-MS purity: 96.24%, m/z 409, (M+, Br pattern).

¹H NMR (DMSO-D₆): δ 8.5 (s, 1H), 7.50 (d, 1H), 7.25 (d, 1H), 6.6 (t, 1H), 4.2-4.0 (m, 4H), 3.45 (d, 2H), 2.2-2.1 (m, 2H), 1.70 (t, 3H), 1.3 (t 3H).

### EXAMPLE 15

### Synthesis of 7-(4-Bromo-2-fluoro-phenylamino)-6-methyl-5-oxo-1,2,3,1-tetrahydro-indolizine-8-carboxylic acid

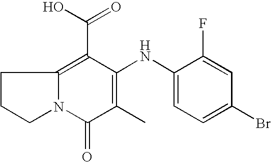

An aqueous NaOH solution (1 ml, 1 N) is added to 7-(4-bromo-2-fluoro-phenylamino)-6-methyl-5-oxo-1,2,3,5-tetrahydro-indolizine-8-carboxylic acid ethyl ester (40 mg, 0.10 mmol) in a solvent mixture of THF:MeOH (3:1, v/v). The resulting reaction mixture is stirred for 3 hours at room temperature. The pH of the reaction mixture is adjusted to 1.5 with 1N aqueous HCl solution and the resulting precipitate is filtered, washed with water (20 ml) and ethyl acetate (10 ml) to afford the title compound in 53% yield.

LC-MS purity: 99.2%, m/z 381 (M+, Br pattern).

¹H NMR (DMSO-D₆): δ 13.30 (s, 1H), 9.10 (s, 1H) 7.5 (d, 1H) 7.20 (d, 1H), 6.5 (s, 1H), 4.05 (t, 2H), 3.5 (t, 2H), 2.10 (t, 2H), 1.6 (s, 3H).

### EXAMPLE 16

### Synthesis of 7-(4-Bromo-2-fluoro-phenylamino)-6-methyl-5-oxo-1,2,3,1-tetrahydro-indolizine-8-carboxylic acid cyclopropylmethoxy-amide

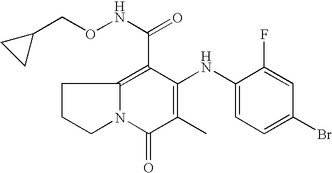

Using identical reaction conditions and reagents as in Example 9,7-(4-bromo-2-fluoro-phenylamino)-6-methyl-5-oxo-1,2,3,5-tetrahydro-indolizine-8-carboxylic acid (300 mg, 0.787 mmol) is converted to the title compound with O-cyclopropylmethyl hydroxylamine in 8% yield, following purification by silica gel chromatography (1% MeOH in CHCl₃).

LC-MS purity: 92.7%, m/z 450 (M+, Br Pattern).

¹H NMR (DMSO-D₆): δ 11.20 (s, 1H), 7.7 (s, 1H), 7.5 (d, 1H), 7.2 (d, 1H), 6.56 (t, 1H), 4.0 (t, 2H), 3.5 (d, 2H), 3.2 (t 2H), 2.18 (q, 2H), 1.78 (s, 3H), 1.0 (s, 1H), 0.5 (d, 2H), 0.2 (d, 2H).

### EXAMPLE 17

### Step 1

### Synthesis of 7-(4-Bromo-2-fluoro-phenylamino)-6-methyl-5-oxo-1,2,3,1-tetrahydro-indolizine-8-carboxylic acid (2-vinyloxy-ethoxy)-amide

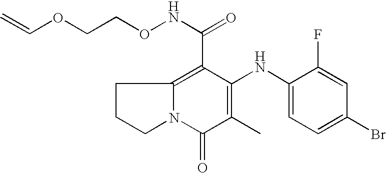

To a stirred solution of 7-(4-bromo-2-fluoro-phenylamino)-6-methyl-5-oxo-1,2,3,5-tetrahydro-indolizine-8-carboxylic acid (300 mg, 0.787 mmol) in 6 ml of dry DMF and 2 ml of dichloromethane is added EDCI (165 mg, 0.865 mmol) and HOBt (116 mg, 0.865 mmol) at 0° C. The resulting reaction mixture is stirred for 2 hrs at 0° C. and is then treated in succession with O-(2-vinyloxy-ethyl)-hydroxylamine (71 mg, 0.787 mmol) and TEA (158 mg, 1.57 mmol). After 12 hours, the reaction mixture is diluted with ethyl acetate and washed with saturated aqueous NaHCO₃ solution. The organic phase is dried (anhydrous Na₂SO₄) and concentrated under reduced pressure. The residual material is chromatographed on silica gel (1% MeOH in CHCl₃) to give 180 mg of the title compound in low purity.

LC MS: 31.7%, m/z=466, (M+Br pattern)

### Step 2

### Synthesis of 7-(4-Bromo-2-fluoro-phenylamino)-6-methyl-5-oxo-1,3,5-tetrahydro-indolizine-8-carboxylic acid (2-hydroxy-ethoxy)-amide

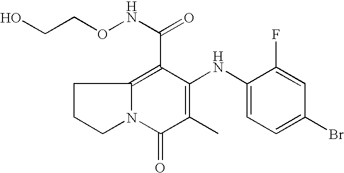

Crude 7-(4-bromo-2-fluoro-phenylamino)-6-fluoro-5-oxo-1,2,3,5-tetrahydro-indolizine-8-carboxylic acid (2-vinyloxy-ethoxy)-amide (180 mg, 0.386 mmol) is dissolved in ethanol containing 1 ml of 1N HCl and stirred for 16 hours at room temperature. The reaction mixture is concentrated and the residue is dissolved in ethyl acetate. The organic phase is washed with brine, concentrated, and the residue is column chromatographed on silica gel (2% MeOH in CHCl₃) to afford the title compound in 30% yield.

LC MS: 96.5%, m/z=440, (M+ Br pattern)

HPLC: 94.05%

¹H NMR (DMSO-D₆): δ 11.20 (s, 1H), 7.65 (s, 1H), 7.48 (d, 1H), 7.18 (d, 1H), 6.5 (t, 1H), 4.0 (t, 2H), 3.7 (t, 2H), 3.5 (s, 3H), 3.2 (t, 2H), 2.14 (quin, 2H), 1.72 (s, 3H).

### EXAMPLE 18

### Synthesis of 7-(4-Bromo-2-fluoro-phenylamino)-6-methyl-5-oxo-1,2,3,5-tetrahydro-indolizine-8-carboxylic acid amide

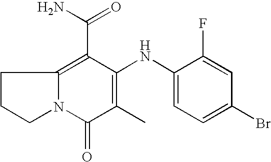

To a solution of 7-(4-bromo-2-fluoro-phenylamino)-6-methyl-5-oxo-1,2,3,5-tetrahydro-indolizine-8-carboxylic acid (200 mg, 0.524 mmol) in 6 ml THF is added a THF solution containing TBTU (292 mg 0.787 mmol) and TEA (79 mg, 0.787 mmol) at 0° C. The reaction mixture is allowed to slowly warm to room temperature and is then stirred for one hour. Ammonium chloride is added and the reaction mixture is stirred at ambient temperature for 12 hours more. The reaction mixture is concentrated and the residue is dissolved in ethyl acetate. The organic phase is washed with aqueous NaHCO₃ solution and concentrated. The title compound is obtained in 25% yield after column chromatography on neutral alumina (2% MeOH in CHCl₃).

LC MS: 93.7%, m/z=380, (M+ Br pattern).

HPLC: 98.7%

¹H NMR (DMSO-D₆): δ 7.7 (s, 1H), 7.5 (dd, 1H), 7.2 (d, 1H), 6.5 (t, 1H), 4.0 (t, 2H), 3.3 (s, 2H), 2.1 (quin, 2H), 1.78 (s, 3H).

### EXAMPLE 19

### Synthesis of 7-(4-Bromo-2-fluoro-phenylamino)-6-methyl-5-oxo-1,2,3,5-tetrahydro-indolizine-8-carboxylic acid methoxy-amide

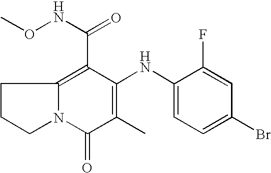

Using the same reaction conditions and reagents as in Example 4,7-(4-bromo-2-fluoro-phenylamino)-6-methyl-5-oxo-1,2,3,5-tetrahydro-indolizine-8-carboxylic acid (200 mg, 0.525 mmol) is transformed to the title compound with methoxylamine hydrochloride (45 mg, 0.55 mmol). The test compound is obtained in 19% yield via column chromatography on silica (1% MeOH in CHCl₃).

LC-MS purity: 96.42%, m/z=410, (M+Br pattern).

HPLC: 97.8%

¹H NMR (DMSO-D₆): δ 11.20 (s, 1H), 7.7 (s, 1H), 7.5-7.4 (dd, 1H), 7.2 (d, 1H), 6.5 (t, 1H), 4.0 (t, 2H), 3.5 (s, 3H), 3.2 (t, 2H), 2.1 (quin, 2H), 1.78 (s, 3H).

### EXAMPLE 20

### Synthesis of 7-(4-Bromo-2-fluoro-phenylamino)-6-methyl-5-oxo-1,2,3,5-tetrahydro-indolizine-8-carboxylic acid ethoxy-amide

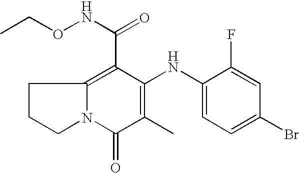

Using the same reaction conditions as in Example 3,7-(4-bromo-2-fluoro-phenylamino)-6-methyl-5-oxo-1,2,3,5-tetrahydro-indolizine-8-carboxylic acid (200 mg, 0.523 mmol) is reacted with ethoxylamine hydrochloride (53 mg, 0.55 mmol) to yield the title compound. The test sample is obtained in 23% yield following column chromatography on silica gel (1% MeOH in CHCl₃).

LC-MS purity: 95.58%, m/z=424, (M+ Br pattern)

HPLC: 98.61%

¹H NMR (DMSO-D₆): δ 11.20 (s, 1H), 7.7 (s, 1H), 7.5-7.4 (dd, 1H), 7.2 (d, 1H) 6.5 (t, 1H), 4.0 (t, 2H), 3.7 (q, 2H), 3.2 (t, 2H), 2.1 (quin, 2H), 1.78 (s, 3H), 1.1 (t, 3H).

### EXAMPLE 21

### Synthesis of 2-(4-bromo-2-fluoro-phenyl-amino)-4-oxo-6,7,8,9-tetrahydro-4H-quinolizine-1-carboxylic acid-cyclopropyl-methoxyamide

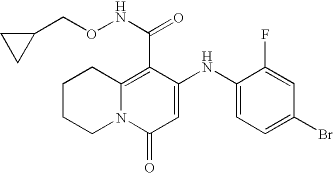

Following the procedure set forth in Example 3, 150 mg of 2-(4-bromo-2-fluoro-phenylamino)-4-oxo-6,7,8,9-tetrahydro-4H-quinolizine-1-carboxylic acid (0.39 mmol) is transformed to the title compound with O-cyclopropylmethylhydroxylamine. The title compound is obtained in 29% yield after column chromatography on silica gel (3% methanol in CHCl₃).

LC-MS purity: 99.3%, m/z=451, (M+ Br pattern)

¹H NMR (DMSO-D₆): δ 11.8 (s, 1H), 7.64 (d, 1H), 7.54 (s, 1H), 7.4 (d, 1H), 7.3 (t, 1H), 5.2 (s, 1H), 3.8 (t, 2H), 3.7 (d, 2H), 2.7 (t, 2H), 1.85-1.65 (m, 4H), 1.1-1.0 (m, 1H), 0.55-0.45 (m, 2H), 0.3-0.2 (m, 2H).

### EXAMPLE 22

### Step 1

### Synthesis of 2-(4-bromo-2-fluoro-phenylamino)-4-oxo-6,7,8,9-tetrahydro-4H-quinolizine-1-carboxylic acid-(2-vinyloxy-ethoxy)-amide

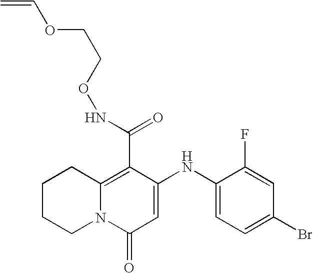

Using reaction conditions identical to those in Example 17, Step 1,2-(4-bromo-2-fluoro-phenylamino)-4-oxo-6,7,8,9-tetrahydro-4H-quinolizine-1-carboxylic acid (200 mg, 0.52 mmol) in 5 ml DMF is converted to the title compound with O-(2-vinyloxy-ethyl)-hydroxylamine (64 mg, 0.62 mmol). The purified product is obtained by column chromatography on silica gel (methanol:chloroform) in 62% yield.

### Step 2

### Synthesis of 2-(4-bromo-2-fluoro-phenylamino)-4-oxo-6,7,8,9-tetrahydro-4H-quinolizine-1-carboxylic acid-(2-hydroxy-ethoxy)-amide

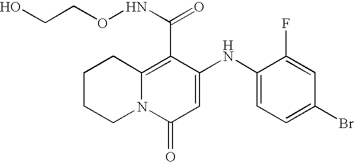

Using reaction conditions identical to those in Example 17, Step 2,2-(4-bromo-2-fluoro-phenylamino)-4-oxo-6,7,8,9-tetrahydro-4H-quinolizine-1-carboxylic acid-(2-vinyloxy-ethoxy)-amide (150 mg, 0.032 mmol) is transformed to the title compound which is obtained in 12% yield following preparative HPLC

LC-MS purity: 98.7%, m/z=441, (M+ Br pattern).

¹H NMR (DMSO-D₆): δ 11.6 (s, 1H), 7.68-7.58 (dd, 2H), 7.46-7.42 (dd, 1H), 7.3 (t, 1H), 5.2 (s, 1H), 4.85 (t, 1H), 4.0 (t, 2H), 3.8 (t, 2H), 3.6 (q, 2H), 2.7 (t, 2H), 1.85-1.65 (m, 4H).

### EXAMPLE 23

### Synthesis of 2-(4-bromo-2-fluoro-phenylamino)-4-oxo-6,7,8,9-tetrahydro-4H-quinolizine-1-carboxylic acid methoxy-amide

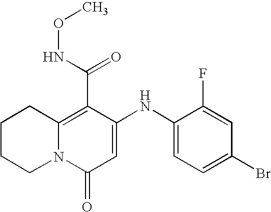

Using the same reaction conditions and reagents as in Example 5,2-(4-bromo-2-fluoro-phenylamino)-4-oxo-6,7,8,9-tetrahydro-4H-quinolizine-1-carboxylic acid (200 mg, 0.52 mmol) is transformed to the title compound with methoxylamine hydrochloride (131 mg, 1.52 mmol). Purification of the product by column chromatography on silica gel (3.5% MeOH in CHCl₃), followed by preparative HPLC, yields 25 mg of the test compound.

LC-MS purity: 96.4%, m/z=409.9, (M+Br pattern).

¹H NMR (DMSO-D₆): δ 11.65 (s, 1H), 7.64 (d, 1H), 7.59 (s, 1H), 7.4 (d, 1H), 7.3 (t, 1H), 5.2 (s, 1H), 3.8 (t, 2H), 3.7 (s, 3H), 2.7 (t, 2H), 1.85-1.65 (m, 4H).

### EXAMPLE 24

### Synthesis of 2-(4-bromo-2-fluoro-phenylamino)-4-oxo-6,7,8,9-tetrahydro-4H-quinolizine-1-carboxylic acid ethoxy-amide

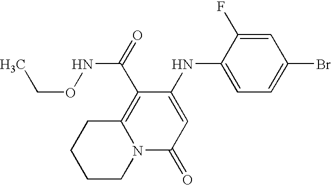

Using the same reaction conditions and reagents as in Example 3,2-(4-bromo-2-fluoro-phenylamino)-4-oxo-6,7,8,9-tetrahydro-4H-quinolizine-1-carboxylic acid (200 mg, 0.52 mmol) is reacted with ethoxylamine hydrochloride (153 mg, 1.5 mmol) to afford 55 mg (25% yield) of the title compound after preparative HPLC.

LC-MS purity: 97.9%, m/z=425.8, (M+Br pattern).

¹H NMR (DMSO-D₆): δ 11.6 (s, 1H), 7.64 (d, 1H), 7.52 (s, 1H), 7.4 (d, 1H), 7.3 (t, 1H), 5.2 (s, 1H), 4.0 (q, 2H), 3.8 (t, 2H), 2.7 (t 2H), 1.85-1.65 (m, 4H), 1.3-1.15 (m, 3H).

### EXAMPLE: 25

### Steps 1 to 4 were Performed in a Manner Similar to What has been Described for Example 1 and Step 5 was Performed in a Manner Similar to What has been Described for Example 2

### Step: 6

### Synthesis of 7-(4-Bromo-2-fluoro-phenylamino)-5-oxo-1,2,3,5-tetrahydro-indolizine-8-carboxylic acid (3-hydroxy-propyl)-amide

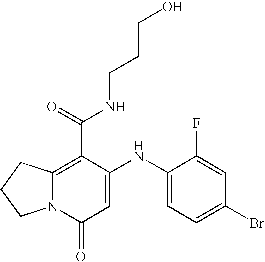

EDCI (390 mg, 0.002 mol) and HOBt (162 mg, 0.002 mol) were added to a stirred solution of 7-(4-bromo-2-fluoro-phenylamino)-5-oxo-1,2,3,5-tetrahydro indolizine-8-carboxylic acid (250 mg, 0.001 mol) in DMF (6 mL) at 0° C. The reaction mixture was stirred for 1 hr at 0° C. To this were added 3-amino-propanol (0.156 ml, 0.002 mol), followed by TEA (1 mL, 0.012 mol). The reaction mixture was stirred overnight at RT. The reaction mixture was partitioned between ethyl acetate and cold water (20 mL). The organic layer was washed with NaHCO₃ solution and concentrated. Purification by preparative HPLC affords 41 mg (14.13% yield) of 7-(4-bromo-2-fluoro-phenylamino)-5-oxo-1,2,3,5-tetrahydro-indolizine-8-carboxylic acid (3-hydroxy-propyl)-amide.

LC-MS purity: 97.2%, m/z 426, 428 (M+Br pattern)

H¹ NMR (DMSO-D₆, 300 MHZ) δ 8.68 (s, 1H), 8.28 (t, 1H), 7.7-7.6 (m, 1H), 7.5-7.3 (m, 2H), 5.49 (s, 1H), 3.9 (t, 2H), 3.5 (t, 2H), 3.4-3.1 (m, 4H), 2.1 (quin, 2H), 1.7-1.5 (q, 2)

### EXAMPLE: 26

### Steps 1 to 4 were Performed in a Manner Similar to What has been Described for Example 8

### Step: 5

### Synthesis of 6-Fluoro-7-(2-fluoro-4-iodo-phenylamino)-5-oxo-1,2,3,1-tetrahydro-indolizine-8-carboxylic acid (2-vinyloxy-ethoxy)-amide

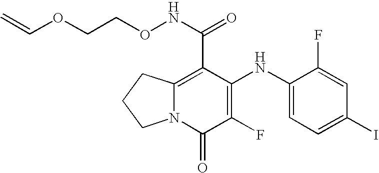

EDCI (530 mg, 0.003 mol) and HOBt (364 mg, 0.003 mol) were added to a stirred solution of 6-fluoro-7-(2-fluoro-4-iodo-phenylamino)-5-oxo-1,2,3,5-tetrahydro-indolizine-8-carboxylic acid (200 mg, 0.46 mmol) in DMF (20 mL) and DCM (10 mL) at 0° C. The reaction mixture was stirred for 2 hrs at 0° C. O-(2-vinyloxy-ethyl)-hydroxylamine (280 mg, 0.003 mol), followed by TEA (272 mg, 0.003 mol) were added into the reaction flask and stirring was continued for 20 hrs at RT. The reaction mixture was partitioned between water and ethyl acetate (20 mL), The organic layer was washed with saturated NaHCO₃ (20 mL), NH₄Cl (20 mL), and brine solution (20 mL), dried over Na₂SO₄ and concentrated. The residual crude product (450 mg) was used for the next step without a further purification.

LCMS purity: 25%, m/z=518, (M+)

### Step: 6

### Synthesis of 6-Fluoro-7-(2-fluoro-4-iodo-phenylamino)-5-oxo-1,2,3,5-tetrahydro-indolizine-8-carboxylic acid (2-hydroxy-ethoxy)-amide

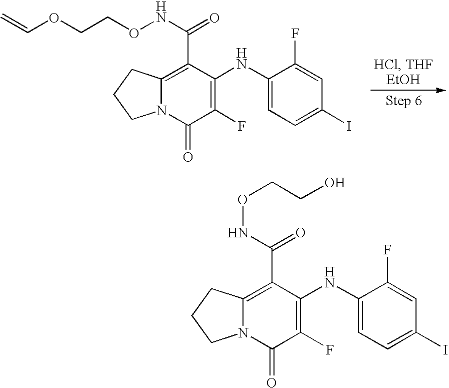

1N HCl (3 mL) was added to a stirred solution of 6-fluoro-7-(2-fluoro-4-iodo-phenylamino)-5-oxo-1,2,3,5-tetrahydro-indolizine-8-carboxylic acid (2-vinyloxy-ethoxy)-amide (450 mg 0.22 mmol) in a 1:1 mixture of THF and EtOH (12 mL). The reaction mixture was stirred for 90 minutes. The solvents from the reaction mixture were distilled and the reaction mixture was dissolved in water (3 mL), pH was adjusted to 6 with 2N NaOH solution and partitioned with EtOAc. The organic layer was dried over Na₂SO₄ and concentrated. Purification by preparative HPLC affords 61 mg (26% yield) of 6-fluoro-7-(2-fluoro-4-iodo-phenylamino)-5-oxo-1,2,3,5-tetrahydro-indolizine-8-carboxylic acid (2-hydroxy-ethoxy)-amide as a white solid.

LCMS purity: 95%, m/z=491.9, (M+)

HPLC: 96.6%

H¹ NMR (DMSO-D₆, 300 MHz) 11.5-11.4 (br s, 1H), 8.2-8.0 (br s, 1H), 7.6 (d, 1H), 7.4 (d, 1H), 6.82-6.72 (m, 1H), 4.0 (t, 2H), 3.8 (t, 2H), 3.6 (t, 2H), 3.2 (t, 2H), 2.2-2.0 (m, 2H)

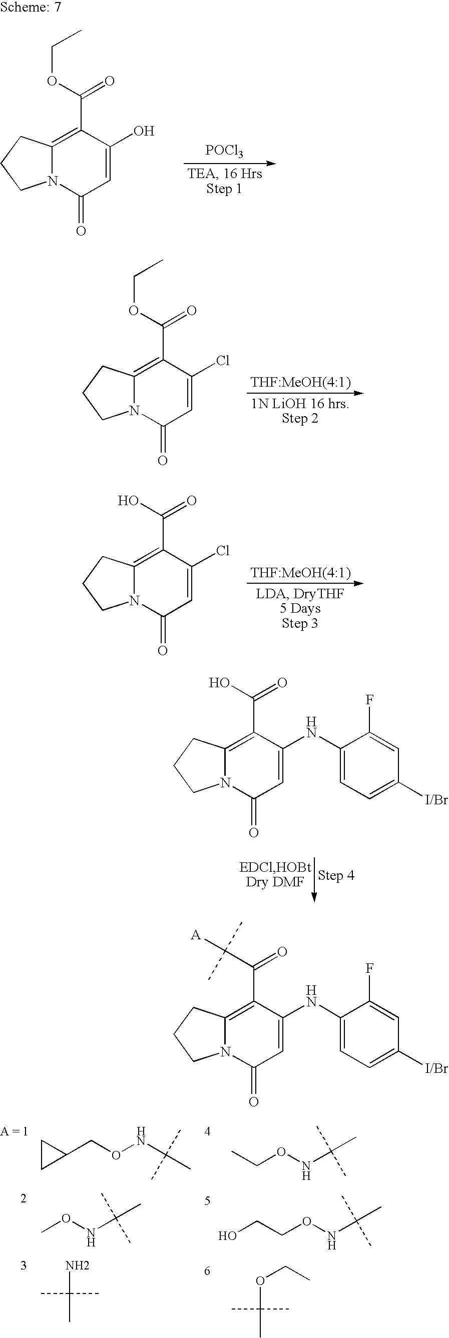

### EXAMPLE: 27

### Step: 1

### Synthesis of 7-Chloro-1,2,3,5-tetrahydro-indolizine-8-carboxylic acid ethyl ester

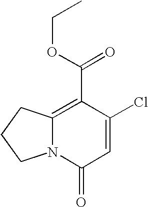

TEA (58.27 mmol, 8.4 mL) was added to a stirred solution 7-hydroxy-5-oxo-1,2,3,5-tetrahydro-indolizine-8-carboxylic acid ethyl ester (13 g, 58.27 mmol) in distilled POCl₃ (32 ml, 349 mmol). The reaction mixture was stirred for 16 hrs at RT under nitrogen atmosphere. POCl₃ was distilled from the reaction mixture and the residue was poured into an ice cold water, basified with saturated K₂CO₃ solution (pH=8.5). The reaction mixture was extracted with EtOAc. The organic layer was dried over anhydrous Na₂SO₄, concentrated and the concentrate was purified by column chromatography (using silica gel, and 75% ethyl acetate in hexane as eluant) to afford 7.5 mg (53.5% yield) of 7-chloro-1,2,3,5-tetrahydro-indolizine-8-carboxylic acid ethyl ester as a yellow solid.

¹H NMR (DMSO D₆, 300 MHz): δ 6.6-6.5 (br s, 1H), 4.4-4.3 (m, 2H), 4.2-4.1 (m, 2H), 3.5-3.3 (t, 2H), 2.3-2.2 (m, 2H), 1.4-1.3 (t, 2H)

### Step: 2

### Synthesis of 7-Chloro-5-oxo-1,2,3,5-tetrahydro-indolizine-8-carboxylic acid

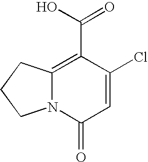

1N LiOH (40 mL) was added to a stirred solution of 7-hydroxy-5-oxo-1,2,3,5-tetrahydro-indoline-8-carboxylic acid ethyl ester (7.5 g, 31.1 mmol) in (4:1) THF:MeOH (75 mL). The reaction mixture was stirred for 15 hrs at room temperature. The reaction mixture was concentrated and acidified with 1N HCl, the precipitate formed was collected, to afford 5.2 g (78.7% yield) of 7-chloro-5-oxo-1,2,3,5-tetrahydro-indolizine-8-carboxylic acid as a white solid.

LC-MS purity: 99%, m/z=212, (M−1)

H¹ NMR (DMSO-D₆, 300 MHz): δ 13.25-13.15 (br s, 1H), 6.4 (s, 1H), 4.0 (t, 3H), 2.15-2.05 (m, 3H).

### Step: 3

### Synthesis of 7-(2-Fluoro-4-iodo-phenylamino)-5-oxo-1,2,3,5-tetrahydro-indolizine-8-carboxylic acid

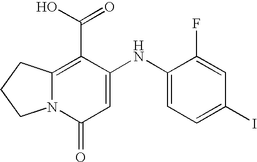

Lithiumdiisopropylamide (16.5 ml, 32.8 mmol) was added to a stirred solution of 2-fluoro-4-iodoaniline (5.6 g, 23.47 mmol) in dry THF (40 mL) at −78° C. under nitrogen atmosphere. This was followed by addition of 7-chloro-5-oxo-1,2,3,5-tetrahydro-indolizine-8-carboxylic acid (2 g, 9.38 mmol) in dry THF (150 mL) and the resulting mixture was stirred first for 30 min at −78° C. and then at RT for the next 5 days. The reaction mixture was concentrated and acidified with 1N HCl till the pH was about 2. The precipitate formed was collected and washed with diethyl ether to afford 2.5 g (65.7% yield) of 7-(2-fluoro-4-iodo-phenylamino)-5-oxo-1,2,3,5-tetrahydro-indolizine-8-carboxylic acid as a white solid.

LC-MS purity: 92.8%, m/z=414.8, (M+1)

H¹ NMR (DMSO-D₆, 300 MHz) δ 13.45-13.35 (br s, 1H), 10.05-9.95 (br s, 1H), 7.8 (dd, 1H), 7.6 (d, 1H), 7.4-7.2 (t, 1H), 5.4-5.3 (s, 1H), 4.0 (t, 2H), 3.5-3.4 (t, 2H), 2.1-2.0 (m, 2H)

### EXAMPLE: 28

### Steps 1 to 3 were Performed in a Manner Similar to What has been Described for Example 27

### Step: 4

### Synthesis of 7-(2-Fluoro-4-iodo-phenylamino)-5-oxo-1,2,3,5-tetrahydro-indolizine-8-carboxylic acid amide

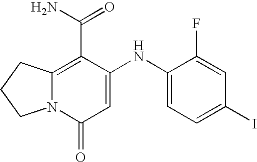

EDCI (0.415 mg, 2.17 mmol) and HOBt (0.293 mg, 1.7 mmol) were added to a stirred solution of 7-(2-fluoro-4-iodo-phenylamino)-5-oxo-1,2,3,5-tetrahydro-indolizine-8-carboxylic acid (0.300 mg, 0.72 mmol) in DMF (5 mL) and TEA (0.05 mL) at 0° C. The reaction mixture was stirred for 30 minutes at 0° C. under nitrogen atmosphere. This was followed by addition N Cl (0.115 mg, 2.17 mmol), followed by TEA (0.3 ml, 2.17 mmol) and the reaction mixture was stirred for 6 hrs at RT. The reaction mixture was diluted with water and extracted with EtOAc. The organic layer was washed with water and brine solution. The precipitate formed was collected to afford 0.040 mg (13% yield) of 7-(2-fluoro-4-iodo-phenylamino)-5-oxo-1,2,3,5-tetrahydro-indolizine-8-carboxylic acid amide as a white solid.

LC-MS purity: 98.7%, m/z=412, (M−1)

H¹ NMR (DMSO-D₆, 300 MHz) δ 9.0 (br s, 2H), 7.7 (dd, 1H), 7.7-7.6 (d, 1H), 7.3-7.2 (t, 1H), 5.5 (s, 1H), 3.9 (t, 2H), 2.1-2.0 (m, 2H)

### EXAMPLE: 29

### Steps 1 to 3 were Performed in a Manner Similar to What has been Described for Example 27

### Step: 4

### Synthesis of 7-(2-Fluoro-4-iodo-phenylamino)-5-oxo-1,2,3,5-tetrahydro-indolizine-8-carboxylic acid cyclopropylmethoxy-amide

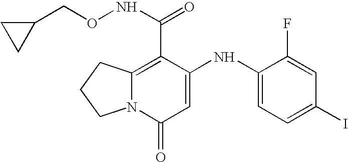

EDCI (0.101 mg, 0.53 mmol) and HOBt (0.072 mg, 0.53 mmol) were added to a stirred solution of 7-(2-fluoro-4-iodo-phenylamino)-5-oxo-1,2,3,5-tetrahydro-indolizine-8-carboxylic acid (0.200 mg, 0.48 mmol) in DMF (4 mL) and TEA (0.1 ml, 1.44 mmol) at RT. The reaction mixture was stirred for 30 minutes at RT under nitrogen atmosphere. This was followed by addition of O-cyclopropylmethyl-hydroxylamine hydrochloride (0.072 mg, 0.57 mol), TEA (0.4 ml, 1.44 mmol) and the reaction mixture was stirred for 18 hrs at RT. The reaction mixture was diluted with water and extracted with EtOAc. The organic layer was washed with water, NaHCO₃ and brine solution, dried over anhydrous Na₂SO₄ and concentrated. The residue was recrystallised with DCM (5 mL) and methanol (1 mL) to afford 0.059 mg (25% yield) of 7-(2-fluoro-4-iodo-phenylamino)-5-oxo-1,2,3,5-tetrahydro-indolizine-8-carboxylicacid cyclopropylmethoxy-amide as a brown solid.

LC-MS purity: 97%, m/z=481.9, (M−1)

H¹ NMR (DMSO-D₆, 300 MHz) δ 11.4-11.3 (br s, 1H), 8.3-8.2 (br s, 1H) 7.7 (dd, 1H) 7.6-7.5, (d, 1H), 7.2 (t, 1H), 5.4 (s, 1H), 3.9 (t 2H), 3.7-3.6 (d, 2H), 3.2-3.1 (m, 2H), 2.1-2.0 (m, 2H), 1.1-1.0 (m, 1H), 0.5-0.4 (m, 2H), 0.3-0.2 (m, 2H)

### EXAMPLE: 30

### Steps 1 to 3 were Performed in a Manner Similar to What has been Described for Example 27

### Step: 4

### Synthesis of 7-(2-Fluoro-4-iodo-phenylamino)-5-oxo-1,2,3,5-tetrahydro-indolizine-8-carboxylic acid ethyl ester

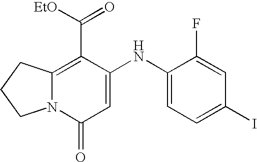

Concentrated H₂SO₄ (0.6 mL) was added to a stirred solution of 7-(2-fluoro-4-iodo-phenylamino)-5-oxo-1,2,3,5-tetra hydro-indolizine-8-carboxylicacid (0.300 mg, 0.72 mol) dissolved in EtOH (5 mL) and the reaction mixture was stirred for 3 days at 85° C. under nitrogen atmosphere. The reaction mixture was concentrated and partitioned between EtOAc and water. The organic layer was washed with NaHCO₃, brine solution, concentrated and washed with diethyl ether. Purification by column chromatography (using silica gel, and 1.5% methanol in chloroform as the eluant) affords 0.095 mg, (29.6% yield) of 7-(2-fluoro-4-iodo-phenylamino)-5-oxo-1,2,3,5-tetrahydro-indolizine-8-carboxylic acid ethyl ester as a white solid.

LC-MS purity: 96%, m/z=443, (M+1)

H¹ NMR (DMSO-D₆, 300 MHz) δ 9.5-9.4 (br s, 1H), 7.8 (dd, 1H) 7.6 (d, 1 μl) 7.3-7.2 (m, 1H), 5.4-5.3 (br s, 1H), 4.4-4.2 (m, 2H), 3.9 (t, 2H), 3.5 (t 2H), 2.1-2.0 (m, 2H), 1.4-1.2 (m, 3H)

### EXAMPLE: 31

### Steps 1 to 3 were Performed in a Manner Similar to What has been Described for Example 27

### Step: 4

### Synthesis of 7-(2-Fluoro-4-iodo-phenylamino)-5-oxo-1,2,3,5-tetrahydro-indolizine-8-carboxylic acid (2-vinyloxy-ethoxy)-amide

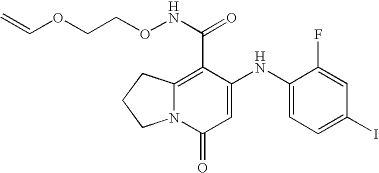

EDCI (0.138 mg, 0.72 mmol) and HOBt (0.097 mg, 0.72 mmol) were added to a stirred solution of 7-(2-fluoro-4-iodo-phenylamino)-5-oxo-1,2,3,5-tetrahydro-indolizine-8-carboxylic acid (0.200 mg, 0.48 mmol) in DMF (5 mL) and TEA (0.13 ml, 0.96 mmol) at RT. This was followed by addition O-(2-vinyloxy-ethyl)-hydroxylamine (0.99 mg, 0.96 mmol), TEA (0.13 ml, 0.96 mmol) and the reaction flask was stirred for 5 hrs at RT under nitrogen atmosphere. The reaction mixture was diluted with water and partitioned with EtOAc. The organic layer was washed with NH₄Cl, NaHCO₃, brine solution, dried over anhydrous Na₂SO₄ and concentrated. Purification by preparative HPLC affords 135 mg (56% yield) of 7-(2-fluoro-4-iodo-phenylamino)-5-oxo-1,2,3,5-tetrahydro-indolizine-8-carboxylic acid (2-vinyloxy-ethoxy)-amide as a brown gummy solid.

### Step: 5

### Synthesis of 7-(2-Fluoro-4-iodo-phenylamino)-5-oxo-1,2,3,5-tetrahydro-indolizine-8-carboxylic acid (2-hydroxy-ethoxy)-amide

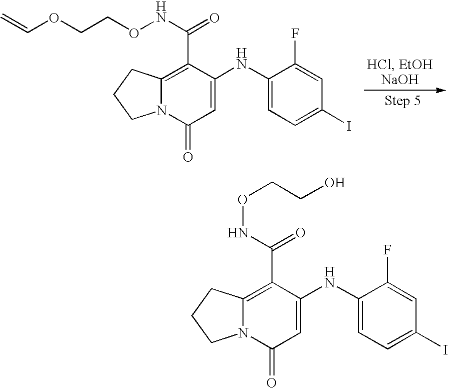

1N HCl (1.5 mL) and EtOH (3 mL) were added to a stirred solution of 7-(2-fluoro-4-iodo-phenylamino)-5-oxo-1,2,3,5-tetrahydro-indolizine-8-carboxylic acid-(2-vinyloxy-ethoxy)-amide (0.135 mg, 0.27 mmol) and the reaction mixture was stirred for 3 hrs at RT. The pH was adjusted to 5-7 with 2N NaOH solution. The reaction mixture was extracted with EtOAc. The organic layer was washed with water, brine solution, dried over anhydrous Na₂SO₄ and concentrated. Purification by preparative HPLC affords 12 mg (10% yield) of 7-(2-fluoro-4-iodo-phenylamino)-5-oxo-1,2,3,5-tetrahydro-indolizine-8-carboxylic acid (2-hydroxy-ethoxy)-amide as a white solid.

LC-MS purity: 98.9%, m/z=473.9, (M+1)

H¹ NMR (DMSO-D₆, 300 MHz) 11.4-11.3 (br s, 1H), 8.3-8.2 (br s, 1H), 7.8-7.7 (dd, 1H) 7.6-7.5 (d, 1H) 7.2-7.1 (m, 1H), 5.4-5.3 (br s, 1H), 4.0-3.8 (m, 4H), 3.6 (t, 2H), 3.2 (t, 2H), 2.1-2.0 (m, 2H)

### EXAMPLE: 32

### Steps 1 to 3 were Performed in a Manner Similar to What has been Described for Example 27

### Step: 4

### 7-(2-Fluoro-4-iodo-phenylamino)-5-oxo-1,2,3,5-tetrahydro-indolizine-8-carboxylic acid methoxy-amide

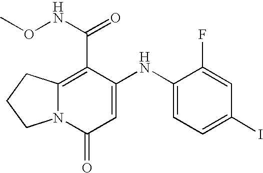

EDCI (0.280 mg, 0.001 mol) and HOBt (0.197 mg, 0.001 mmol) were added to a stirred solution of 7-(2-fluoro-4-iodo-phenylamino)-5-oxo-1,2,3,5-tetrahydro-indolizine-8-carboxylicacid (0.200 mg, 0.0005 mol) in DMF (5 mL), TEA (0.005 mL) and chloroform (2 mL) at 0° C. The reaction mixture was stirred for 1.30 hrs at 0° C. under nitrogen atmosphere. This was followed by addition O-methoxy-hydroxylamine hydrochloride (0.121 mg, 0.001 mol), followed by TEA (0.2 ml, 0.001 mol) and the reaction mixture was stirred for 18 hrs at RT. The reaction mixture was diluted with water and extracted with EtOAc. The organic layer was washed with water, NaHCO₃ and brine solution. The reaction mixture was dried over anhydrous Na₂SO₄ and concentrated. The concentrate was purified by preparative HPLC to afford 6 mg (2.8% yield) of 7-(2-fluoro-4-iodo-phenylamino)-5-oxo-1,2,3,5-tetrahydro-indolizine-8-carboxylic acid methoxy-amide as a white solid.

LC-MS purity: 98%, m/z=443.8, (M+1)

H¹ NMR (DMSO-D₆, 300 MHz) δ 11.4-11.3 (br s, 1H), 8.3-8.2 (br s, 1H) 7.7 (dd, 1H) 7.5, (d, 1H), 7.2-7.1 (t, 1H), 5.3 (s, 1H), 3.9 (t, 2H), 3.8-3.6 (br s, 2H), 3.3-3.2 (m, 2H), 2.1-2.0 (m, 2H)

### EXAMPLE: 33

### Steps 1 to 3 were Performed in a Manner Similar to What has been Described for Example 27

### Step: 4

### Synthesis of 7-(2-Fluoro-4-iodo-phenylamino)-5-oxo-1,2,3,5-tetrahydro-indolizine-8-carboxylic acid ethoxy-amide

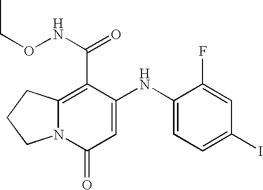

EDCI (0.277 mg, 0.001 mol) and HOBt (0.200 mg, 0.001 mmol) were added to a stirred solution of 7-(2-fluoro-4-iodo-phenylamino)-5-oxo-1,2,3,5-tetrahydro-indolizine-8-carboxylicacid (0.200 mg, 0.0005 mol) in DMF (5 mL), TEA (0.1 mL) and DCM (2 mL) at 0° C. The reaction mixture was stirred for 1.30 hrs at 0° C. under nitrogen atmosphere. This was followed by addition O-ethoxy-hydroxylamine hydrochloride (0.141 mg, 0.001 mol), followed by TEA (0.2 ml, 0.001 mol) and the reaction mixture was stirred for 18 hrs at RT. The reaction mixture was diluted with water and extracted with EtOAc. The organic layer was washed with water, NaHCO₃ and brine solution. The reaction mixture was dried over anhydrous Na₂SO₄ and concentrated. Purification by preparative HPLC affords 13 mg (6% yield) of 7-(2-fluoro-4-iodo-phenylamino)-5-oxo-1,2,3,5-tetrahydro-indolizine-8-carboxylic acid ethoxy-amide as a white solid.

LC-MS purity: 98%, m/z=457.8, (M+1)

H¹ NMR (DMSO-D₆, 300 MHz) δ 11.4-11.3 (br s, 1H), 8.3-8.25 (br s, 1H) 7.8-7.7 (d, 1H) 7.6-7.5, (d, 1H), 7.3-7.1 (t, 1H), 5.4 (s, 1H), 4.0-3.8 (m, 4H), 3.3-3.0 (m, 2H), 2.1-2.0 (t, 2H), 1.3-1.1 (t, 3H)

### EXAMPLE: 34

### Steps 1 to 4 were Performed in a Manner Similar to What has been Described for Example 1 and Step 5 was Performed in a Manner Similar to What has been Described for Example 2

### Step: 6

### Synthesis of 7-(4-Bromo-2-fluoro-phenylamino)-5-oxo-1,2,3,5-tetrahydro-indolizine-8-carboxylic acid (2,2-dimethyl-[1,3]dioxolan-4-ylmethoxy)-amide

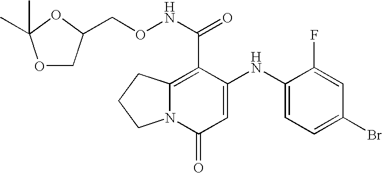

EDCI (155 mg, 0.816 mmol) and HOBt (110 mg, 0.816 mmol) were added to a stirred solution of 7-(4-bromo-2-fluoro-phenylamino)-5-oxo-1,2,3,5-tetrahydro-indolizine-8-carboxylic acid (0.2 g, 0.544 mmol) in DMF (6 mL) at 0° C. The reaction mixture was stirred for 1 hr at 0° C. This was followed by addition O-(4,4-dimethyl-[1,3]dioxolan-2-ylmethyl)-hydroxylamine (180 mg, 0.653 mmol), TEA (0.45 ml, 3.264 mmol). The reaction mixture was stirred at RT overnight. The reaction mixture was partitioned between ethyl acetate (50 mL) and cold water (50 mL). The organic layer was washed with saturated NaHCO₃ solution, dried over Na₂SO₄ and concentrated. Purification by column chromatography (using silica gel, 5% methanol in chloroform as eluant) afford 50 mg (19.13% yield) of 7-(4-bromo-2-fluoro-phenylamino)-5-oxo-1,2,3,5-tetrahydro-indolizine-8-carboxylic acid (2,2-dimethyl-[1,3]dioxolan-4-ylmethoxy)-amide as the required product.

### Step: 7

### Synthesis of 7-(4-Bromo-2-fluoro-phenylamino)-5-oxo-1,2,3,5-tetrahydro-indolizine-8-carboxylic acid (2,3-dihydroxy-propoxy)-amide

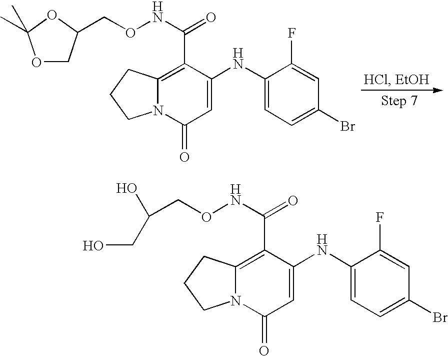

1N HCl (1 mL) was added to a stirred solution of 7-(4-bromo-2-fluoro-phenylamino)-5-oxo-1,2,3,5-tetrahydro-indolizine-8-carboxylic acid (2,2-dimethyl-[1,3]dioxolan-4-ylmethoxy)-amide (50 mg, 0.010 mmol) dissolved in ethanol (2 mL). The reaction mixture was stirred for 3 hrs at RT. Ethanol was distilled and the reaction mixture was partitioned between water and ethylacetate (20 mL). The organic layer was dried over Na₂SO₄, concentrated and the concentrate was purified by the recrystallization using DCM to afford 25 mg (17.35% yield) of 7-(4-bromo-2-fluoro-phenylamino)-5-oxo-1,2,3,5-tetrahydro-indolizine-8-carboxylic acid (2,3-dihydroxy-propoxy)-amide as the required product.

LC-MS: 95.8%; m/z=456 (M+1), 458 (M+2)

HPLC: 97.3%

H¹ NMR (DMSO-D₆, 300 MHz): δ 11.2 (br s, 1H), 8.3-8.2 (br s, 1H), 7.6-7.5 (d, 1H), 7.5-7.3 (m, 2H), 5.3 (s, 1H), 4.9 (d, 1H), 4.6-4.4 (t, 1H), 4.0-3.8 (m, 3H), 3.8-3.5 (m, 2H), 3.4 (m, 2H), 3.3-3.2 (m, 2H), 2.1-2.0 (m, 2H)

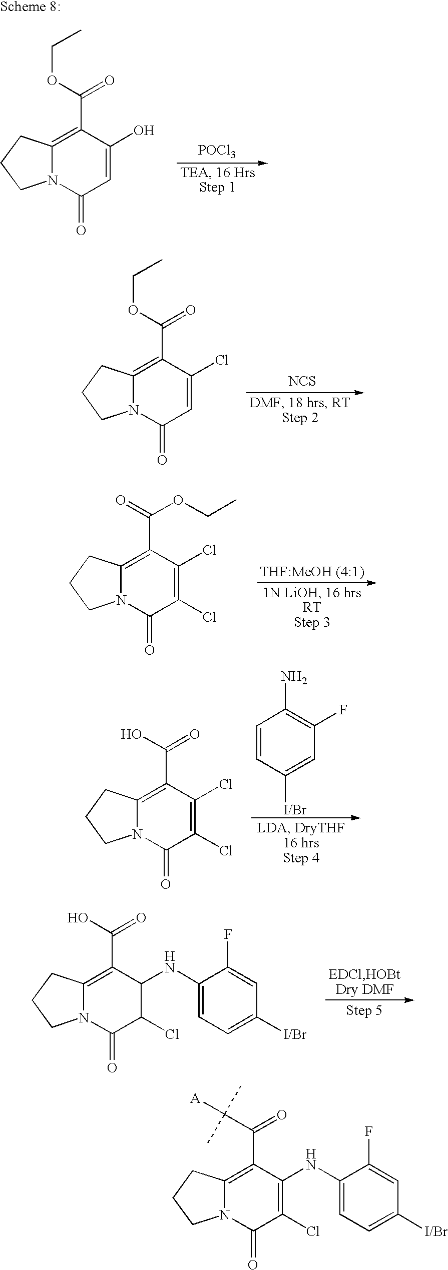

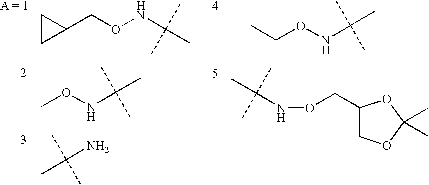

### EXAMPLE: 35

### Step: 1

### Synthesis of 7-Chloro-1,2,3,5-tetrahydro-indolizine-8-carboxylic acid ethyl ester

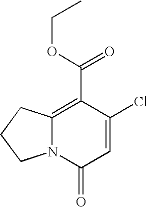

TEA (58.27 mmol, 8.4 mL) were added to a stirred solution of 7-hydroxy-5-oxo-1,2,3,5-tetrahydro-indolizine-8-carboxylic acid ethyl ester (13 g, 58.27 mmol) in distilled POCl₃ (32 ml, 349 mmol) and the reaction mixture was stirred for 16 hrs at room temperature under nitrogen atmosphere. POCl₃ was distilled, the reaction mixture was poured into an ice cold water and basified with saturated K₂CO₃ solution to a pH of about 8.5. The reaction mixture was extracted with EtOAc. The organic layer was dried over anhydrous Na₂SO₄ and concentrated. The concentrate was purified by column chromatography (using silica gel, 75% ethyl acetate in hexane as the eluant) to afford 7.5 mg (53.5% yield) of 7-chloro-1,2,3,5-tetrahydro-indolizine-8-carboxylic acid ethyl ester as a yellow solid.

¹H NMR (DMSO-D₆, 300 M): δ 6.6-6.5 (br s, 1H), 4.4-4.3 (m, 2H), 4.2-4.1 (m, 2H), 3.5-3.3 (t, 2H), 2.3-2.2 (m, 2H), 1.4-1.3 (t, 2H)

### Step: 2

### Synthesis of 6,7-Dichloro-5-oxo-1,2,3,5-tetrahydro-indolizine-8-carboxylic acid ethyl ester

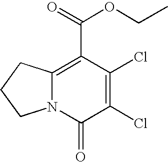

NCS (304 mg, 0.002282 mol) was added to a solution of 7-chloro-1,2,3,5-tetrahydro-indolizine-8-carboxylic acid ethyl ester (500 mg, 0.00201 mol) dissolved in DMF and the reaction mixture was stirred for 18 hrs at RT under nitrogen atmosphere. The reaction mixture was extracted with ethylacetate, washed with water and brine solution. The organic layer was dried over anhydrous Na₂SO₄ and concentrated to afford 400 mg (70% yield) of 6,7-dichloro-5-oxo-1,2,3,5-tetrahydro-indolizine-8-carboxylic acid ethyl ester as the required product.

¹H NMR (DMSO-D₆, 300 MHz): δ 4.3-4.2 (m, 2H), 4.1-4.0 (t, 2H), 3.3 (t, 2H), 2.2-2.1 (m, 2H), 1.3 (t 3H)

LCMS: 78%; m/z=278, M+2

HPLC: 89%

### Step: 3

### Synthesis of 6,7-Dichloro-5-oxo-1,2,3,5-tetrahydro-indolizine-8-carboxylic acid

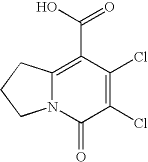

1N LiOH (10 mL) was added to a stirred solution 6,7-dichloro-5-oxo-1,2,3,5-tetrahydro-indolizine-8-carboxylic acid ethyl ester (400 mg, 0.001 mol) in (4:1) THF:MeOH (10 mL) and the reaction mixture was stirred for 3 hrs at RT. The reaction mixture was concentrated and acidified with 1N HCl to a pH of about 2. The precipitate was collected under reduced pressure to afford 375 mg (100% yield) of 6,7-dichloro-5-oxo-1,2,3,5-tetrahydro-indolizine-8-carboxylic acid as the required product.

H¹ NMR (DMSO-D₆, 300 MHZ): δ 13.2 (s, 1H), 4.1-4.0 (t, 2H), 3.3 (t, 2H), 2.2-2.1 (m, 2H)

LCMS: 92%; m/z=248, M+1; 249.7, M+2

HPLC: 90.8%

### Step: 4

### Synthesis of 7-(4-Bromo-2-fluoro-phenylamino)-6-chloro-5-oxo-1,2,3,5-tetrahydro-indolizine-8-carboxylic acid

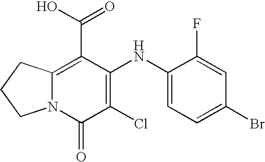

n-Butyl lithium (2 ml, 0.003 mol) were added dropwise to a stirred solution of diisopropyl amine (0.65 ml, 0.005 mol) in dry THF (40 mL) over a period of 5 mins at −78° C. and the reaction mixture was stirred for 30 minutes followed by addition of 4-bromo-2-fluoro-phenylamine (462 mg, 0.002 mol) dissolved in dry THF (5 mL) at −78° C. The reaction mixture was stirred for a further 30 minutes, and was followed by addition of 6,7-dichloro-5-oxo-1,2,3,5-tetrahydro-indolizine-8-carboxylic acid (200 mg, 0.00 mmol) dissolved in dry THF (10 mL) at −78° C. with the stirring over a period of 30 mins. The stirring was continued for a further 16 hrs at RT. THF was distilled and the residual mass was acidified by addition of 1N HCl, followed by ether with stirring for 10 mins. The precipitate formed was collected, washed with ether and dried to afford 232 mg (72% yield) of 7-(4-bromo-2-fluoro-phenylamino)-6-chloro-5-oxo-1,2,3,5-tetrahydro-indolizine-8-carboxylic acid as the required product.

H¹ NMR (DMSO-D₆, 300 MHz) δ 13.3 (s, 1H), 9.6-9.5 (br s, 1H), 7.6-7.5 (dd, 1H), 7.3 (d, 1H), 6.9 (t, 1H), 4.1-4.0 (t, 2H), 3.5-3.4 (t, 2H), 2.2-2.1 (m, 2H)

LCMS: 96%; m/z=400.9, M+1

HPLC=95.5%

### EXAMPLE: 36

### Step: 5

### Synthesis of 7-(4-Bromo-2-fluoro-phenylamino)-6-chloro-5-oxo-1,2,3,5-tetrahydro-indolizine-8-carboxylic acid cyclopropylmethoxy-amide

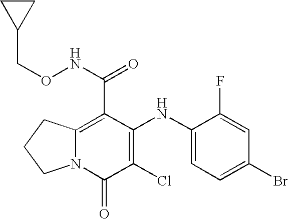

### Procedure:

EDCI (143 mg, 0.001 mol) and HOBt (102 mg, 0.001 mol) were added to a stirred solution of 7-(4-bromo-2-fluoro-phenylamino)-6-chloro-5-oxo-1,2,3,5-tetrahydro-indolizine-8-carboxylic acid (100 mg, 0.0002 mol) in DMF (3 mL) at 0° C. The reaction mixture was stirred for 1.5 hrs at 0° C. This was followed by addition of O-cyclopropylmethyl-hydroxylamine hydrochloride (92 mg, 0.00 mmol), TEA (0.1 ml, 0.00 mmol) at 0° C. The reaction mixture was stirred for 18 hours at RT. The reaction mixture was partitioned between ethylacetate and water. The organic layer was washed with saturated NH₄Cl solution, NaHCO₃ solution, and brine solution, dried over anhydrous Na₂SO₄ and concentrated. Purification by preparative HPLC affords 42 mg (36% yield) of 7-(4-bromo-2-fluoro-phenylamino)-6-chloro-5-oxo-1,2,3,5-tetrahydro-indolizine-8-carboxylic acid cyclopropylmethoxy-amide as the required product.

H¹ NMR (DMSO-D₆, 300 MHz) δ 11.4-11.2 (br s, 1H), 8.04-7.96 (br s, 1H), 7.5 (d, 1H), 7.3 (d, 1H), 6.9 (t, 1H), 4.1-4.0 (t, 2H), 3.2 (d, 2H), 3.1 (t, 2H), 2.2-2.1 (m, 2H), 1.0-0.9 (m, 1H), 0.5 (d, 2H), 0.3 (s, 2H)

LCMS: 100%; m/z=471.7, M+1

HPLC=99%

### EXAMPLE: 37

### Steps 1 to 4 were Performed in a Manner Similar to What has been Described for Example 1

### Step: 5

### Synthesis of 7-(4-Bromo-2-fluoro-phenylamino)-6-chloro-5-oxo-1,2,3,5-tetrahydro-indolizine-8-carboxylic acid methoxy-amide

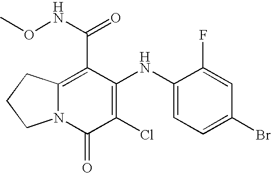

EDCI (286 mg, 0.001 mol) and HOBt (202 mg, 0.001 mol) were added to a stirred solution of 7-(4-bromo-2-fluoro-phenylamino)-6-chloro-5-oxo-1,2,3,5-tetrahydro-indolizine-8-carboxylic acid (200 mg, 0.0005 mol) in dry DMF (5 mL) at 0° C. The reaction mixture was stirred for 1.30 hrs at 0° C. This was followed by addition of O-methoxy-hydroxylamine hydrochloride (125 mg, 0.001 mol), TEA (0.21 ml, 0.001 mol) at 0° C. The reaction mixture was stirred for 16 hrs at RT under nitrogen atmosphere. The reaction mixture was partitioned between EtOAc and water. The organic layer was washed with water, saturated NaHCO₃ solution and brine solution, dried over anhydrous Na₂SO₄, concentrated and the crude product was washed with methanol to afford 0.020 g (9.3% yield) of 7-(4-bromo-2-fluoro-phenylamino)-6-chloro-5-oxo-1,2,3,5-tetrahydro-indolizine-8-carboxylic acid methoxy-amide as a white solid.

H¹ NMR (DMSO-D₆, 300 MHz) δ 11.4-11.2 (br s, 1H), 8.0 (br s, 1H), 7.5 (dd, 1H), 7.3 (d, 1H), 6.9 (t, 1H), 4.0 (t, 2H), 3.4 (s, 3H), 3.1 (t, 2H), 2.1-2.0 (m, 2H)

LCMS: 90%; m/z=431.9, M+1

HPLC: 99%

### EXAMPLE: 38

### Steps 1 to 4 were Performed in a Manner Similar to What has been Described for Example 1

### Step: 5

### Synthesis of 7-(4-Bromo-2-fluoro-phenylamino)-6-chloro-5-oxo-1,2,3,5-tetrahydro-indolizine-8-carboxylic acid amide

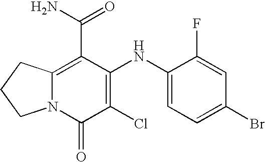

EDCI (286 mg, 0.001 mol) and HOBt (202 mg, 0.0.001 mol) were added to a stirred solution of 7-(4-bromo-2-fluoro-phenylamino)-6-chloro-5-oxo-1,2,3,5-tetrahydro-indolizine-8-carboxylic acid (200 mg, 0.005 mol) in dry DMF (5 mL) at 0° C. The reaction mixture was stirred for 1.30 hrs at 0° C. This was followed by addition of NH₄Cl (80 mg, 0.001 mol), followed by TEA (0.2 ml, 0.001 mol) at 0° C. The reaction mixture was stirred for 16 hrs at RT. The reaction mixture was partitioned between EtOAc and water. The organic layer was washed with water, saturated NaHCO₃ solution and brine solution, dried over anhydrous. Na₂SO₄ and concentrated to afford 0.020 g (9.3% yield) of 7-(4-bromo-2-fluoro-phenylamino)-6-chloro-5-oxo-1,2,3,5-tetrahydro-indolizine-8-carboxylic acid amide as the required product.

H₁NMR (DMSO-D₆, 300 MHz) δ 8.44-8.4 (br s, 1H), 7.64-7.58 (br s, 2H), 7.5 (dd, 1H), 7.2 (d, 1H), 6.9-6.8 (t, 1H), 4.0 (t, 2H), 3.3-3.2 (t, 2H), 2.1-2.0 (m, 2H)

LCMS: 94.2%; m/z=401.9, M+1

HPLC: 94.5%

### EXAMPLE: 39

### Steps 1 to 4 were Performed in a Manner Similar to What has been Described for Example 1

### Step: 5

### Synthesis of 7-(4-Bromo-2-fluoro-phenylamino)-6-chloro-5-oxo-1,2,3,5-tetrahydro-indolizine-8-carboxylic acid ethoxy-amide

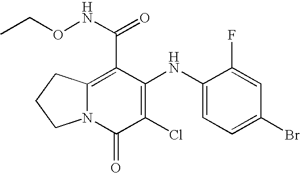

EDCI (286 mg, 0.001 mol) and HOBT (202 mg, 0.001 mol) were added to a stirred solution of 7-(4-bromo-2-fluoro-phenylamino)-6-chloro-5-oxo-1,2,3,5-tetrahydro-indolizine-8-carboxylic acid (200 mg, 0.0005 mol) in dry DMF (5 mL) at 0° C. The reaction mixture was stirred for 1.30 hrs at 0° C. This was followed by addition of O-ethoxy-hydroxylamine hydrochloride (145 mg, 0.001 mol), followed by TEA (0.2 ml, 0.001 mol) at 0° C. The reaction mixture was stirred for 18 hrs at RT under nitrogen atmosphere. The reaction mixture was partitioned between EtOAc and water. The organic layer was washed with water, saturated NaHCO₃ solution and brine solution, dried over anhydrous. Na₂SO₄, concentrated and the crude product was recrystallised using methanol to afford 52 g (23.6% yield) of 7-(4-bromo-2-fluoro-phenylamino)-6-chloro-5-oxo-1,2,3,5-tetrahydro-indolizine-8-carboxylic acid ethoxy-amide as the required product.

H¹ NMR (DMSO-D₆, 300 MHz) δ 11.4-11.2 (br s, 1H), 8.1-7.9 (br s, 1H), 7.5 (dd, 1H), 7.3 (d, 1H), 6.9 (t, 1H), 4.0 (t, 2H), 3.6-3.5 (m, 2H), 3.1 (t, 2H), 2.2-2.0 (m, 2H), 1.1 (t, 3H)

LCMS: 100%; m/z=445.7, M+1; 443.8, M−1

HPLC: 96%

### EXAMPLE: 40

### Steps 1 to 4 were Performed in a Manner Similar to What has been Described for Example 1

### Step: 5

### Synthesis of 7-(4-Bromo-2-fluoro-phenylamino)-6-chloro-5-oxo-1,2,3,5-tetrahydro-indolizine-8-carboxylic acid (2,2-dimethyl-[1,3]dioxolan-4-ylmethoxy)-amide

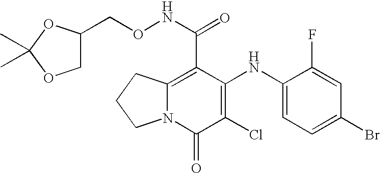

EDCI (572 mg, 0.003 mol) and HOBt (191.7 mg, 0.003 mol) were added to a stirred solution of 7-(4-bromo-2-fluoro-phenylamino)-6-chloro-5-oxo-1,2,3,5-tetrahydro-indolizine-8-carboxylic acid (400 mg, 0.0016 mol) in dry DMF (10 mL) at 0° C. The reaction mixture was stirred for 1.30 hrs at 0° C. This was followed by addition of O-(2,2-dimethyl-[1,3]dioxolan-4-ylmethyl)-hydroxylamine (440 mg, 0.003 mol), TEA (0.4 ml, 0.003 mol) at 0° C. and stirring was continued for 16 hrs at RT. The reaction mixture was partitioned between EtOAc and water. The organic layer was washed with water, saturated NaHCO₃ solution and brine solution, dried over anhydrous. Na₂SO₄, concentrated. Purification by column chromatography (using silica gel, 2% methanol and chloroform as eluant) affords 0.3 g (56.8% yield) of 7-(4-bromo-2-fluoro-phenylamino)-6-chloro-5-oxo-1,2,3,5-tetrahydro-indolizine-8-carboxylic acid (2,2-dimethyl-[1,3]dioxolan-4-ylmethoxy)-amide as the required product.

H¹ NMR (DMSO-D₆, 300 MHz) δ 11.4 (br s, 1H), 7.94-8.06 (br s, 1H), 7.5 (dd, 1H), 7.3-7.24 (d, 1H), 6.9 (t, 1H), 4.2-4.1 (m, 1H), 4.1-4.0 (m, 3H), 3.6-3.5 (m, 3H), 3.1 (t, 2H), 2.1-2.0 (m, 2H), 0.8-0.7 (s, 6H)

LCMS: 65%; m/z=529.9 M+1

### Step: 6

### Synthesis of 7-(4-Bromo-2-fluoro-phenylamino)-6-chloro-5-oxo-1,2,3,5-tetrahydro-indolizine-8-carboxylic acid (2,3-dihydroxy-propoxy)-amide

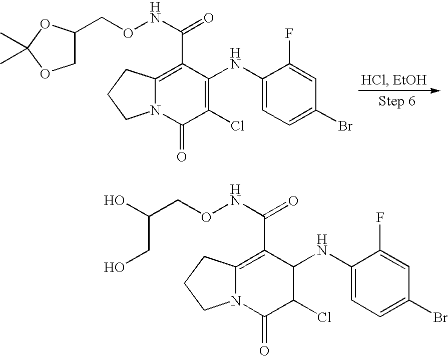

1N HCl (0.6 mL) were added to a stirred solution of 7-(4-bromo-2-fluoro-phenylamino)-6-chloro-5-oxo-1,2,3,5-tetrahydro-indolizine-8-carboxylic acid (2,2-dimethyl-[1,3]dioxolan-4-ylmethoxy)-amide (300 mg, 0.001 mol) dissolved in EtOH (12 mL). The reaction mixture was stirred for 2 hrs at room temperature. Ethanol was distilled and the residual mass was extracted with EtOAc. The organic layer was washed with water, saturated NaHCO₃ solution, brine solution and dried over anhydrous Na₂SO₄ and concentrated. Recrystallization from methanol affords 50 mg (18.5% yield) of 7-(4-bromo-2-fluoro-phenylamino)-6-chloro-5-oxo-1,2,3,5-tetrahydro-indolizine-8-carboxylic acid (2,3-dihydroxy-propoxy)-amide as a white solid.

H¹ NMR (DMSO-D₆, 300 MHz) δ 11.4 (br s, 1H), 8.0 (br s, 1H), 7.5-7.4 (dd, 1H), 7.3-7.2 (d, 1H), 6.9 (t, 1H), 4.8 (d, 1H), 4.6-4.5 (t, 1H), 4.0 (t, 2H), 3.7-3.5 (m, 2H), 3.5-3.4 (m, 1H), 3.2-3.1 (t 2H), 2.1-2.0 (m, 2H).

LCMS: 85%; m/z=491.7, M+1

HPLC=96%

### EXAMPLE: 41

### Steps 1 to 3 were Performed in a Manner Similar to What has been Described for Example 1

### Step: 4

### Synthesis of 6-Chloro-7-(2-fluoro-4-iodo-phenylamino)-5-oxo-1,2,3,5-tetrahydro-indolizine-8-carboxylic acid

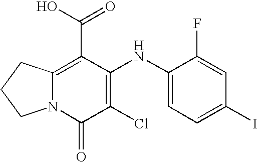

n-Butyl lithium (20 ml, 0.032 mol) was added dropwise for 5 mins to a stirred solution of diisopropyl amine (4.5 ml, 0.032 mol) in dry THF (5 mL) at −78° C. and the reaction mixture was stirred for 30 minutes. This was followed by the addition of 2-fluoro-4-iodo-phenylamine (5.75 g, 0.002 mol) dissolved in dry THF (10 mL) at −78° C. The reaction mixture was stirred for a further 30 minutes and this was followed by addition of 6,7-dichloro-5-oxo-1,2,3,5-tetrahydro-indolizine-8-carboxylic acid (2 g, 0.008 mol) dissolved in dry THF (130 mL) at −78° C. with stirring over a period of 30 mins. The stirring was continued for a further 2 days at RT under nitrogen atmosphere. THF was distilled and the residual reaction mixture was acidified by addition of 1N HCl. Addition of diethyl ether, stirring for 10 mins yields a precipitate which was collected, washed with diethyl ether and dried to afford 2.3 g (63.8% yield) of 6-chloro-7-(2-fluoro-4-iodo-phenylamino)-5-oxo-1,2,3,5-tetrahydro-indolizine-8-carboxylic acid as the required product.

H¹ NMR (DMSO-D₆, 300 MHz) δ 13.6 (s, 1H), 9.5 (br s, 1H), 7.6 (dd, 1H), 7.4 (d, 1H), 6.7 (t, 1H), 4.1-4.0 (t, 2H), 3.5-3.4 (t, 2H), 2.2-2.1 (m, 2H)

LCMS: 92%; m/z=448.7

HPLC: 98%

### EXAMPLE: 42

### Step: 5

### Synthesis of 6-Chloro-7-(2-fluoro-4-iodo-phenylamino)-5-oxo-1,2,3,5-tetrahydro-indolizine-8-carboxylic acid cyclopropylmethoxy-amide

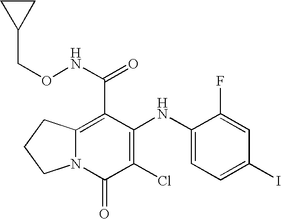

EDCI (256 mg, 0.001 mol) and HOBt (181 mg, 0.001 mol) were added to a stirred solution of 6-chloro-7-(2-fluoro-4-iodo-phenylamino)-5-oxo-1,2,3,5-tetrahydro-indolizine-8-carboxylic acid (200 mg, 0.0004 mol) in dry DMF (5 mL) at 0° C. The reaction mixture was stirred for 1.30 hrs at 0° C. This was followed by addition of O-cyclopropylmethyl-hydroxylamine hydrochloride (165 mg, 0.001 mol), TEA (0.2 ml, 0.001 mol) at 0° C. The reaction mixture was stirred for 16 hrs at RT under nitrogen atmosphere. The reaction mixture was partitioned between EtOAc and water. The organic layer was washed with water, saturated NH₄Cl, saturated NaHCO₃ solution and brine solution, dried over anhydrous Na₂SO₄, concentrated and the concentrate was washed with methanol to afford 55 mg (24% yield) of 6-chloro-7-(2-fluoro-4-iodo-phenylamino)-5-oxo-1,2,3,5-tetrahydro-indolizine-8-carboxylic acid cyclopropylmethoxy-amide as the required product.

H¹ NMR (DMSO-D₆, 300 M) δ 11.25-11.2 (br s, 1H), 8.0-7.94 (br s, 1H), 7.6-7.5 (dd, 1H), 7.4 (d, 1H), 6.8-6.7 (t, 1H), 4.0 (t, 2H), 3.2 (d, 2H), 3.1 (t, 2H), 2.1 (t, 2H), 1.0-0.9 (m, 1H), 0.5 (d, 2H), 0.3 (s, 2H)

LCMS: 94.5%; m/z=517.6

HPLC: 94.79%

### EXAMPLE: 43

### Steps 1 to 3 were Performed in a Manner Similar to What has been Described for Example 1 and Step 4 was Performed in a Manner Similar to What has been Described for Example 6

### Step: 5

### Synthesis of 6-Chloro-7-(2-fluoro-4-iodo-phenylamino)-5-oxo-1,2,3,5-tetrahydro-indolizine-8-carboxylic acid methoxy-amide

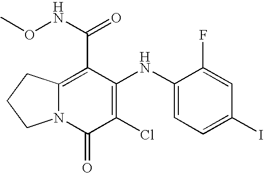

EDCI (256 mg, 0.001 mol) and HOBt (181 mg, 0.001 mol) were added to a stirred solution of 6-chloro-7-(2-fluoro-4-iodo-phenylamino)-5-oxo-1,2,3,5-tetrahydro-indolizine-8-carboxylic acid (200 mg, 0.0004 mol) in dry DMF (10 mL) at 0° C. The reaction mixture was stirred for 1.30 hrs at 0° C. This was followed by addition of O-methyl-hydroxylamine hydrochloride (111 mg, 0.001 mol), followed by TEA (0.2 ml, 0.001 mol) at 0° C. The reaction mixture was stirred for 16 hrs at RT under nitrogen atmosphere. The reaction mixture was partitioned between EtOAc and water. The organic layer was washed with water, saturated NaHCO₃ solution and brine solution, dried over anhydrous Na₂SO₄, concentrated and the concentrate was washed with methanol to afford 105 mg (48% yield) of 6-chloro-7-(2-fluoro-4-iodo-phenylamino)-5-oxo-1,2,3,5-tetrahydro-indolizine-8-carboxylic acid methoxy-amide as the required product.

H₁NMR (DMSO-D₆, 300 M) δ 11.4-11.2 (br s, 1H), 8.02-7.96 (br s, 1H), 7.6-7.5 (dd, 1H), 7.4 (d, 1H), 6.8-6.7 (t, 1H), 4.0 (t, 2H), 3.4 (s, 3H), 3.1 (t, 2H), 2.1-2.0 (m, 2H)

LCMS: 90%; m/z=477.9

HPLC: 96.6%

### EXAMPLE: 44

### Steps 1 to 3 were Performed in a Manner Similar to What has been Described for Example 1 and Step 4 was Performed in a Manner Similar to What has been Described for Example 6

### Step: 5

### Synthesis of 6-Chloro-7-(2-fluoro-4-iodo-phenylamino)-5-oxo-1,2,3,5-tetrahydro-indolizine-8-carboxylic acid amide

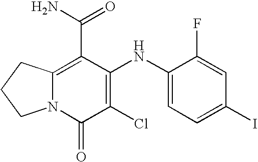

EDCI (256 mg, 0.001 mol) and HOBt (181 mg, 0.001 mol) were added to a stirred solution of 6-chloro-7-(2-fluoro-4-iodo-phenylamino)-5-oxo-1,2,3,5-tetrahydro-indolizine-8-carboxylic acid (200 mg, 0.0004 mol) in dry DMF (5 mL) at 0° C. The reaction mixture was stirred for 1.30 hrs at 0° C. under nitrogen atmosphere. This was followed by addition of NH₄Cl (0.071 g, 0.001 mol), followed by TEA (0.2 ml, 0.001 mol) at 0° C. The reaction mixture was stirred for 16 hrs at RT. The reaction mixture was partitioned between EtOAc and water. The organic layer was washed with water, saturated NaHCO₃ solution and brine solution, dried over anhydrous Na₂SO₄, concentrated and the concentrate was washed with methanol to afford 60 mg (30% yield) of 6-chloro-7-(2-fluoro-4-iodo-phenylamino)-5-oxo-1,2,3,5-tetrahydro-indolizine-8-carboxylic acid amide as the required product.

H¹ NMR (DMSO-D₆, 300 MHz) δ 8.46-8.4 (br s, 1H), 7.68-7.52 (m, 3H), 7.4 (d, 1H), 6.7 (t, 1H), 4.0 (t, 2H), 3.3-3.2 (t, 2H), 2.1-2.0 (m, 2H)

LCMS: 98.68%; i/z=447.8

HPLC: 97.5%

### EXAMPLE: 45

### Steps 1 to 3 were Performed in a Manner Similar to What has been Described for Example 1 and Step 4 was Performed in a Manner Similar to What has been Described for Example 6

### Step: 5

### Synthesis of 6-Chloro-7-(2-fluoro-4-iodo-phenylamino)-5-oxo-1,2,3,5-tetrahydro-indolizine-8-carboxylic acid ethoxy-amide

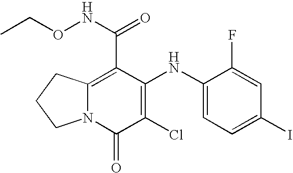

EDCI (256 mg, 0.001 mol) and HOBt (181 mg, 0.001 mol) were added to a stirred solution of 6-chloro-7-(2-fluoro-4-iodo-phenylamino)-5-oxo-1,2,3,5-tetrahydro-indolizine-8-carboxylic acid (200 mg, 0.000446 mol) in dry DMF (5 mL) at 0° C. The reaction mixture was stirred for 1.30 hrs at 0° C. under nitrogen atmosphere. This was followed by addition of O-ethyl-hydroxylamine hydrochloride (130 mg, 0.001 mol), TEA (0.2 ml, 0.001 mol) at 0° C. The reaction mixture was stirred for 18 hrs at RT. The reaction mixture was partitioned between EtOAc and water. The organic layer was washed with water, saturated NaHCO₃ solution and brine solution, dried over anhydrous Na₂SO₄, concentrated and the concentrate was washed with methanol to afford 105 mg (48% yield) of 6-chloro-7-(2-fluoro-4-iodo-phenylamino)-5-oxo-1,2,3,5-tetrahydro-indolizine-8-carboxylic acid ethoxy-amide as the required product.

H¹ NMR (DMSO-D₆, 300 MHz) δ 11.25-11.2 (br s, 1H), 8.02-7.94 (br s, 1H), 7.6-7.5 (d, 1H), 7.4 (d, 1H), 6.8-6.7 (t, 1H), 4.0 (t, 2H), 3.6-3.5 (m, 2H), 3.1 (t, 2H), 2.1-2.0 (m, 2H), 1.1 (t, 3H)

LCMS: 99%; m/z=491.6

HPLC: 96%

### EXAMPLE: 46

### Steps 1 to 3 were Performed in a Manner Similar to What has been Described for Example 1 and Step 4 was Performed in a Manner Similar to What has been Described for Example 6

### Step: 5

### Synthesis of 6-Chloro-7-(2-fluoro-4-iodo-phenylamino)-5-oxo-1,2,3,1-tetrahydro-indolizine-8-carboxylic acid (2,2-dimethyl-[1,3]dioxolan-4-ylmethoxy)-amide

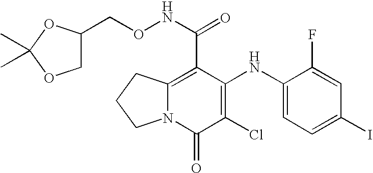

EDCI (256 mg, 0.001 mol) and HOBt (180 mg, 0.001 mol) were added to a stirred solution of 6-chloro-7-(2-fluoro-4-iodo-phenylamino)-5-oxo-1,2,3,5-tetrahydro-indolizine-8-carboxylic acid (200 mg, 0.0004 mol) in dry DMF (5 mL) at 0° C. The reaction mixture was stirred for 1.30 hrs at 0° C. under nitrogen atmosphere. This was followed by addition of O-(2,2-dimethyl-[1,3]dioxolan-4-ylmethyl)-hydroxylamine (196 mg, 0.00 μmol), TEA (0.2 ml, 0.001 mol) at 0° C. The reaction mixture was stirred for 18 hrs at RT. The reaction mixture was partitioned between EtOAc and water. The organic layer was washed with water, saturated NaHCO₃ solution and brine solution, dried over anhydrous Na₂SO₄ and concentrated to afford 170 mg (66% yield) of 6-chloro-7-(2-fluoro-4-iodo-phenylamino)-5-oxo-1,2,3,5-tetrahydro-indolizine-8-carboxylic acid (2,2-dimethyl-[1,3]dioxolan-4-ylmethoxy)-amide as the required product.

LCMS: 61%; m/z=577.8, M+1

### Step: 6

### Synthesis of 6-Chloro-7-(2-fluoro-4-iodo-phenylamino)-5-oxo-1,2,3,1-tetrahydro-indolizine-8-carboxylic acid (2,3-dihydroxy-propoxy)-amide

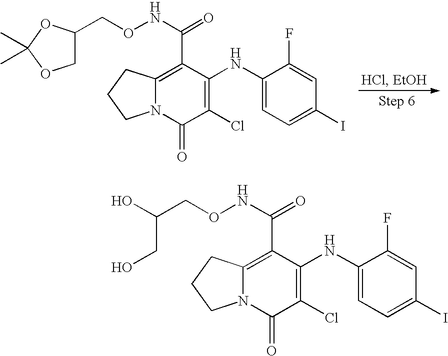

1N HCl (0.6 mL) was added to a stirred solution of 6-chloro-7-(2-fluoro-4-iodo-phenylamino)-5-oxo-1,2,3,5-tetrahydro-indolizine-8-carboxylic acid (2,2-dimethyl-[1,3]dioxolan-4-ylmethoxy)-amide (170 mg, 0.0003 mol) dissolved in EtOH (6 μL). The reaction mixture was stirred for 2 hrs at room temperature. Ethanol was distilled and the crude product was purified by recrystallization using methanol to afford 25 mg (17.35% yield) of 7-(4-bromo-2-fluoro-phenylamino)-6-chloro-5-oxo-1,2,3,5-tetrahydro-indolizine-8-carboxylic acid (2,3-dihydroxy-propoxy)-amide as a white solid.

H¹ NMR (DMSO-D₆, 300 MHz) δ 11.4-11.35 (br s, 1H), 8.0-7.96 (br s, 1H), 7.6-7.5 (dd, 1H), 7.4 (dd, 1H), 7.4 (d, 1H), 6.8 (t, 1H), 4.8 (d, 1H), 4.6 (t, 1H), 4.0 (t, 2H), 3.7-3.4 (m, 1H), 3.1 (t, 2H), 2.1-2.0 (m, 2H)

LCMS: 85%; m/z=491.7, M+1

HPLC: 96%

### EXAMPLE: 47

### Steps 1 to 3 were Performed in a Manner Similar to What has been Described for Example 8

### Step: 4

### Synthesis of 6-Fluoro-7-(2-fluoro-4-trifluoromethyl-phenylamino)-5-oxo-1,2,3,5-tetrahydro-indolizine-8-carboxylic acid

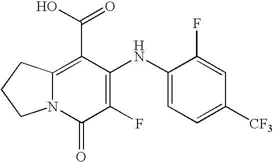

LDA (416 mg, 0.004 mol) was added to a solution of 2-fluoro-4-trifluoromethyl-phenylamine (484 mg, 0.003 mol) in THF (10 mL) at −78° C. and the resulting mixture was stirred for 1 hr at −78° C. This was followed by addition of 7-chloro-6-fluoro-5-oxo-1,2,3,5-tetrahydro-indolizine-8-carboxylic acid (250 mg, 0.001 mol) in THF (30 mL) at −78° C. and stirring was continued for a further 18 hrs at RT. THF from the reaction mixture was distilled and this was followed by addition of 1N HCl (5 mL) and ether (10 mL). The reaction mixture was stirred for 15 minutes and the precipitate was collected to afford 150 mg (37% yield) of 6-fluoro-7-(2-fluoro-4-trifluoromethyl-phenylamino)-5-oxo-1,2,3,5-tetrahydro-indolizine-8-carboxylic acid as the required product.

LC-MS purity: 100%, m/z=375, (M+)

HPLC: 91.4%

¹H NMR (DMSO-D₆, 300 MHz): δ 13.8-13.6 (br s, 1H), 9.6 (s, 1H), 7.7 (d, 1H), 7.52 (d, 1H), 7.15 (q, 1H), 4.1 (t, 2H), 3.5 (t, 2H), 2.2-2.1 (m, 2H).

### EXAMPLE: 48

### Steps 1 to 3 were Performed in a Manner Similar to What has been Described for Example 8 and Step 4 was Performed in a Manner Similar to What has been Described for Example 47

### Step: 5

### Synthesis of 6-Fluoro-7-(2-fluoro-4-trifluoromethyl-phenylamino)-5-oxo-1,2,3,5-tetrahydro-indolizine-8-carboxylic acid cyclopropylmethoxy-amide

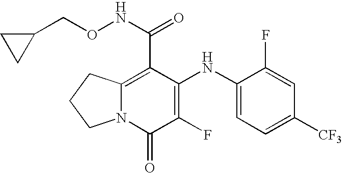

EDCI (138 mg, 0.001 mol) and HOBt (98 mg, 0.001 mol) were added to a stirred solution of 6-fluoro-7-(2-fluoro-4-trifluoromethyl-phenylamino)-5-oxo-1,2,3,5-tetrahydro-indolizine-8-carboxylic acid (90 mg, 0.0002 mol) in DMF (6 mL) and the reaction mixture was stirred for 30 mins at RT. This was followed by addition of O-cyclopropylmethyl-hydroxylamine hydrochloride (90 mg, 0.001 mol) and TEA (73 mg, 0.001 mol). The reaction mixture was stirred for 20 hrs at RT. The reaction mixture was partitioned between water and ethyl acetate (20 mL). The organic layer was washed with saturated NaHCO₃ (20 mL), NH₄Cl (20 mL), and brine solution (20 mL), dried over Na₂SO₄, concentrated and the concentrate was dissolved in methanol (0.5 mL) and diethyl ether (10 mL). The precipitate formed was collected to afford 35 mg (33% yield) of 6-fluoro-7-(2-fluoro-4-trifluoromethyl-phenylamino)-5-oxo-1,2,3,5-tetrahydro-indolizine-8-carboxylic acid cyclopropylmethoxy-amide as the required product.

LCMS purity: 100%, m/z=443, (M+)

HPLC: 94.3%

¹H NMR (DMSO-D₆, 300 MHz): δ 11.40 (s, 1H), 8.4 (s, 1H), 7.65 (d, 1H), 7.43 (d, 1H), 7.12-7.02 (m, 1H), 4.1 (t, 2H), 3.5 (d, 2H), 3.2 (t, 2H), 2.2-2.1 (m, 2H), 1.05-0.95 (m, 1H), 0.52-0.42 (m, 2H), 0.25-0.15 (m, 2H).

### EXAMPLE: 49

### Steps 1 to 3 were Performed in a Manner Similar to What has been Described for Example 8

### Step: 3a

### Synthesis of 2-Fluoro-4-thiocyanato-phenylamine

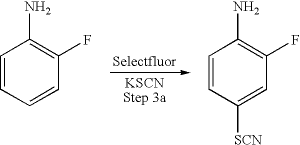

2-Fluoro-phenylamine (2 g, 0.018 mol) were added to a solution of selectfluor reagent (5.9 g, 0.017 mol) and KSCN (1.81 g, 0.019 mol) in acetonitrile and the resulting reaction mass was stirred for 70 hrs at RT. The solvent was distilled and the reaction mass was dissolved in water (300 mL), extracted twice with DCM (75 mL) and the organic layer was washed with water (100 mL) and brine solution (100 mL). The reaction mixture was dried over Na₂SO₄, concentrated and the concentrate was purified by column chromatography (using silica gel, 5-10% of ethylacetate in hexane as eluant) to afford 740 mg (25% yield) of 2-fluoro-4-thiocyanato-phenylamine as a pale yellow liquid.

¹H NMR (DMSO-D₆, 300 MHz): δ7.3-7.15 (m, 2H), 6.8 (t, 1H), 4.15-4.0 (br s, 2H)

### Step: 3b

### Synthesis of 2-Fluoro-4-methylsulfanyl-phenylamine

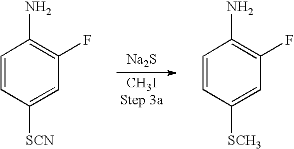

Na₂S (1.04 g, 0.011 mol) in water (2.2 mL) was added to a solution of 2-fluoro-4-thiocyanato-phenylamine (730 mg, 0.004 mol) in ethanol (12 mL) and the reaction mixture was stirred for 2 hrs at 50° C. This was followed by addition of CH₃I (683 mg, 0.0047 mol) in ethanol (2 mL) and the stirring was continued for a further 3 hrs. The reaction mass was diluted with ethylacetate, this was followed by addition of water (50 mL) and extraction with ethylacetate. The organic layer was washed with water (20 mL), brine solution (20 mL) and concentrated to afford 610 mg (89% yield) of 2-fluoro-4-methylsulfanyl-phenylamine as the required product.

¹H NMR (DMSO-D₆, 300 MHz): δ 7.2-6.92 (m, 2H), 6.72 (t, 1H), 3.8-3.6 (br s, 2H), 2.4 (s, 3H)

### Step: 4

### Synthesis of 6-Fluoro-7-(2-fluoro-4-methylsulfanyl-phenylamino)-5-oxo-1,2,3,5-tetrahydro-indolizine-8-carboxylic acid

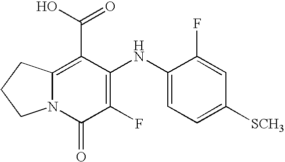

LDA (316 mg, 0.00296 mol) was added to a solution of 2-fluoro-4-methylsulfanyl-phenylamine (322 mg, 0.002 mol) in THF (10 mL) at −78° C. and the resulting mixture was stirred for 1.30 hrs at −78° C. This was followed by addition of 7-chloro-6-fluoro-5-oxo-1,2,3,5-tetrahydro-indolizine-8-carboxylic acid (190 mg, 0.001 mol) in THF (30 mL) at −78° C. and the stirring was continued for a further 24 hrs at RT. THF was distilled from the reaction mixture and this was followed by addition of 1N HCl (12 mL) and ether (10 mL). The reaction mixture was stirred for 15 minutes and the precipitate was collected to afford 128 mg of 6-fluoro-7-(2-fluoro-4-methylsulfanyl-phenylamino)-5-oxo-1,2,3,5-tetrahydro-indolizine-8-carboxylic acid along with the starting material which was used in the next step without further purification.

LC-MS purity: 50%, m/z=353, (M+)

### Step: 5

### Synthesis of 6-Fluoro-7-(2-fluoro-4-methylsulfanyl-phenylamino)-5-oxo-1,2,3,5-tetrahydro-indolizine-8-carboxylic acid cyclopropylmethoxy-amide

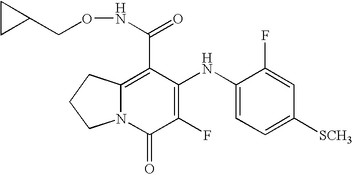

EDCI (227 mg, 0.001 mol) and HOBt (160 mg, 0.001 mol) were added to a stirred solution of 6-fluoro-7-(2-fluoro-4-methylsulfanyl-phenylamino)-5-oxo-1,2,3,5-tetrahydro-indolizine-8-carboxylic acid (120 mg, 0.0003 mol) in DMF (5 mL) and the reaction mixture was stirred for 1 hr at RT. This was followed by addition of O-cyclopropylmethyl-hydroxylamine hydrochloride (147 mg, 0.001 mol) and TEA (120 mg, 0.001 mol). The reaction mixture was stirred for 16 hrs at RT. The reaction mixture was partitioned between ethylacetate and water. The organic layer was washed with saturated NaHCO₃, NH₄Cl, brine solution, dried over Na₂SO₄ and concentrated. The concentrate was dissolved in methanol (0.5 mL) and diethyl ether (10 mL). The precipitate was collected to afford 7 mg (9.8% yield) of 6-fluoro-7-(2-fluoro-4-methylsulfanyl-phenylamino)-5-oxo-1,2,3,5-tetrahydro-indolizine-8-carboxylic acid cyclopropylmethoxy-amide as the required product.

LCMS purity: 89.5%, m/z=422, (M+)

HPLC: 91%

¹H NMR (DMSO-D₆, 300 MHz): δ 11.4 (s, 1H), 8.0 (s, 1H), 7.2 (d, 1H), 7.0 (s, 2H), 4.0 (t, 2H), 3.5 (d, 2H), 3.2 (t, 2H), 2.5 (s, 3H), 2.14-2.04 (m, 2H), 1.05-0.95 (m, 1H), 0.55-0.45 (m, 2H), 0.25-0.15 (m, 2H).

### EXAMPLE: 50

### Steps 1 to 3 were Performed in a Manner Similar to What has been Described for Example 8 and Step 4 was Performed in a Manner Similar to What has been Described for Example 11

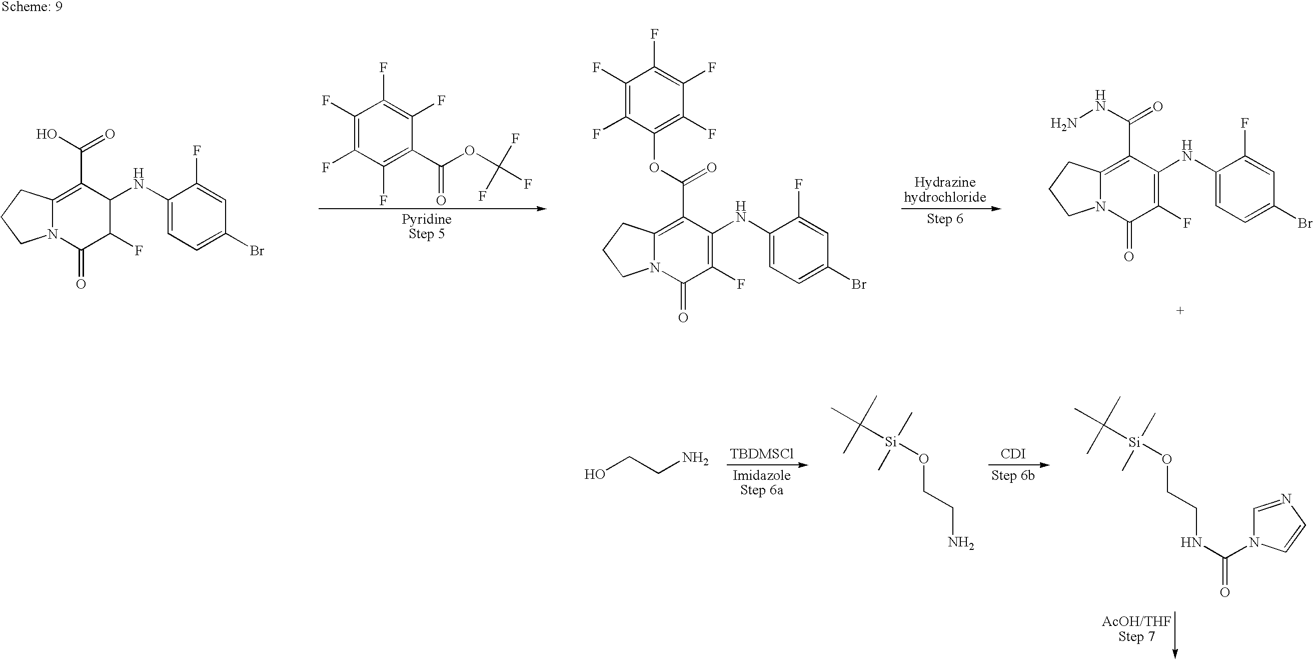

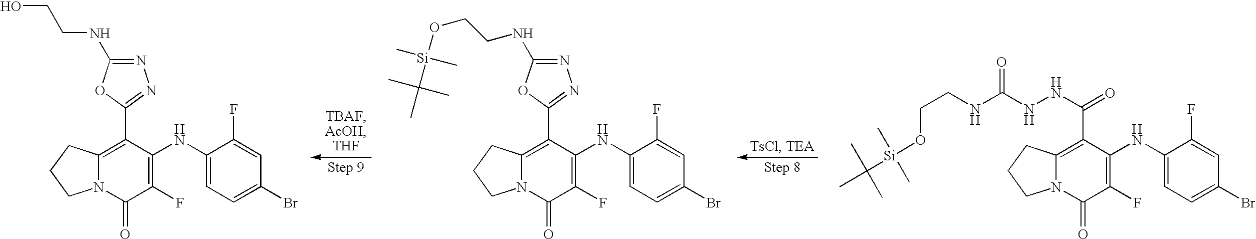

### Step: 5

### Synthesis of 7-(4-Bromo-2-fluoro-phenylamino)-6-fluoro-5-oxo-1,2,3,5-tetrahydro-indolizine-8-carboxylic acid pentafluorophenyl ester

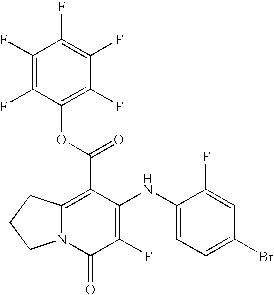

2,3,4,5,6-Pentafluoro-benzoic acid trifluoromethyl ester (136 mg, 0.0005 mol) and pyridine (38 mg, 0.0005 mol) were added to a solution of 7-(4-bromo-2-fluoro-phenylamino)-6-fluoro-5-oxo-1,2,3,5-tetrahydro-indolizine-8-carboxylic acid (170 mg, 0.0004 mol) in DMF (3 mL) and the reaction mixture was stirred for 4 hrs at RT. The reaction mixture was partitioned between ethylacetate and water. The organic layer was washed with NaHCO₃, twice with 1M HCl solution and brine solution. The organic layer was dried over Na₂SO₄ and concentrated to afford 256 mg of 7-(4-bromo-2-fluoro-phenylamino)-6-fluoro-5-oxo-1,2,3,5-tetrahydro-indolizine-8-carboxylic acid pentafluorophenyl ester as the crude product which was used for the next step without further purification.

### Step: 6

### Synthesis of 7-(4-Bromo-2-fluoro-phenylamino)-6-fluoro-5-oxo-1,2,3,1-tetrahydro-indolizine-8-carboxylic acid hydrazide

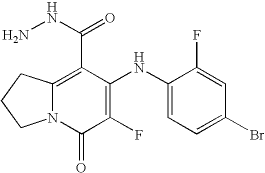

TEA (98 mg, 0.001 mol) was added to a stirred solution of hydrazine hydrochloride (35 mg, 0.00 μmol) in DCM (5 mL) and the reaction mixture was stirred for 1 hr. This was followed by addition of 7-(4-bromo-2-fluoro-phenylamino)-6-fluoro-5-oxo-1,2,3,5-tetrahydro-indolizine-8-carboxylic acid pentafluorophenyl ester (251 mg, 0.0005 mol) and the stirring was continued for a further 8 hrs. The reaction mixture was partitioned between ethylacetate and water. The organic layer was washed twice with water, saturated NaHCO₃ and twice with brine solution. The organic layer was dried over Na₂SO₄ and concentrated to afford 138 mg (55% yield) of 7-(4-bromo-2-fluoro-phenylamino)-6-fluoro-5-oxo-1,2,3,5-tetrahydro-indolizine-8-carboxylic acid hydrazide as the required product.

LCMS purity: 78%, m/z=399, 401, (M+)

### Step: 6a

### Synthesis of 2-(tert-Butyl-dimethyl-silanyloxy)-ethylamine

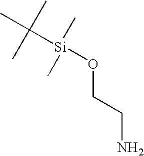

Imidazole (23.4 g, 0.344 mol) was added to a solution of 2-amino-ethanol (20 g, 0.327 mol) in DMF (400 mL) and the reaction mixture was cooled to 0° C. This was followed by addition of tert-butyl-chloro-dimethyl-silane (51.8 g, 0.344 mol) and the reaction mixture was stirred for 3 hrs at RT. The residual mass was diluted with water (1 L) and extracted twice with ethylacetate (300 mL). Ethyl acetate layer was washed with water, 0.1N HCl (100 mL) and brine solution (100 mL). The organic layer was dried over Na₂SO₄, concentrated and the crude product was purified by column chromatography (using silica gel, 30-40% ethylacetate in hexane as the eluting system) to afford 18. Ig (31% yield) of 2-(tert-butyl-dimethyl-silanyloxy)-ethylamine as the required product.

LCMS purity: 92%, m/z=176, (M+)

### Step: 6b

### Synthesis of Imidazole-1-carboxylic acid [2-(tert-butyl-dimethyl-silanyloxy)-ethyl]-amide

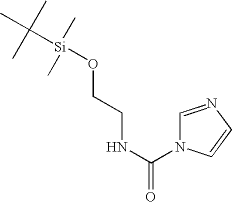

2-(tert-Butyl-dimethyl-silanyloxy)-ethylamine (7.3 g, 0.042 mol) in DCM (150 mL) was added to a solution of CDI (10.11 g, 0.062 mol) in THF (60 mL) at RT and the reaction mixture was stirred for 8 hrs at 50° C. The solvent from the reaction mixture distilled and the residual crude product was purified by column chromatography (using silica gel, 20-40% ethylacetate in hexane as the eluting system) to afford 2. Ig (19% yield) of imidazole-1-carboxylic acid [2-(tert-butyl-dimethyl-silanyloxy)-ethyl]-amide as the required product.

LCMS purity: 94.5%, m/z=270, (M+)

### Step: 7

### Synthesis of 7-(4-Bromo-2-fluoro-phenylamino)-6-fluoro-5-oxo-1,2,3,5-tetrahydro-indolizine-8-carboxylic acid N′-2-(tert-Butyl-dimethyl-silanyloxy)-ethyl-amino-carbonyl-hydrazide

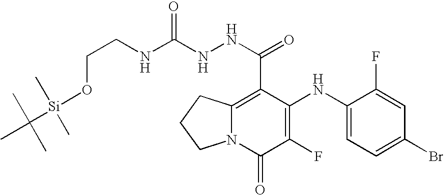

Acetic acid (63 mg, 0.0003 mol) and 7-(4-bromo-2-fluoro-phenylamino)-6-fluoro-5-oxo-1,2,3,5-tetrahydro-indolizine-8-carboxylic acid hydrazide (135 mg, 0.0003 mol) were added to a solution of imidazole-1-carboxylic acid [2-(tert-butyl-dimethyl-silanyloxy)-ethyl]-amide (91 mg, 0.0003 mol) in THF (10 mL) and the reaction mixture was stirred for 14 hrs at RT. The solvent from the reaction mixture was distilled to afford 213 mg of 7-(4-bromo-2-fluoro-phenylamino)-6-fluoro-5-oxo-1,2,3,5-tetrahydro-indolizine-8-carboxylic acid N′-2-(tert-butyl-dimethyl-silanyloxy)-ethyl-amino-carbonyl-hydrazide as the crude product which was used for the next step without a further purification.

LCMS purity: 49%, m/z=600, (M+)

### Step: 8

### Synthesis of 7-(4-Bromo-2-fluoro-phenylamino)-8-{5-[2-(tert-butyl-dimethyl-silanyloxy)-ethylamino]-[1,3,4]oxadiazol-2-yl}-6-fluoro-2,3-dihydro-1H-indolizin-5-one

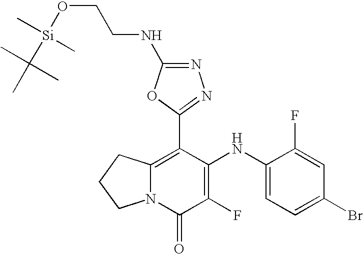

Tosyl chloride (63 mg, 0.0003 mol) and TEA (84 mg, 0.0008 mol) were added to stirred a solution of 7-(4-bromo-2-fluoro-phenylamino)-6-fluoro-5-oxo-1,2,3,5-tetrahydro-indolizine-8-carboxylic acid N′-2-(tert-butyl-dimethyl-silanyloxy)-ethyl-amino-carbonyl-hydrazide (200 mg, 0.0003 mol) in DCM (8 mL) and the reaction mixture was stirred for 12 hrs at RT. DCM from the reaction mixture was distilled and the residual mixture was diluted with water and extracted with ethylacetate. The organic layer was dried over Na₂SO₄, concentrated to afford 18.1 g (31% yield) of 7-(4-bromo-2-fluoro-phenylamino)-8-{5-[2-(tert-butyl-dimethyl-silanyloxy)-ethylamino]-[1,3,4]oxadiazol-2-yl}-6-fluoro-2,3-dihydro-1H-indolizin-5-one as the crude product which was used for the next step without a further purification.

### Step: 9

### Synthesis of 7-(4-Bromo-2-fluoro-phenylamino)-6-fluoro-8-[5-(2-hydroxy-ethylamino)-[1,3,4]oxadiazol-2-yl]-2,3-dihydro-1H-indolizin-5-one

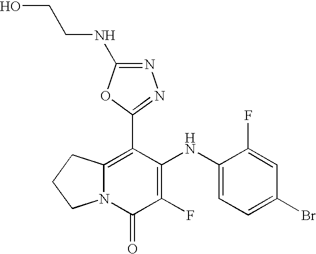

Acetic acid (25 mg, 0.0004 mol) and tetra butyl ammonium fluoride (168 mg, 0.0006 mol) were added to a solution of 7-(4-bromo-2-fluoro-phenylamino)-8-{5-[2-(tert-butyl-dimethyl-silanyloxy)-ethylamino]-[1,3,4]oxadiazol-2-yl}-6-fluoro-2,3-dihydro-1H-indolizin-5-one (250 mg, 0.0004 mol) in THF (6 mL) at 0° C. and the reaction mixture was stirred for 3 hrs at RT. The reaction mixture was diluted with ethylacetate and water. The organic layer was washed with NaHCO₃ solution, 1M HCl and brine solution, dried over Na₂SO₄ and concentrated. The crude product was dissolved in DCM (2 mL), this was followed by addition of diethyl ether, the precipitate formed was collected and dried to afford 27.5 mg (14% yield) of 7-(4-bromo-2-fluoro-phenylamino)-6-fluoro-8-[5-(2-hydroxy-ethylamino)-[1,3,4]oxadiazol-2-yl]-2,3-dihydro-1H-indolizin-5-one as the required product.

LCMS purity: 92.2%, m/z=468, 470 (M+)

HPLC: 95%

¹H NMR (DMSO-D₆, 300 MHz): δ 9.1 (s, 1H), 7.82 (t, 1H), 7.55 (d, 1H), 7.32 (s, 1H), 7.12 (m, 1H), 4.8 (t, 1H), 4.1 (t, 2H), 3.6-3.5 (q, 12H), 3.4-3.2 (m, 4H), 2.3-2.1 (m, 2H).

### EXAMPLE: 51

### Steps 1 to 4 were Performed in a Manner Similar to What has been Described for Example 8

### Step: 5

### Synthesis of 7-(4-Bromo-2-fluoro-phenylamino)-6-fluoro-5-oxo-1,2,3,5-tetrahydro-indolizine-8-carboxylic acid methoxy-amide

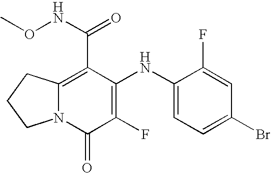

EDCI (296 mg, 0.0015 mol) and HOBt (209 mg, 0.0015 mol) were added to a stirred solution of 7-(4-bromo-2-fluoro-phenylamino)-6-fluoro-5-oxo-1,2,3,5-tetrahydro-indolizine-8-carboxylic acid (200 mg, 0.0005 mol) in DMF (5 mL) and the reaction mixture was stirred for 1 hr at RT. This was followed by addition of O-methyl-hydroxylamine (130 mg, 0.002 mol) and TEA (156 mg, 0.002 mol). The reaction mixture was stirred for 19 hrs at RT. The reaction mixture was partitioned between water and ethyl acetate. The organic layer was washed with saturated NaHCO₃, saturated NH₄Cl, and brine solution, dried over Na₂SO₄ and concentrated. The concentrate was dissolved in IPA (2 mL), this was followed by the addition of diethyl ether (15 mL). The precipitate formed was collected to afford 57 mg (27% yield) of 7-(4-bromo-2-fluoro-phenylamino)-6-fluoro-5-oxo-1,2,3,5-tetrahydro-indolizine-8-carboxylic acid methoxy-amide as the required product.

LCMS purity: 97%, m/z 413, 415 (M+, Br pattern)

HPLC: 99%

¹H NMR (DMSO-D₆, 300 MHz): δ 11.42 (s, 1H), 8.1 (s, 1H), 7.5 (d, 1H), 7.3 (d, 1H), 6.98 (m, 1H), 4.00 (t, 2H), 3.6 (s, 3H), 3.3 (t, 2H), 2.10 (m, 2H)

### EXAMPLE: 52

### Steps 1 to 4 were Performed in a Manner Similar to What has been Described for Example 8

### Step: 5

### Synthesis of 7-(4-Bromo-2-fluoro-phenylamino)-6-fluoro-5-oxo-1,2,3,5-tetrahydro-indolizine-8-carboxylic acid ethoxy-amide

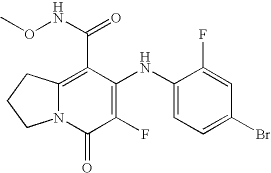

EDCI (296 mg, 0.002 mol) and HOBt (209 mg, 0.002 mol) were added to a stirred solution of 7-(4-bromo-2-fluoro-phenylamino)-6-fluoro-5-oxo-1,2,3,5-tetrahydro-indolizine-8-carboxylic acid (200 mg, 0.0005 mol) in DMF (5 mL) and the reaction mixture was stirred for 1 hr at RT. This was followed by addition of O-ethyl-hydroxylamine (152 mg, 0.002 mol) and TEA (156 mg, 0.002 mol). The reaction mixture was stirred for 18 hrs at RT. The reaction mixture was partitioned between water and ethyl acetate. The organic layer was washed with saturated NaHCO₃, saturated NH₄Cl, and brine solution, dried over Na₂SO₄ and concentrated. The concentrate was dissolved in methanol (1 mL), ether (10 mL) was added to this and the precipitate formed was collected to afford 97 mg (44% yield) of 7-(4-bromo-2-fluoro-phenylamino)-6-fluoro-5-oxo-1,2,3,5-tetrahydro-indolizine-8-carboxylic acid ethoxy-amide as the required product.

LCMS purity: 97%, m/z 428, 430 (M+, Br pattern)

HPLC: 97.6%

¹H NMR (DMSO-D₆, 300 MHz): δ 11.4 (s, 1H), 8.08 (s, 1H), 7.52 (d, 1H), 7.28 (d, 1H), 6.98 (m, 1H), 4.00 (t, 2H), 3.8 (q, 2H), 3.2 (t, 2H), 2.10 (m, 2H), 1.12 (t, 3H)

### EXAMPLE: 53

### Steps 1 to 4 were Performed in a Manner Similar to What has been Described for Example 8

### Step: 5

### Synthesis of 7-(4-Bromo-2-fluoro-phenylamino)-6-fluoro-5-oxo-1,2,3,5-tetrahydro-indolizine-8-carboxylic acid (2-vinyloxy-ethoxy)-amide

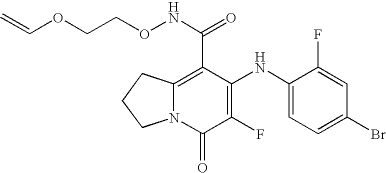

EDCI (148 mg, 0.001 mol) and HOBt (104 mg, 0.001 mol) were added to a stirred solution of 7-(4-bromo-2-fluoro-phenylamino)-6-fluoro-5-oxo-1,2,3,5-tetrahydro-indolizine-8-carboxylic acid (100 mg, 0.0003 mol) in DMF (3 mL) and the reaction mixture was stirred for 1.30 hr at RT. This was followed by addition of O-(2-vinyloxy-ethyl)-hydroxylamine (80 mg, 0.001 mol) and TEA (78 mg, 0.001 mol). The reaction mixture was stirred for 19 hrs at RT. The reaction mixture was partitioned between water and ethyl acetate. The organic layer was washed with saturated NaHCO₃, saturated NH₄Cl, and brine solution, dried over Na₂SO₄ and concentrated to afford 110 mg of the crude product which was used in the next step without a further purification.

### Step: 6

### Synthesis of 7-(4-Bromo-2-fluoro-phenylamino)-6-fluoro-5-oxo-1,2,3,5-tetrahydro-indolizine-8-carboxylic acid (2-hydroxy-ethoxy)-amide

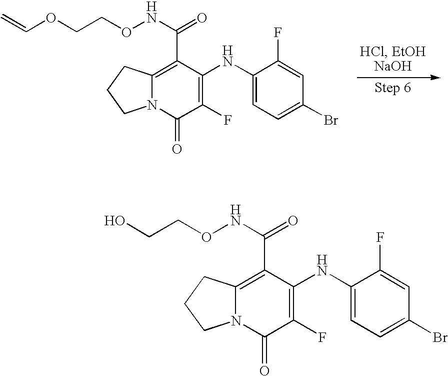

1N HCl (1.6 mL) were added to a stirred solution of 7-(4-bromo-2-fluoro-phenylamino)-6-fluoro-5-oxo-1,2,3,5-tetrahydro-indolizine-8-carboxylic acid (2-vinyloxy-ethoxy)-amide (110 mg, 0.0002 mol) in a 1:1 mixture of THF and EtOH (2 mL). The reaction mixture was stirred for 1 hr. The reaction mixture was diluted with ethylacetate; pH was adjusted to 5 using 2N NaOH and extracted with EtOAc.

The organic layer was dried over Na₂SO₄ and concentrated. The concentrate was dissolved in 2 mL of IPA, 10 mL of ether was added to this and the precipitate formed was collected to afford 5 mg (7% yield) of 7-(4-bromo-2-fluoro-phenylamino)-6-fluoro-5-oxo-1,2,3,5-tetrahydro-indolizine-8-carboxylic acid (2-hydroxy-ethoxy)-amide as the required product.

LCMS purity: 91.8%, m/z=443.9, 445.9 (M+, Br pattern)

HPLC: 98.2%

¹H NMR (DMSO-D₆, 300 MHz): δ 11.42 (s, 1H), 8.1 (s, 1H), 7.5 (d, 1H), 7.3 (d, 1H), 6.98 (m, 1H), 4.8 (t, 1H), 4.00 (t, 2H), 3.8 (t, 2H) 3.55 (t, 2H), 3.2 (t, 2H), 2.12 (m, 2H)

### EXAMPLE: 54

### Steps 1 to 3 were Performed in a Manner Similar to What has been Described for Example 8

### Step: 4

### Synthesis of 7-(4-Bromo-2-methyl-phenylamino)-6-fluoro-5-oxo-1,2,3,5-tetrahydro-indolizine-8-carboxylic acid

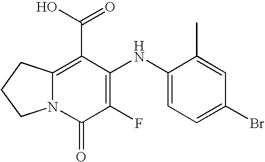

LDA (2.33 g, 0.01 mol) was added to a solution of 4-bromo-2-methyl-phenylamine (1.4 mg, 0.008 mol) in THF (10 mL) at −78° C. and the resulting mixture was stirred for 1 hr at −78° C. This was followed by addition of 7-chloro-6-fluoro-5-oxo-1,2,3,5-tetrahydro-indolizine-8-carboxylic acid (700 mg, 0.003 mol) in THF (50 mL) at −78° C. and the stirring was continued for a further 20 hrs at RT. THF was distilled and this was followed by addition of 1N HCl (20 mL), water (25 mL) and ether (10 mL). The precipitate formed was collected to afford 281 mg (24% yield) of 7-(4-bromo-2-methyl-phenylamino)-6-fluoro-5-oxo-1,2,3,5-tetrahydro-indolizine-8-carboxylic acid.

LCMS purity: 96%, m/z 380, 382 (M+, Br Pattern)

HPLC: 95.89%

¹H NMR (DMSO-D₆, 300 MHz): δ 13.70 (s, 1H), 9.4 (s, 1H), 7.4 (s, 1H), 7.3 (d, 1H), 6.8 (m, 1H), 4.04 (t, 2H), 3.48 (t, 2H), 2.25 (s, 3H), 2.10 (m, 2H).

### EXAMPLE: 55

### Steps 1 to 3 were Performed in a Manner Similar to What has been Described for Example 8 and Step 4 was Performed in a Manner Similar to What has been Described for Example 54

### Step: 5

### Synthesis of 7-(4-Bromo-2-methyl-phenylamino)-6-fluoro-5-oxo-1,2,3,5-tetrahydro-indolizine-8-carboxylic acid cyclopropylmethoxy-amide

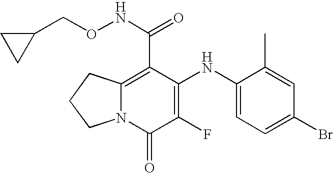

EDCI (346 mg, 0.002 mol) and HOBt (244 mg, 0.002 mol) were added to a stirred solution of 7-(4-bromo-2-methyl-phenylamino)-6-fluoro-5-oxo-1,2,3,5-tetrahydro-indolizine-8-carboxylic acid (230 mg, 0.001 mol) in DMF (3 mL) and the reaction mixture was stirred for 1 hr at RT. This was followed by addition of O-cyclopropylmethyl-hydroxylamine (224 mg, 0.002 mol) and TEA (183 mg, 0.002 mol). The reaction mixture was stirred for 24 hrs at RT. The reaction mixture was partitioned between water and ethyl acetate. The organic layer was washed with saturated NaHCO₃, saturated NH4Cl, and brine solution, dried over Na₂SO₄ and concentrated. The concentrate was dissolved in 5 mL of methanol, 25 mL of diethyl ether was added into this and the precipitate formed was collected to afford 40 mg (14.7% yield) of 7-(4-bromo-2-methyl-phenylamino)-6-fluoro-5-oxo-1,2,3,5-tetrahydro-indolizine-8-carboxylic acid cyclopropylmethoxy-amide as the required product.

LCMS purity: 95%, m/z 450, 452 (M+, Br pattern)

HPLC: 96.1%

¹H NMR (DMSO-D₆, 300 MHz): δ 11.30 (s, 1H), 7.78 (s, 1H), 7.4 (s, 1H), 7.22 (d, 1H), 6.88 (m, 1H), 4.00 (t, 2H), 3.4 (d, 2H), 3.20 (t, 2H), 2.2 (s, 3H) 2.10 (m, 2H) 1.00 (m, 1H), 0.50 (m, 2H), 0.20 (m, 2H)

### EXAMPLE: 56

### Steps 1 to 3 were Performed in a Manner Similar to What has been Described for Example 8

### Step: 4

### Synthesis of 7-(4-Bromo-2-methyl-phenylamino)-5-oxo-1,2,3,5-tetrahydro-indolizine-8-carboxylic acid

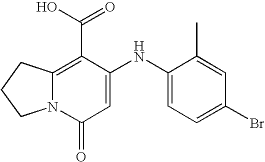

LDA (2.7 g, 0.0253 mol) were added to a solution of 4-bromo-2-methyl-phenylamine (3.28 mg, 0.018 mol) in THF (30 mL) at −78° C. and the resulting mixture was stirred for 45 mins at −78° C. This was followed by addition of 7-chloro-5-oxo-1,2,3,5-tetrahydro-indolizine-8-carboxylic acid (1.5 g, 0.01 mol) in THF (90 mL) at −78° C. and the stirring was continued for a further 21 hrs at RT. THF was distilled and this was followed by addition of 60 mL of 1N HCl (pH=1), water (115 mL) and diethylether (115 mL). The precipitate formed was collected to afford 610 mg (36% yield) of 7-(4-bromo-2-methyl-phenylamino)-5-oxo-1,2,3,5-tetrahydro-indolizine-8-carboxylic acid.

LCMS purity: 93%, m/z 363, 365 (M+, Br Pattern)

HPLC: 95.3%

¹H NMR (DMSO-D₆, 300 MHz): δ 13.30 (s, 1H), 9.8 (s, 1H), 7.6 (s, 1H), 7.42 (d, 1H), 7.2 (m, 1H), 5.08 (s, 1H), 3.8 (t, 2H), 3.48 (t, 2H), 2.35 (s, 3H), 2.15 (m, 2H).

### EXAMPLE: 57

### Steps 1 to 3 were Performed in a Manner Similar to What has been Described for Example 8 and Step 4 was Performed in a Manner Similar to What has been Described for Example 56

### Step: 5

### Synthesis of 7-(4-Bromo-2-methyl-phenylamino)-5-oxo-1,2,3,5-tetrahydro-indolizine-8-carboxylic acid cyclopropylmethoxy-amide

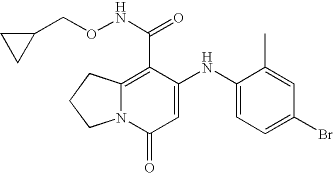

EDCI (473 mg, 0.002 mol) and HOBt (334 mg, 0.002 mol) were added to a stirred solution of 7-(4-bromo-2-methyl-phenylamino)-5-oxo-1,2,3,5-tetrahydro-indolizine-8-carboxylic acid (300 mg, 0.001 mol) in DMF (3 mL) and the reaction mixture was stirred for 1 hr at RT. This was followed by addition of O-cyclopropylmethyl-hydroxylamine (306 mg, 0.002 mol) and TEA (250 mg, 0.002 mol). The reaction mixture was stirred for 26 hrs at RT. The reaction mixture was partitioned between EtOAc and water. The organic layer was washed with saturated NH₄Cl, saturated NaHCO₃ solution and brine solution, dried over Na₂SO₄ and concentrated. The concentrate was dissolved in 2.5 mL of methanol, 10 mL of diethyl ether was added into this and the precipitate formed was collected to afford 47 mg (13% yield) of 7-(4-Bromo-2-methyl-phenylamino)-5-oxo-1,2,3,5-tetrahydro-indolizine-8-carboxylic acid cyclopropylmethoxy-amide as the required product.

LCMS purity: 96%, m/z 432, 434 (M+, Br pattern)

HPLC: 92.1%

¹H NMR (DMSO-D₆, 300 MHz): δ 11.36 (s, 1H), 8.02 (s, 1H), 7.58 (s, 1H), 7.4 (d, 1H), 7.15 (m, 1H), 5.02 (s, 1H) 3.8 (t, 2H), 3.7 (d, 2H), 3.20 (t, 2H), 2.2 (s, 3H) 2.16 (m, 2H), 1.10 (m, 1H), 0.52 (m, 2H), 0.30 (m, 2H)

### EXAMPLE: 58

### Steps 1 to 4 were Performed in a Manner Similar to What has been Described for Example 8

### Step: 5

### Synthesis of 2-{1-[6-Fluoro-7-(2-fluoro-4-iodo-phenylamino)-5-oxo-1,2,3,5-tetrahydro-indolizine-8-carbonyl]-3-hydroxy-azetidin-3-yl}-piperidine-1-carboxylic acid tert-butyl ester

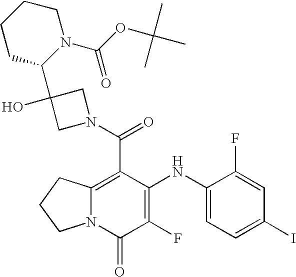

EDCI (154 mg, 0.001 mol) and HOBt (100 mg, 0.001 mol) were added to a stirred solution of 6-fluoro-7-(2-fluoro-4-iodo-phenylamino)-5-oxo-1,2,3,5-tetrahydro-indolizine-8-carboxylic acid (140 mg, 0.0003 mol) in DMF (4 mL) and the reaction mixture was stirred for 1 hr at RT. This was followed by addition of 2-(3-hydroxy-azetidin-3-yl)-piperidine-1-carboxylic acid tert-butyl ester (S-isomer) (166 mg, 0.001 mol) and TEA (98 mg, 0.001 mol). The reaction mixture was stirred for 16 hrs at RT. The reaction mixture was partitioned between EtOAc and water. The organic layer was washed with saturated NH₄Cl, saturated NaHCO₃ solution and brine solution, dried over Na₂SO₄ and concentrated. The concentrate was purified by column chromatography (using silica gel, 2-3% methanol in DCM as eluant) to afford 180 mg (82.9% yield) of 2-{1-[6-fluoro-7-(2-fluoro-4-iodo-phenylamino)-5-oxo-1,2,3,5-tetrahydro-indolizine-8-carbonyl]-3-hydroxy-azetidin-3-yl}-piperidine-1-carboxylic acid tert-butyl ester (S-isomer) as the required product.

### Step: 6

### Synthesis of 6-Fluoro-7-(2-fluoro-4-iodo-phenylamino)-8-(3-hydroxy-3-piperidin-2-yl-azetidine-1-carbonyl)-2,3-dihydro-1H-indolizin-5-one hydrochloride

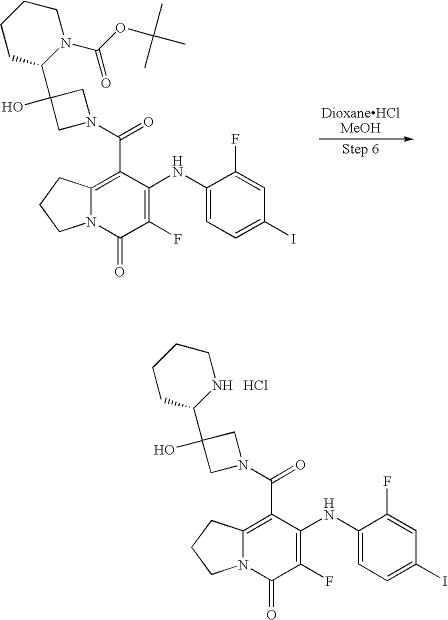

4N Dioxane in HCl (2.5 mL) were added to a solution of 2-{1-[6-fluoro-7-(2-fluoro-4-iodo-phenylamino)-5-oxo-1,2,3,5-tetrahydro-indolizine-8-carbonyl]-3-hydroxy-azetidin-3-yl}-piperidine-1-carboxylic acid tert-butyl ester (S-isomer) (50 mg, 0.000 μmol) in methanol (2 mL) and the resulting mixture was stirred for 1 hr at RT. The solvents were distilled from the reaction mixture and the residue was triturated with ether to afford 34 mg (75% yield) of 6-fluoro-7-(2-fluoro-4-iodo-phenylamino)-8-(3-hydroxy-3-piperidin-2-yl-azetidine-1-carbonyl)-2,3-dihydro-1H-indolizin-5-one hydrochloride (S-isomer) as the required product.

LCMS purity: 95.5%, m/z=570.9 (M+)

HPLC: 91.6%

¹H NMR (DMSO-D₆, 300 MHz): δ 8.4-8.2 (br s, 1H), 8.1 (s, 1H), 7.6 (d, 1H), 7.4 (d, 1H), 7.02-6.92 (m, 1H), 4.2-4.1 (m, 1H), 4.10-3.95 (m, 3H), 3.9-3.8 (m, 1H), 3.75-3.65 (m, 1H), 3.5-3.45 (m, 1H), 3.2-3.1 (m, 2H), 3.08 (t 2H), 2.9-2.8 (m, 1H), 2.2-2.08 (m, 2H), 1.75-1.65 (m, 4H), 1.5-1.35 (m, 2H)

### EXAMPLE: 59

### Steps 1 to 3 were Performed in a Manner Similar to What has been Described for Example 8 and Step 4 was Performed in a Manner Similar to What has been Described for Example 11

### Step: 5

### Synthesis of 2-{1-[7-(4-Bromo-2-fluoro-phenylamino)-6-fluoro-5-oxo-1,2,3,5-tetrahydro-indolizine-8-carbonyl]-3-hydroxy-azetidin-3-yl}-piperidine-1-carboxylic acid tert-butyl ester

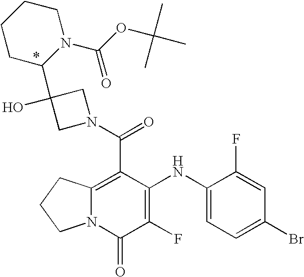

EDCI (185 mg, 0.001 mol) and HOBt (131 mg, 0.001 mol) were added to a stirred solution of 7-(4-bromo-2-fluoro-phenylamino)-6-fluoro-5-oxo-1,2,3,5-tetrahydro-indolizine-8-carboxylic acid (250 mg, 0.001 mol) in DMF (5 mL) and the reaction mixture was stirred for 1 hr at RT. This was followed by addition of 2-(3-hydroxy-azetidin-3-yl)-piperidine-1-carboxylic acid tert-butyl ester (racemic mixture) (199 mg, 0.001 mol) and TEA (196 mg, 0.002 mol). The reaction mixture was stirred for 16 hrs at RT. The reaction mixture was partitioned between EtOAc and water. The organic layer was washed with saturated NH₄Cl (10 mL), saturated NaHCO₃ solution (10 mL) and brine solution (10 mL), dried over Na₂SO₄ and concentrated. The concentrate was dissolved in 3 mL of ethylacetate to yield a precipitate which was collected to afford 200 mg (49.6% yield) of 2-{1-[7-(4-bromo-2-fluoro-phenylamino)-6-fluoro-5-oxo-1,2,3,5-tetrahydro-indolizine-8-carbonyl]-3-hydroxy-azetidin-3-yl}-piperidine-1-carboxylic acid tert-butyl ester (racemic mixture) as the required product.

HPLC: 98.4%

### Step: 6

### Synthesis of 7-(4-Bromo-2-fluoro-phenylamino)-6-fluoro-8-(3-hydroxy-3-piperidin-2-yl-azetidine-1-carbonyl)-2,3-dihydro-1H-indolizin-5-one

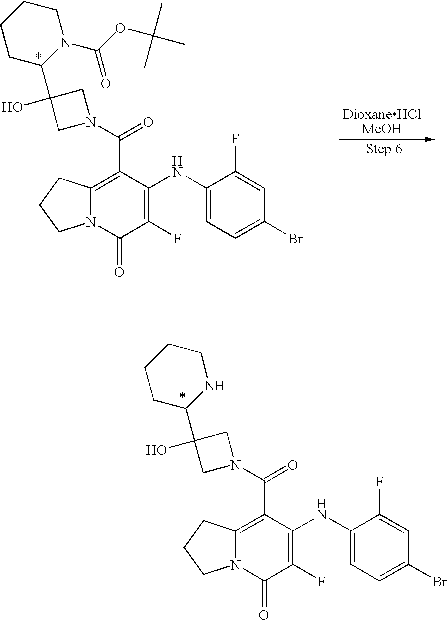

4N Dioxane in HCl (4 mL) were added to a solution of 2-{1-[7-(4-bromo-2-fluoro-phenylamino)-6-fluoro-5-oxo-1,2,3,5-tetrahydro-indolizine-8-carbonyl]-3-hydroxy-azetidin-3-yl}-piperidine-1-carboxylic acid tert-butyl ester (racemic mixture) (75 mg, 0.0001 mol) in methanol (1 mL) and the resulting mixture was stirred for 1 hr at RT. The solvents were distilled from the reaction mixture. Trituration with 5 mL of diethyl ether affords a precipitate which was collected to afford 48 mg (71.6% yield) of 7-(4-bromo-2-fluoro-phenylamino)-6-fluoro-8-(3-hydroxy-3-piperidin-2-yl-azetidine-1-carbonyl)-2,3-dihydro-1H-indolizin-5-one (racemic mixture) as the required product.

LC-MS purity: 97%, m/z 523, 525 (M+, Br Pattern)

¹H NMR (DMSO-D₆): δ 8.3-8.2 (br s, 1H), 8.12 (s, 1H), 7.52 (d, 1H), 7.3 (d, 1H), 7.16-7.02 (m, 1H), 4.2-4.1 (m, 1H), 4.10-3.90 (m, 4H), 3.75-3.65 (m, 1H), 3.5-3.45 (m, 1H), 3.2-3.1 (m, 2H), 3.08 (t, 2H), 2.9-2.8 (m, 1H), 2.2-2.08 (m, 2H), 1.8-1.5 (m, 4H), 1.45-1.3 (m, 2H)

### EXAMPLE: 60

### Steps 1 to 4 were Performed in a Manner Similar to What has been Described for Example 8

### Step: 5

### Synthesis of 2-{1-[6-Fluoro-7-(2-fluoro-4-iodo-phenylamino)-5-oxo-1,2,3,5-tetrahydro-indolizine-8-carbonyl]-3-hydroxy-azetidin-3-yl}-piperidine-1-carboxylic acid tert-butyl ester

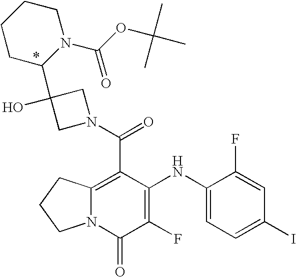

EDCI (165 mg, 0.001 mol) and HOBt (178 mg, 0.001 mol) were added to a stirred solution of 6-fluoro-7-(2-fluoro-4-iodo-phenylamino)-5-oxo-1,2,3,5-tetrahydro-indolizine-8-carboxylic acid (250 mg, 0.001 mol) in DMF (5 mL) and the reaction mixture was stirred for 1 hr at RT. This was followed by addition of 2-(3-hydroxy-azetidin-3-yl)-piperidine-1-carboxylic acid tert-butyl ester (racemic mixture) (180 mg, 0.001 mol) and TEA (175 mg, 0.002 mol). The reaction mixture was stirred for 16 hrs at RT. The reaction mixture was partitioned between EtOAc and water. The organic layer was washed with saturated NH₄Cl (10 mL), saturated NaHCO₃ solution (10 mL) and brine solution (10 mL), dried over Na₂SO₄ and concentrated. The concentrate (100 mg) was used for the next step without further purification.

### Step: 6

### Synthesis of 6-Fluoro-7-(2-fluoro-4-iodo-phenylamino)-8-(3-hydroxy-3-piperidin-2-yl-azetidine-1-carbonyl)-2,3-dihydro-1H-indolizin-5-one

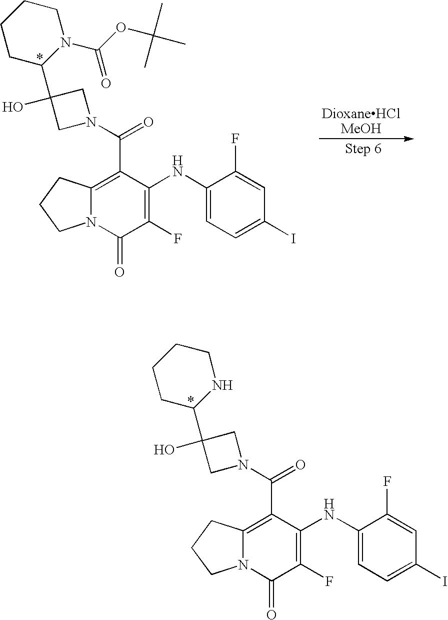

4N Dioxane in HCl (5 mL) was added to a solution of 2-{1-[6-fluoro-7-(2-fluoro-4-iodo-phenylamino)-5-oxo-1,2,3,5-tetrahydro-indolizine-8-carbonyl]-3-hydroxy-azetidin-3-yl}-piperidine-1-carboxylic acid tert-butyl ester (racemic mixture) (100 mg, 0.000 μmol) in methanol (1 mL) and the resulting mixture was stirred for 1 hr at RT. The solvents were distilled from the reaction mixture to yield a precipitate which was purified by preparative HPLC to afford 15 mg (16.6% yield) of 6-fluoro-7-(2-fluoro-4-iodo-phenylamino)-8-(3-hydroxy-3-piperidin-2-yl-azetidine-1-carbonyl)-2,3-dihydro-1H-indolizin-5-one (racemic mixture) as the required product.

LC-MS purity: 95%, m/z 571 (M+)

¹H NMR (DMSO-D₆): δ 8.6-8.4 (br s, 1H), 8.2 (s, 1H), 7.6 (d, 1H), 7.44 (d, 1H), 6.98-6.9 (m, 1H), 4.2-4.1 (m, 1H), 4.10-3.95 (m, 3H), 3.9-3.8 (m, 1H), 3.75-3.65 (m, 1H), 3.5-3.45 (m, 1H), 3.2-3.1 (m, 2H), 3.08 (t, 2H), 2.9-2.8 (m, 1H), 2.2-2.08 (m, 2H), 1.75-1.65 (m, 4H), 1.5-1.35 (m, 2H)

### EXAMPLE: 61

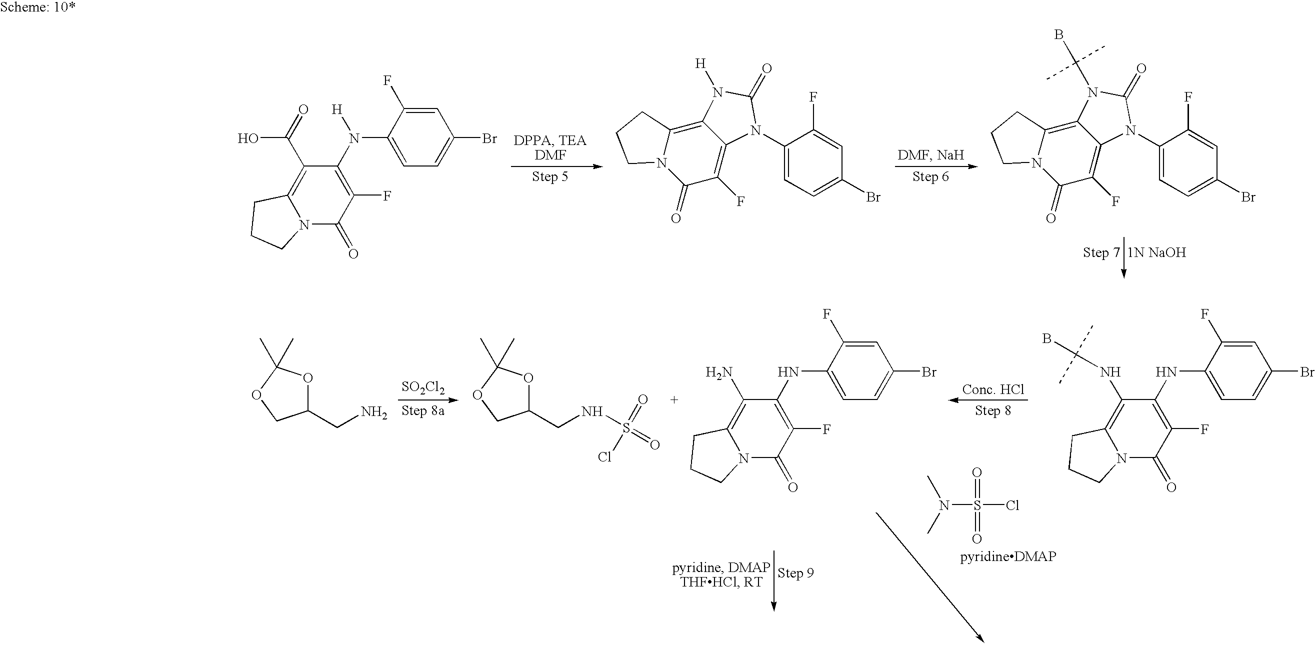

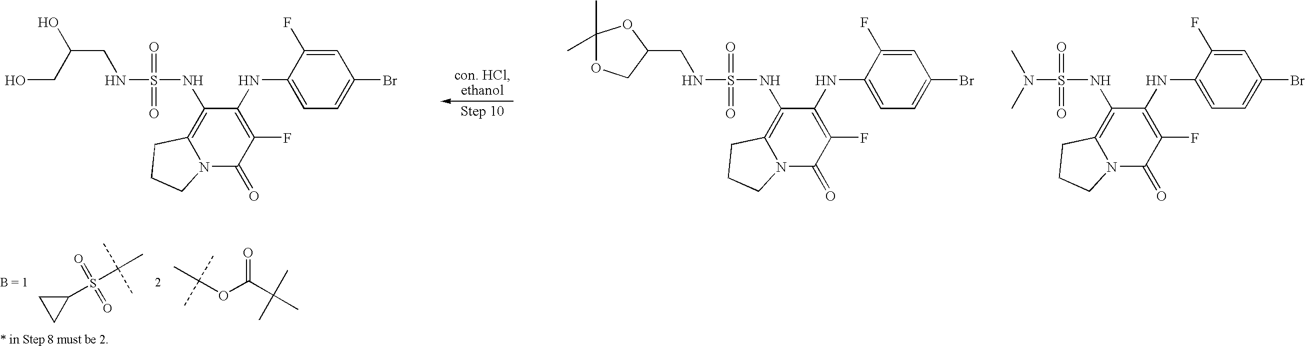

### Steps 1 to 3 were Performed in a Manner Similar to What has been Described for Example 8 and Step 4 was Performed in a Manner Similar to What has been Described for Example 11

### Step: 5

### Synthesis of 3-(4-Bromo-2-fluoro-phenyl)-4-fluoro-1,6,7,8-tetrahydro-3H-1,3,5a-triaza-as-indacene-2,5-dione

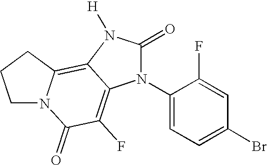

TEA (0.12 mL, 0.01 mol) and DPPA (0.18 mL, 0.001 mol) were added to a solution of 7-(4-bromo-2-fluoro-phenylamino)-6-fluoro-5-oxo-1,2,3,5-tetrahydro-indolizine-8-carboxylic acid (300 mg, 0.001 mol) in DMF (5 mL) and the reaction mixture was stirred for 4 hrs at RT under nitrogen atmosphere. This was followed by addition of toluene (5 mL) and the reaction mixture was heated to 90° C. for 2 hrs. The reaction mixture was concentrated under reduced pressure, added water to yield a precipitate which was collected and dried to afford 250 mg (84% yield) of 3-(4-bromo-2-fluoro-phenyl)-4-fluoro-1,6,7,8-tetrahydro-3H-1,3,5a-triaza-as-indacene-2,5-dione as the required product.

### Step: 6

### Synthesis of 3-(4-Bromo-2-fluoro-phenyl)-1-cyclopropanesulfonyl-4-fluoro-1,6,7,8-tetrahydro-3H-1,3,5a-triaza-as-indacene-2,5-dione

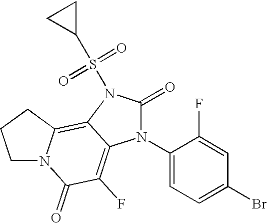

60% NaH (30 mg, 0.001 mol) were added to a stirred solution of 3-(4-bromo-2-fluoro-phenyl)-4-fluoro-1,6,7,8-tetrahydro-3H-1,3,5a-triaza-as-indacene-2,5-dione (0.2 g, 0.001 mol) in dry DMF (4 mL) at 0-5° C. under nitrogen atmosphere and the resulting mixture was stirred for 1 hr at RT. This was followed by dropwise addition of cyclopropanesulfonyl chloride (110 mg, 0.001 mol) in dry THF over a period of 10 mins at 0° C. and the stirring was continued for the next 16 hrs at RT. The crude product was used for the next step without a further purification.

### Step: 7

### Synthesis of Cyclopropanesulfonic acid [7-(4-bromo-2-fluoro-phenylamino)-6-fluoro-5-oxo-1,2,3,5-tetrahydro-indolizin-8-yl]-amide

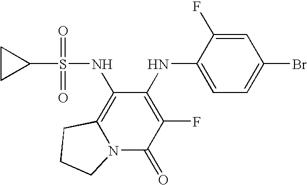

1N aqueous NaOH (6 mL) were added to 3-(4-bromo-2-fluoro-phenyl)-1-cyclopropanesulfonyl-4-fluoro-1,6,7,8-tetrahydro-3H-1,3,5a-triaza-as-indacene-2,5-dione and the resulting mixture was heated to 65° C. for 4 hrs. Ice cold water was added to the reaction mixture, neutralized with 5% ice cold HCl to pH of about 4 and the reaction mixture was partitioned between ethylacetate and water. The organic layer was dried over Na₂SO₄, concentrated under reduced pressure and the concentrate was purified by column chromatography (using silica gel, 2% methanol in DCM as eluant) to afford 21 mg (8.5% yield) of cyclopropanesulfonic acid [7-(4-bromo-2-fluoro-phenylamino)-6-fluoro-5-oxo-1,2,3,5-tetrahydro-indolizin-8-yl]-amide as the required product.

LCMS purity: 98.89%, m/z=461.9 (M+)

HPLC: 93.6%

¹H NMR (DMSO-D₆, 300 MHz): δ 8.9 (s, 1H), 7.65-7.25 (m, 3H), 4.1 (t, 2H), 3.2 (t, 2H), 2.8-2.7 (m, 1H), 2.2-2.1 (m, 2H), 0.95-0.85 (m, 4H)

### EXAMPLE: 62

### Steps 1 to 4 were Performed in a Manner Similar to What has been Described for Example 8 and Step 5 was Performed in a Manner Similar to What has been Described for Example 61

### Step: 6

### Synthesis of 3-(4-Bromo-2-fluoro-phenyl)-4-fluoro-2,5-dioxo-2,3,5,6,7,8-hexahydro-1,3,5a-triaza-as-indacene-1-carboxylic acid tert-butyl ester

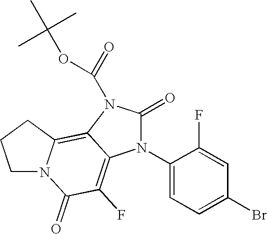

60% NaH (0.4 g, 0.01 mol) were added to a stirred solution of 3-(4-bromo-2-fluoro-phenyl)-4-fluoro-1,6,7,8-tetrahydro-3H-1,3,5a-triaza-as-indacene-2,5-dione (2.5 g, 0.007 mol) in dry DMF (20 mL) at RT under nitrogen atmosphere. The resulting mixture was stirred for 30 mins. This was followed by dropwise addition of BOC anhydride (1.9 g, 0.009 mol) in dry THF over a period 5 mins of at 0° C. and the reaction mixture was stirred for 4 hr at RT. The crude product was used in the next step without further purification.

### Step: 7

### Synthesis of [7-(4-Bromo-2-fluoro-phenylamino)-6-fluoro-5-oxo-1,2,3,5-tetrahydro-indolizin-8-yl]-carbamic acid tert-butyl ester

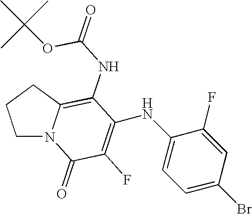

1N aqueous NaOH (15 mL) were added to 3-(4-bromo-2-fluoro-phenyl)-4-fluoro-2,5-dioxo-2,3,5,6,7,8-hexahydro-1,3,5a-triaza-as-indacene-1-carboxylic acid tert-butyl ester at 0° C. and the resulting mixture was stirred at RT for 6 hrs. The reaction mass was extracted with ethylacetate. The organic layer was washed with water, dried over Na₂SO₄, concentrated under reduced pressure to afford 1.15 g (28% yield) of [7-(4-bromo-2-fluoro-phenylamino)-6-fluoro-5-oxo-1,2,3,5-tetrahydro-indolizin-8-yl]-carbamic acid tert-butyl ester as the required product.

¹HNMR (CDCl₃, 300 MHz): δ 7.45-6.7 (m, 3H), 6.1 (s, 1H), 5.65 (s, 1H), 4.25 (t, 2H), 3.15 (t, 2H), 2.25-2.0 (m, 2H), 1.45 (s, 9H).

### Step: 8

### Synthesis of 8-Amino-7-(4-bromo-2-fluoro-phenylamino)-6-fluoro-2,3-dihydro-1H-indolizin-5-one

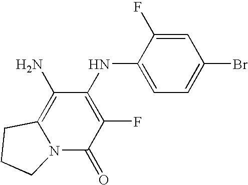

1N conc. HCl (4 mL) were added to a stirred solution of [7-(4-bromo-2-fluoro-phenylamino)-6-fluoro-5-oxo-1,2,3,5-tetrahydro-indolizin-8-yl]-carbamic acid tert-butyl ester (0.9 g, 0.002 mol) in THF (10 mL) and the reaction mixture was stirred for 4 hrs at RT. The reaction mixture was concentrated under reduced pressure, added saturated NaHCO₃ solution and extracted with ethylacetate. The organic layer dried over anhydrous Na₂SO₄ and concentrated under reduced pressure to afford 360 mg (72% yield) of 8-amino-7-(4-bromo-2-fluoro-phenylamino)-6-fluoro-2,3-dihydro-1H-indolizin-5-one as the required product.

¹HNMR (DMSO, 300 MHz): δ 7.85-6.85 (m, 3H), 4.2 (s, 2H), 4.12 (t, 2H), 3.1 (t, 2H), 2.25-2.0 (m, 2H)

### Step: 9

### Synthesis of N-[7-(4-Bromo-2-fluoro-phenylamino)-6-fluoro-5-oxo-1,2,3,5-tetrahydro-indolizin-8-yl]-N,N-dimethyl-amino-sulfonamide

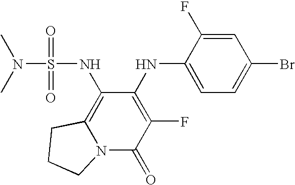

Pyridine (2 mL) were added to a solution of 8-amino-7-(4-bromo-2-fluoro-phenylamino)-6-fluoro-2,3-dihydro-1H-indolizin-5-one (200 mg, 0.001 mol) and the reaction mixture was stirred for 5 mins under nitrogen atmosphere. This was followed by addition of DMAP (5 mg, 0.0004 mol), cooled the reaction mass to 0° C., added N,N-dimethyl-sulfonyl chloride (85 mg, 0.001 mol) and continued stirring for next 16 hrs at RT. The reaction was monitored by TLC (100% ethylacetate) which showed the presence of starting material. The reaction mixture was heated to 50° C. for 2 hrs. The reaction mixture was partitioned between ethylacetate (3×50 mL) and water. The organic layer was washed with saturated NH₄Cl solution, dried over Na₂SO₄, concentrated under reduced pressure and the concentrate was purified by column chromatography (using neutral alumina, ethylacetate as eluant) to afford 22 mg (8% yield) of N-[7-(4-bromo-2-fluoro-phenylamino)-6-fluoro-5-oxo-1,2,3,5-tetrahydro-indolizin-8-yl]-N,N-dimethyl-amino-sulfonamide as the required product.

LCMS purity: 96.189%, m/z 463 (M+)

HPLC: 98%

¹H NMR (DMSO-D₆, 300 MHz): δ 8.7 (s, 1H), 7.6-6.85 (m, 3H), 4.15 (t, 2H), 3.29 (t, 2H), 2.7 (s, 6H), 2.2-2.1 (m, 2H)

### EXAMPLE: 63

### Steps 1 to 4 were Performed in a Manner Similar to What has been Described for Example 8, Step 5 was Performed in a Manner Similar to What has been Described for Example 61 and Steps 6 to 8 were Performed in a Manner Similar to What has been Described for Example 62.

### Step: 8a

### Synthesis of N-(2,2-Dimethyl-[1,3]dioxolan-4-ylmethyl)-chloro-sulfonamide

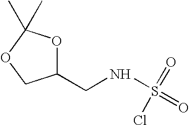

C-(2,2-Dimethyl-[1,3]dioxolan-4-yl)-methylamine (300 mg, 0.00229 mol) and DMAP (295 mg, 0.0024 mol) in dry DCM were added to a stirred solution of sulfuryl chloride (320 mg, 0.0023 mol) in DCM at −78° C. and the resulting mixture was stirred at −78° C. for 1 hr, at −50° C. for 2 hrs and at RT for 2 hrs. The product formed was used for next step.

### Step: 9

### Synthesis of N-[7-(4-Bromo-2-fluoro-phenylamino)-6-fluoro-5-oxo-1,2,3,5-tetrahydro-indolizin-8-yl]-C-(2,2-Dimethyl-[1,3]dioxolan-4-yl)-methyl amine-sulfonamide

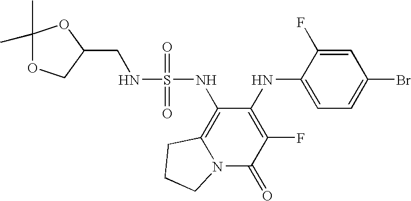

N-(2,2-Dimethyl-[1,3]dioxolan-4-ylmethyl)-chloro-sulfonamide (0.001 mol) was added dropwise to a stirred solution of 8-amino-7-(4-bromo-2-fluoro-phenylamino)-6-fluoro-2,3-dihydro-1H-indolizin-5-one (200 mg, 0.001 mol) in dry pyridine (3 mL) and DMAP (50 mg, 0.0004 mol) over a period of 10 mins at 0° C. and the reaction mixture was heated to 40° C. for 16 hrs The reaction mixture was concentrated under reduced pressure and the concentrate was partitioned between ethylacetate and water. The organic layer was concentrated and the concentrate was purified by column chromatography (using neutral alumina, DCM as eluant) to afford 26 mg (5% yield) of N-[7-(4-bromo-2-fluoro-phenylamino)-6-fluoro-5-oxo-1,2,3,5-tetrahydro-indolizin-8-yl]-C-(2,2-dimethyl-[1,3]dioxolan-4-yl)-methylamine-sulfonamide as the required product.

¹H NMR (DMSO, 300 MHz): δ 8.7 (s, 1H), 7.6-6.7 (m, 3H), 4.1-3.8 (m, 4H), 3.65-3.5 (m, 1H), 3.3-3.2 (m, 1H), 3.2 (t, 2H), 3.1-2.9 (m, 1H), 2.9-2.6 (m, 1H), 2.1 (t, 2H), 1.2 (d, 6H)

### Step: 10

### Synthesis of 2,3-Dihydroxy-propane-amino-sulfonicacid-[7-(4-bromo-2-fluoro-phenylamino)-6-fluoro-5-oxo-1,2,3,1-tetrahydro-indolizin-8-yl]-amide

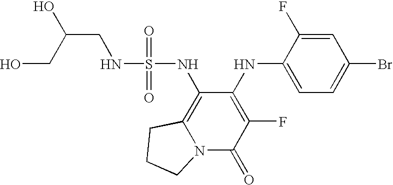

Conc. HCl (1 mL) were added to a stirred solution of N-[7-(4-bromo-2-fluoro-phenylamino)-6-fluoro-5-oxo-1,2,3,5-tetrahydro-indolizin-8-yl]-C-(2,2-Dimethyl-[1,3]dioxolan-4-yl)-methylamine-sulfonamide (26 mg, 0.00005 mol) in ethanol (4 mL) at 20° C. and the reaction mixture was stirred for 4 hrs at RT. The reaction mixture was concentrated under reduced pressure, added diethyl ether, decanted and dried under reduced pressure to afford 16 mg (70% yield) of 2,3-dihydroxy-propane-amino-sulfonicacid-[7-(4-bromo-2-fluoro-phenylamino)-6-fluoro-5-oxo-1,2,3,5-tetrahydro-indolizin-8-yl]-amide as the required product.

LCMS purity: 97.1%, m/z=509 (M+)

HPLC: 96.8%

¹H NMR (DMSO-D₆, 300 MHz): δ 8.7 (s, 1H), 7.6-6.85 (m, 3H), 4.15 (t, 2H), 3.4 (t, 2H), 3.25-3.15 (m, 2H), 3.1-3.0 (m, 2H), 2.85-2.75 (m, 1H)

### EXAMPLE: 64

### Steps 1 to 4 were Performed in a Manner Similar to What has been Described for Example 8, Step 5 was Performed in a Manner Similar to What has been Described for Example 61 and Steps 6 to 8 were Performed in a Manner Similar to What has been Described for Example 62.

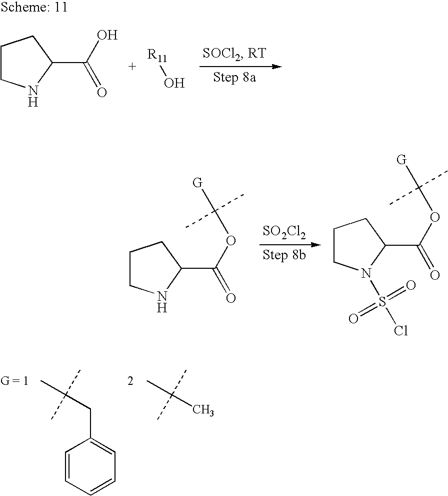

### Step: 8a

### Synthesis of Pyrrolidine-2-carboxylic acid benzyl ester hydrochloride

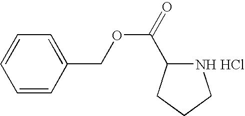

Thionyl chloride (8 mL) and pyrrolidine-2-carboxylic acid (3 g, 0.026 mol) were added to benzyl alcohol (20 mL) at −10° C. under nitrogen atmosphere and the reaction mixture was stirred at RT for 16 hrs. The reaction mass was diluted with dry diethyl ether and stirred at RT for 2 hrs to yield a precipitate which was washed with excess diethyl ether, decanted and dried under reduced pressure to afford 4 g (66% yield) of pyrrolidine-2-carboxylic acid benzyl ester hydrochloride as the required product.

¹H NMR (CDCl₃, 300 MHz): δ 10.9 (s, 1H), 9.2 (s, 1H), 7.2-7.6 (s, 5H), 5.2 (t, 2H), 4.9 (s, 1H), 4.5 (s, 1H), 3.5 (t, 2H), 2.4-2.3 (m, 1H), 2.2-2.1 (m, 2H)

### Step: 8b

### Synthesis of 1-Chlorosulfonyl-pyrrolidine-2-carboxylic acid benzyl ester

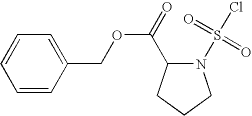

DMAP (0.5 g, 0.004 mol) and TEA (1.6 mg, 0.016 mol) were added to a stirred solution of pyrrolidine-2-carboxylic acid benzyl ester hydrochloride (3 g, 0.015 mol) in dry toluene (40 mL) at RT and the resulting mixture was stirred for 20 mins. The reaction mixture was cooled to −10° C., followed by dropwise addition of sulfuryl chloride (2 g, 0.015 mol) over a period of 15 mins and continued stirring for 3 hrs at RT. The reaction mass was quenched with saturated NH₄Cl solution, extracted with DCM and the organic layer was dried over Na₂SO₄ and concentrated to afford 1.2 g of 1-chlorosulfonyl-pyrrolidine-2-carboxylic acid benzyl ester as the crude product.

¹H NMR (DMSO-D₆, 300 MHz): δ 10.9 (s, 1H), 9.2 (s, 1H), 7.6-7.2 (br s, 5H), 5.2 (t, 2H), 4.9 (s, 1H), 4.5 (s, 1H), 3.5 (t, 2H), 2.4 (m, 1H), 2.2 (m, 2H)

### Step: 9

### Synthesis of 1-[7-(4-Bromo-2-fluoro-phenylamino)-6-fluoro-5-oxo-1,2,3,5-tetrahydro-indolizin-8-ylsulfamoyl]-pyrrolidine-2-carboxylic acid benzyl ester

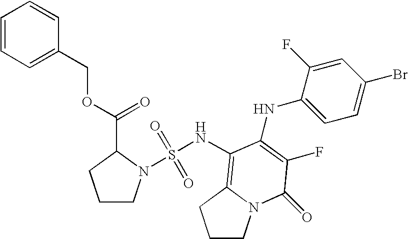

Pyridine (3 mL) and DMAP (20 mg, 0.0002 mol) were added to 8-amino-7-(4-bromo-2-fluoro-phenylamino)-6-fluoro-2,3-dihydro-1H-indolizin-5-one (110 mg, 0.0003 mol) under nitrogen atmosphere an the reaction mixture was cooled to 0° C. This was followed by dropwise addition of 1-chlorosulfonyl-pyrrolidine-2-carboxylic acid benzyl ester (300 mg, 0.001 mol) in DCM over a period of 15 mins, stirred at RT for 1 hr and heated to 60° C. for 16 hrs. The reaction mass was concentrated under reduced pressure and the concentrate was partitioned between ethylacetate and water. The organic layer was dried over Na₂SO₄ and purified by column chromatography (using silica gel, 100% ethylacetate as eluant) to afford 65 mg (33% yield) of 1-[7-(4-bromo-2-fluoro-phenylamino)-6-fluoro-5-oxo-1,2,3,5-tetrahydro-indolizin-8-ylsulfamoyl]-pyrrolidine-2-carboxylic acid benzyl ester as the required product.

¹H NMR (DMSO-D₆, 300 M): δ 7.4-7.15 (m, 8H), 6.85-6.75 (m, 1H), 6.7-6.6 (s, 1H), 5.2 (q, 3H), 4.65-4.55 (m, 1H), 4.25-4.2 (m, 3H), 3.65-3.55 (m, 3H), 3.4 (t, 2H), 3.3 (t 2H), 3.25-3.15 (m, 1H), 2.4 (t, 2H), 2.2 (t, 2H), 2.15-2.05 (m, 8H)

### Step: 10

### Synthesis of 1-[7-(4-Bromo-2-fluoro-phenylamino)-6-fluoro-5-oxo-1,2,3,5-tetrahydro-indolizin-8-ylsulfamoyl]-pyrrolidine-2-carboxylic acid

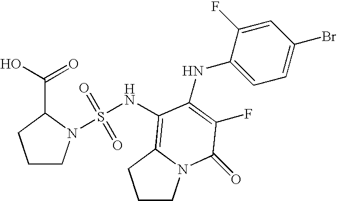

LiOH solution (20 mg, 0.0004 mol) were added to a stirred solution of 1-[7-(4-bromo-2-fluoro-phenylamino)-6-fluoro-5-oxo-1,2,3,5-tetrahydro-indolizin-8-ylsulfamoyl]-pyrrolidine-2-carboxylic acid benzyl ester (65 mg, 0.0001 mol) in methanol:THF (2:3) and the resulting mixture was stirred at RT for 3 hrs. The reaction mass was concentrated under reduced pressure, diluted with water, neutralized with 10% HCl to a pH of about 2 and the precipitate formed was collected and dried to afford 20 mg (36% yield) of 1-[7-(4-bromo-2-fluoro-phenylamino)-6-fluoro-5-oxo-1,2,3,5-tetrahydro-indolizin-8-ylsulfamoyl]-pyrrolidine-2-carboxylic acid as the required product.

HPLC: 91.17%

¹H NMR (DMSO-D₆, 300 MHz): 12.85 (s, 1H), 8.9 (s, 1H), 7.6-7.2 (m, 3H), 4.3-4.2 (m, 1H), 4.1 (t, 2H), 3.1 (t, 2H), 2.15-2.05 (m, 2H), 1.75-1.65 (m, 2H)

### EXAMPLE: 65

### Steps 1 to 4 were Performed in a Manner Similar to What has been Described for Example 8, Step 5 was Performed in a Manner Similar to What has been Described for Example 61 and Steps 6 to 8 were Performed in a Manner Similar to What has been Described for Example 62.

### Step: 8

### Synthesis of 1-Chlorosulfonyl-pyrrolidine-2-carboxylic acid methyl ester

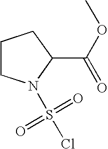

DMAP (0.5 g, 0.00409 mol) and TEA (2.54 g, 0.0251 mol) were added to a stirred solution of pyrrolidine-2-carboxylic acid methyl ester hydrochloride (4 g, 0.024 mol) in dry toluene (50 mL) at RT and the resulting mixture was stirred for 10 mins. The reaction mixture was cooled to −20° C., followed by dropwise addition of sulfuryl chloride (3.3 g, 0.024 mol) over a period of 30 mins and the stirring was continued for 1 hr at −10° C. and for a further 2 hrs at RT. The reaction mass was diluted with DCM and washed with aqueous NH₄Cl solution. The organic layer was, dried over Na₂SO₄ and concentrated to afford 1.2 g (24% yield) of 1-chlorosulfonyl-pyrrolidine-2-carboxylic acid methyl ester as the required product.

### Step: 9

### Synthesis of 1-[7-(4-Bromo-2-fluoro-phenylamino)-6-fluoro-5-oxo-1,2,3,5-tetrahydro-indolizin-8-ylsulfamoyl]-pyrrolidine-2-carboxylic acid methyl ester

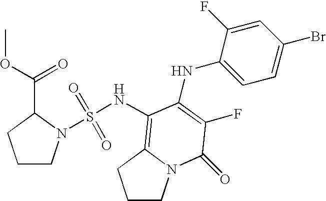

DMAP (50 mg, 0.0004 mol) were added to a stirred solution of 8-amino-7-(4-bromo-2-fluoro-phenylamino)-6-fluoro-2,3-dihydro-1H-indolizin-5-one (300 mg, 0.001 mol) in dry pyridine (5 mL) and under nitrogen atmosphere an the reaction mixture was cooled to 0° C. This was followed by dropwise addition of 11-chlorosulfonyl-pyrrolidine-2-carboxylic acid methyl ester (1 g, 0.004 mol) in DCM over a period of 10 mins and the resulting mixture was stirred at RT for 4 hrs. The reaction mixture was heated to 65° C. for 16 hrs. The reaction mass was concentrated under reduced pressure and the concentrate was partitioned between ethylacetate and water. The organic layer was washed with brine solution, concentrated under reduced pressure and the concentrate was purified by column chromatography (using silica gel, 70% ethylacetate in hexane as eluant) to afford 110 mg (24% yield) of 1-[7-(4-bromo-2-fluoro-phenylamino)-6-fluoro-5-oxo-1,2,3,5-tetrahydro-indolizin-8-ylsulfamoyl]-pyrrolidine-2-carboxylic acid methyl ester as the required product.

### Step: 10

### Synthesis of 2-Hydroxymethyl-pyrrolidine-1-sulfonic acid [7-(4-bromo-2-fluoro-phenylamino)-6-fluoro-5-oxo-1,2,3,5-tetrahydro-indolizin-8-yl]-amide

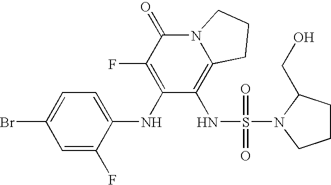

NaBH₄ (25 mg, 0.00065 mol) was added to a stirred solution of 1-[7-(4-bromo-2-fluoro-phenylamino)-6-fluoro-5-oxo-1,2,3,5-tetrahydro-indolizin-8-ylsulfamoyl]-pyrrolidine-2-carboxylic acid methyl ester (110 mg, 0.0002 mol) in dry THF (3 mL) under nitrogen atmosphere and the resulting mixture was heated at 60° C. This was followed by dropwise addition of methanol (2 mL) over a period of 5 mins while the temperature was maintained at 60° C. for 1 hr. The reaction mass was concentrated under reduced pressure, added ice cold water, neutralized with 5% dil. HCl, extracted with ethylacetate and the organic layer was dried over Na₂SO₄ and concentrated. The crude product was dissolved in 1:9 methanol: DCM, added ether and the precipitate formed was collected to afford 75 g (70% yield) of 2-Hydroxymethyl-pyrrolidine-1-sulfonic acid [7-(4-bromo-2-fluoro-phenylamino)-6-fluoro-5-oxo-1,2,3,5-tetrahydro-indolizin-8-yl]-amide as the required product.

LCMS purity: 99.66%, m/z 521 (M+2)

HPLC: 95.4%

¹H NMR (DMSO-D₆, 300 MHz): δ 8.7 (s, 1H), 7.6-7.25 (m, 3H), 4.15 (t, 2H), 3.65 (m, 1H), 3.1-3.2 (m, 4H), 2.15-2.05 (m, 2H), 1.85-1.75 (m, 4H)

### EXAMPLE: 66

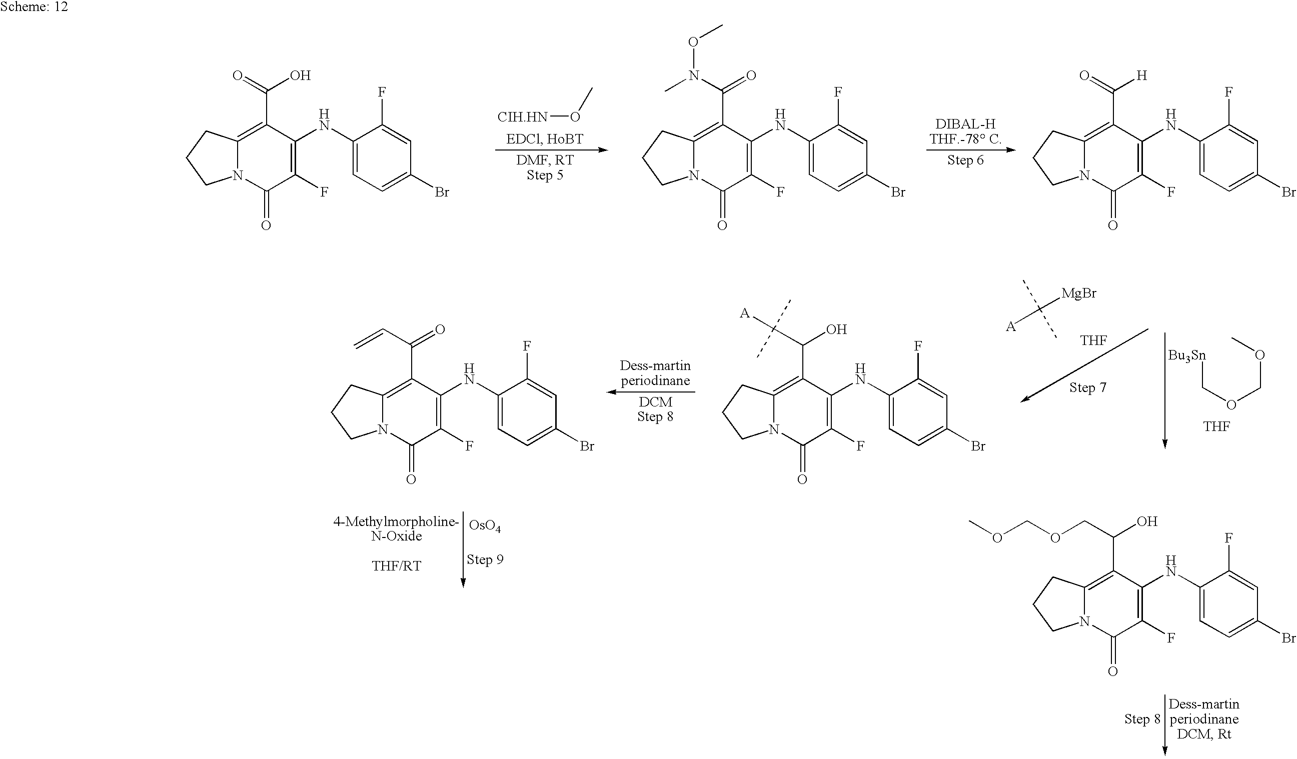

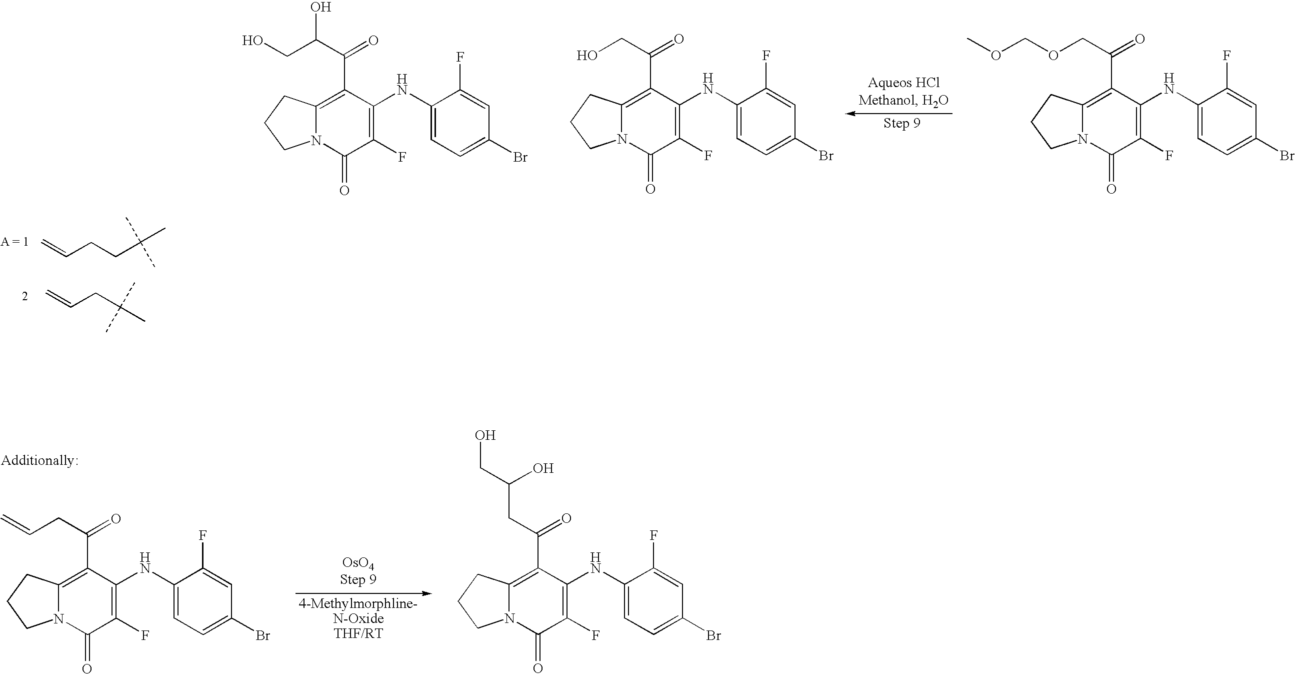

### Steps 1 to 3 were Performed in a Manner Similar to What has been Described for Example 8 and Step 4 was Performed in a Manner Similar to What has been Described for Example 11

### Step: 5

### Synthesis of 7-(4-Bromo-2-fluoro-phenylamino)-6-fluoro-5-oxo-1,2,3,5-tetrahydro-indolizine-8-carboxylic acid methoxy-methyl-amide

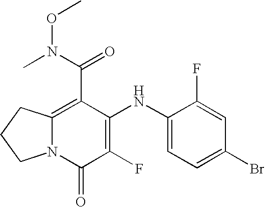

EDCI (0.99 g, 0.005 mol) and HOBt (0.702 g, 0.005 mol) were added to a stirred solution of 7-(4-bromo-2-fluoro-phenylamino)-6-fluoro-5-oxo-1,2,3,5-tetrahydro-indolizine-8-carboxylic acid (1 g, 0.003 mol) in DMF (50 mL) and the reaction mixture was stirred for 1.30 hr at RT. This was followed by addition of O,N-dimethyl-hydroxylamine hydrochloride (0.506 g, 0.005 mol) and TEA (2.16 mL, 0.016 mol) under nitrogen atmosphere. The reaction mixture was stirred for 16 hrs at RT. The reaction mixture was partitioned between ethylacetate and water. The organic layer was washed with saturated NH₄Cl, brine solution, dried over anhy. Na₂SO₄ and concentrated to afford 0.800 mg (72.7% yield) of 7-(4-bromo-2-fluoro-phenylamino)-6-fluoro-5-oxo-1,2,3,5-tetrahydro-indolizine-8-carboxylic acid methoxy-methyl-amide as the required product.

LCMS purity: 96.9%, m/z=428 (M+1)

¹H NMR (DMSO-D₆, 300 M): δ 8.0-7.95 (br s, 1H), 7.5-7.4 (dd, 1H), 7.3-7.2 (dd, 1H), 7.0 (t, 1H), 4.1-3.9 (m, 2H), 3.6 (s, 3H), 3.4 (s, 3H), 3.0-2.9 (m, 2H), 2.15-2.05 (m, 2H)

### Step: 6

### Synthesis of 7-(4-Bromo-2-fluoro-phenylamino)-6-fluoro-5-oxo-1,2,3,1-tetrahydro-indolizine-8-carbaldehyde

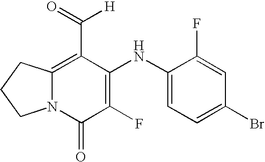

DIBAL-H (1.0 M solution in toluene) (5.7 mL, 5.7 mmol) were added to a solution of 7-(4-bromo-2-fluoro-phenylamino)-6-fluoro-5-oxo-1,2,3,5-tetrahydro-indolizine-8-carboxylic acid methoxy-methyl-amide (0.7 g, 1.635 mmol) in dry THF (20 mL) at −78° C. and the reaction mixture was stirred for 2 hrs at −78° C. The reaction mass was quenched with saturated NH₄Cl and extracted with ethyl acetate. The aqueous layer was extracted with ethylacetate and the organic layer was washed with water, brine (10 mL), dried over anhydrous Na₂SO₄ and concentrated. The concentrate was purified by column chromatography (using silica gel, 2% methanol in DCM as eluant) to afford 0.2 g, (33.16% yield) of 7-(4-bromo-2-fluoro-phenylamino)-6-fluoro-5-oxo-1,2,3,5-tetrahydro-indolizine-8-carbaldehyde as the required product.

¹H NMR (DMSO-D₆, 300 MHz): δ 9.83-9.8 (d, 1H), 9.6 (d, 1H), 7.6 (dd, 1H), 7.4-7.3 (d, 1H), 7.2-7.15 (m, 1H), 4.1-4.0 (t, 2H), 3.5 (t, 2H), 2.2 (t, 2H)

### Step: 7

### Synthesis of 7-(4-Bromo-2-fluoro-phenylamino)-6-fluoro-8-(1-hydroxy-but-3-enyl)-2,3-dihydro-1H-indolizin-5-one

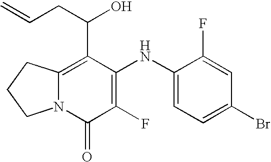

Allyl magnesium bromide (11.65 mL, 0.011653 mol) were added to a solution of 7-(4-bromo-2-fluoro-phenylamino)-6-fluoro-5-oxo-1,2,3,5-tetrahydro-indolizine-8-carbaldehyde (0.430 g, 0.001 mol) in dry THF (10 mL) at −78° C. under nitrogen atmosphere. The reaction mixture was stirred for 2 hrs at RT. The reaction mixture was quenched with saturated NH₄Cl solution and extracted with ethylacetate. The organic layer was washed with water, brine solution, dried over anhydrous Na₂SO₄ and concentrated. The concentrate was purified by column chromatography (using silica gel, 1-1.5% methanol in DCM as eluant) to afford 0.250 mg (52.4% yield) of 7-(4-bromo-2-fluoro-phenylamino)-6-fluoro-8-(1-hydroxy-but-3-enyl)-2,3-dihydro-1H-indolizin-5-one as the required product.

LCMS: 84.4%, m/z=411 (M+)

HPLC: 90%

¹H NMR (DMSO-D₆, 300 MHz): δ 8.2-8.1 (br s, 1H), 7.6-7.5 (dd, 1H), 7.3 (d, 1H), 6.8-6.7 (m, 1H), 6.4 (d, 1H), 5.7 (m, 1H), 5.0-4.8 (m, 2H), 4.7-4.6 (d, 1H), 4.0-3.9 (m, 2H), 3.1-2.9 (m, 2H), 3.0-2.9 (m, 1H), 2.5-2.4 (m, 1H), 2.3-2.2 (m, 1H), 2.1 (t, 2H).

### EXAMPLE: 67

### Steps 1 to 3 were Performed in a Manner Similar to What has been Described for Example 8, Step 4 was Performed in a Manner Similar to What has been Described for Example 111, and Steps 5 to 6 were Performed in a Manner Similar to What has been Described for Example 66.

### Step: 7

### Synthesis of 7-(4-Bromo-2-fluoro-phenylamino)-6-fluoro-8-(1-hydroxy-allyl)-2,3-dihydro-1H-indolizin-5-one

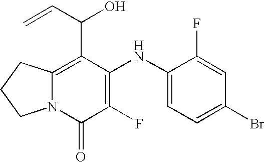

Vinyl magnesium bromide (1M solution in THF) (1.62 mL, 0.002 mol) were added to a solution of 7-(4-bromo-2-fluoro-phenylamino)-6-fluoro-5-oxo-1,2,3,5-tetrahydro-indolizine-8-carbaldehyde (0.1 g, 0.0003 mol) in dry THF (10 mL) at −78° C. under nitrogen atmosphere. The reaction mixture was stirred for 2 hrs at RT. The reaction mixture was quenched with saturated NH₄Cl solution at −78° C. and extracted with ethylacetate at RT. The organic layer was washed with water, brine solution, dried over anhydrous Na₂SO₄ and concentrated. The concentrate was purified by preparative HPLC to afford 10 mg (9% yield) of 7-(4-bromo-2-fluoro-phenylamino)-6-fluoro-8-(1-hydroxy-allyl)-2,3-dihydro-1H-indolizin-5-one as the required product.

LCMS: 96.4%, m/z=399 (M+2), 398 (M+1)

HPLC: 98%

¹H NMR (DMSO-D₆, 300 M): δ 8.0-7.8 (br s, 1H), 7.6-7.4 (d, 1H), 7.6-7.3 (d, 1H), 6.6 (t, 1H), 6.6-6.5 (br s, 1H), 5.8-6.0 (m, 1H), 5.2 (d, 2H), 5.0 (d, 1H), 4.0 (t, 2H), 3.1 (m, 2H), 2.1 (t, 2H).

### EXAMPLE: 68

### Steps 1 to 3 were Performed in a Manner Similar to What has been Described for Example 8, Step 4 was Performed in a Manner Similar to What has been Described for Example 11, Steps 5 to 6 were Performed in a Manner Similar to What has been Described for Example 66 and Step 7 was Performed in a Manner Similar to What has been Described for Example 67.

### Step: 8

### Synthesis of 8-Acryloyl-7-(4-bromo-2-fluoro-phenylamino)-6-fluoro-2,3-dihydro-1H-indolizin-5-one

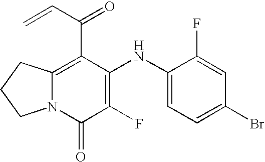

Dess-martin periodinane (0.582 g, 0.001 mol) were added to a stirred solution of 7-(4-bromo-2-fluoro-phenylamino)-6-fluoro-8-(1-hydroxy-allyl)-2,3-dihydro-1H-indolizin-5-one (0.440 g, 0.001 mol) in DCM (10 mL) and the reaction mixture was stirred for 12 hrs at RT. This was followed by addition of 0.8 g of NaHCO₃ dissolved in 10 mL of water and 2.48 g of sodium thiosulfate. 5H₂O dissolved in 10 mL of water and continued stirring for next 5 mins. The reaction mixture was extracted with DCM and the organic layer was washed with saturated NaHCO₃ solution, brine, dried over anhydrous Na₂SO₄ and concentrated to afford 0.400 g of 8-acryloyl-7-(4-bromo-2-fluoro-phenylamino)-6-fluoro-2,3-dihydro-1H-indolizin-5-one as the crude product which was used for the next step without a further purification.

### Step: 9

### Synthesis of 7-(4-Bromo-2-fluoro-phenylamino)-8-(2,3-dihydroxy-propionyl)-6-fluoro-2,3-dihydro-1H-indolizin-5-one

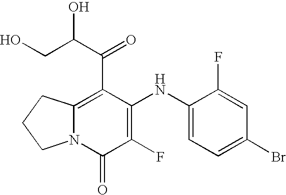

4-Methyl-morpholine-N-oxide (0.118 g, 0.001012 mol) and osmium tetra oxide (0.025 g, 0.0001 mol) were added to a stirred solution of 8-acryloyl-7-(4-bromo-2-fluoro-phenylamino)-6-fluoro-2,3-dihydro-1H-indolizin-5-one (0.4 g, 0.001 mol) in THF (10 mL) under nitrogen atmosphere and the resulting mixture was stirred for 2 hrs at RT. The reaction mixture was partitioned between ethylacetate and water. The organic layer was dried over anhydrous Na₂SO₄, concentrated and the concentrate was purified by column chromatography (using silica gel, 1.5% methanol in DCM as eluant) to get a yellow solid which was further purified by preparative HPLC to afford 10 mg (2.3% yield) of 7-(4-bromo-2-fluoro-phenylamino)-8-(2,3-dihydroxy-propionyl)-6-fluoro-2,3-dihydro-1H-indolizin-5-one as the required product.

LCMS: m/z 431 (M+2)

HPLC: 90%

¹H NMR (DMSO-D₆, 300 MHz): δ 8.6-8.5 (br s, 1H), 7.5-7.4 (dd, 1H), 7.3-7.2 (dd, 1H), 6.9-6.8 (m, 1H), 5.3 (d, 1H), 5.0 (t, 1H) 4.5-4.4 (m, 1H), 4.1-4.0 (m, 2H), 3.5 (t, 2H), 3.3-3.2 (m, 1H), 3.1-3.0 (m, 1H), 2.1 (t, 2H)

### EXAMPLE: 69

### Steps 1 to 3 were Performed in a Manner Similar to What has been Described for Example 8, Step 4 was Performed in a Manner Similar to What has been Described for Example 11, and Steps 5 to 6 were Performed in a Manner Similar to What has been Described for Example 66.

### Step: 7

### Synthesis of 7-(4-Bromo-2-fluoro-phenylamino)-6-fluoro-8-(1-hydroxy-2-methoxymethoxy-ethyl)-2,3-dihydro-1H-indolizin-5-one

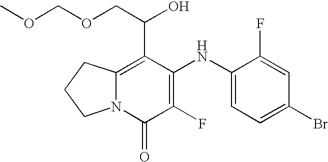

n-Butyl lithium (2.5M solution in hexane) (6.6 mL, 6.775 mmol) was added dropwise to a stirred solution of tributyl-methoxymethoxymethyl-stannane (3.72 g, 10.17 mmol) in dry THF at −78° C. over a period of 5 mins and the resulting mixture was stirred for 5 mins. This was followed by addition of 7-(4-bromo-2-fluoro-phenylamino)-6-fluoro-5-oxo-1,2,3,5-tetrahydro-indolizine-8-carbaldehyde (250 mg, 0.678 mmol) in THF at −78° C. and with continued stirring for a further 40 mins at −78° C. The reaction mixture was quenched with saturated NH₄Cl solution at −78° C., warmed to RT and diluted with ethylacetate. The organic layer was washed with brine solution, dried over anhydrous Na₂SO₄ and concentrated. The concentrate was used for next step without further purification.

### Step: 8

### Synthesis of 7-(4-Bromo-2-fluoro-phenylamino)-6-fluoro-8-(2-methoxymethoxy-acetyl)-2,3-dihydro-1H-indolizin-5-one

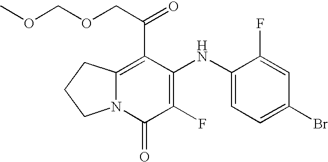

Dess-martin periodinane (0.180 g, 0.0004 mol) were added to a stirred solution of 7-(4-bromo-2-fluoro-phenylamino)-6-fluoro-8-(1-hydroxy-2-methoxymethoxy-ethyl)-2,3-dihydro-1H-indolizin-5-one (0.160 g, 0.0004 mol) in DCM (10 mL) and the reaction mixture was stirred for 1.30 hrs at RT. This was followed by addition of 10 mL of saturated NaHCO₃ containing 300 mg of sodium thiosulfate. 5H₂O and continued stirring for next 10 mins. The residual mixture was extracted with ethylacetate and the organic layer was washed with saturated NaHCO₃ solution, brine, dried over anhydrous Na₂SO₄ and concentrated. The concentrate was purified by column chromatography (using silica gel, 60-65% ethylacetate in hexane as eluant) to afford 100 mg (62.8% yield) of 7-(4-bromo-2-fluoro-phenylamino)-6-fluoro-8-(2-methoxymethoxy-acetyl)-2,3-dihydro-1H-indolizin-5-one as the required product.

LCMS purity: 76.1%, m/z=444 (M+1), 445 (M+2)

HPLC: 80%

### Step: 9

### Synthesis of 7-(4-Bromo-2-fluoro-phenylamino)-6-fluoro-8-(2-hydroxy-acetyl)-2,3-dihydro-1H-indolizin-5-one

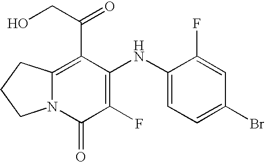

10% aqueous HCl (4 mL) and water (3 mL) were added to a stirred solution of 7-(4-bromo-2-fluoro-phenylamino)-6-fluoro-8-(2-methoxymethoxy-acetyl)-2,3-dihydro-1H-indolizin-5-one (100 mg, 0.0002 mol) in methanol (3 mL) and the resulting mixture was stirred for 18 hrs at RT. The reaction mixture was neutralized with saturated NaHCO₃ solution, diluted with ethylacetate and the organic layer was washed with brine, dried over anhydrous Na₂SO₄ and concentrated. The concentrate was purified by preparative HPLC to afford 14 mg (15.7% yield) of 7-(4-bromo-2-fluoro-phenylamino)-6-fluoro-8-(2-hydroxy-acetyl)-2,3-dihydro-1H-indolizin-5-one as the required product.

LCMS: 98.4%, m/z=401 (M+2)

HPLC: 98.7%

¹H NMR (DMSO-D₆, 300 MHz): δ 9.0-8.8 (br s, 1H), 7.5 (dd, 1H), 7.3 (d, 1H), 6.9-6.8 (m, 1H), 5.2 (t, 1H), 4.3-4.0 (m, 2H), 4.0 (t, 2H), 3.3-3.2 (m, 2H), 2.1 (t, 2H)

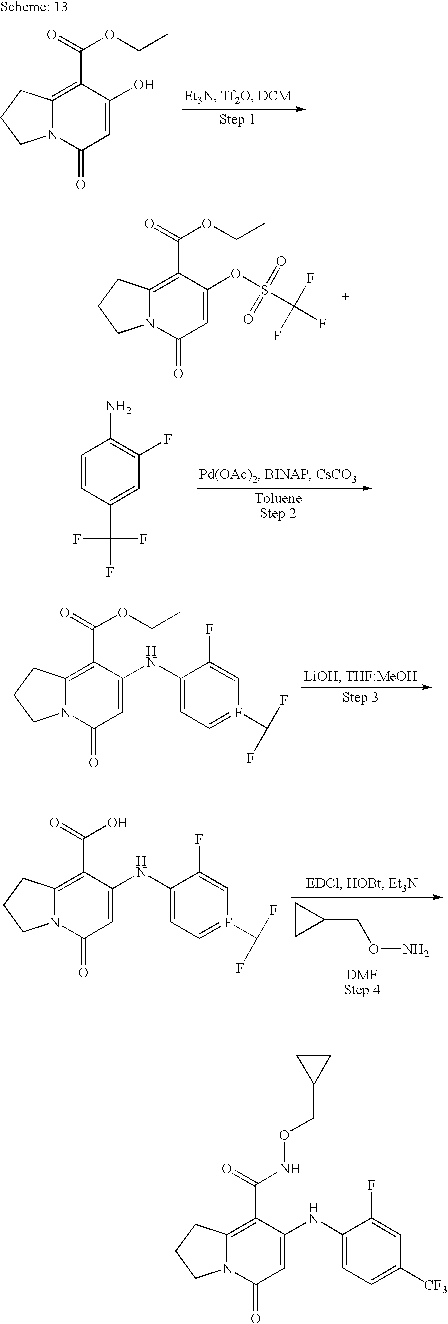

### EXAMPLE: 70

### Step: 1

### Synthesis of 5-oxo-7-trifluoromethanesulfonyloxy-1,2,3,5-tetrahydro-indolizine-8-carboxyllic acid ethyl ester

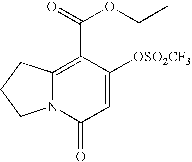

TEA (5.082 g, 0.0502242 mol) was added to a solution of 7-hydroxy-6-methyl-5-oxo-1,2,3,5-tetrahydro-indolizine-8-carboxylic acid ethyl ester (7.0 g, 0.031 mol) dissolved in DCM (70 mL) and the reaction mixture was cooled to −78° C. Triflic anhydride (11.51 g, 0.041 mol) were added to the reaction mixture and the reaction mixture was stirred for 16 hours at ambient temperature. The reaction mixture was washed with sodium bicarbonate solution (20 mL) and the organic layer was dried and concentrated. The concentrate was purified by column chromatography (using silica gel, 5% MeOH in CHCl₃ as eluant) to afford 7.78 g (73.0% yield) of 5-oxo-7-trifluoromethanesulfonyloxy-1,2,3,5-tetrahydro-indolizine-8-carboxyllic acid ethyl ester as the required product.

¹H NMR (CDCl₃, 300 M): 6.3 (s, 1H), 4.4 (q, 2H), 4.2 (t, 2H), 3.5 (t, 2H), 2.35 (q, 3H), 1.4 (t, 3H).

### Step: 2

### Synthesis of 7-(2-Fluoro-4-trifluoromethyl-phenylamino)-5-oxo-1,2,3,5-terahydro-indolizine-8-carboxyllic acid ethyl ester

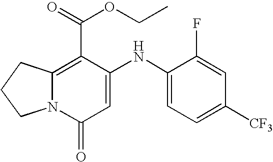

Palladium acetate (0.063 g, 0.003 mol), BINAP (0.263 g, 0.0003 mol), cesium carbonate (1.37 g, 0.004 mol) were dissolved in toluene and the resulting mixture was sparged for 30 mins with nitrogen. This was followed by addition of 5-oxo-7-trifluoromethanesulfonyloxy-1,2,3,5-tetrahydro-indolizine-8-carboxyllic acid ethyl ester (1 g, 0.003 mol) and 2-fluoro-4-trifluoromethyl aniline (0.549 g, 0.003 mol) and the reaction flask was again sparged for another 15 mins. The reaction mixture was heated at 110° C. for 1.30 hrs. The reaction mixture was filtered through celite and the filtrate was concentrated. The concentrate was purified by column chromatography (using silica gel of mesh size 60-120, 70% MeOH in CHCl₃ as eluant) to afford 0.4 g (37% yield) of 7-(2-fluoro-4-trifluoromethyl-phenylamino)-5-oxo-1,2,3,5-terahydro-indolizine-8-carboxyllic acid ethyl ester as the required product.

¹H NMR (DMSO-D₆): 7.6 (t, 1H), 7.4 (t, 2H), 6.0 (s, 1H), 4.4 (q, 2H), 4.2 (t, 2H), 3.5 (t, 2H), 2.2 (q, 3H), 1.4 (t, 3H)

### EXAMPLE: 71

### Steps 1 and 2 were Performed in a Manner Similar to What has been Described for Example 70

### Step: 3

### Synthesis of 7-(2-Fluoro-4-trifluoromethyl-phenylamino)-5-oxo-1,2,3,5-terahydro-indolizine-8-carboxyllic acid

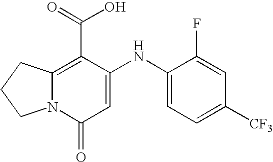

LiOH (0.07 g, 0.002 mol) was added to a stirred solution of 7-(2-fluoro-4-trifluoromethyl-phenylamino)-5-oxo-1,2,3,5-terahydro-indolizine-8-carboxylic acid ethyl ester (0.30 g, 0.0008 mol) dissolved in MeOH:THF (6 mL) and the reaction mixture was stirred at RT for 4 hrs. The reaction mixture was concentrated and the concentrate was acidified with 10% HCl to yield a precipitate which was collected and dried to afford 0.270 g (95.74% yield) of 7-(2-fluoro-4-trifluoromethyl-phenylamino)-5-oxo-1,2,3,5-terahydro-indolizine-8-carboxyllic acid as the required product.

¹H NMR (DMSO-D₆, 300 MHz): 13.4 (s, 1H), 10.4 (s, 1H), 7.85-7.75 (m, 2H), 7.6 (d, 1H), 5.7 (s, 1H), 4.0 (t, 2H), 3.5 (t, 2H), 2.1 (t, 2H)

### EXAMPLE: 72

### Steps 1 to 2 were Performed in a Manner Similar to What has been Described for Example 70 and Step 3 was Performed in a Manner Similar to What has been Described for Example 71

### Step: 4

### Synthesis of 7-(2-Fluoro-4-trifluoromethyl-phenylamino)-5-oxo-1,2,3,5-terahydro-indolizine-8-carboxyllic acid cyclopropylmethoxy amide

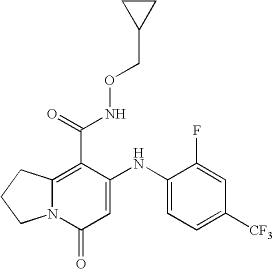

EDCI (0.322 g, 0.002 mol), HOBt (0.23 g, 0.002 mol) and 7-(2-fluoro-4-trifluoromethyl-phenylamino)-5-oxo-1,2,3,5-terahydro-indolizine-8-carboxyllic acid (0.2 g, 0.00 mmol) was dissolved in DMF in an inert atmosphere and the reaction mixture was stirred at RT for 30 mins. This was followed by addition of cyclopropylmethyl hydroxylamine hydrochloride (0.21 g, 0.002 mol) and TEA (0.17 g, 0.002 mol) and the reaction mixture was stirred at RT for 16 hrs. Water was added to the reaction mixture and the reaction mixture was quenched with saturated NH₄Cl and extracted with ethyl acetate. The organic layer was washed with saturated bicarbonate solution, brine solution and dried over Na₂SO₄ to yield a precipitate which was triturated with ether to afford 0.1 g (41.8% yield) of 7-(2-fluoro-4-trifluoromethyl-phenylamino)-5-oxo-1,2,3,5-terahydro-indolizine-8-carboxyllic acid cyclopropylmethoxy amide as the pure product.

¹H NMR (DMSO-D₆, 300 MHz): 11.4 (s, 1H), 8.7 (s, 1H), 7.8 (d, 1H), 7.65-7.55 (m, 2H), 5.8 (s, 1H), 3.8 (t, 2H), 3.6 (d, 2H), 3.2 (t, 2H), 2.2 (t, 2H), 1.25-1.15 (m, 1H), 0.65-0.55 (m, 2H), 0.45-0.35 (m, 2H).

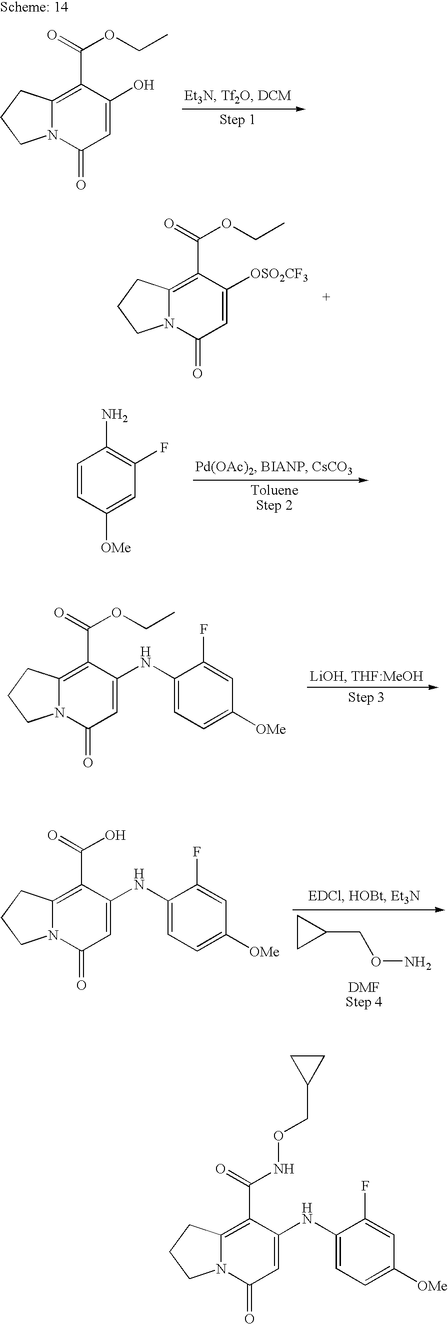

### EXAMPLE: 73

### Step 1 was Performed in a Manner Similar to What has been Described for Example 70

### Step: 2 Synthesis of 7-(2-Fluoro-4-methoxy-phenylamino)-5-oxo-1,2,3,5-terahydro-indolizine-8-carboxyllic acid ethyl ester

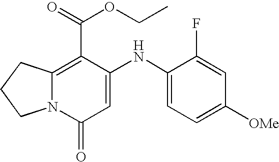

Palladium acetate (0.06 g, 0.003 mol), BINAP (0.26 g, 0.0002 mol), cesium carbonate (1.37 g, 0.004 mol) were dissolved in toluene and the resulting mixture was sparged for 30 mins with nitrogen. This was followed by addition of 5-oxo-7-trifluoromethanesulfonyloxy-1,2,3,5-tetrahydro-indolizine-8-carboxyllic acid ethyl ester (1 g, 0.003 mol) and 2-fluoro-4-methoxy aniline (0.54 g, 0.003 mol) and the reaction flask was again sparged for another 15 mins. The reaction mixture was heated at 110° C. for 1.30 hrs. The reaction mixture was filtered through celite and the filtrate was concentrated. The concentrate was purified by column chromatography (using silica gel, 70% MeOH in CHCl₃ as eluant) to afford 0.230 g (23.6% yield) of 7-(2-fluoro-4-methoxy-phenylamino)-5-oxo-1,2,3,5-terahydro-indolizine-8-carboxyllic acid ethyl ester as the required product.

¹H NMR (DMSO-D₆, 300 MHz): 9.2 (s, 1H), 7.2 (t, 1H), 6.85-6.75 (m, 2H), 5.5 (s, 1H), 4.4 (q, 2H), 4.2 (t, 2H), 3.8 (s, 3H), 3.5 (t 2H), 2.2 (q, 2H), 1.4 (t, 3H).

### Step: 3

### Synthesis of 7-(2-Fluoro-4-methoxy-phenylamino)-5-oxo-1,2,3,5-terahydro-indolizine-8-carboxyllic acid

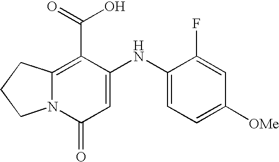

LiOH (0.05 g, 0.001 mol) was added to a stirred solution of 7-(2-fluoro-4-methoxy-phenylamino)-5-oxo-1,2,3,5-terahydro-indolizine-8-carboxyllic acid ethyl ester (0.19 g, 0.00 μmol) dissolved in MeOH:THF (6 mL) and the resulting mixture was stirred at RT for 4 hrs. The solvents were distilled and the crude product was acidified with 10% HCl to yield a precipitate which was collected and dried to afford 0.130 g (76.023% yield) of 7-(2-fluoro-4-methoxy-phenylamino)-5-oxo-1,2,3,5-terahydro-indolizine-8-carboxyllic acid.

¹H NMR (DMSO-D₆, 300 MHz): 13.45-13.35 (br s, 1H), 9.6 (s, 1H), 7.3 (t, 1H), 7.0 (d, 1H), 6.8 (d, 1H), 5.0 (s, 1H), 4.0 (t 2H), 3.8 (s, 3H), 3.5 (t 2H), 2.15-2.05 (m, 2H).

### Step: 4

### Synthesis of 7-(2-Fluoro-4-methoxy-phenylamino)-5-oxo-1,2,3,5-terahydro-indolizine-8-carboxyllic acid cyclopropylmethoxy amide

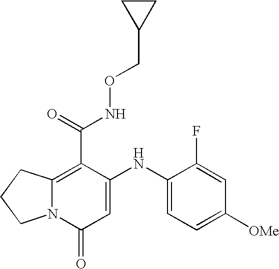

EDCI (0.22 g, 0.001 mol), HOBt (0.15 g, 0.001 mol) and 7-(2-fluoro-4-methoxy-phenylamino)-5-oxo-1,2,3,5-terahydro-indolizine-8-carboxyllic acid (0.12 g, 0.0004 mol) was dissolved in DMF in an inert atmosphere and the reaction mixture was stirred at RT for 30 mins. This was followed by addition of cyclopropylmethyl hydroxylamine hydrochloride (0.140 g, 0.001 mol) and TEA (0.114 g, 0.001 mol) and the reaction mixture was stirred at RT overnight. Water was added to the reaction mixture, followed by saturated NH₄Cl and extraction with ethyl acetate. The organic layer was washed with saturated bicarbonate solution, brine solution, dried over Na₂SO₄ to yield a precipitate, which was triturated with ether to afford 0.09 g (61.6% yield) of 7-(2-fluoro-4-methoxy-phenylamino)-5-oxo-1,2,3,5-terahydro-indolizine-8-carboxyllic acid cyclopropylmethoxy amide as the required product.

¹H NMR (DMSO-D₆, 300 M): 11.4 (s, 1H), 7.9 (s, 1H), 7.2 (t, 1H), 6.9 (d, 1H), 6.8 (d, 1H), 5.0 (s, 1H), 3.9 (t, 2H), 3.8 (s, 3H), 3.7 (d, 2H), 3.2 (t 2H), 2.05-2.0 (m, 2H), 1.05-1.0 (m, 1H), 0.65 (d, 2H), 0.2 (d, 2H).

### EXAMPLE: 74

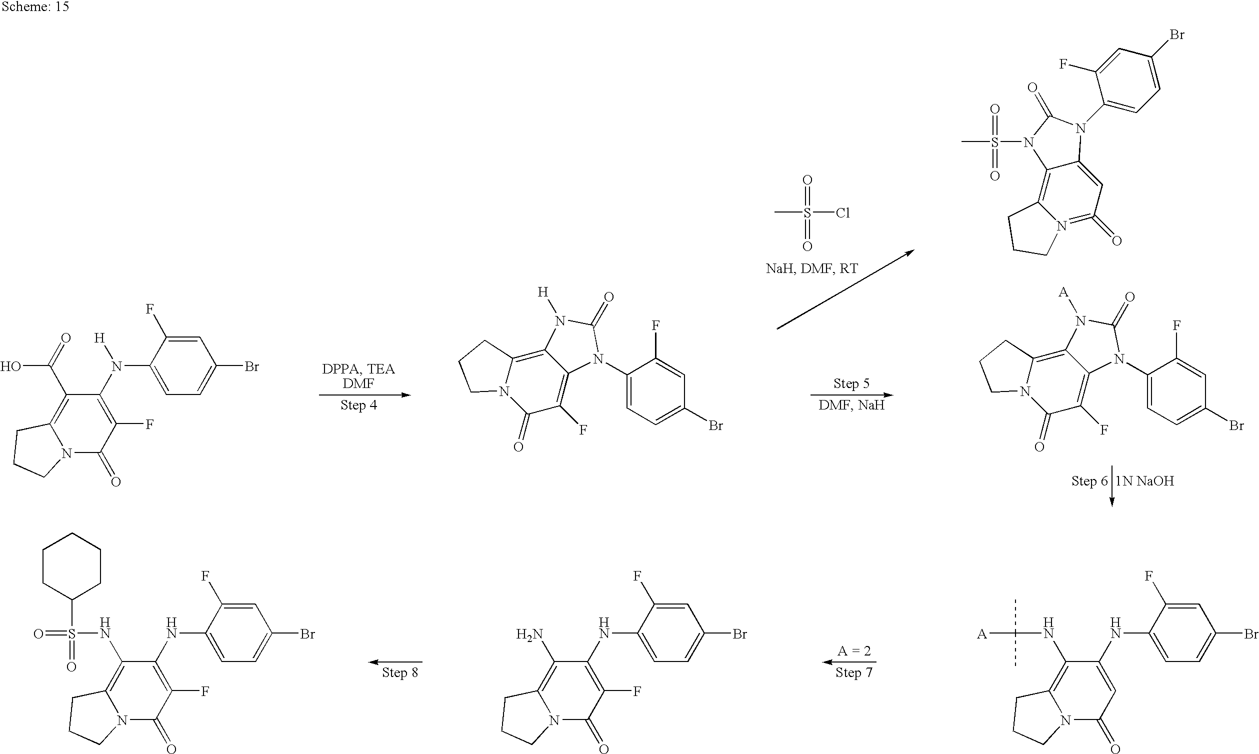

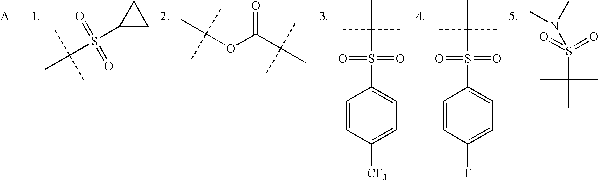

### Steps 1 to 2 were Performed in a Manner Similar to What has been Described for Example 27

### Step: 3

### Synthesis of 7-(4-Bromo-2-fluoro-phenylamino)-5-oxo-1,2,3,5-tetrahydro-indolizine-8-carboxylic acid

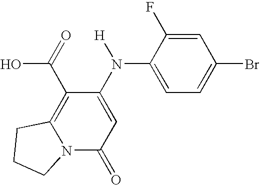

LDA (50 mL) were added to a solution of 2-fluoro-4-bromo-phenylamine (7 g, 0.037 mol) in THF (550 mL) at −78° C. and the resulting mixture was stirred for 1 hr. This was followed by addition of 7-chloro-5-oxo-1,2,3,5-tetrahydro-indolizine-8-carboxylic acid (5.5 g, 0.025 mol) in dry THF (200 mL) at −78° C. and the reaction mass was stirred at room temperature for 16 hrs. The reaction mixture was concentrated under reduced pressure, neutralized with dil HCl. Addition of diethyl ether and stirring for 1 hr yielded a precipitate which was collected to afford 5.2 g (56% yield) of 7-(4-bromo-2-fluoro-phenylamino)-5-oxo-1,2,3,5-tetrahydro-indolizine-8-carboxylic acid as the required product.

### Step: 4

### Synthesis of 3-(4-Bromo-2-fluoro-phenyl)-1,6,7,8-tetrahydro-3H-1,3,5a-triaza-as-indacene-2,5-dione

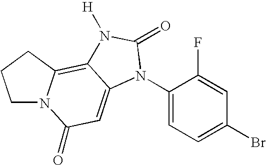

TEA (1.52 mg, 0.015 mol) were added to a solution of 7-(4-bromo-2-fluoro-phenylamino)-5-oxo-1,2,3,5-tetrahydro-indolizine-8-carboxylic acid (5 g, 0.014 mol) in dry DMF (8 mL) under nitrogen atmosphere and the reaction mixture was stirred for 30 mins. This was followed by dropwise addition of DPPA (4.14 g, 0.015 mol) over a period of 15 mins at 10° C. with stirring. The stirring was continued for a further 5 hrs at RT. This was followed by addition of toluene (80 mL) and the reaction mass was heated to 90° C. for 4 hrs. The reaction mass was concentrated under reduced pressure, and was followed by addition of chilled water. The precipitate formed was collected, dried to afford 3.4 g (70% yield) of 3-(4-bromo-2-fluoro-phenyl)-1,6,7,8-tetrahydro-3H-1,3,5a-triaza-as-indacene-2,5-dione as the required product.

### Step: 5

### Synthesis of 3-(4-Bromo-2-fluoro-phenyl)-1-methanesulfonyl-1,6,7,8-tetrahydro-3H-1,3,5a-triaza-as-indacene-2,5-dione

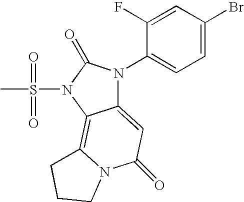

60% NaH (70 mg, 0.003 mol) was added to a stirred solution of 3-(4-bromo-2-fluoro-phenyl)-1,6,7,8-tetrahydro-3H-1,3,5a-triaza-as-indacene-2,5-dione (300 mg, 0.001 mol) in dry DMF (10 mL) at 0-5° C. under nitrogen atmosphere and the resulting mixture was stirred for 1 hr at RT. This was followed by dropwise addition of methanesulfonyl chloride (150 mg, 0.001 mol) in dry THF over a period of 5 mins at 0° C. and with stirring for the next 16 hrs at RT. Ice cold water was added into the reaction flask with stirring for 5 mins, pH was adjusted to 5. Extraction with ethylacetate, followed by drying over Na₂SO₄ and concentration under reduced pressure affords 160 mg (47% yield) of 3-(4-bromo-2-fluoro-phenyl)-1-methanesulfonyl-1,6,7,8-tetrahydro-3H-1,3,5a-triaza-as-indacene-2,5-dione as the required product.

HPLC: 93.7%

¹H NMR (DMSO-D₆, 300 MHz): 6, 7.9-7.45 (m, 3H), 5.45 (s, 1H), 3.9 (t, 2H), 3.65 (s, 3H), 3.3 (t, 2H), 2.15-2.05 (m, 2H)

### EXAMPLE: 75

### Steps 1 to 2 were Performed in a Manner Similar to What has been Described for Example 27 and Steps 3 to 4 were Performed in a Manner Similar to What has been Described for Example 74

### Step: 5

### Synthesis of 3-(4-Bromo-2-fluoro-phenyl)-1-cyclopropanesulfonyl-1,6,7,8-tetrahydro-3H-1,3,5a-triaza-as-indacene-2,5-dione

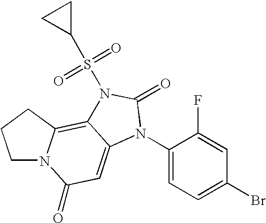

60% NaH (20 mg) was added to a stirred solution of 3-(4-bromo-2-fluoro-phenyl)-1,6,7,8-tetrahydro-3H-1,3,5a-triaza-as-indacene-2,5-dione (200 mg, 0.001 mol) in dry DMF (5 mL) at 0-5° C. under nitrogen atmosphere and the resulting mixture was stirred for 1 hr at RT. This was followed by dropwise addition of cyclopropanesulfonyl chloride (150 mg, 0.002 mol) in dry THF over a period of 10 mins at 0° C. and the stirring was continued for the next 16 hrs at RT. To the reaction mixture were added NaH (20 mg) at 0° C., stirred for 15 mins, followed by addition of cyclopropanesulfonyl chloride (150 mg, 0.002 mol) in dry DMF and continued stirring for a further 5 hrs at RT. The crude product was used for the next step without further purification.

### Step: 6

### Synthesis of Cyclopropanesulfonic acid [7-(4-bromo-2-fluoro-phenylamino)-5-oxo-1,2,3,5-tetrahydro-indolizin-8-yl]-amide

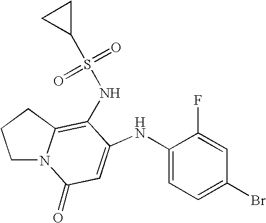

1N aqueous NaOH (7 mL) was added to 3-(4-bromo-2-fluoro-phenyl)-1-cyclopropanesulfonyl-1,6,7,8-tetrahydro-3H-1,3,5a-triaza-as-indacene-2,5-dione and the resulting mixture was heated to 75° C. for 4 hrs. Ice cold water was added to the reaction mixture, neutralization with 5% ice cold HCl to a pH of about 3 was followed by partitioning between ethylacetate and water. The organic layer was dried over Na₂SO₄, concentrated, purified by column chromatography (using silica gel, 3% methanol in DCM as eluant) to afford 17 mg (7% yield) of cyclopropanesulfonic acid [7-(4-bromo-2-fluoro-phenylamino)-5-oxo-1,2,3,5-tetrahydro-indolizin-8-yl]-amide as the required product.

LC-MS purity: 98.5%

HPLC: 97%

¹H NMR (DMSO-D₆, 300 MHz): δ 8.75 (s, 1H), 7.65-7.25 (m, 3H), 5.25 (s, 1H), 3.9 (t, 2H), 3.2 (t, 2H), 2.9-2.8 (m, 1H), 2.2-2.1 (m, 2H), 0.95-0.85 (m, 4H)

### EXAMPLE: 76

### Steps 1 to 2 were Performed in a Manner Similar to What has been Described for Example 27 and Steps 3 to 4 were Performed in a Manner Similar to What has been Described for Example 74

### Step: 5

### Synthesis of 3-(4-Bromo-2-fluoro-phenyl)-1-(4-fluoro-benzenesulfonyl)-1,6,7,8-tetrahydro-3H-1,3,5a-triaza-as-indacene-2,5-dione

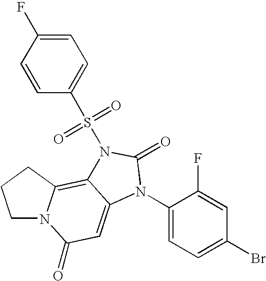

60% NaH (50 mg, 0.001 mol) was added to a stirred solution of 3-(4-bromo-2-fluoro-phenyl)-1,6,7,8-tetrahydro-3H-1,3,5a-triaza-as-indacene-2,5-dione (300 mg, 0.001 mol) in dry DMF (6 mL) at 0-5° C. under nitrogen atmosphere and the resulting mixture was stirred for 1 hr at RT. This was followed by dropwise addition of 4-fluoro-benzenesulfonyl chloride (200 mg, 0.001 mol) in dry THF over a period of 10 mins and the stirring was continued for the next 16 hrs at RT. The crude product was used for the next step without further purification.

### Step: 6

### Synthesis of N-[7-(4-Bromo-2-fluoro-phenylamino)-5-oxo-1,2,3,5-tetrahydro-indolizin-8-yl]-4-fluoro-benzenesulfonamide

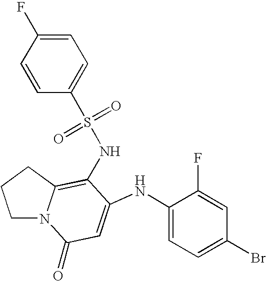

1N NaOH solution (6 mL) was added to 3-(4-bromo-2-fluoro-phenyl)-1-(4-fluoro-benzenesulfonyl)-1,6,7,8-tetrahydro-3H-1,3,5a-triaza-as-indacene-2,5-dione and the resulting mixture was heated to 60° C. for 1 hr. Ice cold water was added to the reaction mixture, neutralization with 5% HCl to a pH of about 4 was followed by partitioning between ethylacetate and water. The organic layer was washed with NaHCO₃, dried over Na₂SO₄, concentrated, purified by column chromatography (using silica gel, 2% methanol in DCM as eluant) to afford 20 mg (6% yield) of N-[7-(4-bromo-2-fluoro-phenylamino)-5-oxo-1,2,3,5-tetrahydro-indolizin-8-yl]-4-fluoro-benzenesulfonamide as the required product.

LC-MS purity: 97.5%

HPLC: 96.274%

¹H NMR (DMSO-D₆, 300 MHz): δ 9.25 (s, 1H), 7.0-7.85 (m, 7H), 5.25 (s, 1H), 3.9 (t, 2H), 2.75 (t, 2H), 1.95-1.8 (m, 1H)

### EXAMPLE: 77

### Steps 1 to 2 were Performed in a Manner Similar to What has been Described for Example 27 and Steps 3 to 4 were Performed in a Manner Similar to What has been Described for Example 74

### Step: 5

### Synthesis of 3-(4-Bromo-2-fluoro-phenyl)-2,5-dioxo-2,3,5,6,7,8-hexahydro-1,3,5a-triaza-as-indacene-1-carboxylic acid tert-butyl ester

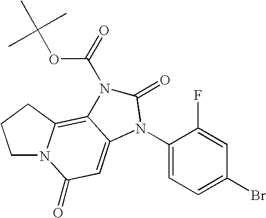

60% NaH (67 mg, 0.003 mol) was added to a stirred solution of 3-(4-bromo-2-fluoro-phenyl)-4-fluoro-1,6,7,8-tetrahydro-3H-1,3,5a-triaza-as-indacene-2,5-dione (2.5 mg, 0.001 mol) in dry DMF (10 mL) at 0° C. under nitrogen atmosphere. This was followed by dropwise addition of BOC anhydride (260 mg, 0.001 mol) in dry THF over a period 5 mins of at 0° C. and the resulting mixture was stirred for 4 hr at RT. The crude product was used in the next step without further purification.

### Step: 6

### Synthesis of [7-(4-Bromo-2-fluoro-phenylamino)-5-oxo-1,2,3,5-tetrahydro-indolizin-8-yl]-carbamic acid tert-butyl ester

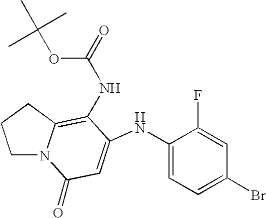

1N aqueous NaOH (6 mL) was added to 3-(4-bromo-2-fluoro-phenyl)-2,5-dioxo-2,3,5,6,7,8-hexahydro-1,3,5a-triaza-as-indacene-1-carboxylic acid tert-butyl ester and the resulting mixture was heated to 65° C. for 3 hrs. Ice cold water was added to the reaction mixture, neutralized with 5% HCl to a pH of about 7 and the reaction mixture was partitioned between ethylacetate and water. The organic layer was dried over Na₂SO₄, concentrated under reduced pressure and the concentrate was purified by column chromatography (using neutral alumina, 3% methanol in DCM as eluant) to afford 165 mg (43.8% yield) of [7-(4-bromo-2-fluoro-phenylamino)-5-oxo-1,2,3,5-tetrahydro-indolizin-8-yl]-carbamic acid tert-butyl ester as the required product.

HPLC: 94.2%

¹H NMR (DMSO-D₆, 300 MHz): δ 8.15 (s, 1H), 7.9-7.45 (m, 3H), 5.15 (s, 1H), 3.9 (t, 2H), 2.9 (t, 2H), 2.2-2.1 (m, 2H), 1.45 (s, 9H)

### EXAMPLE: 78

### Steps 1 to 2 were Performed in a Manner Similar to What has been Described for Example 27, Steps 3 to 4 were Performed in a Manner Similar to What has been Described for Example 74, and Steps 5 to 6 were Performed in a Manner Similar to What has been Described for Example 77.

### Step: 7

### Synthesis of 8-Amino-7-(4-bromo-2-fluoro-phenylamino)-2,3-dihydro-1H-indolizin-5-one

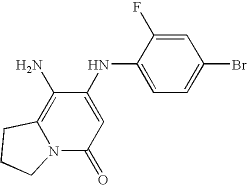

TFA (1 mL) was added dropwise to a stirred solution of [7-(4-bromo-2-fluoro-phenylamino)-5-oxo-1,2,3,5-tetrahydro-indolizin-8-yl]-carbamic acid tert-butyl ester (200 mg, 0.0005 mol) in dry DCM (5 mL) over a period of 5 mins at −10° C. and the resulting mixture was stirred for 4 hrs at RT. The reaction mass was concentrated under reduced pressure. Addition of water, neutralization with NaHCO₃ solution and extraction with ethylacetate and drying over Na₂SO₄ affords 120 mg (79% yield) of 8-amino-7-(4-bromo-2-fluoro-phenylamino)-2,3-dihydro-1H-indolizin-5-one as the required product.

LCMS purity: 88.3%

### Step: 8

### Synthesis of Cyclohexanesulfonic acid [7-(4-bromo-2-fluoro-phenylamino)-5-oxo-1,2,3,5-tetrahydro-indolizin-8-yl]-amide

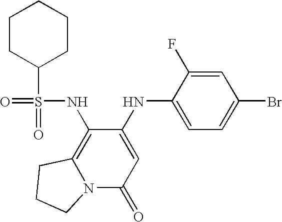

Pyridine in DCM (4 mL) was added to a solution of 8-amino-7-(4-bromo-2-fluoro-phenylamino)-2,3-dihydro-1H-indolizin-5-one (110 mg, 0.0003 mol) and the reaction mixture was stirred for 10 mins under nitrogen atmosphere. This was followed by addition of DMAP (5 mg, 0.0004 mol), cooled the reaction mass to 0-5° C., added cyclohexyl sulfonyl chloride (80 mg, 0.0004 mol) in DCM dropwise over a period of 10 mins and continued stirring for the next 16 hrs at RT. The reaction mixture was partitioned between ethylacetate and water and the organic layer was washed with water and 1N dil. HCl. The organic layer was dried over Na₂SO₄, concentrated and the concentrate was purified by column chromatography (using silica gel, 2% methanol in DCM as eluant) to afford 10 mg (8.5% yield) of cyclohexanesulfonic acid [7-(4-bromo-2-fluoro-phenylamino)-5-oxo-1,2,3,5-tetrahydro-indolizin-8-yl]-amide as the required product.

LC-MS purity: 96.8%, m/z=484, (M+1)

HPLC: 93.4%

¹H NMR (DMSO-D₆, 300 MHz): δ 7.8 (s, 1H), 7.65-7.25 (m, 3H), 5.95 (s, 1H), 4.1 (t, 2H), 3.2 (t, 2H), 3.15-3.05 (m, 1H), 2.25-2.15 (m, 1H), 2.2-2.1 (m, 2H), 1.85-1.75 (m, 2H), 1.65-1.6 (m, 4H), 1.3-1.25 (m, 2H)

### EXAMPLE: 79

### Steps 1 to 2 were Performed in a Manner Similar to What has been Described for Example 27 and Steps 3 to 4 were Performed in a Manner Similar to What has been Described for Example 74

### Step: 5

### Synthesis of 3-(4-Bromo-2-fluoro-phenyl)-1-(4-trifluoromethyl-benzenesulfonyl)-1,6,7,8-tetrahydro-3H-1,3,5a-triaza-as-indacene-2,5-dione

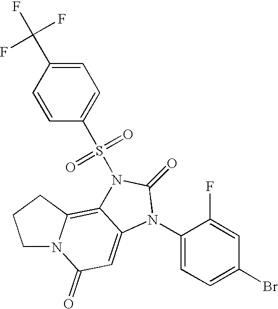

60% NaH (31 mg, 0.001 mol) was added to a stirred solution of 3-(4-bromo-2-fluoro-phenyl)-1,6,7,8-tetrahydro-3H-1,3,5a-triaza-as-indacene-2,5-dione (200 mg, 0.001 mol) in dry DMF (4 mL) at 0° C. and the resulting mixture was stirred for 1 hr at RT. This was followed by dropwise addition of 4-trifluoromethyl-benzenesulfonyl chloride (180 mg, 0.001 mol) in dry THF over a period of 10 mins at 0° C. Continued stirring for the next 2 days at RT affords a crude product which was used for the next step without further purification.

### Step: 6

### Synthesis of N-[7-(4-Bromo-2-fluoro-phenylamino)-5-oxo-1,2,3,1-tetrahydro-indolizin-8-yl]-4-trifluoromethyl-benzenesulfonamide

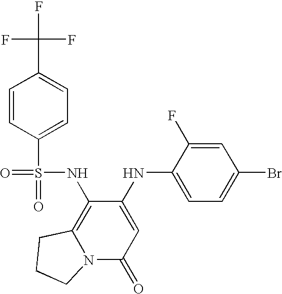

1N aqueous NaOH (15 mL) was added to 3-(4-bromo-2-fluoro-phenyl)-1-(4-trifluoromethyl-benzenesulfonyl)-1,6,7,8-tetrahydro-3H-1,3,5a-triaza-as-indacene-2,5-dione and the resulting mixture was heated to 65° C. for 2 hrs. The dil. HCl (pH=4) were added to the reaction mass at RT, then the PH was adjusted to 7 using aqueous Na₂CO₃ and extracted the reaction mass with ethylacetate. The organic layer was washed with brine, dried over Na₂SO₄, concentrated under reduced pressure and the concentrate was purified by column chromatography (using silica gel, 1.5% methanol in DCM) to afford 102 mg (33% yield) of as the required product.

LC-MS purity: 98.9%, m/z=548, (M+2)

HPLC: 99.6%

¹H NMR (DMSO-D₆, 300 MHz): δ 9.4 (s, 1H), 7.85-6.85 (m, 7H), 5.25 (s, 1H), 3.9 (t, 2H), 2.85 (t, 2H), 2.0-1.9 (m, 1H)

### EXAMPLE: 80

### Steps 1 to 2 were Performed in a Manner Similar to What has been Described for Example 27, Steps 3 to 4 were Performed in a Manner Similar to What has been Described for Example 74, Steps 5 to 6 were Performed in a Manner Similar to What has been Described for Example 77, and Step 7 was Performed in a Manner Similar to What has been Described for Example 78.

### Step: 8

### Synthesis of N-[7-(4-Bromo-2-fluoro-phenylamino)-5-oxo-1,2,3,5-tetrahydro-indolizin-8-yl]-N,N-dimethylaminosulfonamide

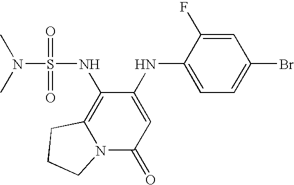

N,N-Dimethyl sulfonyl chloride (40 mg, 0.0003 mol) was added to a solution of 8-amino-7-(4-bromo-2-fluoro-phenylamino)-2,3-dihydro-1H-indolizin-5-one (80 mg, 0.0002 mol) in dry THF and DMAP (30 mg, 0.0002 mol) at −35° C. and the reaction mixture was stirred at RT for 2 hrs. This was followed by addition of dry pyridine (1.5 mL) and the reaction mixture was heated to 40° C. for 4 hrs. The reaction mass was concentrated under reduced pressure and the concentrate was purified by preparative HPLC to afford 6 mg (9% yield) of N-[7-(4-bromo-2-fluoro-phenylamino)-5-oxo-1,2,3,5-tetrahydro-indolizin-8-yl]-N,N-dimethylaminosulfonamide as the required product.

HPLC: 91.4%

¹H NMR (DMSO-D₆, 300 MHz): δ 7.15 (s, 1H), 7.85-7.25 (m, 3H), 5.9 (s, 1H), 4.2 (t, 2H), 3.29 (t, 2H), 2.9 (s, 6H), 2.25-2.15 (m, 2H)

### Scheme: 16

### EXAMPLE: 81

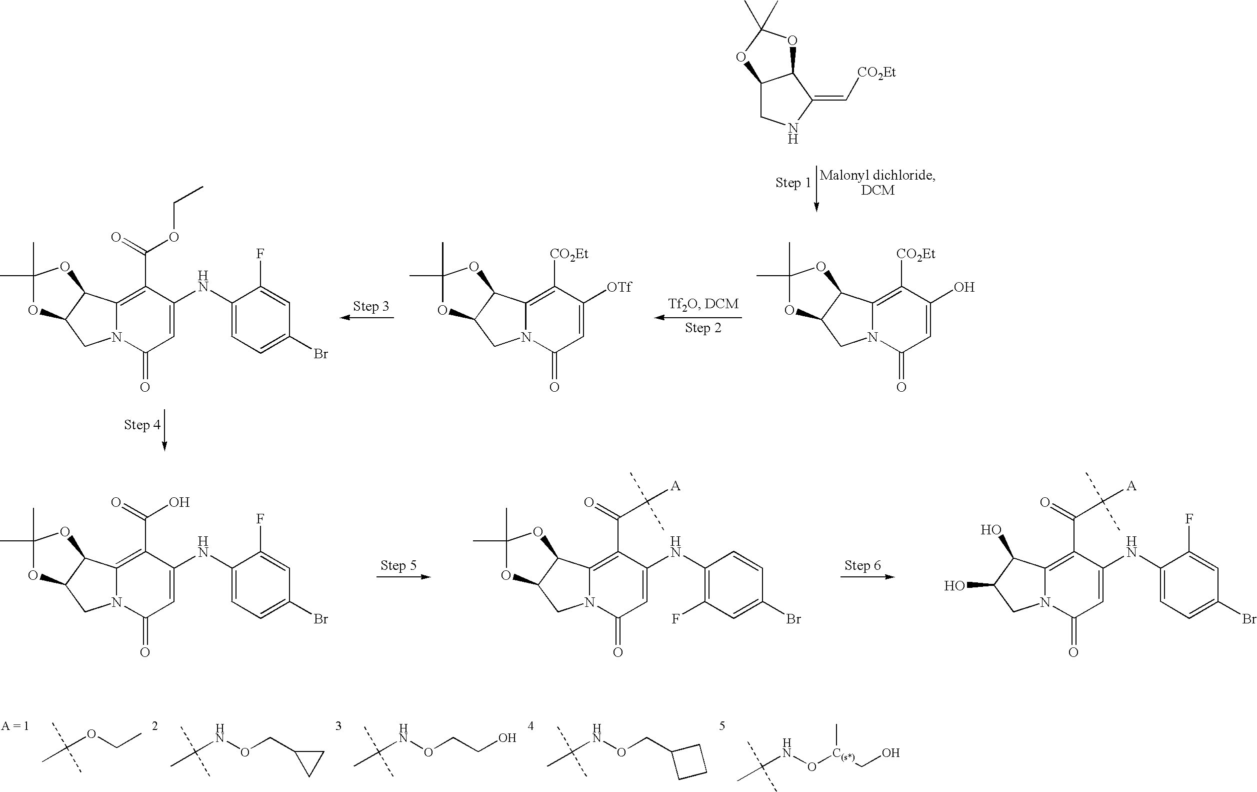

### Step: 1

### Synthesis of 5-Hydroxy-2,2-dimethyl-7-oxo-3a,7,8,8a-tetrahydro-1,3-dioxa-7a-aza-cyclopenta[a]indene-4-carboxylic acid ethyl ester

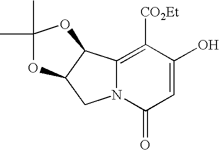

Malonyl chloride (8.10 g, 0.057 mol) was added dropwise to a solution of (2,2-dimethyl-tetrahydro-[1,3]dioxolo[4,5-c]pyrrol-4-ylidene)-acetic acid ethyl ester (J. Chem. Soc., Perkins Transactions 1: Organic and Bio-organic Chemistry, pgs 2371-2376, (1987)) (10.2 g, 0.048 mol) in DCM (300 mL) over a period of 30 mins and the reaction mixture was stirred at RT for 3 hrs. The reaction mass was quenched with 5 mL of TEA and the solvent was concentrated. The concentrate was purified by column chromatography (using silica gel, 30-45% ethylacetate in hexane as eluant) to afford 9.25 g (69% yield) of 5-hydroxy-2,2-dimethyl-7-oxo-3a,7,8,8a-tetrahydro-1,3-dioxa-7a-aza-cyclopenta[a]indene-4-carboxylic acid ethyl ester as the required product.

¹H NMR (CDCl₃, 300 MHz): 11.25-11.15 (br s, 1H), 6.0 (s, 1H), 6.0-5.95 (m, 1H), 5.05-4.95 (m, 1H), 4.45-4.35 (m, 3H), 4.15-4.05 (m, 1 μl), 1.45-1.35 (m, 6H), 1.3 (s, 3H).

### Step: 2

### Synthesis of 2,2-Dimethyl-7-oxo-5-trifluoro methanesulfonyloxy-3a,7,8,8a-tetrahydro-1,3-dioxa-7a-aza-cyclopenta[a]indene-4-carboxylic acid ethyl ester

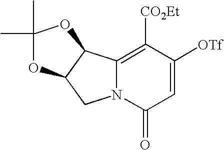

TEA (4.9 g, 0.049 mol) was added to a solution of 5-hydroxy-2,2-dimethyl-7-oxo-3a,7,8,8a-tetrahydro-1,3-dioxa-7a-aza-cyclopenta[a]indene-4-carboxylic acid ethyl ester (9.25 g, 0.033 mol) in DCM (200 mL) at −70° C. This was followed by dropwise addition of triflic anhydride (12.06 g, 0.0427 mol) in DCM (500 mL) over a period of 1 hr and the reaction mixture was stirred at RT for 1 hr. The reaction mass was partitioned between ethylacetate and water. The organic layer was washed with brine solution and concentrated. The concentrate was purified by column chromatography (using silica gel, as eluant) to afford 9 g (64.3% yield) of 2,2-dimethyl-7-oxo-5-trifluoromethanesulfonyloxy-3a,7,8,8a-tetrahydro-1,3-dioxa-7a-aza-cyclopenta[a]indene-4-carboxylic acid ethyl ester as the required product.

¹H NMR (CDCl₃, 300 MHz): 6.4 (s, 1H), 6.0 (d, 1H), 5.0 (t, 1H), 4.45-4.35 (m, 3H), 4.15-4.05 (m, 1H), 1.45-1.35 (m, 6H), 1.3 (s, 3H).

### Step: 3

### Synthesis of 5-(4-Bromo-2-fluoro-phenylamino)-2,2-dimethyl-7-oxo-3a,7,8,8a-tetrahydro-1,3-dioxa-7a-aza-cyclopenta[a] indene-4-carboxylic acid ethyl ester

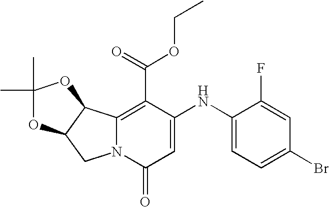

Palladium acetate (0.47 g, 0.002 mol), BINAP (1.96 g, 0.003 mol), cesium carbonate (10.26 g, 0.032 mol) were dissolved in toluene (200 mL) and the resulting mixture was sparged for 30 mins with nitrogen. This was followed by addition of 2,2-dimethyl-7-oxo-5-trifluoromethanesulfonyloxy-3a,7,8,8a-tetrahydro-1,3-dioxa-7a-aza-cyclopenta[a]indene-4-carboxylic acid ethyl ester (9 g, 0.021 mol) and 2-fluoro-4-bromo aniline (4.4 g, 0.023 mol) with continued sparging for a further 15 mins. The reaction mixture was heated to 90° C. for 1.30 hrs. The reaction mixture was cooled and diluted with 200 mL of ethylacetate, washed with water, brine solution, dried over Na₂SO₄ and concentrated. The concentrate was purified by column chromatography (using silica gel, 10-70% ethylacetate in hexane as eluant) to afford 5 g (51.5% yield) of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid ethyl ester as the required product.

LCMS: m/z=467 (M+H)

¹H NMR (CDCl₃, 300 M): 9.3 (s, 1H), 7.45-7.35 (m, 1H), 7.35-7.25 (m, 2H), 6.0 (d, 1H), 5.8 (s, 1H), 5.0 (t, 1H), 4.45-4.35 (m, 2H), 4.15-4.05 (m, 1H), 1.45-1.35 (m, 6H), 1.3 (s, 3H).

### Step: 4

### Synthesis of 5-(4-Bromo-2-fluoro-phenylamino)-2,2-dimethyl-7-oxo-3a,7,8,8a-tetrahydro-1,3-dioxa-7a-aza-cyclopenta[a]indene-4-carboxylic acid

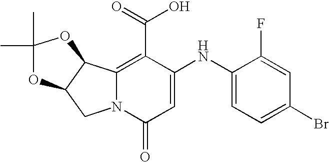

LiOH (0.22 g, 0.005 mol) in water was added to a stirred solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid ethyl ester (1 g, 0.002 mol) dissolved in MeOH: THF (1:4) and the resulting mixture was stirred at RT for 2 hrs. The reaction mixture was concentrated and the residue was partitioned between ethylacetate and water and the aqueous layer was acidified with 10% citric acid solution (pH 2.5). The precipitate formed was collected and dried under reduced pressure to afford the crude product which was purified by preparative HPLC to afford 50 mg (58.5% yield) of 5-(4-Bromo-2-fluoro-phenylamino)-2,2-dimethyl-7-oxo-3a,7,8,8a-tetrahydro-1,3-dioxa-7a-aza-cyclopenta[a]indene-4-carboxylic acid as the required product.

LCMS purity: 99.5%, m/z=439.0 (M−H)

HPLC: 99.3%

¹H NMR (DMSO-D₆, 300 MHz): 13.65-13.55 (br s, 1H), 9.55-9.45 (br s, 1H), 7.7 (dd, 1H), 7.55-7.45 (m, 2H), 6.0 (d, 1H), 5.4 (s, 1H), 4.9 (t, 1H), 4.05-3.95 (m, 2H), 1.4 (s, 3H), 1.2 (s, 3H).

### EXAMPLE: 82

### Steps 1 to 12 were Performed in a Manner Similar to What has been Described for Example 81

### Step: 5

### Synthesis of 5-(4-Bromo-2-fluoro-phenylamino)-2,2-dimethyl-7-oxo-3a,7,8,8a-tetrahydro-1,3-dioxa-7a-aza-cyclopenta[a] indene-4-carboxylic acid cyclopropylmethoxy-amide

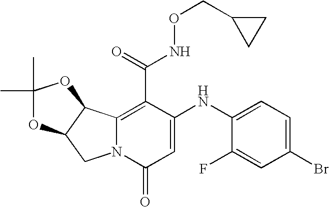

EDCI (262.4 mg, 1.369 mmol), HOBt (184.61 mg, 1.369 mmol) and DIPEA (387 mg, 2.736 mmol) were added to a stirred solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid (200 mg, 0.456 mmol) in DMF (5 mL) and DCM (5 mL). This was followed by addition of O-cyclopropylmethyl-hydroxylamine (168.38 mg, 1.369 mmol) and the reaction mixture was stirred for 16 hrs at RT. The reaction mixture was partitioned between water and ethylacetate. The organic layer was washed with saturated NaHCO₃, brine solution, dried over Na₂SO₄ and concentrated to afford 120 mg (51.7% yield) of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid cyclopropylmethoxy-amide as the required product.

LCMS purity: 82.9%, m/z=508.1 (M+H).

HPLC: 79.5%

¹H NMR (DMSO-D₆, 300 MHz): 10.5 (s, 1H), 9.3 (s, 1H), 7.2-7.4 (m, 3H), 5.9 (s, 1H), 5.6 (d, 1H), 5.0 (t, 1H), 4.45-4.35 (m, 1H), 4.15-4.05 (m, 1H), 3.95-3.85 (m, 1H), 3.85-3.75 (m, 1H), 1.6 (s, 3H), 1.5 (s, 3H), 0.85-0.75 (m, 1H), 0.65-0.55 (m, 2H), 0.45-0.35 (m, 2H).

### Step: 6

### Synthesis of 7-(4-Bromo-2-fluoro-phenylamino)-1,2-dihydroxy-5-oxo-1,2,3,5-tetrahydro-indolizine-8-carboxylic acid cyclopropylmethoxy-amide

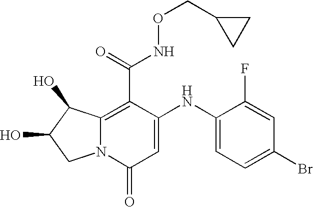

Conc. HCl (3 mL) was added to a solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid cyclopropylmethoxy-amide (120 mg) dissolved in methanol (3 mL) and the resulting mixture was stirred at RT for 3 hrs. The reaction mixture was concentrated and the concentrate was triturated twice with ethylacetate (2×0.5 mL). The dried residue affords 20 mg (18.2% yield) of 7-(4-bromo-2-fluoro-phenylamino)-1,2-dihydroxy-5-oxo-1,2,3,5-tetrahydro-indolizine-8-carboxylic acid cyclopropylmethoxy-amide as the required product.

LCMS purity: 95.3%, m/z=470 (M+H).

HPLC: 94.1%

¹H NMR (DMSO-D₆, 300 MHz): 11.4 (s, 1H), 8.1 (s, 1H), 7.6 (dd, 1H), 7.45-7.35 (m, 2H), 5.4 (s, 1H), 5.1 (d, 1H), 4.35-4.25 (m, 1H), 3.95-3.85 (m, 1H), 3.8-3.7 (m, 3H), 1.25-1.15 (m, 1H), 0.6 (d, 2H), 0.4 (d, 2H)

### EXAMPLE: 83

### Steps 1 to 12 were Performed in a Manner Similar to What has been Described for Example 81

### Step: 5

### Synthesis of 5-(4-Bromo-2-fluoro-phenylamino)-2,2-dimethyl-7-oxo-3a,7,8,8a-tetrahydro-1,3-dioxa-7a-aza-cyclopenta[a] indene-4-carboxylic acid (2-hydroxy-ethoxy)-amide

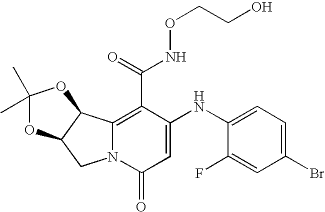

EDCI (262.4 mg, 1.369 mmol), HOBt (186.6 mg, 1.369 mmol), TEA (279 mg, 2.736 mmol) and O-(2-tert-butoxy-ethyl)-hydroxylamine (182 mg, 1.369 mmol) were added to a stirred solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid (200 mg, 0.456 mmol) in DMF (5 mL) and DCM (5 mL). The reaction mixture was stirred at RT for 16 hrs. The reaction mixture was partitioned between water and ethylacetate. The organic layer was washed with saturated NaHCO₃, brine solution, dried over Na₂SO₄ and concentrated. The concentrate was purified by column chromatography (using silica gel, 2% methanol in DCM as eluant) to afford 100 mg (39.5% yield) of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid (2-hydroxy-ethoxy)-amide as the required product.

### Step: 6

### Synthesis of 7-(4-Bromo-2-fluoro-phenylamino)-1,2-dihydroxy-5-oxo-1,2,3,5-tetrahydro-indolizine-8-carboxylic acid (2-hydroxy-ethoxy)-amide

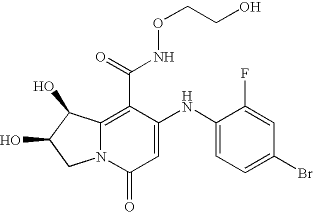

Conc. HCl (3 mL) was added to a solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid (2-hydroxy-ethoxy)-amide (100 mg, 0.18 mmol) in methanol (3 mL) and the reaction mixture was stirred at RT for 3 hrs. The reaction mixture was concentrated and the concentrate was triturated with ethylacetate, The precipitate collected was purified by preparative HPLC to afford 50 mg (61.3% yield) of 7-(4-bromo-2-fluoro-phenylamino)-1,2-dihydroxy-5-oxo-1,2,3,5-tetrahydro-indolizine-8-carboxylic acid (2-hydroxy-ethoxy)-amide as the required product.

LCMS purity: 98.9%, m/z=458.0 (M+H).

HPLC: 98.7%

¹H NMR (DMSO-D₆, 300 MHz): 11.4 (s, 1H), 8.2 (s, 1H), 7.7 (d, 1H), 7.45-7.35 (m, 2H), 5.8 (s, 1H), 5.4 (d, 1H), 5.3 (s, 1H), 5.1 (t, 1H), 4.8 (t, 1H), 4.3 (t, 1H), 4.0 (t, 2H), 3.7-3.9 (m, 2H), 3.65-3.55 (m, 2H).

### EXAMPLE: 84

### Steps 1 to 12 were Performed in a Manner Similar to What has been Described for Example 81

### Step: 5

### Synthesis of 5-(4-Bromo-2-fluoro-phenylamino)-2,2-dimethyl-7-oxo-3a,7,8,8a-tetrahydro-1,3-dioxa-7a-aza-cyclopenta[a]indene-4-carboxylic acid cyclobutylmethoxy-amide

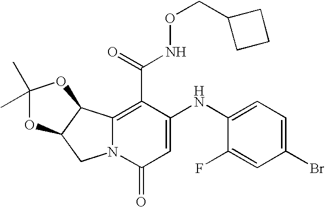

EDCI (262.4 mg, 1.369 mmol), HOBt (61 mg, 0.456 mmol), TEA (279 mg, 2.736 mmol) and O-cyclobutylmethyl-hydroxylamine (138 mg, 1.369 mmol) were added to a stirred solution of 5-(4-Bromo-2-fluoro-phenylamino)-2,2-dimethyl-7-oxo-3a,7,8,8a-tetrahydro-1,3-dioxa-7a-aza-cyclopenta[a]indene-4-carboxylic acid (200 mg, 0.456 mmol) in DMF (5 mL) and DCM (5 mL). The reaction mixture was stirred at RT for 16 hrs. The reaction mixture was partitioned between water and ethylacetate (2×25 mL). The organic layer was washed with saturated NaHCO₃, brine solution, dried over Na₂SO₄ and concentrated. The concentrate was purified by column chromatography (using silica gel, 2% methanol in DCM as eluant) to afford 140 mg (61.4% yield) of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid cyclobutylmethoxy-amide as the required product.

LCMS: m/z=522.1 (M+H).

¹H NMR (DMSO-D₆, 300 MHz): 10.4 (s, 1H), 9.2 (s, 1H), 7.2-7.4 (m, 3H), 5.8 (s, 1H), 5.6 (d, 1H), 5.1 (t, 1H), 4.4 (d, 1H), 3.95-4.05 (m, 3H), 2.85-2.75 (m, 1H), 2.15-2.05 (m, 2H), 1.95-1.85 (m, 4H), 1.5 (s, 6H)

### Step: 6

### Synthesis of 7-(4-Bromo-2-fluoro-phenylamino)-1,2-dihydroxy-5-oxo-1,2,3,5-tetrahydro-indolizine-8-carboxylic acid cyclobutylmethoxy-amide

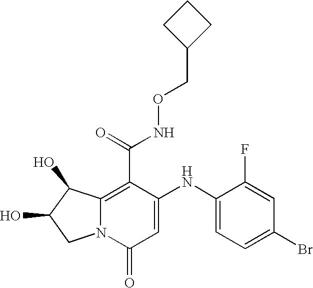

Conc. HCl (3 mL) was added to a solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid cyclobutylmethoxy-amide (130 mg, 0.249 mmol) dissolved in methanol (3 mL) and the resulting mixture was stirred at RT for 3 hrs. The reaction mixture was concentrated and the concentrate was triturated with ethylacetate. The precipitate formed was collected, purified by preparative HPLC to afford 35 mg (29.1% yield) of 7-(4-bromo-2-fluoro-phenylamino)-1,2-dihydroxy-5-oxo-1,2,3,5-tetrahydro-indolizine-8-carboxylic acid cyclobutylmethoxy-amide as the required product.

¹H NMR (DMSO-D₆, 300 MHz): 11.4 (s, 1H), 8.1 (s, 1H), 7.7 (d, 1H), 7.45-7.35 (m, 2H), 5.3 (s, 1H), 5.1 (s, 1H), 4.35-4.25 (m, 1H), 3.85-3.75 (m, 6H), 3.8-3.7 (m, 3H), 2.65-2.55 (m, 1H), 2.05-1.95 (m, 2H), 1.85-1.75 (m, 4H).

### EXAMPLE: 85

### Steps 1 to 12 were Performed in a Manner Similar to What has been Described for Example 81

### Step: 5

### Synthesis of 5-(4-Bromo-2-fluoro-phenylamino)-2,2-dimethyl-7-oxo-3a,7,8,8a-tetrahydro-1,3-dioxa-7a-aza-cyclopenta[a] indene-4-carboxylic acid (3-tert-butoxy-2-methyl-propoxy)-amide

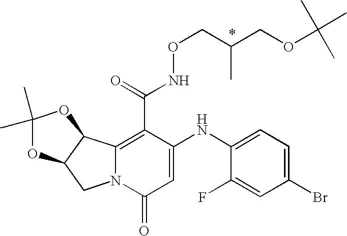

EDCI (262.4 mg, 1.369 mmol), HOBt (186.6 mg, 1.369 mmol), TEA (279 mg, 2.736 mmol) and O-(3-tert-butoxy-2-methyl-propyl)-hydroxylamine (220 mg, 1.369 mmol) were added to a stirred solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid (200 mg, 0.456 mmol) in DMF (5 mL) and DCM (5 mL). The reaction mixture was stirred at RT for 16 hrs. The reaction mixture was partitioned between water and ethylacetate. The organic layer was washed with saturated NaHCO₃, brine solution, dried over anhydrous Na₂SO₄ and concentrated. The concentrate was purified by column chromatography (using silica gel, 0-2% methanol in DCM as eluant) to afford 100 mg (37.8% yield) of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid (3-tert-butoxy-2-methyl-propoxy)-amide as the required product.

LCMS purity: 76.9%, m/z=568.1 (M+H)

HPLC: 52.3%

### Step: 6

### Synthesis of 7-(4-Bromo-2-fluoro-phenylamino)-1,2-dihydroxy-5-oxo-1,2,3,5-tetrahydro-indolizine-8-carboxylic acid (3-hydroxy-2-methyl-propoxy)-amide

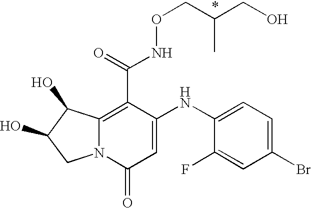

Conc. HCl (3 mL) was added to a solution of 5-(4-bromo-2-fluoro-phenylamino)-2,2-dimethyl-7-oxo-3a,7,8,8a-tetrahydro-1,3-dioxa-7a-aza-cyclopenta[a]indene-4-carboxylic acid (3-tert-butoxy-2-methyl-propoxy)-amide (120 mg, 0.21 mmol) dissolved in methanol (3 mL) and the resulting mixture was stirred at RT for 2 hrs. The reaction mixture was concentrated and the concentrate was triturated with ethylacetate to yield a precipitate. Purification by preparative HPLC affords 50 mg (48.5% yield) of 7-(4-bromo-2-fluoro-phenylamino)-1,2-dihydroxy-5-oxo-1,2,3,5-tetrahydro-indolizine-8-carboxylic acid (3-hydroxy-2-methyl-propoxy)-amide as the required product.

¹H NMR (DMSO-D₆, 300 M): 11.4 (s, 1H), 8.1 (d, 1H), 7.7 (dd, 1H), 7.45-7.35 (m, 2H), 5.85-5.75 (m, 1H), 5.4 (t, 1H), 5.3 (s, 1H), 5.15-5.05 (m, 1H), 4.7 (dt, 1H), 4.35-4.25 (m, 1H), 4.15-4.05 (m, 1H), 3.8 (dd, 1H), 3.7 (dd, 1H), 3.55-3.45 (m, 2H), 1.2 (d, 3H).

### Scheme: 17

### Steps 1-4 Same as Example: 8

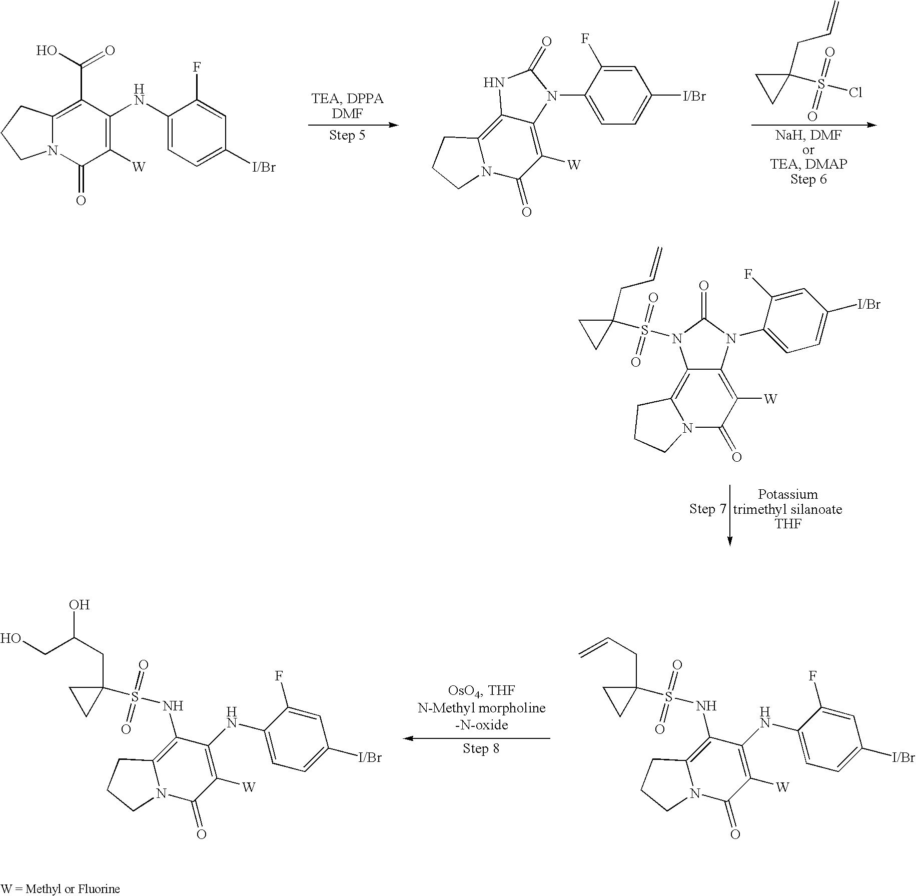

### EXAMPLE: 86

### Steps 1 to 4 were Performed in a Manner Similar to What has been Described for Example 8

### Step: 5

### Synthesis of 4-Fluoro-3-(2-fluoro-4-iodo-phenyl)-1,6,7,8-tetrahydro-3H-1,3,5a-triaza-as-indacene-2,5-dione

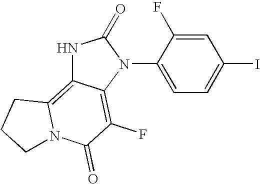

TEA (1.30 mL, 9.259 mmol) and DPPA (2.0 mL, 9.259 mmol) were added to a stirred solution of 6-fluoro-7-(2-fluoro-4-iodo-phenylamino)-5-oxo-1,2,3,5-tetrahydro-indolizine-8-carboxylic acid (4.0 g, 9.259 mmol) in DMF (30 mL) at 0° C. and the reaction mixture was stirred for 4 hrs at RT under nitrogen atmosphere. The reaction mixture was heated to 65° C. overnight. The reaction was monitored by TLC (15% MeOH in DCM). The resulting reaction mixture was cooled, addition of water facilitated the formation of a precipitate which was collected and dried under reduced pressure to afford 3.65 g of the product (91% yield).

LCMS purity: 98.96%, m/z=429.9 (M+1)

HPLC: 84.6%

¹H NMR (DMSO-D₆, 300 MHz): δ 11.3 (s, 1H), 7.9-7.25 (m, 3H), 4.0 (t, 2H), 3.1 (t, 2H), 2.3-2.2 (m, 2H)

### Step: 6

### Synthesis of 1-(1-Allyl-cyclopropanesulfonyl)-4-fluoro-3-(2-fluoro-4-iodo-phenyl)-1,6,7,8-tetrahydro-3H-1,3,5a-triaza-as-indacene-2,5-dione

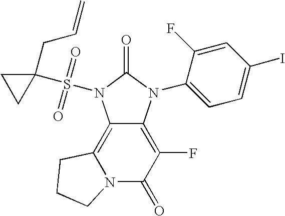

60% NaH (0.37 g, 9.324 mol) was added to a stirred solution of 4-fluoro-3-(2-fluoro-4-iodo-phenyl)-1,6,7,8-tetrahydro-3H-1,3,5a-triaza-as-indacene-2,5-dione (2.0 g, 4.662 mmol) in dry DMF (20 mL) at 0° C., under nitrogen atmosphere and the resulting mixture was stirred for 20 mins at 0° C. This was followed by the addition of 1-allyl-cyclopropanesulfonyl chloride (1.26 g, 6.993 mol) at 0° C. and stirring was continued for the next 18 hrs at RT. The reaction was monitored by TLC (10% MeOH in DCM). The reaction mixture was partitioned between water and ethylacetate. The organic layer was washed with water, dried over Na₂SO₄ and concentrated. Purification by column chromatography on silica gel (60% ethylacetate in hexane) afforded 1.22 g of the product (46% yield).

LCMS purity: 77.1%, m/z=573.9 (M+1)

¹H NMR (CDCl₃, 300 MHz): δ 7.62 (d, 2H), 7.15 (t, 1H), 5.7-5.6 (m, 1H), 5.1 (d, 2H), 4.4 (t, 2H), 3.5 (t, 2H), 2.7-2.6 (m, 2H), 2.3-2.2 (m, 2H), 1.9-1.8 (m, 1H), 1.8-1.7 (m, 1H), 1.2-1.1 (m, 2H)

### Step: 7

### Synthesis of 1-Allyl-cyclopropanesulfonic acid [6-fluoro-7-(2-fluoro-4-iodo-phenylamino)-5-oxo-1,2,3,5-tetrahydro-indolizin-8-yl]-amide

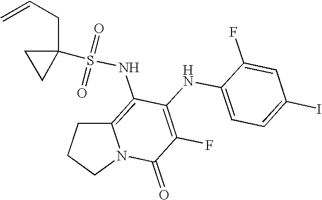

Potassium trimethyl silanoate (0.23 g, 1.776 mmol) was added to a solution of 1-(1-allyl-cyclopropanesulfonyl)-4-fluoro-3-(2-fluoro-4-iodo-phenyl)-1,6,7,8-tetrahydro-3H-1,3,5a-triaza-as-indacene-2,5-dione (0.50 g, 0.888 mmol) in THF (10 mL). The reaction mixture was refluxed at 65° C. for 30 mins. The reaction was monitored by TLC (10% MeOH in DCM). The reaction mixture was partitioned between water and ethylacetate. The organic layer was dried over Na₂SO₄ and concentrated. The concentrate was washed with ether to afford 405 mg of the product (83% yield).

LCMS purity: 92. %, m/z 548 (M+1)

HPLC: 95.8%

¹H NMR (CDCl₃, 300 M): δ 7.4-7.3 (m, 2H), 6.7 (s, 1H), 6.6-6.5 (m, 1H), 5.8-56 (m, 1H), 5.2-5.1 (m, 2H), 4.2 (t, 2H), 3.3 (t, 2H), 2.7 (d, 2H), 2.3-2.1 (m, 2H), 1.3 (t, 2H), 0.8 (t, 2H)

### Step: 8

### Synthesis of 1-(2,3-Dihydroxy-propyl)-cyclopropanesulfonic acid [6-fluoro-7-(2-fluoro-4-iodo-phenylamino)-5-oxo-1,2,3,5-tetrahydro-indolizin-8-yl]-amide

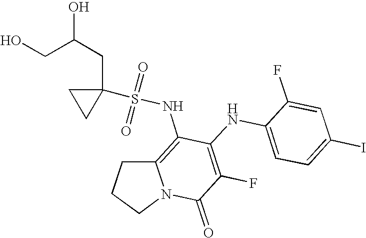

N-Methyl-morpholine-N-oxide (0.078 g, 0.676 mmol) and OsO₄ (0.02 g, 0.067 mmol) were added to a stirred solution of 1-allyl-cyclopropanesulfonic acid [6-fluoro-7-(2-fluoro-4-iodo-phenylamino)-5-oxo-1,2,3,5-tetrahydro-indolizin-8-yl]-amide (0.37 g, 0.676 mmol) in THF (10 mL) and the resulting mixture was stirred at RT overnight. The reaction was monitored by TLC (10% MeOH in DCM). The reaction mixture was partitioned between water and ethylacetate. The organic layer was washed with water, brine solution, dried over Na₂SO₄ and concentrated. Purification by column chromatography on silica gel (5% methanol in chloroform) afforded 86 mg of the product (22% yield).

LCMS purity: 96.2%, m/z=582.1 (M+1)

HPLC: 98.7%

¹H NMR (DMSO-D₆, 300 MHz): δ 8.95-8.85 (br s, 1H), 7.9-7.8 (br s, 1H), 7.6 (d, 1H), 7.45 (d, 1H), 6.9-6.7 (m, 1H), 4.8-4.6 (m, 2H), 4.2-4.0 (m, 2H), 3.8-3.7 (m, 1H), 3.4-3.2 (m, 3H), 2.3 (d, 1H), 2.1 (t, 2H), 1.6-1.4 (m, 1H), 1.1-0.8 (m, 3H)

### EXAMPLE: 87

### Steps 1 to 3 were Performed in a Manner Similar to What has been Described for Example 14, and Step 4 was Performed in a Manner Similar to What has been Described for Example 15

### Step-5

### Synthesis of 3-(4-Bromo-2-fluoro-phenyl)-4-methyl-1,6,7,8-tetrahydro-3H-1,3,5a-triaza-as-indacene-2,5-dione

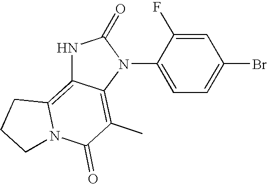

Using the same reaction conditions as in step 5 of Example 86, 7-(4-bromo-2-fluoro-phenylamino)-6-methyl-5-oxo-1,2,3,5-tetrahydro-indolizine-8-carboxylic acid (2.75 g, 7.21784 mmol) in DMF (20 mL) was reacted with TEA (1.0 mL, 7.218 mmol) and DPPA (1.55 μL, 7.218 mmol) to afford 2.2 g of the product (80% yield).

¹H NMR (DMSO-D₆, 300 MHz): δ 11.0 (s, 1H), 7.8 (dd, 1H), 7.6 (m, 2H), 4.0 (t, 2H), 3.0 (t, 2H), 2.2 (t 2H), 1.5 (s, 3H)

### Step-6

### Synthesis of 1-(1-Allyl-cyclopropanesulfonyl)-3-(4-bromo-2-fluoro-phenyl)-4-methyl-1,6,7,8-tetrahydro-3H-1,3,5a-triaza-as-indacene-2,5-dione

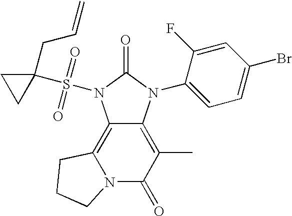

TEA (0.15 mL, 1.06 mmol) was added to stirred solution of 3-(4-bromo-2-fluoro-phenyl)-4-methyl-1,6,7,8-tetrahydro-3H-1,3,5a-triaza-as-indacene-2,5-dione (0.20 g, 0.53 μmol) in DCM (8 mL) at 0° C. This was followed by the addition of 1-Allyl-cyclopropanesulfonyl chloride (0.196 g, 1.06 mmol) and DMAP (0.013 g, 0.106 mmol) at 0° C. and the resulting mixture was stirred at RT overnight. The reaction was monitored by TLC (10% MeOH in DCM). The reaction mixture was partitioned between water and DCM. The organic layer was washed with water, brine solution, dried over Na₂SO₄ and concentrated. Purification by column chromatography on silica gel (70% ethylacetate in hexane) afforded 70 mg of the product (25% yield).

¹H NMR (DMSO-D₆, 300 MHz): δ 7.5-7.3 (m, 3H), 5.9-5.6 (m, 1H), 5.1-4.9 (m, 3H), 4.2 (t, 2H), 3.5 (t 2H), 2.7 (t, 2H), 2.3-2.1 (m, 2H), 2.1-2.0 (m, 2H), 1.9-1.8 (m, 1H), 1.8-1.7 (m, 2H), 1.2-1.1 (m, 4H)

### Step-7

### Synthesis of 1-Allyl-cyclopropanesulfonic acid [7-(4-bromo-2-fluoro-phenylamino)-6-methyl-5-oxo-1,2,3,5-tetrahydro-indolizin-8-yl]-amide

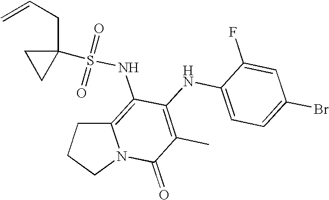

Using the same reaction conditions as in step 7 of Example 86, 1-(1-allyl-cyclopropanesulfonyl)-3-(4-bromo-2-fluoro-phenyl)-4-methyl-1,6,7,8-tetrahydro-3H-1,3,5a-triaza-as-indacene-2,5-dione (0.065 g, 0.124 mmol) in THF (4.0 mL) was reacted with potassium trimethyl silanoate (0.032 g, 0.249 mmol) to afford 40 mg of the product (59% yield).

LCMS purity: 87.3%, m/z=497.9 (M+)

¹H NMR (DMSO-D₆, 300 MHz): δ 8.9-8.8 (br s, 1H), 7.5 (d, 1H), 7.35 (s, 1H), 7.2 (d, 1H), 6.4 (t, 1H), 5.7-5.5 (m, 1H), 5.1-4.9 (m, 2H), 4.1 (t, 2H), 3.2 (t 2H), 2.6 (d, 2H), 2.2-2.1 (m, 2H), 1.7 (s, 3H), 1.05 (t, 2H), 0.75 (t, 2H)

### Step-8

### Synthesis of 1-(2,3-Dihydroxy-propyl)-cyclopropanesulfonic acid [7-(4-bromo-2-fluoro-phenylamino)-6-methyl-5-oxo-1,2,3,5-tetrahydro-indolizin-8-yl]-amide

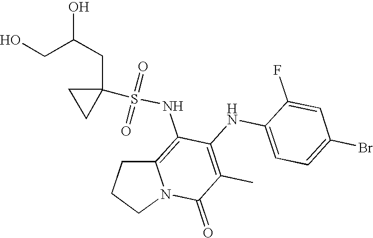

Using the same reaction conditions as in step 8 of Example 86, 1-Allyl-cyclopropanesulfonic acid [7-(4-bromo-2-fluoro-phenylamino)-6-methyl-5-oxo-1,2,3,5-tetrahydro-indolizin-8-yl]-amide (0.025 g, 0.050 mmol) in THF (4.0 mL) was reacted with N-methyl-morpholine-N-oxide (0.006 g, 0.050 mmol), OsO₄ (0.001 g, 0.005 mmol) to afford the crude product. Purification by column chromatography on silica gel (5% methanol in DCM), followed by preparative HPLC afforded 8 mg of the product (26% yield).

LCMS purity: 96.32%, m/z=530 (M+)

HPLC: 93.5%

¹H NMR (CDCl₃-D₆, 300 MHz): δ 7.45-7.4 (br s, 1H), 7.21 (d, 1H), 7.15 (d, 1H), 7.1-7.0 (br s, 1H), 6.45 (t, 1H), 4.25 (t, 2H), 4.1-3.9 (m, 1H), 3.7-3.6 (m, 1H), 3.5-3.4 (m, 1H), 3.3 (t, 2H), 2.4-2.1 (m, 3H), 1.8-1.6 (m, 4H), 1.6-1.3 (m, 2H), 0.9 (t, 2H)

### EXAMPLE: 88

### Steps 1 to 3 were Performed in a Manner Similar to What has been Described for Example 8, Step 4 was Performed in a Manner Similar to What has been Described for Example 11 and Step 5 was Performed in a Manner Similar to What has been Described for Example 61.

### Step-6

### Synthesis of 1-(1-Allyl-cyclopropanesulfonyl)-3-(4-bromo-2-fluoro-phenyl)-4-fluoro-1,6,7,8-tetrahydro-3H-1,3,5a-triaza-as-indacene-2,5-dione

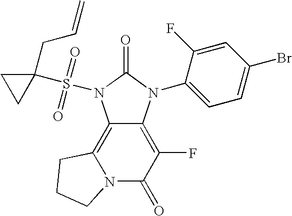

Using the same reaction conditions as in step 6 of Example 86, 3-(4-bromo-2-fluoro-phenyl)-4-fluoro-1,6,7,8-tetrahydro-3H-1,3,5a-triaza-as-indacene-2,5-dione (1.0 g, 2.624 mmol) in DMF (12 mL) was reacted with NaH (0.21 g, 5.249 mmol) and 1-allyl-cyclopropanesulfonyl chloride (0.71 g, 3.937 mmol) to afford the crude product. Purification by column chromatography on silica gel (5% methanol in DCM) afforded 300 mg of the product (21% yield).

LCMS purity: 97.3%, m/z=526 (M+)

HPLC: 95.2%

¹H NMR (CDCl₃, 300 M): δ 7.5-7.4 (m, 2H), 7.35 (d, 1H), 5.8-5.6 (m, 1H), 5.1 (d, 2H), 4.25 (t, 2H), 3.45 (t, 2H), 2.8-2.6 (m, 2H), 2.3-2.1 (m, 2H), 2.0-1.7 (m, 2H), 1.3-1.1 (m, 2H)

### Step-7

### Synthesis of 1-Allyl-cyclopropanesulfonic acid [7-(4-bromo-2-fluoro-phenylamino)-6-fluoro-5-oxo-1,2,3,5-tetrahydro-indolizin-8-yl]-amide

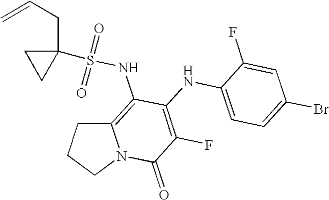

Using the same reaction conditions as in step 7 of Example 86, 1-(1-allyl-cyclopropanesulfonyl)-3-(4-bromo-2-fluoro-phenyl)-4-fluoro-1,6,7,8-tetrahydro-3H-1,3,5a-triaza-as-indacene-2,5-dione (0.26 g, 0.494 mmol) in THF (8 mL) was reacted with potassium trimethyl silanoate (0.12 g, 0.989 mmol) to afford 195 mg of the product (79.2% yield).

LCMS purity: 96.4%, m/z 500.0 (M+)

HPLC: 98.5%

H¹ NMR (CDCl₃, 300 MHz): δ 7.25-7.1 (m, 2H), 6.8-6.1 (m, 1H), 6.65 (s, 1H), 6.4 (s, 1H), 5.8-5.6 (m, 1H), 5.2-5.1 (m, 2H), 4.2 (t 2H), 3.3 (t, 2H), 2.7 (d, 2H), 2.3-2.1 (quin, 2H), 1.3 (t, 2H), 0.85 (t, 2H)

### Step-8

### Synthesis of 1-(2,3-Dihydroxy-propyl)-cyclopropanesulfonic acid [7-(4-bromo-2-fluoro-phenylamino)-6-fluoro-5-oxo-1,2,3,5-tetrahydro-indolizin-8-yl]-amide

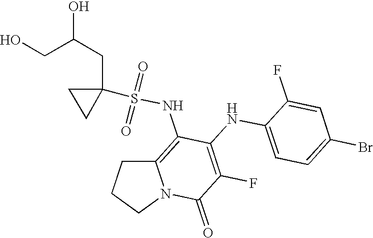

Using the same reaction conditions as in step 8 of Example 86, 1-allyl-cyclopropanesulfonic acid [7-(4-bromo-2-fluoro-phenylamino)-6-fluoro-5-oxo-1,2,3,5-tetrahydro-indolizin-8-yl]-amide (0.19 g, 0.38 mmol) in THF (4.0 mL) was reacted with N-methyl-morpholine-N-oxide (0.045 g, 0.380 mmol), OsO₄ (0.01 g, 0.038 mmol) to afford the crude product. Purification by column chromatography on silica gel (7% methanol in chloroform) afforded 68 mg of the product (34% yield).

LCMS purity: 96.8%, m/z=534.0 (M+)

HPLC: 99.7%

¹H NMR (DMSO-D₆, 300 MHz): δ 8.95-8.85 (br s, 1H), 7.9-7.8 (br s, 1H), 7.55 (d, 1H), 7.3 (d, 1H), 7.1-6.9 (m, 1H), 4.8-4.6 (m, 2H), 4.1 (t, 2H), 3.8-3.7 (br s, 1H), 3.3-3.1 (m, 4H), 2.3 (d, 1H), 2.1 (m, 2H), 1.6-1.5 (m, 1H), 1.2-0.8 (m, 4H)

### EXAMPLE: 89

### Steps 1 to 4 were Performed in a Manner Similar to What has been Described for Example 8

### Step-5

### Synthesis of 6-Fluoro-7-(2-fluoro-4-iodo-phenylamino)-5-oxo-1,2,3,5-tetrahydro-indolizine-8-carboxylic acid (2,2-dimethyl-[1,3]dioxolan-4-ylmethoxy)-amide

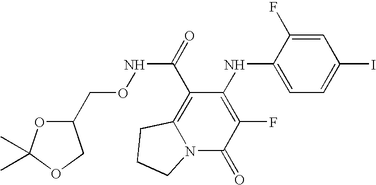

EDCI (1.45 mg, 0.008 mol) and HOBt (1.03 g, 0.008 mol) were added to a solution of 6-fluoro-7-(2-fluoro-4-iodo-phenylamino)-5-oxo-1,2,3,5-tetrahydro-indolizine-8-carboxylic acid (1.1 g, 0.003 mol) in DMF (20 mL). The reaction mixture was stirred at RT for 1 hr. This was followed by the addition of O-(2,2-dimethyl-[1,3]dioxolan-4-ylmethyl)-hydroxylamine (1.12 g, 0.008 mol) and TEA (0.77 g, 0.008 mol). The resulting mixture was stirred at RT for 18 hrs. The reaction mixture was partitioned between ethyl acetate (100 mL) and water (150 mL). The organic layer was washed with saturated NaHCO₃ solution (50 mL), NH₄Cl, brine solution, dried over Na₂SO₄ and concentrated under reduced pressure to afford 780 mg of the crude compound which was used in the next step without further purification.

### Step-6

### Synthesis of Synthesis of 6-Fluoro-7-(2-fluoro-4-iodo-phenylamino)-5-oxo-1,2,3,5-tetrahydro-indolizine-8-carboxylic acid (2,3-dihydroxy-propoxy)-amide

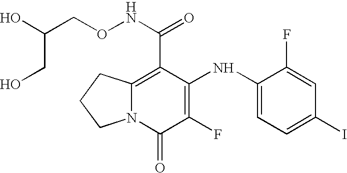

2N HCl (5 ml) was added to a solution of 6-fluoro-7-(2-fluoro-4-iodo-phenylamino)-5-oxo-1,2,3,5-tetrahydro-indolizine-8-carboxylic acid (2,3-dihydroxy-propoxy)-amide (780 mg, 0.001 mol) in methanol (20 ml). The reaction mixture was stirred at RT for 1 hr. The reaction mixture was concentrated under reduced pressure, followed by the addition of 1N NaOH till the pH is about 8 and extracted with ethyl acetate (2×100 ml). The organic layer was dried over Na₂SO₄ and concentrated under reduced pressure to afford 250 mg of the crude product. Purification by preparative HPLC afforded 18 mg of the product (2.4% yield).

LC-MS purity: 96%, m/z=521.9, (M+)

¹H NMR (DMSO-D₆, 300 MHz): δ 11.5 (s, 1H), 8.06 (s, 1H), 7.58 (d, 1H), 7.42 (d, 1H), 6.80 (t, 1H), 4.9-4.4 (m, 2H), 4.00 (t, 2H), 3.9-3.8 (m, 1H), 3.7-3.6 (m, 3H), 3.14-3.12 (m, 3H) 2.18-2.04 (m, 2H)

### EXAMPLE: 90

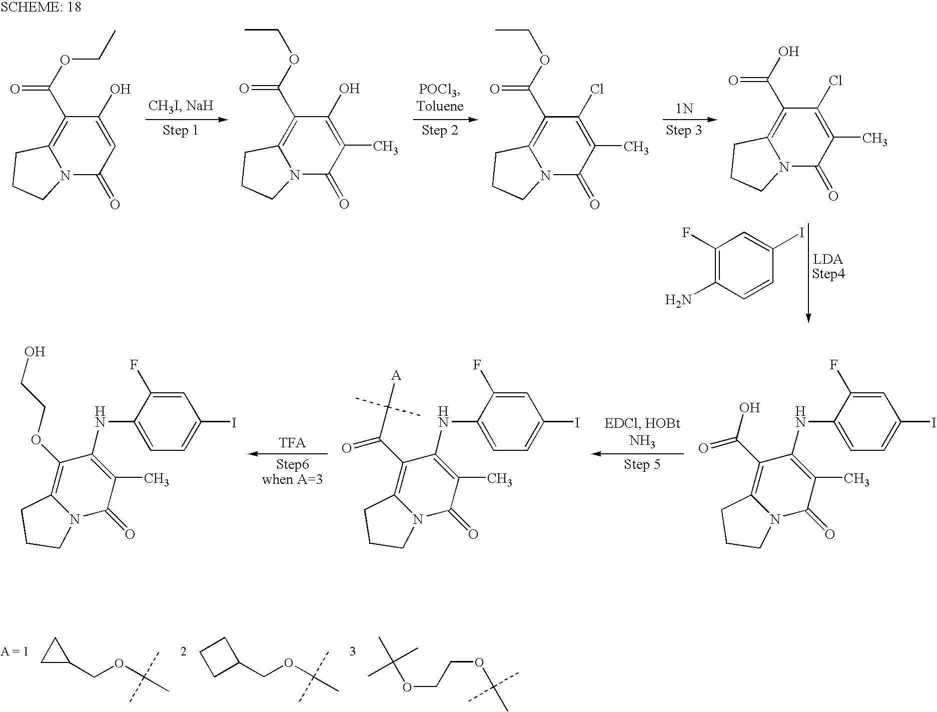

### Step 1 was Performed in a Manner Similar to What has been Described for Example 18

### Synthesis of 7-Chloro-6-methyl-5-oxo-1,2,3,5-tetrahydro-indolizine-8-carboxylic acid ethyl ester

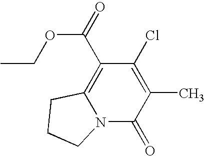

Phosphorusoxy chloride (5.8 g, 0.038 mol) was added to a solution of 7-hydroxy-6-methyl-5-oxo-1,2,3,5-tetrahydro-indolizine-8-carboxylic acid ethyl ester (2 g, 0.008 mol) in toluene (75 mL) at RT. The reaction mixture was stirred for 2 hrs at 110° C. The reaction was monitored by TLC (60% ethyl acetate in hexane). The reaction mixture was concentrated under reduced pressure, followed by the addition of ice water (50 μL) and saturated K₂CO₃ solution (75 mL) (pH=10). The resulting reaction mixture was extracted with ethyl acetate (3×50 mL). The combined organic layers were dried over Na₂SO₄ and concentrated under reduced pressure to afford 1.6 g of crude compound. Purification by recrystallization using hexane afforded 1.54 g of the product (% yield).

### Step-3

### Synthesis of 7-Chloro-6-methyl-5-oxo-1,2,3,5-tetrahydro-indolizine-8-carboxylic acid

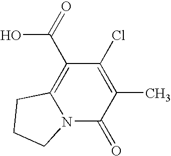

1N LiOH solution (25 mL) was added to a solution of 7-chloro-6-methyl-5-oxo-1,2,3,5-tetrahydro-indolizine-8-carboxylic acid ethyl ester (1.5 g, 0.006 mol) in 75 ml of THF:MeOH (4:1). The reaction mixture was stirred at RT for 18 hours. The reaction was monitored by TLC (20% methanol in DCM). The reaction mixture was concentrated under reduced pressure, followed by the addition of 1N HCl (100 mL) till the pH is about 1. The precipitate was collected and dried under reduced pressure to afford 1.12 g of the product (84% yield).

LC-MS purity: 98%, m/z=226 (M−)

¹H NMR (DMSO-D₆, 300 MHz): δ 13.2 (s, 1H), 4.00 (t, 2H), 3.28 (t, 2H), 2.2-2.02 (m, 5H)

### Step-4

### Synthesis of 7-(2-Fluoro-4-iodo-phenylamino)-6-methyl-5-oxo-1,2,3,5-tetrahydro-indolizine-8-carboxylic acid

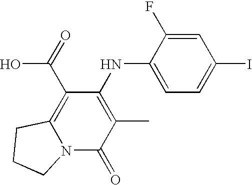

LDA (1.86 g, 0.017 mol) was added to a solution of 2-fluoro-4-iodo-phenylamine (2.9 g, 0.012 mol) in dry THF (20 mL) at −78° C. The reaction mixture was stirred for 45 minutes at −78° C. This was followed by addition of 7-Chloro-6-methyl-5-oxo-1,2,3,5-tetrahydro-indolizine-8-carboxylic acid (1.1 g, 0.005 mol) in THF (90 mL) at −78° C. and stirring was continued for a further 20 hrs at RT. The reaction was monitored by TLC (20% methanol in DCM). The reaction mixture was concentrated under reduced pressure, followed by the addition of 2N HCl solution (50 mL) and diethyl ether (50 mL). The reaction mixture was stirred for 15 minutes. The precipitate was collected, washed with diethyl ether (2×20 mL) and dried under reduced pressure to afford 820 mg of the product (41% yield).

LC-MS purity: 94%, m/z=429 (M+)

¹H NMR (DMSO-D₆, 300 MHz): δ 13.30 (s, 1H), 9.36 (s, 1H), 7.60 (d, 1H), 7.38 (s, 1H), 6.40 (t, 1H), 4.04 (t, 2H), 3.48 (t, 2H), 2.18-2.02 (m, 2H), 1.64 (s, 3H)

### Step-5

### Synthesis of 7-(2-Fluoro-4-iodo-phenylamino)-6-methyl-5-oxo-1,2,3,5-tetrahydro-indolizine-8-carboxylic acid cyclopropyl methoxy amide

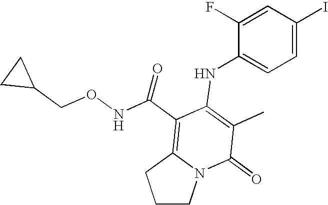

EDCI (334 mg, 0.002 mol) and HOBt (236 mg, 0.002 mol) were added to a solution of 7-(2-fluoro-4-iodo-phenylamino)-6-methyl-5-oxo-1,2,3,5-tetrahydro-indolizine-8-carboxylic acid (250 mg, 0.001 mol) in DMF (10 mL). The reaction mixture was stirred at RT for 1 hr. This was followed by the addition of O-cyclopropylmethyl-hydroxylamine hydrochloride (216 mg, 0.002 mol) and TEA (176 mg, 0.002 mol). The resulting mixture was stirred at RT for 2 hrs. The reaction was monitored by TLC (10% methanol in DCM). The reaction mixture was partitioned between ethyl acetate (75 mL) and water (125 mL). The organic layer was washed with saturated NH₄Cl (50 mL), NaHCO₃, brine solution, dried over Na₂SO₄ and concentrated under reduced pressure to afford 210 mg of the crude compound. Purification by recrystallization using methanol (2 mL) and diethyl ether (20 mL) afforded 140 mg of the product (48.2% yield).

LC-MS purity: 97%, m/z=498, (M+)

¹H NMR (DMSO-D₆, 300 MHz): δ 11.22 (s, 1H), 7.7 (s, 1H), 7.56 (d, 1H), 7.32 (d, 1H), 6.40 (t, 1H), 4.02 (t, 2H), 3.48 (d, 2H), 3.2 (t, 2H), 2.18-2.04 (m, 2H), 1.68 (s, 3H), 1.04-0.094 (m, 1H), 0.58-0.46 (m, 2H), 0.26-0.18 (m, 2H).

### EXAMPLE: 91

### Step 1 was Performed in a Manner Similar to What has been Described for Example 14, and Step 2 to 4 were Performed in a Manner Similar to What has been Described for Example 90

### Step-5

### Synthesis of 7-(2-Fluoro-4-iodo-phenylamino)-6-methyl-5-oxo-1,2,3,5-tetrahydro-indolizine-8-carboxylic acid (2-tert-butoxy-ethoxy)-amide

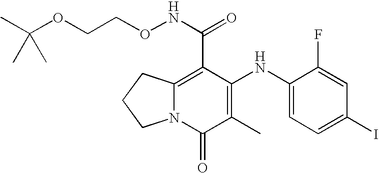

EDCI (334 mg, 0.002 mol) and HOBt (236 mg, 0.002 mol) were added to a solution of 7-(2-fluoro-4-iodo-phenylamino)-6-methyl-5-oxo-1,2,3,5-tetrahydro-indolizine-8-carboxylic acid (250 mg, 0.001 mol) in DMF (10 mL). The reaction mixture was stirred at RT for 30 minutes. This was followed by the addition of O-(2-tert-butoxy-ethyl)-hydroxylamine (233 mg, 0.002 mol) and TEA (176 mg, 0.002 mol). The resulting mixture was stirred at RT for 16 hrs. The reaction was monitored by TLC (10% methanol in DCM). The reaction mixture was partitioned between ethyl acetate (100 mL) and water (100 mL). The organic layer was washed with saturated NH₄Cl (50 mL), NaHCO₃, brine solution, dried over Na₂SO₄ and concentrated under reduced pressure to afford 300 mg of the crude compound. Purification by column chromatography on silica gel (2% methanol in DCM) afforded 228 mg of the product (71% yield).

LC-MS purity: 96%, m/z 544, (M+)

### Step-6

### Synthesis of 7-(2-Fluoro-4-iodo-phenylamino)-6-methyl-5-oxo-1,2,3,5-tetrahydro-indolizine-8-carboxylic acid (2-hydroxy-ethoxy)-amide

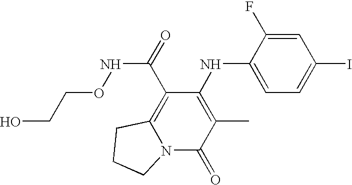

Trifluoro acetic acid (2.5 mL) was added to a solution of 7-(2-fluoro-4-iodo-phenylamino)-6-methyl-5-oxo-1,2,3,5-tetrahydro-indolizine-8-carboxylic acid (2-tert-butoxy-ethoxy)-amide (220 mg, 0.0004 mol) in DCM (2.5 mL) at 0° C. The reaction mixture was stirred for 2 hours. The reaction was monitored by TLC (10% methanol in DCM). The reaction mixture was concentrated under reduced pressure, followed by the addition of NaHCO₃ till the pH is about 8 and extracted with ethyl acetate. The combined organic layers were washed with 5% NaHCO₃ solution, dried over Na₂SO₄ and concentrated under reduced pressure. The concentrate was washed with diethyl ether and ethyl acetate to afford 89 mg of the product (45% yield).

LC-MS purity: 93%, m/z=488, (M+)

¹H NMR (DMSO-D₆, 300 MHz): δ 11.24 (s, 1H), 7.68 (s, 1H), 7.54 (d, 1H), 7.32 (d, 1H), 6.40 (t, 1H), 4.7 (s, 1H), 4.00 (t, 2H), 3.7 (t, 2H), 3.55-3.42 (m, 2H), 3.18 (t, 2H), 2.18-2.04 (m, 2H), 1.72 (s, 3H)

### EXAMPLE: 92

### Step 1 was Performed in a Manner Similar to What has been Described for Example 14 and Step 2 to 4 were Performed in a Manner Similar to What has been Described for Example 90

### Step-5

### Synthesis of 7-(2-Fluoro-4-iodo-phenylamino)-6-methyl-5-oxo-1,2,3,5-tetrahydro-indolizine-8-carboxylic acid cyclobutylmethoxy-amide

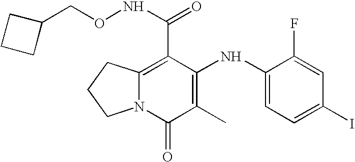

EDCI (167 mg, 0.001 mol) and HOBt (118 mg, 0.001 mol) were added to a solution of 7-(2-fluoro-4-iodo-phenylamino)-6-methyl-5-oxo-1,2,3,5-tetrahydro-indolizine-8-carboxylic acid (125 mg, 0.0003 mol) in DMF (8 mL). The reaction mixture was stirred at RT for 1 hr. This was followed by the addition of O-cyclobutylmethyl-hydroxylamine (88 mg, 0.001 mol) and TEA (88 mg, 0.001 mol). The resulting mixture was stirred at RT for 2 hrs. The reaction was monitored by TLC (10% methanol in DCM). The reaction mixture was partitioned between ethyl acetate (50 mL) and water (75 mL). The organic layer was washed with saturated NH₄Cl (50 mL), NaHCO₃, brine solution, dried over Na₂SO₄ and concentrated under reduced pressure to afford 150 mg of the crude compound. Purification by recrystallization using ethyl acetate (10 mL) and diethyl ether (5 mL) afforded 85 mg of the product (57% yield).

LC-MS purity: 94%, m/z 512, (M+)

¹H NMR (DMSO-D₆, 300 MHz): δ 11.2 (s, 1H), 7.68 (s, 1H), 7.58 (d, 1H), 7.34 (d, 1H), 6.40 (t, 1H), 4.00 (t, 2H), 3.56 (d, 2H), 3.18 (t, 2H), 2.18-2.04 (m, 2H), 2.02-1.92 (m, 3H), 1.9-1.62 (m, 7H).

### EXAMPLE: 93

### Steps and 3 were Performed in a Manner Similar to What has been Described for Example 14, and Step 4 was Performed in a Manner Similar to What has been Described for Example 15

### Step-5

### Synthesis of 3-(2-Fluoro-4-iodo-phenyl)-4-methyl-1,6,7,8-tetrahydro-3H-1,3,5a-triaza-as-indacene-2,5-dione

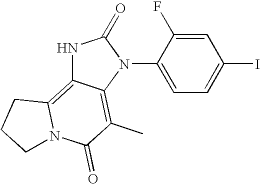

Using the same reaction conditions as in step 5 of Example 86, 7-(2-fluoro-4-iodo-phenylamino)-6-methyl-5-oxo-1,2,3,5-tetrahydro-indolizine-8-carboxylic acid (3.14 g, 7.34 mmol) in DMF (30 mL) was reacted with TEA (1.03 mL, 7.34 mmol) and DPPA (1.59 mL, 7.34 mmol) to afford 1.85 g of the product (59% yield).

LCMS purity: 97.0%, m/z 426.0 (M+)

¹H NMR (DMSO-D₆, 300 MHz): δ 11.1 (s, 1H), 7.9 (d, 1H), 7.7 (d, 1H), 7.4-7.1 (m, 2H), 3.9 (t, 2H), 3.1 (t, 2H), 2.4-2.2 (m, 2H), 1.45 (s, 3H)

### Step-6

### Synthesis of 1-(1-Allyl-cyclo propanesulfonyl)-3-(2-fluoro-4-iodo-phenyl)-4-methyl-1,6,7,8-tetrahydro-3H-1,3,5a-triaza-as-indacene-2,5-dione

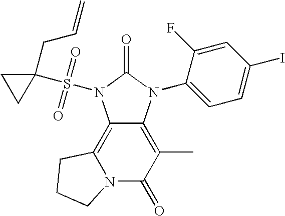

Using the same reaction conditions as in step 6 of Example 109 (current set), 3-(2-fluoro-4-iodo-phenyl)-4-methyl-1,6,7,8-tetrahydro-3H-1,3,5a-triaza-as-indacene-2,5-dione (1.80 g, 4.24 mmol) in DMF (20 mL) was reacted with NaH (0.34 g, 8.47 mmol) and 1-allyl-cyclopropanesulfonyl chloride (1.14 g, 6.35 mmol) to afford the crude product. Purification by column chromatography on silica gel (50% ethylacetate in hexane) afforded 490 mg of the product (20% yield).

LCMS purity: 83.9%, m/z=569.9 (M+)

¹H NMR (CDCl₃, 300 MHz): δ 7.7-7.6 (m, 2H), 7.15 (t, 1H), 5.7-5.6 (m, 1H), 5.1-4.9 (m, 2H), 4.2 (t, 2H), 3.5 (t, 2H), 2.7 (t, 2H), 2.3-2.1 (m, 3H), 1.9-1.8 (m, 1H), 1.8-1.6 (m, 1H), 1.3-1.11 (m, 2H)

### Step-7

### Synthesis of 1-Allyl-cyclo propanesulfonic acid [7-(2-fluoro-4-iodo-phenylamino)-6-methyl-5-oxo-1,2,3,5-tetrahydro-indolizin-8-yl]-amide

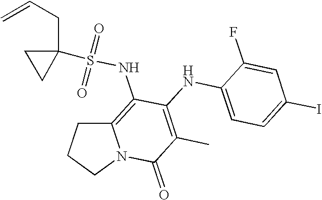

Using the same reaction conditions as in step 7 of Example 109 (current set), 1-(1-Allyl-cyclopropanesulfonyl)-3-(2-fluoro-4-iodo-phenyl)-4-methyl-1,6,7,8-tetrahydro-3H-1,3,5a-triaza-as-indacene-2,5-dione (0.48 g, 0.84 mmol) in THF (12 mL) was reacted with potassium trimethyl silanoate (0.21 g, 1.69 mmol) to afford 310 mg of the product (63% yield).

LCMS purity: 85.2%, m/z=544.0 (M+)

HPLC: 86.7%

¹H NMR (DMSO-D₆, 300 MHz): δ 8.8 (s, 1H), 7.6 (d, 1H), 7.35 (d, 2H), 6.3 (t, 1H), 5.7-5.5 (m, 1H), 5.1 (d, 2H), 4.1 (t, 2H), 3.25 (t, 2H), 2.6 (d, 2H), 2.1 (quin, 2H), 1.7 (s, 3H), 1.1 (t, 2H), 0.75 (t, 2H)

### Step-8

### Synthesis of 1-(2,3-Dihydroxy-propyl)-cyclopropanesulfonic acid [7-(2-fluoro-4-iodo-phenylamino)-6-methyl-5-oxo-1,2,3,5-tetrahydro-indolizin-8-yl]-amide

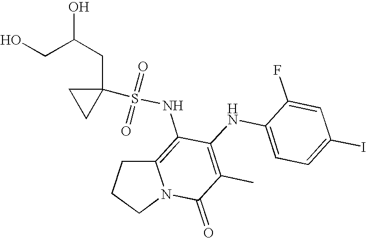

Using the same reaction conditions as in step 8 of Example 109 (current set), 1-allyl-cyclopropanesulfonic acid [7-(2-fluoro-4-iodo-phenylamino)-6-methyl-5-oxo-1,2,3,5-tetrahydro-indolizin-8-yl]-amide (0.25 g, 0.46 mmol) in THF (10 mL) was reacted with N-methyl-morpholine-N-oxide (0.054 g, 0.46 mmol), OsO₄ (0.01 g, 0.046 mmol) to afford the crude product. Purification by column chromatography on silica gel (5% methanol in CHCl₃), followed by preparative HPLC afforded 95 mg of the product (36% yield).

LCMS purity: 96.7%, m/z=577.8 (M+)

HPLC: 97.2%

¹H NMR (DMSO-D₆, 300 MHz): δ 8.75 (s, 1H), 7.6-7.3 (m, 3H), 6.3 (t, 1H), 4.6 (t, 2H), 4.1 (t, 2H), 3.6-3.5 (m, 1H), 3.3-3.2 (m, 4H), 2.1-2.0 (m, 3H), 1.75-1.6 (m, 5H), 1.3-1.0 (m, 4H)

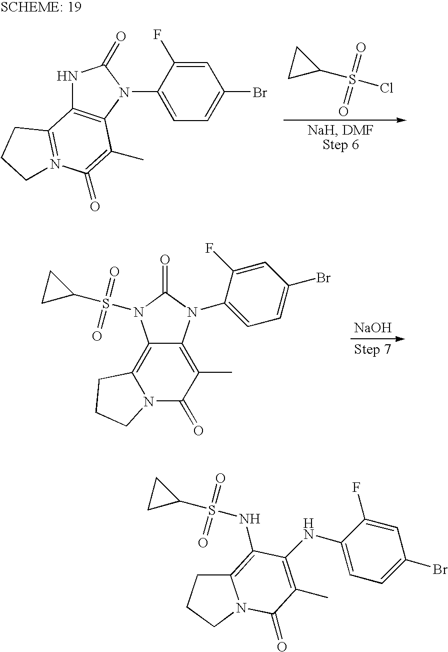

### EXAMPLE: 94

### Steps 1 to 3 were Performed in a Manner Similar to What has been Described for Example 14, Step 4 was Performed in a Manner Similar to What has been Described for Example 15, and Step 5 was Performed in a Manner Similar to What has been Described for Example 87.

### Step-6

### Synthesis of 3-(4-Bromo-2-fluoro-phenyl)-1-cyclopropanesulfonyl-4-methyl-1,6,7,8-tetrahydro-3H-1,3,5a-triaza-as-indacene-2,5-dione

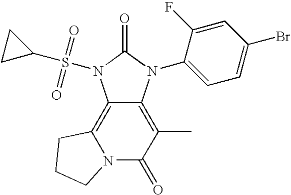

Sodium hydride (0.04 gm, 0.001 mol) was added to a solution of 3-(4-bromo-2-fluoro-phenyl)-4-methyl-1,6,7,8-tetrahydro-3H-1,3,5a-triaza-as-indacene-2,5-dione (0.2 gm, 0.0005 mol) in dry DMF (3 ml) at 0° C. This was followed by the addition of cyclopropanesulfonyl chloride (0.11 gm, 0.0008 mol) at 0° C. and the reaction mixture was stirred at RT overnight. The reaction was monitored by TLC (5% MeOH in CHCl₃). Addition of ice, neutralization with dil. HCl, extraction with ethyl acetate followed by concentration afforded 0.25 g of the crude product which was used in the next step without further purification.

### Step-7

### Synthesis of Cyclopropanesulfonic acid [7-(4-bromo-2-fluoro-phenylamino)-6-methyl-5-oxo-1,2,3,5-tetrahydro-indolizin-8-yl]-amide

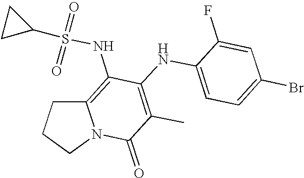

6 ml of 1N NaOH solution was added to 3-(4-bromo-2-fluoro-phenyl)-1-cyclopropanesulfonyl-4-methyl-1,6,7,8-tetrahydro-3H-1,3,5a-triaza-as-indacene-2,5-dione (0.25 g) in DMF and heated the mixture to 65° C. for 1.30 hrs. Addition of ice, neutralization with dil. HCl, extraction with ethyl acetate followed by concentration and purification by column chromatography on silica gel (2% methanol in CHCl₃) afforded 0.090 g of the product (37.34% yield).

¹H NMR (DMSO-D₆, 300 MHz): δ 8.9 (s, 1H), 7.5 (d, 1H), 7.4 (s, 1H), 7.2 (d, 1H), 6.5 (t, 1H), 4.0 (t, 2H), 3.3 (t, 2H), 2.5 (m, 1H), 2.1 (q, 2H), 1.7 (s, 3H), 0.9 (m, 4H)

The compounds of the above Examples were evaluated as inhibitors of the MAP kinase pathway in a BRAF-MEK-ERK enzymatic cascade assay and in a cell viability assay, the results of which are collated in Table 1. It is recognized that Other compounds described herein can be made by a person of ordinary skill in the art using methods known to him and/or described herein.

TABLE 1

| Compound | | % Inhibition | GI₅₀ |
|---|---|---|---|
| No. | STRUCTURE | (10 μM) | (μM) |
| 1 | 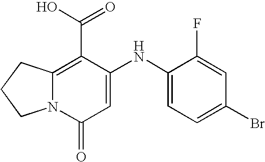 | 65 | — |
| 2 | 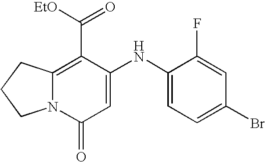 | 85 | — |
| 3 | 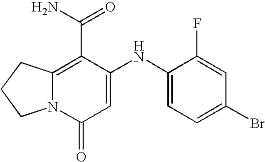 | 92 | — |
| 4 | 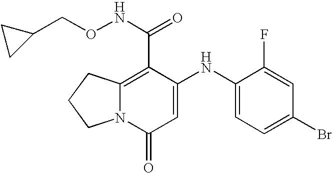 | 100 | 0.014 |
| 5 | 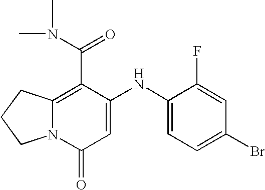 | 9 | — |
| 6 | 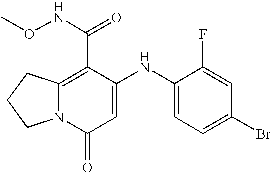 | 98 | 0.15 |
| 7 | 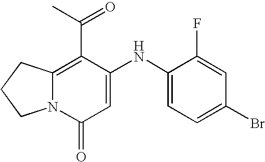 | 37 | — |
| 8 | 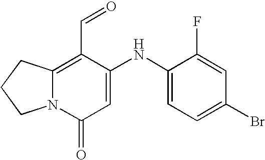 | 91 | — |
| 9 | 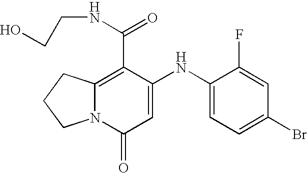 | 45 | — |
| 10 | 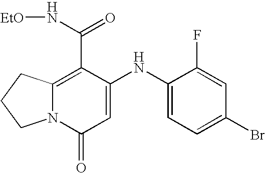 | 98 | 0.04 |
| 11 | 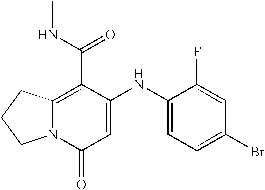 | 41 | — |
| 12 | 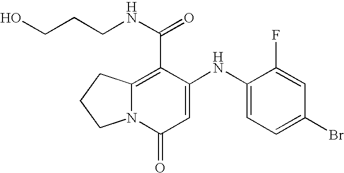 | 65 | — |
| 13 | 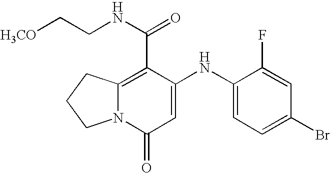 | 13 | — |
| 14 | 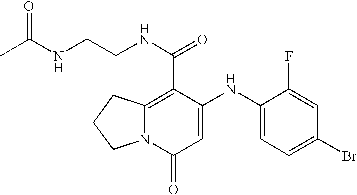 | 24 | — |
| 15 | 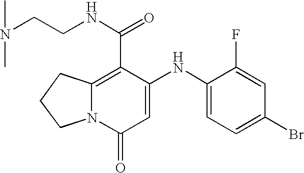 | 24 | — |
| 16 | 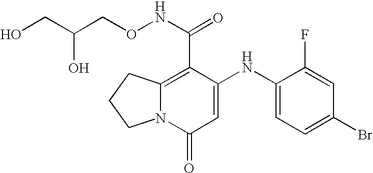 | 99 | — |
| 17 | 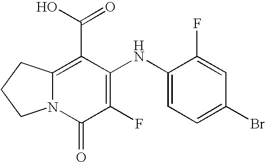 | 98 | — |
| 18 | 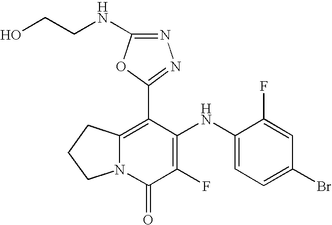 | 98 | — |
| 19 | 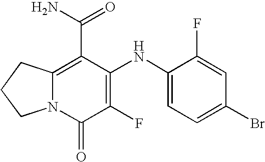 | 99 | — |
| 20 | 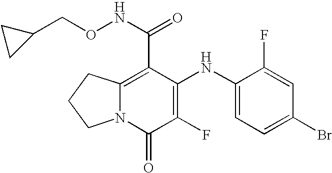 | 100 | 0.055 |
| 21 | 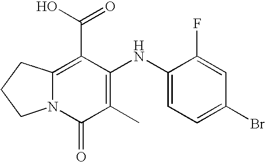 | 95 | — |
| 22 | 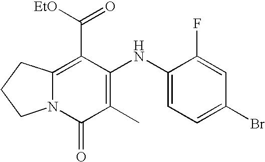 | 82 | — |
| 23 | 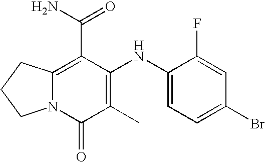 | 97 | — |
| 24 | 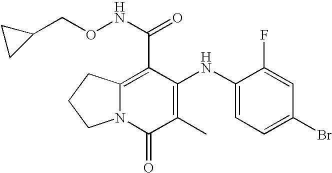 | 100 | 0.036 |
| 25 | 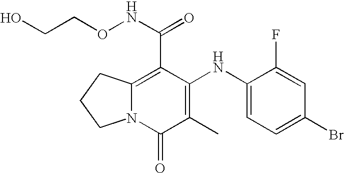 | 100 | 0.01 |
| 26 | 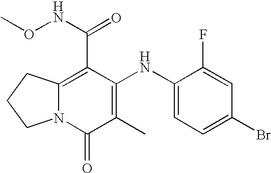 | 98 | — |
| 27 | 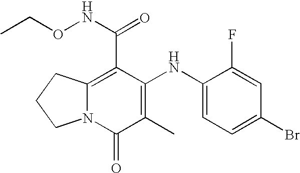 | 98 | 0.06 |
| 28 | 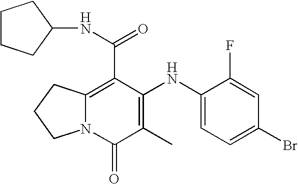 | 16 | — |
| 29 | 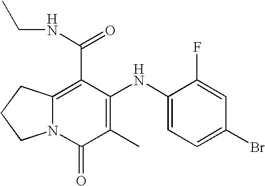 | 31 | — |
| 30 | 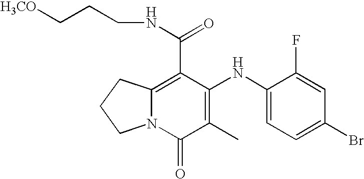 | 11 | — |
| 31 | 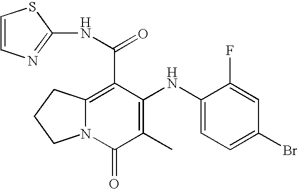 | 28 | — |
| 32 | 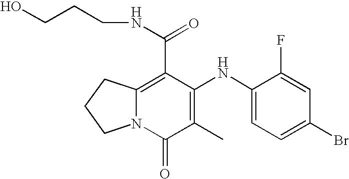 | 31 | — |
| 33 | 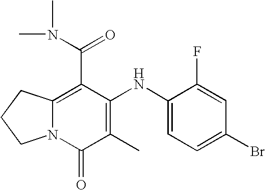 | 2 | — |
| 34 | 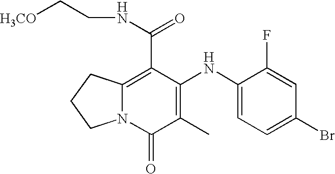 | 21 | — |
| 35 | 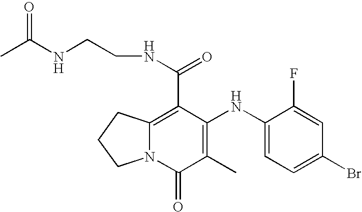 | 14 | — |
| 36 | 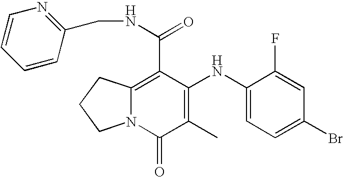 | 02 | — |
| 37 | 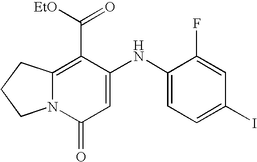 | 99 | — |
| 38 | 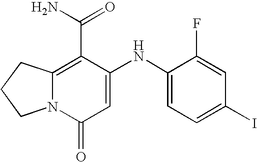 | 100 | 0.073 |
| 39 | 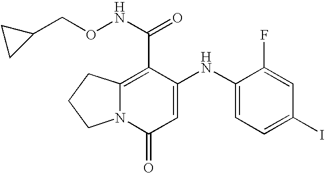 | 100 | 0.0008 |
| 40 | 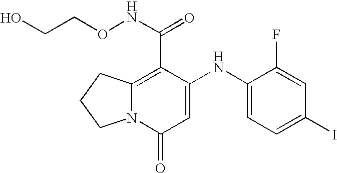 | 100 | 0.0007 |
| 41 | 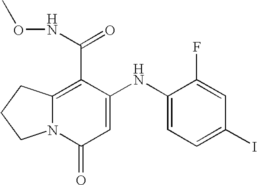 | 100 | 0.009 |
| 42 | 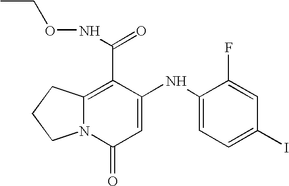 | 98 | 0.0036 |
| 43 | 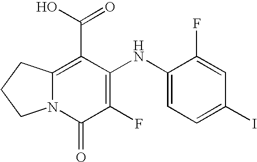 | 100 | — |
| 44 | 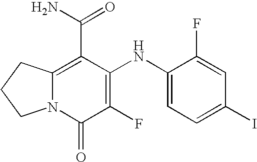 | 99 | — |
| 45 | 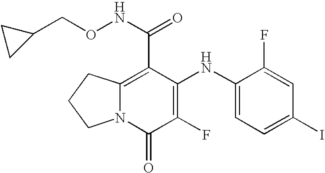 | 100 | 0.0004 |
| 46 | 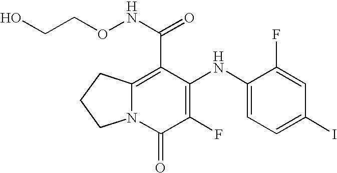 | 100 | 0.0067 |
| 47 | 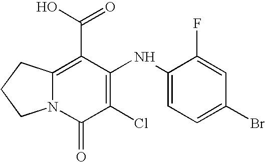 | 94 | — |
| 48 | 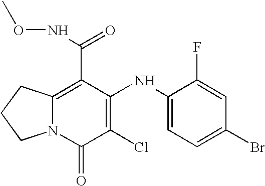 | 91 | — |
| 49 | 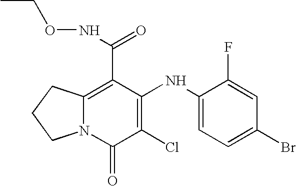 | 94 | — |
| 50 | 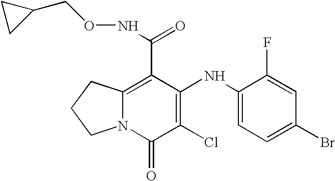 | 96 | — |
| 51 | 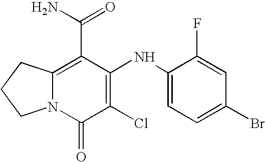 | 91 | — |
| 52 | 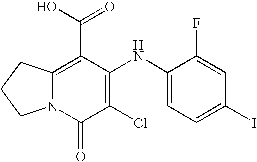 | — | — |
| 53 | 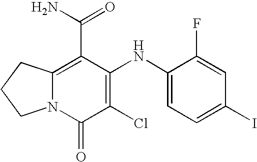 | — | — |
| 54 | 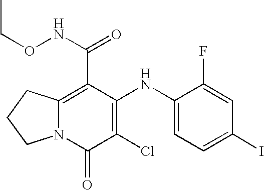 | — | — |
| 55 | 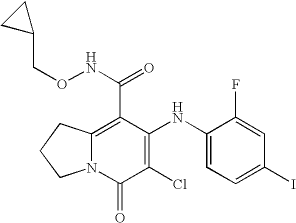 | — | — |
| 56 | 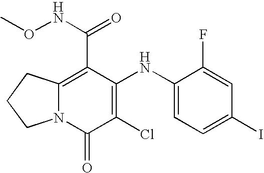 | — | — |
| 57 | 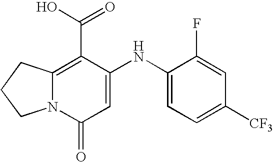 | 45 | — |
| 58 | 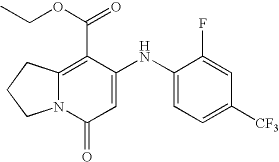 | 43 | — |
| 59 | 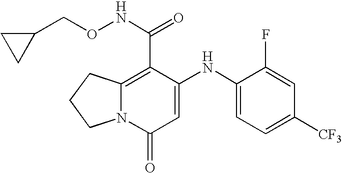 | 95 | — |
| 60 | 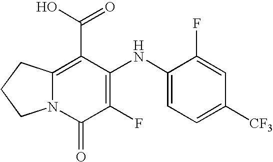 | 91 | — |
| 61 | 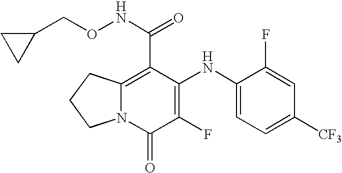 | 97 | — |
| 62 | 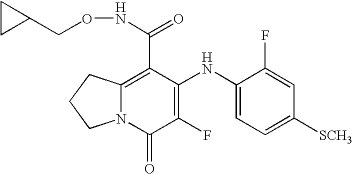 | — | — |
| 63 | 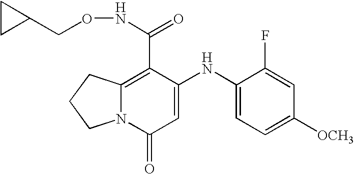 | 89 | — |
| 64 | 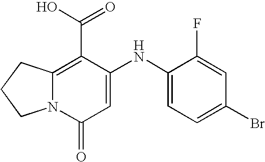 | 10 | — |
| 65 | 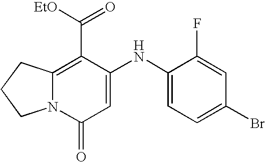 | 25 | — |
| 66 | 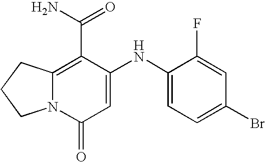 | 24 | — |
| 67 | 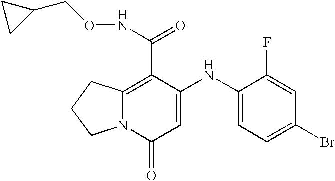 | 68 | — |
| 68 | 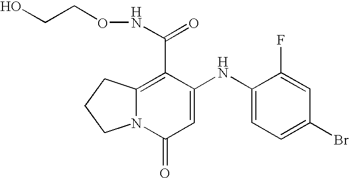 | 81 | — |
| 69 | 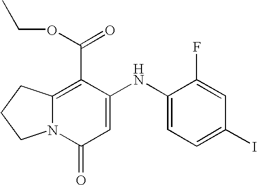 | 93 | — |
| 70 | 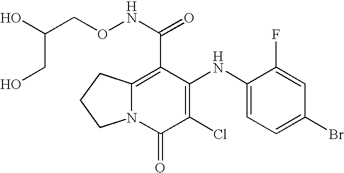 | 88 | |
| 71 | 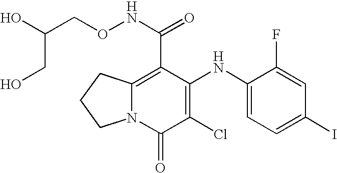 | 100 | |
| 72 | 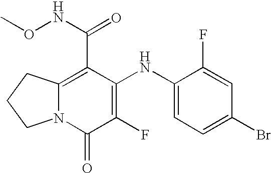 | 100 | |
| 73 | 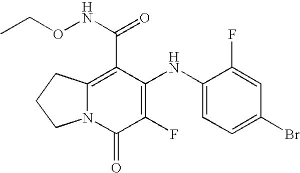 | 100 | |
| 74 | 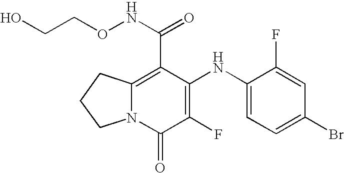 | 98 | |
| 75 | 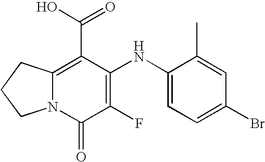 | 92 | |
| 76 | 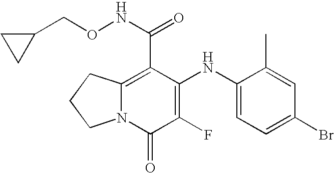 | 98 | |
| 77 | 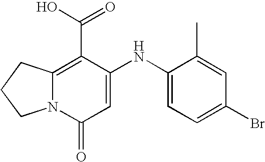 | 70 | |
| 78 | 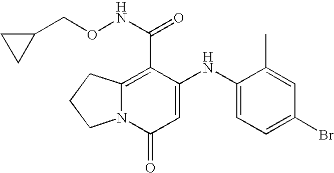 | 97 | |
| 79 | 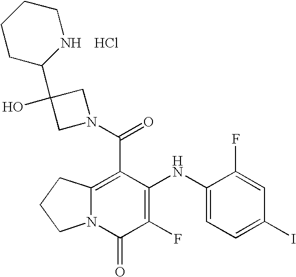 | 95 | |
| 80 | 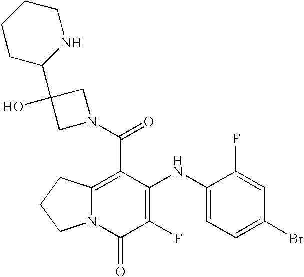 | 71 | |
| 81 | 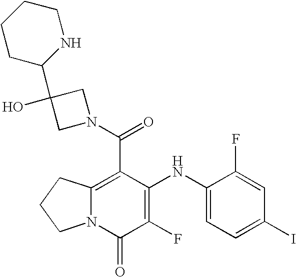 | 95 | |
| 82 | 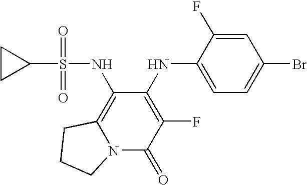 | 99 | |
| 83 | 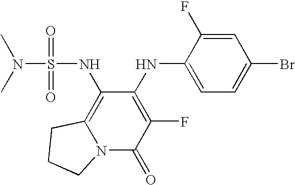 | 93 | |
| 84 | 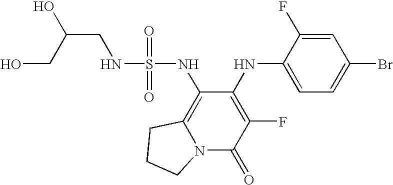 | 94 | |
| 85 | 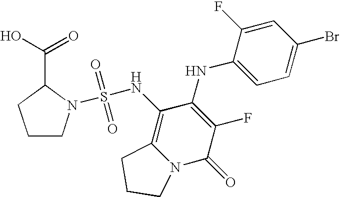 | 96 | |
| 86 | 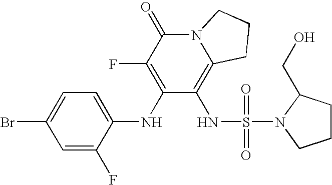 | 99 | |
| 87 | 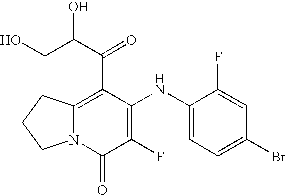 | 88 | |
| 88 | 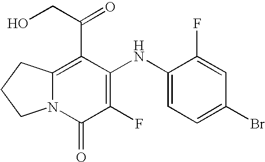 | 100 | |
| 89 | 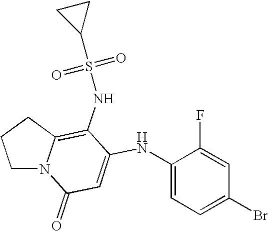 | 100 | |
| 90 | 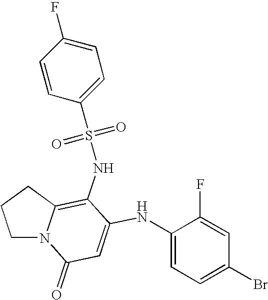 | 97 | |
| 91 | 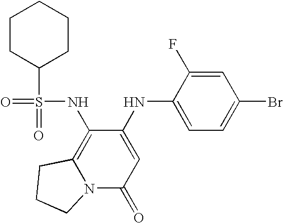 | 90 | |
| 92 | 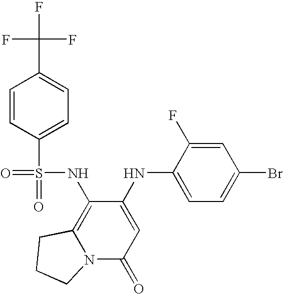 | 60 | |
| 93 | 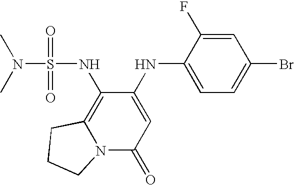 | 78 | |
| 94 | 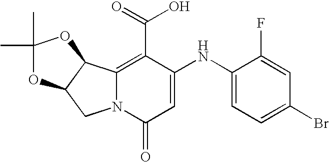 | 58 | |
| 95 | 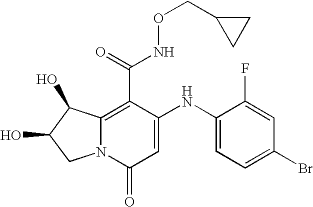 | 92 | |
| 96 | 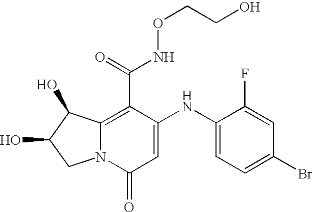 | 95 | |
| 97 | 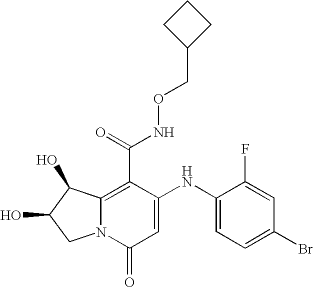 | 81 | |
| 98 | 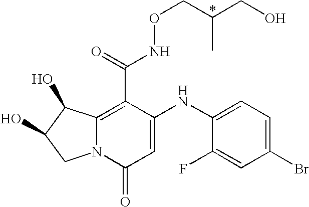 | 75 | |
| 99 | 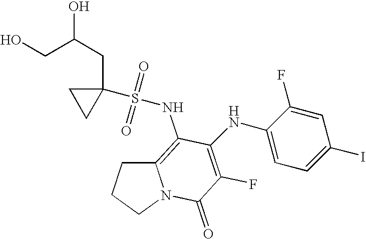 | 100 | |
| 100 | 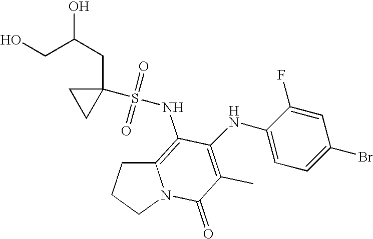 | 100 | |
| 101 | 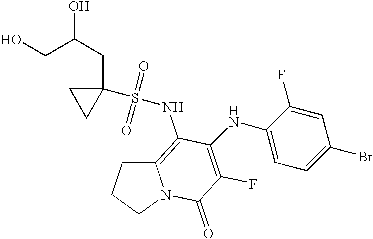 | 100 | |
| 102 | 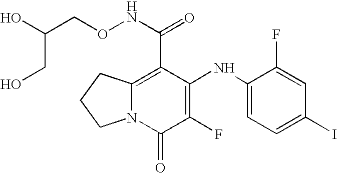 | 100 | |
| 103 | 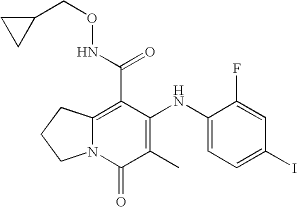 | 100 | |
| 104 | 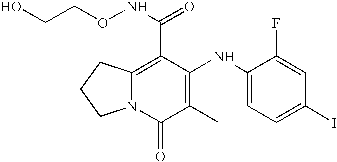 | 100 | |
| 105 | 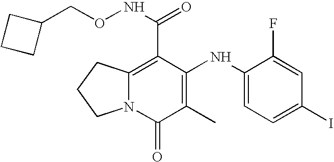 | 100 | |
| 106 | 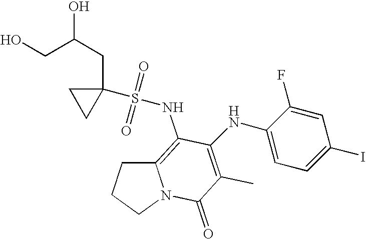 | 99 | |
| 107 | 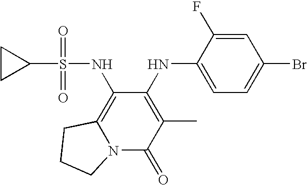 | 100 | |

The examples provided in the description are only to describe the invention, hence it should not be construed to limit the scope of the invention.

We claim:

1. A compound of formula I: 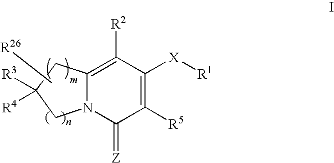
and pharmaceutically acceptable salts thereof, wherein
X represents C₁₋₃-alkylene, —N(R⁶)—, —O—, or —S(O)ₚ—;
R¹ represents aryl, heteroaryl, cycloalkyl or heterocycloalkyl, wherein said rings are optionally substituted by one or more groups independently selected from List 1;
R² represents H, cyano, or the group —Y—R⁷;
R³ and R⁴ independently represent H, C₁₋₆-alkyl, C₁₋₆-haloalkyl, C₁₋₆-hydroxyalkyl, hydroxyl, C₁₋₆-alkoxy, amino, C₁₋₆-alkylamino, diC₁₋₆-alkylamino, or R³ additionally represents monocyclic cycloalkyl or monocyclic heterocycloalkyl, where said rings are optionally substituted by one or more groups independently selected from List 1;
R⁵ represents H, halogen, C₁₋₃-alkyl, or C₁₋₃-haloalkyl;
Y represents a group selected from -D-, -E-, -D-E-, or -E-D-;
D represents a group selected from —N(R⁸)—, —CO—, —CO₂—, —SO—, —SO₂—, CON(R⁹)O—, —CON(R¹⁰)—, —N(R¹¹)SO₂—, —N(R²⁴)SO₂NR²⁵—, —SO₂N(R¹²), —N(R¹³)CO—, —N(R¹⁴)CON(R¹⁵)—, —N(R¹⁶)CO—, or —C(═NH)N(R¹⁷)—;
E represents a monocyclic arylene, heteroarylene, cycloalkylene or heterocycloalkylene, wherein said rings are optionally substituted by one or more groups independently selected from List 1;
R⁷ represents H, C₁₋₆-alkyl, C₂₋₆-alkenyl C₂₋₆-alkynyl, cycloalkyl, aryl, heterocycloalkyl, or heteroaryl, wherein R⁷ when not H is optionally substituted by one to three groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆haloalkyl, amino, C₁₋₆alkylamino, di-C₁₋₆alkylamino, C₁₋₆acylamino, C₁₋₆acylC₁₋₆ alkylamino, monocyclic cycloalkyl or monocyclic heterocycloalkyl, where said rings may be optionally substituted by one or two groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆-haloalkyl, amino, C₁₋₆-alkylamino, di-C₁₋₆-alkylamino, C₁₋₆-acylamino and C₁₋₆-acylC₁₋₆-alkylamino;
Z is O or N(R¹⁸);
List 1 is selected from hydroxyl, cyano, nitro, C₁₋₆-alkyl, C₂₋₆-alkenyl, C₂₋₆-alkynyl, C₁₋₆-alkoxy, C₂₋₆-alkenyloxy, C₁₋₆-alkynyloxy, halogen, C₁₋₆-alkylcarbonyl, carboxy, C₁₋₆-alkoxycarbonyl, amino, C₁₋₆-alkylamino, di-C₁₋₆-alkylamino, C₁₋₆-alkylaminocarbonyl, di-C₁₋₆-alkylaminocarbonyl, C₁₋₆-alkylcarbonylamino, C₁₋₆ alkylcarbonyl(C₁₋₆-alkyl)amino, C₁₋₆-alkylsulfonylamino, C₁₋₆-alkylsulfonyl(C₁₋₆-alkyl)amino, C₁₋₆-thioalkyl, C₁₋₆-alkylsulfinyl, C₁₋₆-alkylsulfanyl, C₁₋₆-alkylsulfonyl, aminosulfonyl, C₁₋₆-alkylaminosulfonyl and di-C₁₋₆-alkylaminosulfonyl, where each of the afore-mentioned hydrocarbon groups may be optionally substituted by one or more halogen, hydroxyl, C₁₋₆-alkoxy, amino, C₁₋₆-alkylamino, di-C₁₋₆-alkylamino or cyano;
R²⁶ represents H, C₁₋₆-alkyl, C₁₋₆-haloalkyl, C₁₋₆-hydroxyalkyl, hydroxyl, C₁₋₆-alkoxy, amino, C₁₋₆-alkylamino, or diC₁₋₆-alkylamino;
R⁶, R⁸, R⁹, R¹⁰, R¹¹, R¹², R¹³, R¹⁴, R¹⁵, R¹⁶, R¹⁷, R¹⁸, R²⁴, and R²⁵ are independently H or C₁₋₆-alkyl;
m and n are independently 0, 1, 2, or 3; and m+n=2 or 3;
p is 0, 1, or 2; and wherein
Alkyl or alkylene means a straight chain, branched and/or cyclic hydrocarbon from 1 to 20 carbon atoms. Alkyl moieties having from 1 to 5 carbons are referred to as “lower alkyl” and examples include, but are not limited to, methyl, ethyl, propyl, isopropyl, n-butyl, t-butyl, and isobutyl.
Cycloalkyl or cycloalkylene represents a 3-14 membered monocyclic or bicyclic carbocyclic ring, wherein the monocyclic or one of the bicyclic rings is saturated or partially unsaturated and may optionally further comprise a —C(O)— ring member, and the other ring may be aromatic, saturated or partially unsaturated and may include one to three ring members selected from —C(═O), —N(R²⁰)q-, —O— and S(O)r where R²⁰ is H or C₁₋₆-alkyl, q is 0-1 and r is 0-2;
Aryl or arylene represents a 6-14 membered monocyclic or bicyclic carbocyclic ring, wherein the monocyclic or one of the bicyclic rings is aromatic and the other ring may be aromatic, saturated or partially unsaturated and may include one to three ring members selected from —C(O), —N(R¹⁹)q-, —O— and S(O)r where R¹⁹ is H or C₁₋₆alkyl, q is 0-1 and r is 0-2;
Heteroaryl or heteroarylene represents a 5-14 membered monocyclic or bicyclic ring, wherein the monocyclic or one of the bicyclic rings is an aromatic group comprising either (a) 1 to 4 nitrogen atoms, (b) one oxygen or one sulphur atom or (c) 1 oxygen atom or 1 sulphur atom and 1 or 2 nitrogen atoms, and the other ring may be aromatic, saturated or partially unsaturated, and may include one to three ring members selected from —C(O), —N(R²¹)q-, —O— and S(O)r where R²¹ is H or C₁₋₆-alkyl, q is 0-1 and r is 0-2; and
Heterocycloalkyl or heterocycloalkylene represents a 3-14 membered monocyclic or bicyclic ring, wherein the monocyclic or one of the bicyclic rings is a saturated or partially unsaturated group comprising one or two ring members selected from —N(R²²)—, —O— and —S(O)ᵣ— and may optionally further comprise a —C(O)— ring member, and the other ring may be aromatic, saturated or partially unsaturated, and may include one to three ring members selected from C(═O), N(R²³)q-, —O— and S(O)r where R²² or R²³ is H or C₁₋₆-alkyl, q is 0-1 and r is 0-2.

2. The compound according to claim 1, where X represents —N(H)—.

3. The compound according to claim 1, where R¹ represents phenyl substituted in the 2- and 4-positions.

4. The compound according to claim 1, where R¹ represents 4-bromo-2-fluorophenyl, or 4-iodo-2-fluorophenyl.

5. The compound according to claim 1, where Y represents D, and D represents a group selected from —C(O)—, —CO₂—, C(O)N(H)O—, —C(O)N(C₁₋₆-alkyl)O—, —C(O)N(H)— or —C(O)N(C₁₋₆-alkyl)-.

6. The compound according to claim 1, where R² represents —COH—, CO₂H, —CO₂Et, CON(H or CH₃)OR^{7a}, where R^{7a} represents methyl, ethyl, cyclopropylmethyl, 2-ethenyloxyethyl, 2-hydroxyethyl, or 2,3-dihydroxypropyl, —CON(H or CH₃)—R^{7b}, where R^{7b} represents H, methyl, ethyl, cyclopropylmethyl, 2-methoxyethyl, 2-hydroxyethyl, 3-hydroxypropyl, acetylaminomethyl, 2-dimethylaminoethyl, cyclopentyl or 2-thiazolyl, or R² represents oxadiazolylamino.

7. The compound according to claim 1, where R² represents CONHOR^{7a} where R^{7a} represents cyclopropylmethyl, or 2-hydroxyethyl.

8. The compound according to claim 1, where -E- represents cycloalkyl, a 5-membered heteroarylene or 5-membered heterocycloalkylene which may be substituted or unsubstituted.

9. The compound according to claim 1, where E represents cyclopentyl, thiazole, or oxadiazole which may be substituted or unsubstituted.

10. The compound according to claim 1, where R³ and R⁴ represent H.

11. The compound according to claim 1, where R⁵ is H, methyl, ethyl, chloro, or fluoro.

12. The compound according to claim 1, where R⁵ is methyl.

13. The compound according to claim 1, where Z is O.

14. The compound according to claim 1, where m and n are both 1. or one of m and n is 1 and the other is 2.

15. A compound of formula Id: 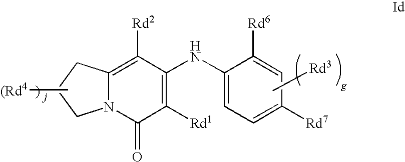
and salts thereof, wherein:
Rd¹ represents H, halogen, C₁₋₃-alkyl, or C₁₋₃-haloalkyl;
Rd² represents H, cyano, or the group —Y-Rd⁵;
Rd³ and Rd⁴ independently represent hydroxyl, cyano, nitro, C₁₋₆-alkyl, C₂₋₆alkenyl, C₂₋₆ alkynyl, C₁₋₆-alkoxy, C₂₋₆-alkenyloxy, C₂₋₆-alkynyloxy, halogen, C₁₋₆-alkylcarbonyl, carboxy, C₁₋₆-alkoxycarbonyl, amino, C₁₋₆-alkylamino, di-C₁₋₆-alkylamino, C₁₋₆-alkylaminocarbonyl, di-C₁₋₆ alkylaminocarbonyl, C₁₋₆-alkylcarbonylamino, C₁₋₆-alkylcarbonyl(C₁₋₆-alkyl)amino, C₁₋₆-alkylsulfonylamino, C₁₋₆-alkylsulfonyl(C₁₋₆-alkyl)amino, C₁₋₆-thioalkyl, C₁₋₆-alkylsulfanyl, C₁₋₆ alkylsulfanyl, C₁₋₆-alkylsulfonyl, aminosulfonyl, C₁₋₆-alkylaminosulfonyl and di-C₁₋₆ alkylaminosulfonyl, where each of the afore-mentioned hydrocarbon groups may be optionally substituted by one or more halogen, hydroxyl, C₁₋₆-alkoxy, amino, C₁₋₆-alkylamino, di-C₁₋₆-alkylamino or cyano;
Y represents a group selected from D-, -E-, -D-E-, or E-D-;
D represents a group selected from —N(Rd⁸)—, —CO—, —CO₂—, —SO—, —SO₂—, CON(Rd⁹)O—, —CON(Rd¹⁰)-, —N(Rd¹¹)SO₂—, —N(Rd¹²)SO₂NRd¹³-, —SO₂N(Rd¹⁴)-, —N(Rd¹⁵)CO—, —N(Rd¹⁶)CON(Rd¹⁷)—, —N(Rd¹⁸)CO—, or —C(═NH)N(Rd¹⁹)-;
E represents a monocyclic arylene, heteroarylene, cycloalkylene or heterocycloalkylene, wherein said rings are optionally substituted by one or more groups independently selected from List 1 as defined herein;
Rd⁵ represents H, C₁₋₆-alkyl, C₂₋₆-alkenyl C₂₋₆-alkynyl, cycloalkyl, aryl, heterocycloalkyl, or heteroaryl, wherein Rd⁵ when not H is optionally substituted by one to three groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆haloalkyl, amino, C₁₋₆alkylamino, di-C₁₋₆alkylamino, C₁₋₆acylamino, C₁₋₆acylC₁₋₆ alkylamino, monocyclic cycloalkyl or monocyclic heterocycloalkyl, where said rings may be optionally substituted by one or two groups independently selected from halogen, cyano, hydroxyl, C₁₋₆-alkoxy, C₂-C₆-alkenyloxy, C₂-C₆-alkynyloxy, C₁₋₆-thioalkyl, C₁₋₆-haloalkyl, amino, C₁₋₆-alkylamino, di-C₁₋₆-alkylamino, C₁₋₆-acylamino and C₁₋₆-acylC₁₋₆-alkylamino;
Rd⁶ and Rd⁷ independently represent hydroxyl, cyano, nitro, C₁₋₆-alkyl, C₂₋₆-alkenyl, C₂₋₆ alkynyl, C₁₋₆-alkoxy, C₂₋₆-alkenyloxy, C₂₋₆-alkynyloxy, halogen, C₁₋₆-alkylcarbonyl, carboxy, C₁₋₆ alkoxycarbonyl, amino, C₁₋₆-alkylamino, di-C₁₋₆-alkylamino, C₁₋₆-alkylaminocarbonyl, di-C₁₋₆-alkylaminocarbonyl, C₁₋₆-alkylcarbonylamino, C₁₋₆-alkylcarbonyl(C₁₋₆-alkyl)amino, C₁₋₆ alkylsulfonylamino, C₁₋₆-alkylsulfonyl(C₁₋₆-alkyl)amino, C₁₋₆-thioalkyl, C₁₋₆-alkylsulfinyl, C₁₋₆ alkylsulfanyl, C₁₋₆-alkylsulfonyl, aminosulfonyl, C₁₋₆-alkylaminosulfonyl and di-C₁₋₆ alkylaminosulfonyl, where each of the afore-mentioned hydrocarbon groups may be optionally substituted by one or more halogen, hydroxyl, C₁₋₆-alkoxy, amino, C₁₋₆-alkylamino, di-C₁₋₆ alkylamino or cyano;
j and g independently represent 0, 1, 2, or 3;
Rd⁸, Rd⁹, Rd¹⁰, Rd¹¹, Rd¹², Rd¹³, Rd¹⁴, Rd¹⁵, Rd¹⁶, Rd¹⁷, Rd¹⁸, and Rd¹⁹ are independently H or C₁₋₆-alkyl;
Alkyl or alkylene means a straight chain, branched and/or cyclic hydrocarbon from 1 to 20 carbon atoms. Alkyl moieties having from 1 to 5 carbons are referred to as “lower alkyl” and examples include, but are not limited to, methyl, ethyl, propyl, isopropyl, n-butyl, t-butyl, and isobutyl.
Cycloalkyl or cycloalkylene represents a 3-14 membered monocyclic or bicyclic carbocyclic ring, wherein the monocyclic or one of the bicyclic rings is saturated or partially unsaturated and may optionally further comprise a —C(O)— ring member, and the other ring may be aromatic, saturated or partially unsaturated and may include one to three ring members selected from C(═O), N(R²⁰)q-, —O— and S(O)r where R²⁰ is H or C₁₋₆-alkyl, q is 0-1 and r is 0-2;
Aryl or arylene represents a 6-14 membered monocyclic or bicyclic carbocyclic ring, wherein the monocyclic or one of the bicyclic rings is aromatic and the other ring may be aromatic, saturated or partially unsaturated and may include one to three ring members selected from —C(O), —N(R¹⁹)q-, —O— and S(O)r where R¹⁹ is H or C₁₋₆-alkyl, q is 0-1 and r is 0-2;
Heteroaryl or heteroarylene represents a 5-14 membered monocyclic or bicyclic ring, wherein the monocyclic or one of the bicyclic rings is an aromatic group comprising either (a) 1 to 4 nitrogen atoms, (b) one oxygen or one sulphur atom or (e) 1 oxygen atom or 1 sulphur atom and 1 or 2 nitrogen atoms, and the other ring may be aromatic, saturated or partially unsaturated, and may include one to three ring members selected from —C(O), —N(R²¹)q-, —O— and S(O)r where R²¹ is H or C₁₋₆-alkyl, q is 0-1 and r is 0-2; and
Heterocycloalkyl or heterocycloalkylene represents a 3-14 membered monocyclic or bicyclic ring, wherein the monocyclic or one of the bicyclic rings is a saturated or partially unsaturated group comprising one or two ring members selected from N(R²²)—, —O— and —S(O)ᵣ— and may optionally further comprise a C(O)— ring member, and the other ring may be aromatic, saturated or partially unsaturated, and may include one to three ring members selected from —C(═O), —N(R²³)q-, —O— and S(O)r where R²² or R²³ is H or C₁₋₆-alkyl q is 0-1 and r is 0-2.

16. The compound according to claim 1 for use in therapy.

17. A method of treating a disease, disorder or syndrome associated with MEK inhibition, said method comprising administering a compound according to claim 1 or its prodrug or pharmaceutical composition comprising the compound of formula 1 or its prodrug and pharmaceutically acceptable excipients to a subject in need thereof.

18. The method of treating as claimed in claim 17, wherein the disease, disorder or syndrome is hyperproliferative in a subject wherein subject is an animal including humans, selected from a group comprising cancer and inflammation.

19. The compound of formula I, method of treating disease, disorder or syndrome associated with MEK inhibition substantially as herein described along with examples.

20. A pharmaceutical composition comprising a compound of formula I according to claim 1 and a pharmaceutically acceptable carrier or excipient.

21. A pharmaceutical composition comprising a compound of formula I according to claim 1 in combination with a second active agent, and a pharmaceutically acceptable carrier or excipient.

22. A compound selected from
7-(4-Bromo-2-fluoro-phenylamino)-5-oxo-1,2,3,5-tetrahydro-indolizine-8-carboxylic acid ethyl ester;
7-(4-Bromo-2-fluoro-phenylamino)-5-oxo-1,2,3,5-tetrahydro-indolizine-8-carboxylic acid;
7-(4-Bromo-2-fluoro-phenylamino)-5-oxo-1,2,3,5-tetrahydro-indolizine-8-carboxylic acid cyclopropyl-methoxy-amide;
7-(4-Bromo-2-fluoro-phenylamino)-5-oxo-1,2,3,5-tetrahydro-indolizine-8-carboxylic acid dimethyl amide;
7-(4-bromo 2-fluoro-phenylamino)-5-oxo-1,2,3,5-tetrahydro indolizine 8-carboxylic acid methoxyl amide;
7-(4-bromo 2-fluoro-phenylamino)-5-oxo-1,2,3,5-tetrahydro indolizine 8-carboxylic acid amide;
7-(4-bromo 2-fluoro-phenylamino)-5-oxo-1,2,3,5-tetrahydro indolizine 8-carboxylic acid ethoxy amide;
7-(4-bromo 2-fluoro-phenylamino)-5-oxo-1,2,3,5-tetrahydro indolizine 8-carboxylic acid (2-hydroxy ethyl)amide;
7-(4-bromo-2-fluoro-phenylamino)-5-oxo-1,2,3,5-tetrahydro-indolizine-8-carboxylic acid methyl amide;
7-(4-Bromo-2-fluoro-phenylamino)-5-oxo-1,2,3,5-tetrahydro-indolizine-8-carboxylic acid methoxy-ethyl-amide;
7-(4-Bromo-2-fluoro-phenylamino)-5-oxo-1,2,3,5-tetrahydro-indolizine-8-carbaldehyde
6-Fluoro-7-(2-fluoro-4-iodo-phenylamino)-5-oxo-1,2,3,5-tetrahydro-indolizine-8-carboxylic acid cyclopropyl methoxy-amide;
6-Fluoro-7-(2-fluoro-4-iodo-phenylamino)-5-oxo-1,2,3,5-tetrahydro-indolizine-8-carboxylic acid amide;
7-(4-Bromo-2-fluoro-phenylamino)-6-fluoro-5-oxo-1,2,3,5-tetrahydro-indolizine-8-carboxylic acid;
7-(4-Bromo-2-fluoro-phenylamino)-6-fluoro-5-oxo-1,2,3,5-tetrahydro-indolizine-8-carboxylic acid cyclopropyl methoxy-amide;
7-(4-Bromo-2-fluoro-phenylamino)-6-fluoro-5-oxo-1,2,3,5-tetrahydro-indolizine-8-carboxylic acid amide;
7-(4-Bromo-2-fluoro-phenylamino)-6-methyl-5-oxo-1,2,3,5-tetrahydro-indolizine-8-carboxylic acid;
7-(4-Bromo-2-fluoro-phenylamino)-6-methyl-5-oxo-1,2,3,5-tetrahydro-indolizine-8-carboxylic acid cyclopropyl methoxy-amide;
7-(4-Bromo-2-fluoro-phenylamino)-6-methyl-5-oxo-1,2,3,5-tetrahydro-indolizine-8-carboxylic acid (2-vinyl oxy-ethoxy)-amide;
7-(4-Bromo-2-fluoro-phenylamino)-6-methyl-5-oxo-1,2,3,5-tetrahydro-indolizine-8-carboxylic acid amide;
7-(4-Bromo-2-fluoro-phenylamino)-6-methyl-5-oxo-1,2,3,5-tetrahydro-indolizine-8-carboxylic acid methoxy-amide;
7-(4-Bromo-2-fluoro-phenylamino)-6-methyl-5-oxo-1,2,3,5-tetrahydro-indolizine-8-carboxylic acid ethoxy-amide;
2-(4-bromo-2-fluoro-phenylamino)-4-oxo-6,7,8,9-tetrahydro-4H-quinolizine-1-carboxylic acid ethyl ester;
2-(4-bromo-2-fluoro-phenyl-amino)-4-oxo-6,7,8,9-tetrahydro-4H-quinolizine-1-carboxylic acid;
2-(4-bromo-2-fluoro-phenyl-amino)-4-oxo-6,7,8,9-tetrahydro-4H-quinolizine-1-carboxylic acid-cyclopropyl-methoxyamide;
2-(4-bromo-2-fluoro-phenylamino)-4-oxo-6,7,8,9-tetrahydro-4H-quinolizine-1-carboxylic acid amide;
2-(4-bromo-2-fluoro-phenylamino)-4-oxo-6,7,8,9-tetrahydro-4H-quinolizine-1-carboxylic acid-(2-vinyloxy-ethoxy)-amide;
2-(4-bromo-2-fluoro-phenylamino)-4-oxo-6,7,8,9-tetrahydro-4H-quinolizine-1-carboxylic acid methoxy amide;
2-(4-bromo-2-fluoro-phenylamino)-4-oxo-6,7,8,9-tetrahydro-4H-quinolizine-1-carboxylic acid ethoxy amide;
7-(4-bromo-2-fluorophenylamino)-6-methyl-5-oxo-1,2,3,5-tetrahydro-indolizine-8-carboxylic acid (2-hydroxyethoxy)amide;
7-(4-Bromo-2-fluoro-phenylamino)-5-oxo-1,2,3,5-tetrahydro-indolizine-8-carboxylic acid (3-hydroxy-propyl)-amide;
7-(4-Bromo-2-fluoro-phenylamino)-5-oxo-1,2,3,5-tetrahydro-indolizine-8-carboxylic acid (2-methoxy-ethyl)-amide;
7-(4-Bromo-2-fluoro-phenylamino)-5-oxo-1,2,3,5-tetrahydro-indolizine-8-carboxylic acid (2-acetylamino-ethyl)-amide;
7-(4-Bromo-2-fluoro-phenylamino)-5-oxo-1,2,3,5-tetrahydro-indolizine-8-carboxylic acid (2-dimethylamino-ethyl)-amide;
6-Fluoro-7-(2-fluoro-4-iodo-phenylamino)-5-oxo-1,2,3,5-tetrahydro-indolizine-8-carboxylic acid (2-hydroxy-ethoxy)-amide;
7-(4-Bromo-2-fluoro-phenylamino)-6-methyl-5-oxo-1,2,3,5-tetrahydro-indolizine-8-carboxylic acid cyclopentylamide;
7-(4-Bromo-2-fluoro-phenylamino)-6-methyl-5-oxo-1,2,3,5-tetrahydro-indolizine-8-carboxylic acid ethylamide;
7-(4-Bromo-2-fluoro-phenylamino)-6-methyl-5-oxo-1,2,3,5-tetrahydro-indolizine-8-carboxylic acid (3-methoxy-propyl)-amide;
7-(2-Fluoro-4-iodo-phenylamino)-5-oxo-1,2,3,5-tetrahydro-indolizine-8-carboxylic acid;
7-(2-Fluoro-4-iodo-phenylamino)-5-oxo-1,2,3,5-tetrahydro-indolizine-8-carboxylic acid amide;
7-(2-Fluoro-4-iodo-phenylamino)-5-oxo-1,2,3,5-tetrahydro-indolizine-8-carboxylic acid cyclopropylmethoxy-amide;
7-(2-Fluoro-4-iodo-phenylamino)-5-oxo-1,2,3,5-tetrahydro-indolizine-8-carboxylic acid ethyl ester;
7-(2-Fluoro-4-iodo-phenylamino)-5-oxo-1,2,3,5-tetrahydro-indolizine-8-carboxylic acid (2-hydroxy-ethoxy)-amide;
7-(2-Fluoro-4-iodo-phenylamino)-5-oxo-1,2,3,5-tetrahydro-indolizine-8-carboxylic acid methoxy-amide;
7-(2-Fluoro-4-iodo-phenylamino)-5-oxo-1,2,3,5-tetrahydro-indolizine-8-carboxylic acid ethoxy-amide;
7-(4-Bromo-2-fluoro-phenylamino)-5-oxo-1,2,3,5-tetrahydro-indolizine-8-carboxylic acid (2,3-dihydroxy-propoxy)-amide;
7-(4-Bromo-2-fluoro-phenylamino)-6-chloro-5-oxo-1,2,3,5-tetrahydro-indolizine-8-carboxylic acid;
7-(4-Bromo-2-fluoro-phenylamino)-6-chloro-5-oxo-1,2,3,5-tetrahydro-indolizine-8-carboxylic acid cyclopropylmethoxy-amide;
7-(4-Bromo-2-fluoro-phenylamino)-6-chloro-5-oxo-1,2,3,5-tetrahydro-indolizine-8-carboxylic acid methoxy-amide;
7-(4-Bromo-2-fluoro-phenylamino)-6-chloro-5-oxo-1,2,3,5-tetrahydro-indolizine-8-carboxylic acid amide;
7-(4-Bromo-2-fluoro-phenylamino)-6-chloro-5-oxo-1,2,3,5-tetrahydro-indolizine-8-carboxylic acid ethoxy-amide;
7-(4-Bromo-2-fluoro-phenylamino)-6-chloro-5-oxo-1,2,3,5-tetrahydro-indolizine-8-carboxylic acid (2,3-dihydroxy-propoxy)-amide;
6-Chloro-7-(2-fluoro-4-iodo-phenylamino)-5-oxo-1,2,3,5-tetrahydro-indolizine-8-carboxylic acid;
6-Chloro-7-(2-fluoro-4-iodo-phenylamino)-5-oxo-1,2,3,5-tetrahydro-indolizine-8-carboxylic acid cyclopropylmethoxy-amide;
6-Chloro-7-(2-fluoro-4-iodo-phenylamino)-5-oxo-1,2,3,5-tetrahydro-indolizine-8-carboxylic acid methoxy-amide;
6-Chloro-7-(2-fluoro-4-iodo-phenylamino)-5-oxo-1,2,3,5-tetrahydro-indolizine-8-carboxylic acid amide;
6-Chloro-7-(2-fluoro-4-iodo-phenylamino)-5-oxo-1,2,3,5-tetrahydro-indolizine-8-carboxylic acid ethoxy-amide;
6-Chloro-7-(2-fluoro-4-iodo-phenylamino)-5-oxo-1,2,3,5-tetrahydro-indolizine-8-carboxylic acid (2,3-dihydroxy-propoxy)-amide;
7-(4-Bromo-2-fluoro-phenylamino)-6-methyl-5-oxo-1,2,3,5-tetrahydro-indolizine-8-carboxylic acid thiazol-2-ylamide;
7-(4-Bromo-2-fluoro-phenylamino)-6-methyl-5-oxo-1,2,3,5-tetrahydro-indolizine-8-carboxylic acid (3-hydroxy-propyl)-amide;
7-(4-Bromo-2-fluoro-phenylamino)-6-methyl-5-oxo-1,2,3,5-tetrahydro-indolizine-8-carboxylic acid dimethylamide;
7-(4-Bromo-2-fluoro-phenylamino)-6-methyl-5-oxo-1,2,3,5-tetrahydro-indolizine-8-carboxylic acid (2-methoxy-ethyl)-amide;
7-(4-Bromo-2-fluoro-phenylamino)-6-methyl-5-oxo-1,2,3,5-tetrahydro-indolizine-8-carboxylic acid (2-acetylamino-ethyl)-amide;
7-(4-Bromo-2-fluoro-phenylamino)-6-methyl-5-oxo-1,2,3,5-tetrahydro-indolizine-8-carboxylic acid (pyridin-2-ylmethyl)-amide;
6-Fluoro-7-(2-fluoro-4-trifluoromethyl-phenylamino)-5-oxo-1,2,3,5-tetrahydro-indolizine-8-carboxylic acid;
6-Fluoro-7-(2-fluoro-4-trifluoromethyl-phenylamino)-5-oxo-1,2,3,5-tetrahydro-indolizine-8-carboxylic acid cyclopropylmethoxy-amide;
6-Fluoro-7-(2-fluoro-4-methylsulfanyl-phenylamino)-5-oxo-1,2,3,5-tetrahydro-indolizine-8-carboxylic acid cyclopropylmethoxy-amide;
7-(4-Bromo-2-fluoro-phenylamino)-6-fluoro-8-[5-(2-hydroxy-ethylamino)-[1,3,4]oxadiazol-2-yl]-2,3-dihydro-1H-indolizin-5-one;
7-(4-Bromo-2-fluoro-phenylamino)-6-fluoro-5-oxo-1,2,3,5-tetrahydro-indolizine-8-carboxylic acid methoxy-amide;
7-(4-Bromo-2-fluoro-phenylamino)-6-fluoro-5-oxo-1,2,3,5-tetrahydro-indolizine-8-carboxylic acid ethoxy-amide;
7-(4-Bromo-2-fluoro-phenylamino)-6-fluoro-5-oxo-1,2,3,5-tetrahydro-indolizine-8-carboxylic acid (2-hydroxy-ethoxy)-amide;
7-(4-Bromo-2-methyl-phenylamino)-6-fluoro-5-oxo-1,2,3,5-tetrahydro-indolizine-8-carboxylic acid;
7-(4-Bromo-2-methyl-phenylamino)-6-fluoro-5-oxo-1,2,3,5-tetrahydro-indolizine-8-carboxylic acid cyclopropylmethoxy-amide;
7-(4-Bromo-2-methyl-phenylamino)-5-oxo-1,2,3,5-tetrahydro-indolizine-8-carboxylic acid;
7-(4-Bromo-2-methyl-phenylamino)-5-oxo-1,2,3,5-tetrahydro-indolizine-8-carboxylic acid cyclopropylmethoxy-amide;
6-Fluoro-7-(2-fluoro-4-iodo-phenylamino)-8-(3-hydroxy-3-piperidin-2-yl-azetidine-1-carbonyl)-2,3-dihydro-1H-indolizin-5-one hydrochloride;
7-(4-Bromo-2-fluoro-phenylamino)-6-fluoro-8-(3-hydroxy-3-piperidin-2-yl-azetidine-1-carbonyl)-2,3-dihydro-1H-indolizin-5-one;
6-Fluoro-7-(2-fluoro-4-iodo-phenylamino)-8-(3-hydroxy-3-piperidin-2-yl-azetidine-1-carbonyl)-2,3-dihydro-1H-indolizin-5-one;
Cyclopropanesulfonic acid [7-(4-bromo-2-fluoro-phenylamino)-6-fluoro-5-oxo-1,2,3,5-tetrahydro-indolizin-8-yl]-amide;
N-[7-(4-Bromo-2-fluoro-phenylamino)-6-fluoro-5-oxo-1,2,3,5-tetrahydro-indolizin-8-yl]-N,N-dimethyl-amino-sulfonamide;
2,3-Dihydroxy-propane-amino-sulfonicacid-[7-(4-bromo-2-fluoro-phenylamino)-6-fluoro-5-oxo-1,2,3,5-tetrahydro-indolizin-8-yl]-amide;
1-[7-(4-Bromo-2-fluoro-phenylamino)-6-fluoro-5-oxo-1,2,3,5-tetrahydro-indolizin-8-ylsulfamoyl]-pyrrolidine-2-carboxylic acid;
2-Hydroxymethyl-pyrrolidine-1-sulfonic acid [7-(4-bromo-2-fluoro-phenylamino)-6-fluoro-5-oxo-1,2,3,5-tetrahydro-indolizin-8-yl]-amide;
7-(4-Bromo-2-fluoro-phenylamino)-6-fluoro-8-(1-hydroxy-but-3-enyl)-2,3-dihydro-1H-indolizin-5-one;
7-(4-Bromo-2-fluoro-phenylamino)-6-fluoro-8-(1-hydroxy-allyl)-2,3-dihydro-1H-indolizin-5-one;
7-(4-Bromo-2-fluoro-phenylamino)-8-(2,3-dihydroxy-propionyl)-6-fluoro-2,3-dihydro-1H-indolizin-5-one;
7-(4-Bromo-2-fluoro-phenylamino)-6-fluoro-8-(2-hydroxy-acetyl)-2,3-dihydro-1H-indolizin-5-one;
7-(2-Fluoro-4-trifluoromethyl-phenylamino)-5-oxo-1,2,3,5-tetrahydro-indolizine-8-carboxylic acid ethyl ester;
7-(2-Fluoro-4-trifluoromethyl-phenylamino)-5-oxo-1,2,3,5-tetrahydro-indolizine-8-carboxylic acid;
7-(2-Fluoro-4-trifluoromethyl-phenylamino)-5-oxo-1,2,3,5-tetrahydro-indolizine-8-carboxylic acid cyclopropylmethoxy-amide;
7-(2-Fluoro-4-methoxy-phenylamino)-5-oxo-1,2,3,5-tetrahydro-indolizine-8-carboxylic acid cyclopropylmethoxy-amide;
7-(4-Bromo-2-fluoro-phenylamino)-5-oxo-1,2,3,5-tetrahydro-indolizine-8-carbaldehyde oxime;
7-(4-Bromo-2-fluoro-phenylamino)-8-(3-hydroxy-3-pyridin-2-yl-azetidine-1-carbonyl)-2,3-dihydro-1H-indolizin-5-one;
7-(4-Bromo-2-fluoro-phenylamino)-8-(3-hydroxy-azetidine-1-carbonyl)-2,3-dihydro-1H-indolizin-5-one;
3-(4-Bromo-2-fluoro-phenyl)-1-methanesulfonyl-1,6,7,8-tetrahydro-3H-1,3,5a-triaza-as-indacene-2,5-dione;
Cyclopropanesulfonic acid [7-(4-bromo-2-fluoro-phenylamino)-5-oxo-1,2,3,5-tetrahydro-indolizin-8-yl]-amide;
N-[7-(4-Bromo-2-fluoro-phenylamino)-5-oxo-1,2,3,5-tetrahydro-indolizin-8-yl]-4-fluoro-benzenesulfonamide;
[7-(4-Bromo-2-fluoro-phenylamino)-5-oxo-1,2,3,5-tetrahydro-indolizin-8-yl]-carbamic acid tert-butyl ester;
Cyclohexanesulfonic acid [7-(4-bromo-2-fluoro-phenylamino)-5-oxo-1,2,3,5-tetrahydro-indolizin-8-yl]-amide;
N-[7-(4-Bromo-2-fluoro-phenylamino)-5-oxo-1,2,3,5-tetrahydro-indolizin-8-yl]-4-trifluoromethyl-benzenesulfonamide;
N-[7-(4-Bromo-2-fluoro-phenylamino)-5-oxo-1,2,3,5-tetrahydro-indolizin-8-yl]-N,N-dimethylaminosulfonamide;
5-(4-Bromo-2-fluoro-phenylamino)-2,2-dimethyl-7-oxo-3a,7,8,8a-tetrahydro-1,3-dioxa-7a-aza-cyclopenta[a]indene-4-carboxylic acid;
7-(4-Bromo-2-fluoro-phenylamino)-1,2-dihydroxy-5-oxo-1,2,3,5-tetrahydro-indolizine-8-carboxylic acid ethyl ester;
7-(4-Bromo-2-fluoro-phenylamino)-1,2-dihydroxy-5-oxo-1,2,3,5-tetrahydro-indolizine-8-carboxylic acid cyclopropylmethoxy-amide;
7-(4-Bromo-2-fluoro-phenylamino)-1,2-dihydroxy-5-oxo-1,2,3,5-tetrahydro-indolizine-8-carboxylic acid (2-hydroxy-ethoxy)-amide;
7-(4-Bromo-2-fluoro-phenylamino)-1,2-dihydroxy-5-oxo-1,2,3,5-tetrahydro-indolizine-8-carboxylic acid cyclobutylmethoxy-amide;
7-(4-Bromo-2-fluoro-phenylamino)-1,2-dihydroxy-5-oxo-1,2,3,5-tetrahydro-indolizine-8-carboxylic acid (3-hydroxy-2-methyl-propoxy)-amide;
1-(2,3-Dihydroxy-propyl)-cyclopropanesulfonic acid [6-fluoro-7-(2-fluoro-4-iodo-phenylamino)-5-oxo-1,2,3,5-tetrahydro-indolizin-8-yl]-amide;
1-(2,3-Dihydroxy-propyl)-cyclopropanesulfonic acid [7-(4-bromo-2-fluoro-phenylamino)-6-methyl-5-oxo-1,2,3,5-tetrahydro-indolizin-8-yl]-amide;
1-(2,3-Dihydroxy-propyl)-cyclopropanesulfonic acid [7-(4-bromo-2-fluoro-phenylamino)-6-fluoro-5-oxo-1,2,3,5-tetrahydro-indolizin-8-yl]-amide;
6-Fluoro-7-(2-fluoro-4-iodo-phenylamino)-5-oxo-1,2,3,5-tetrahydro-indolizine-8-carboxylic acid (2,3-dihydroxy-propoxy)-amide;
7-(2-Fluoro-4-iodo-phenylamino)-6-methyl-5-oxo-1,2,3,5-tetrahydro-indolizine-8-carboxylic acid cyclopropyl methoxy amide;
7-(2-Fluoro-4-iodo-phenylamino)-6-methyl-5-oxo-1,2,3,5-tetrahydro-indolizine-8-carboxylic acid (2-hydroxy-ethoxy)-amide;
7-(2-Fluoro-4-iodo-phenylamino)-6-methyl-5-oxo-1,2,3,5-tetrahydro-indolizine-8-carboxylic acid cyclobutylmethoxy-amide;
1-(2,3-Dihydroxy-propyl)-cyclopropanesulfonic acid [7-(2-fluoro-4-iodo-phenylamino)-6-methyl-5-oxo-1,2,3,5-tetrahydro-indolizin-8-yl]-amide; and
Cyclopropanesulfonic acid [7-(4-bromo-2-fluoro-phenylamino)-6-methyl-5-oxo-1,2,3,5-tetrahydro-indolizin-8-yl]-amide, or pharmaceutically acceptable salts thereof.
